(19) United States
(12) Patent Application Publication
(10) Pub. No.: US 2025/0213687 A1
(43) Pub. Date: Jul. 3, 2025

(54) THERAPEUTIC T CELL PRODUCT

(71) Applicants: Tikva Allocell Pte. Ltd., Singapore (SG); Agency for Science, Technology and Research, Singapore (SG)

(72) Inventors: Pei Yun Teo, Singapore (SG); Lionel Jianrong Low, Singapore (SG); Youngrock Jung, Singapore (SG)

(73) Assignees: Tikva Allocell Pte. Ltd., Singapore (SG); Agency for Science, Technology and Research, Singapore (SG)

(21) Appl. No.: 18/856,476

(22) PCT Filed: Apr. 12, 2024
(86) PCT No.: PCT/EP2023/059506

§ 371 Date: Oct. 11, 2024

Related U.S. Application Data
(60) Provisional application No. 63/446,520, filed on Feb. 17, 2023.
(60) Provisional application No. 63/330,718, filed on Apr. 13, 2022.

(51) Int. Cl. A61K 40/11; A61K 40/31; A61K 40/42; A61P 35/00; C07K 14/81; C07K 16/28; C12N 5/0783

(52) U.S. Cl. CPC A61K 40/11; A61K 40/31; A61K 40/4211; A61K 40/4215; A61P 35/00; C07K 14/8121; C07K 16/2803; C07K 16/2878; C12N 5/0636; C07K 2319/02; C07K 2319/03; C12N 2510/00

(57) ABSTRACT

Immune cells comprising modification to increase the expression or activity of SERPINB9 are disclosed. Also disclosed are compositions comprising such cells and methods of using such cells and compositions.

This application claims priority from U.S. 63/330,718 filed 13 Apr. 2022 and U.S. 63/446,520 filed 17 Feb. 2023, the contents and elements of which are each herein incorporated by reference for all purposes.

### TECHNICAL FIELD

The present disclosure relates to the field of molecular biology, more specifically cell therapy. The present disclosure also relates to methods of medical treatment and prophylaxis.

### BACKGROUND

Adoptive cell therapies have emerged as a powerful and effective treatment for various cancers. For instance, CD19 chimeric antigen receptor (CAR)-expressing T cells have been very effective against B-cell malignancies and virus specific T-cells (VSTs) have shown great potential in treating patients with Epstein-Barr virus (EBV) associated lymphomas and viral infections in hematopoietic stem cell transplant (HSCT) recipients.

To avoid graft-vs-host disease (GVHD) or graft rejection, most cell therapies are autologous. However, many challenges are associated with such highly personalized treatments as they are generated from cells derived from patients with cancer or genetic diseases. Moreover, the high costs and long ‘needle-to-needle’ time limit the use of autologous cell therapy.

On the other hand, off-the-shelf third party derived cells serve as a readily available source of therapy for patients at point of care and overcome many of the challenges associated with customized cell products. However, unwanted host immune reaction against allogeneic third party derived therapeutic cells limit the bioavailability and the effectiveness of treatment. Efforts have been made to increase the persistence of, and reduce the allogeneic elimination of, the therapeutic product by, for instance, expressing an allogeneic defence receptor (ADR) that targets and kills alloreactive host T and NK cells (1, 2), the genetic knockout of HLA molecules (3) and CD52 (4, 5) as well as cell encapsulation methods to hide the therapeutic from the host immune system (6). However, many of these approaches exert undesirable effects on the host, such as the elimination of alloreactive or pathogen-specific immune cells, which can possibly result in opportunistic infection. There remains a strong need for a strategy to protect the therapeutic cell without harming host immunity.

Granzyme B (GzmB) is a serine proteinase that has been described as a key cytotoxic molecule utilized by T cells or natural killer (NK) cells for the clearance of allogeneic cells or pathogen-infected cells (7-9). In addition, granzyme B produced within the therapeutic cells (i.e. intrinsic granzyme B) after stimulation is also associated with homeostatic cell death (Bird et al., Cell Death Differ. (2014) 21, 876-887). GzmB cleaves its target proteins such as pro-apoptotic Bcl-2 family member Bid or apoptotic caspase substrates (e.g., DNA-PK, PARP and NuMA) to cause mitochondrial instability and caspase activation respectively, resulting in apoptosis of the targeted cells (10, 11). SERPINB9 wildtype (also referred to herein as SB9(WT)) inhibits GzmB by acting as a pseudo-substrate, which forms a stable covalent bond with GzmB, preventing the activation of apoptosis (12). The glutamic acid at the P1 location (340E) of the reactive centre loop (RCL) of SB9 is crucial for its specificity to GzmB, but limits its interaction to other caspases involved in Fas-mediated apoptosis (13). A substitution of glutamic acid to aspartic acid (E340D), forming the variant referred to herein as SB9(CAS), has been shown to broaden SB9 inhibition of both GzmB and Fas-mediated apoptosis (13). SB9 wildtype was also observed to be reactive oxygen species (ROS) sensitive and a conversion of C341S and C342S resulted in a functional SB9, forming the variant referred to herein as SB9(ROS), that resists ROS inactivation while maintaining GzmB inhibition (14).

### SUMMARY

In a first aspect, the present disclosure provides an immune cell comprising modification to increase the expression or activity of SERPINB9. In particular, the present disclosure provides an immune cell for use in a method of treatment or prophylaxis by adoptive cell transfer, comprising modification to increase the expression or activity of SERPINB9.

In some embodiments, the immune cell comprises exogenous nucleic acid encoding a SERPINB9 polypeptide. In some embodiments, the exogenous nucleic acid encoding a SERPINB9 polypeptide is, or is comprised in, an expression vector; optionally wherein the expression vector is a retroviral expression vector. In some embodiments, the SERPINB9 polypeptide comprises, or consists of, the amino acid sequence of SEQ ID NO: 1, 4, 5, 6 or 7, or a variant thereof having at least 85% amino acid sequence identity to SEQ ID NO: 1, 4, 5, 6 or 7.

In some embodiments, the immune cell is an effector immune cell; optionally wherein the effector immune cell is a T cell or a natural killer (NK) cell.

In some embodiments, the immune cell comprises nucleic acid encoding a chimeric antigen receptor (CAR). In some embodiments, the CAR comprises an antigen-binding domain that binds to a cancer-associated antigen selected from: CD30, CD19, CD20, CD22, B7H3, c-Met, ROR1R, CD4, CD7, CD38, BCMA, Mesothelin, EGFR, GPC3, MUC1, HER2, GD2, CEA, EpCAM, LeY and PSCA; optionally wherein the CAR comprises an antigen-binding domain that binds to CD30.

In some embodiments, the immune cell is a virus-specific T cell or an activated T cell (ATC).

In some embodiments, the immune cell is a virus-specific T cell. In some embodiments, the virus-specific T cell is specific for a virus selected from Epstein-Barr virus (EBV), adenovirus, cytomegalovirus (CMV), human papilloma virus (HPV), influenza virus, measles virus, hepatitis B virus (HBV), hepatitis C virus (HCV), human immunodeficiency virus (HIV), lymphocytic choriomeningitis virus (LCMV), herpes simplex virus (HSV), BK virus (BKV) or varicella zoster virus (VZV). In some embodiments, the virus is EBV.

The present disclosure also provides a pharmaceutical composition, comprising an immune cell according to the present disclosure, and a pharmaceutically-acceptable carrier, diluent, excipient or adjuvant.

The present disclosure also provides an immune cell or a pharmaceutical composition according the present disclosure, for use in a method of medical treatment or prophylaxis.

The present disclosure also provides the use of an immune cell or of a pharmaceutical composition according to the present disclosure, in the manufacture of a medicament for use in a method of medical treatment or prophylaxis.

The present disclosure also provides a method of treating or preventing a disease or condition in a subject, comprising administering to a subject a therapeutically- or prophylactically-effective quantity of an immune cell or of a pharmaceutical composition according to the present disclosure.

The present disclosure also provides a method for reducing the activity of a serine protease or a caspase in a cell, comprising modifying the cell to increase the expression or activity of SERPINB9.

The present disclosure also provides a method for increasing the resistance of a cell to the activity of a serine protease or a caspase, comprising modifying the cell to increase the expression or activity of SERPINB9.

The present disclosure also provides a method for increasing the resistance of a cell to cell killing by granzyme B, comprising modifying the cell to increase the expression or activity of SERPINB9.

The present disclosure also provides a method for increasing the resistance of a cell to apoptosis mediated by a death receptor, comprising modifying the cell to increase the expression or activity of SERPINB9.

In some embodiments in accordance with various aspects of the present disclosure, modifying the cell to increase the expression or activity of SERPINB9 comprises introducing nucleic acid encoding a SERPINB9 polypeptide into the cell. In some embodiments, the nucleic acid encoding a SERPINB9 polypeptide is, or is comprised in, an expression vector; optionally wherein the expression vector is a retroviral expression vector. In some embodiments, the SERPINB9 polypeptide comprises, or consists of, the amino acid sequence of SEQ ID NO: 1, 4, 5, 6 or 7, or a variant thereof having at least 85% amino acid sequence identity to SEQ ID NO: 1, 4, 5, 6 or 7.

In some embodiments, the cell is an effector immune cell; optionally wherein the effector immune cell is a T cell or a natural killer (NK) cell.

In some embodiments, the cell comprises nucleic acid encoding a chimeric antigen receptor (CAR). In some embodiments, the CAR comprises an antigen-binding domain that binds to a cancer-associated antigen selected from: CD30, CD19, CD20, CD22, B7H3, c-Met, ROR1R, CD4, CD7, CD38, BCMA, Mesothelin, EGFR, GPC3, MUC1, HER2, GD2, CEA, EpCAM, LeY and PSCA; optionally wherein the CAR comprises an antigen-binding domain that binds to CD30.

In some embodiments, the cell is a virus-specific T cell. In some embodiments, the virus-specific T cell is specific for a virus selected from Epstein-Barr virus (EBV), adenovirus, cytomegalovirus (CMV), human papilloma virus (HPV), influenza virus, measles virus, hepatitis B virus (HBV), hepatitis C virus (HCV), human immunodeficiency virus (HIV), lymphocytic choriomeningitis virus (LCMV), herpes simplex virus (HSV), BK virus (BKV) or varicella zoster virus (VZV). In some embodiments, the virus is EBV.

The present disclosure also provides an immune cell for use in treating or preventing a cancer, wherein:
the immune cell comprises nucleic acid encoding a CAR comprising: (i) an antigen-binding domain that binds to CD30 or CD19, (ii) a transmembrane domain, and (iii) a signalling domain comprising an immunoreceptor tyrosine-based activation motif (ITAM); and
the immune cell comprises modification to increase the expression or activity of SERPINB9.

The present disclosure also provides the use of an immune cell in the manufacture of a medicament for use in treating or preventing a cancer, wherein:
the immune cell comprises nucleic acid encoding a CAR comprising: (i) an antigen-binding domain that binds to CD30 or CD19, (ii) a transmembrane domain, and (iii) a signalling domain comprising an immunoreceptor tyrosine-based activation motif (ITAM); and
the immune cell comprises modification to increase the expression or activity of SERPINB9.

The present disclosure also provides a method of treating or preventing a cancer in a subject, comprising administering to a subject a therapeutically- or prophylactically-effective quantity of an immune cell, wherein:
the immune cell comprises nucleic acid encoding a CAR comprising: (i) an antigen-binding domain that binds to CD30 or CD19, (ii) a transmembrane domain, and (iii) a signalling domain comprising an immunoreceptor tyrosine-based activation motif (ITAM); and
the immune cell comprises modification to increase the expression or activity of SERPINB9.

In some embodiments, the immune cell is a virus-specific T cell; optionally wherein the immune cell is an Epstein-Barr virus (EBV)-specific T cell.

The present disclosure also provides an immune cell for use in treating or preventing a cancer, wherein:
the immune cell is a virus-specific T cell; optionally wherein the immune cell is an Epstein-Barr virus (EBV)-specific T cell; and
the immune cell comprises modification to increase the expression or activity of SERPINB9.

The present disclosure also provides the use of an immune cell in the manufacture of a medicament for use in treating or preventing a cancer, wherein:
the immune cell is a virus-specific T cell; optionally wherein the immune cell is an Epstein-Barr virus (EBV)-specific T cell; and
the immune cell comprises modification to increase the expression or activity of SERPINB9.

The present disclosure also provides a method of treating or preventing a cancer in a subject, comprising administering to a subject a therapeutically- or prophylactically-effective quantity of an immune cell, wherein:
the immune cell is a virus-specific T cell; optionally wherein the immune cell is an Epstein-Barr virus (EBV)-specific T cell; and
the immune cell comprises modification to increase the expression or activity of SERPINB9.

In some embodiments, the immune cell comprises nucleic acid encoding a CAR comprising: (i) an antigen-binding domain that binds to CD30 or CD19, (ii) a transmembrane domain, and (iii) a signalling domain comprising an immunoreceptor tyrosine-based activation motif (ITAM).

In some embodiments in accordance with various aspects of the present disclosure, the subject (i.e. the subject treated/to be treated) is allogeneic with respect to the immune cell (i.e. the immune cell administered/to be administered, in accordance with treatment/prophylaxis).

In some embodiments in accordance with various aspects of the present disclosure, the immune cell comprises exogenous nucleic acid encoding a SERPINB9 polypeptide. In some embodiments, the exogenous nucleic acid encoding a SERPINB9 polypeptide is, or is comprised in, an expression vector; optionally wherein the expression vector is a retroviral expression vector. In some embodiments, the SERPINB9 polypeptide comprises, or consists of, the amino acid sequence of SEQ ID NO:1, 4, 5, 6 or 7, or a variant thereof having at least 85% amino acid sequence identity to SEQ ID NO:1, 4, 5, 6 or 7.

### DESCRIPTION

The present disclosure is based on the inventors' unexpected finding that upregulating the expression/activity of SERPINB9 in immune cells increases their persistence/survival in the presence of allogeneic effector immune cells. This is thought to be a result of inhibition of the activity of granzyme B in such cells. Accordingly, upregulating the expression/activity of SERPINB9 in cells to be employed in methods for the treatment of disease by adoptive cell transfer increases their persistence in the recipient subject, particularly in the case of allotransplantation.

Cells modified to upregulate the expression/activity of SERPINB9 are shown herein to proliferate/expand in vitro to a similar extent as equivalent cells lacking such modification, and where such cells are effector immune cells they display similar effector activity to equivalent cells lacking modification to upregulate the expression/activity of SERPINB9. Cells modified in this way also have a comparable toxicity profile (i.e. to an allogeneic recipient subject) compared to equivalent cells lacking such modification.

### SERPINB9

SERPINB9 (also known as Cytoplasmic antiproteinase 3 (CAP-3/CAP3) or Peptidase inhibitor 9 (PI-9) is the protein identified by UniProt P50453. Human SERPINB9 has the amino acid sequence shown in SEQ ID NO:1. SERPINB9 is a member of the Serpin family of protease inhibitors. SERPINB9 is an intracellular inhibitor of cytotoxic lymphocyte serine protease granzyme B.

The structure and function of SERPINB9 is reviewed e.g. in Kaiserman et al., Cell Death Differ. (2010) 17(4):586-95, Bird et al., Mol Cell Biol. (1998), 18(11):6387-98 and Bird et al., Cell Death Differ. (2014) 21, 876-887, all of which are hereby incorporated by reference in their entirety. SERPINB9 resides within the nuclei and cytoplasm of cells, and is endogenously expressed in immune privileged sites (e.g. the placenta and lung), T cells, antigen presenting cells, and hematopoietic stem cells. In T cells, SERPINB9 has been reported to prevent fratricide by misdirected granzyme B at the immune synapse. SERPINB9 also inhibits serine protease (e.g. granzyme B) activity against the cells producing the enzyme(s). That is, SERPINB9 protects activated, serine protease (e.g. granzyme B)-expressing effector immune cells from the action of the serine proteases they produce—i.e. SERPINB9 protects cells from autolysis, by serine proteases (e.g. granzyme B) of autogenous origin.

Serpins present an exposed reactive centre loop (RCL) to their target serine proteases, which then cleave the peptide bond between two P1 and P1′ residues of the serpin. This cleavage triggers a conformational change in the serpin, irreversibly trapping the protease in a covalently bound complex. Residues around the P1 contribute to protease binding, and mutation of serpin RCLs have been shown to abrogate inhibition and/or alter target specificity.

The RCL of human SERPINB9 is formed by positions 334 to 348 of SEQ ID NO:1, and is shown in SEQ ID NO:2. The P1 residue of the human SERPINB9 is the glutamate residue at position 340 of SEQ ID NO:1, and the P1′ residue of the human SERPINB9 is the cysteine residue at position 341 of SEQ ID NO:1.

The modifications T327R and E340A in human SERPINB9 have been shown to abolish the ability of SERPINB9 to inhibit granzyme B in vitro (see Bird et al., Mol Cell Biol. (1998), 18(11):6387-98). The E340A modification removes the crucial glutamate residue at P1, and the T327R modification disrupts the conserved proximal hinge domain required for serpin loop mobility and inhibitory function.

Wildtype SERPINB9 has also been reported to inhibit caspase activity (see e.g. Annand et al., Biochem. J. (1999) 342(Pt 3): 655-665). The modification E340D has been shown to reduce granzyme B inhibition by SERPINB9, but to expand its target specificity and increase its inhibitory activity against caspases, and thus inhibit Fas-mediated apoptosis (see Bird et al., Mol Cell Biol. (1998), 18(11):6387-98).

The modifications C341S and C342S have been shown to reduce reactive oxygen species (ROS)-mediated inactivation of SERPINB9, while retaining serine protease inhibition activity (see Mangan et al., J. Biol. Chem. (2016) 291(7):3626-38).

In this specification, ‘SERPINB9’ refers to SERPINB9 from any species and includes isoforms, fragments, variants or homologues from any species.

A ‘fragment’ generally refers to a fraction of the reference protein. A fragment of SERPINB9 may have a minimum length of one of 10, 20, 30, 40, 50, 100, 150, 200, 250, 300 or 350 amino acids, and may have a maximum length of one of 0, 30, 40, 50, 100, 150, 200, 250, 300 or 350 amino acids. An ‘isoform’ generally refers to a variant of the reference protein expressed by the same species as the species of the reference protein. A ‘homologue’ generally refers to a variant of the reference protein produced by a different species as compared to the species of the reference protein. Homologues include orthologues.

A ‘variant’ generally refers to a protein having an amino acid sequence comprising one or more amino acid substitutions, insertions, deletions or other modifications relative to the amino acid sequence of the reference protein, but retaining a considerable degree of amino acid sequence identity (e.g. at least 70%) to the amino acid sequence of the reference protein.

In some embodiments, the SERPINB9 is SERPINB9 from a mammal (e.g. a primate (rhesus, cynomolgous, or human) and/or a rodent (e.g. rat or murine) SERPINB9. Isoforms, fragments, variants or homologues of SERPINB9 may optionally be characterised as having at least 70%, preferably one of 80%, 85%, 90%, 91%, 92%, 93%, 94%, 95%, 96%, 97%, 98%, 99% or 100% amino acid sequence identity to the amino acid sequence of an immature or mature SERPINB9 isoform from a given species, e.g. human.

In some embodiments, the SERPINB9 is human SERPINB9. In some embodiments, the SERPINB9 is cynomolgous macaque SERPINB9. In some embodiments, the SERPINB9 is mouse SERPINB9 (also known as ‘SP16’).

Isoforms, fragments, variants or homologues may optionally be functional isoforms, fragments, variants or homologues, e.g. having a functional activity of the reference SERPINB9 (e.g. human SERPINB9), as determined by analysis by a suitable assay for the functional activity (e.g. protease inhibition (e.g. serine protease (e.g. granzyme B) and/or cysteine protease (e.g. caspase) inhibition)).

In some embodiments, SERPINB9 comprises, or consists of, an amino acid sequence having at least 70% amino acid sequence identity, e.g. one of ≥75%, ≥80%, ≥85%, ≥90%, ≥91%, ≥92%, ≥93%, ≥94%, ≥95%, ≥96%, ≥97%, ≥98%, ≥99% or 100% amino acid sequence identity, to the amino acid sequence of SEQ ID NO:1.

Aspects and embodiments of the present disclosure relate to SERPINB9 variants. As referred to herein, a ‘SERPINB9 variant’ refers to a SERPINB9 comprising one or more (e.g. one of 1, 2, 3, 4, 5, 6, 7, 8, 9 or 10) modifications relative to a reference SERPINB9.

A ‘modification’ refers to a difference relative to a reference amino acid sequence. A reference amino acid sequence may be the amino acid sequence encoded by the most common nucleotide sequence of the gene encoding the relevant protein. In embodiments herein (and also in the art more generally), a ‘modification’ may also be referred to as a ‘substitution’ or a ‘mutation’.

In preferred embodiments, a SERPINB9 variant according to the present disclosure comprises an amino acid sequence having at least 70% amino acid sequence identity to SEQ ID NO:1, and comprises one or more (e.g. one of 1, 2, 3, 4, 5, 6, 7, 8, 9 or 10) modifications relative to SEQ ID NO:1.

A modification typically comprises substitution of an amino acid residue with a non-identical ‘replacement’ amino acid residue. A replacement amino acid residue of a modification according to the present disclosure may be a naturally-occurring amino acid residue (i.e. encoded by the genetic code) which is non-identical to the amino acid residue at the relevant position of the amino acid sequence prior to modification, selected from: alanine (Ala), arginine (Arg), asparagine (Asn), aspartic acid (Asp), cysteine (Cys), glutamine (Gln), glutamic acid (Glu), glycine (Gly), histidine (His), isoleucine (Ile): leucine (Leu), lysine (Lys), methionine (Met), phenylalanine (Phe), proline (Pro), serine (Ser), threonine (Thr), tryptophan (Trp), tyrosine (Tyr), and valine (Val). In some embodiments, a replacement amino acid residue of a modification may be a non-naturally occurring amino acid residue—i.e. an amino acid residue other than those recited in the preceding sentence. Examples of non-naturally occurring amino acid residues include norleucine, ornithine, norvaline, homoserine, aib, and other amino acid residue analogues such as those described in Ellman, et al., Meth. Enzym. 202 (1991) 301-336.

SERPINB9 variants according to the present disclosure may comprise modification at specified positions of the amino acid sequence of a SERPINB9.

Herein, when reference is made to a position of human wildtype SERPINB9 (i.e. having the amino acid sequence of SEQ ID NO:1), the corresponding position in homologous sequences to human wildtype SERPINB9 are also contemplated. Corresponding positions to those identified in human wildtype SERPINB9 can be identified by sequence alignment, which can be performed e.g. using sequence alignment software such as ClustalOmega (SOding, J. 2005, Bioinformatics 21, 951-960).

In some embodiments, a SERPINB9 variant according to the present disclosure comprises modification at one or more of the following positions (numbered relative to SEQ ID NO:1): 340, 341 and 342. In some embodiments, a SERPINB9 variant comprises modification at position 340. In some embodiments, a SERPINB9 variant comprises modification at position 341 and/or 342. In some embodiments, a SERPINB9 variant comprises modification at position 340, 341 and/or 342.

In some embodiments, a SERPINB9 variant comprises 340D. In some embodiments, a SERPINB9 variant comprises 341S. In some embodiments, a SERPINB9 variant comprises 342S.

In some embodiments, a SERPINB9 variant comprises an amino acid sequence according to SEQ ID NO:3, wherein the amino acid sequence is non-identical to SEQ ID NO:2. In some embodiments, a SERPINB9 variant comprises, or consists of, an amino acid sequence according to SEQ ID NO:4, wherein the amino acid sequence is non-identical to SEQ ID NO:1.

In some embodiments, a SERPINB9 according to the present disclosure comprises, or consists of an amino acid sequence having the amino acid sequence of SEQ ID NO:1, 4, 5, 6 or 7, or an amino acid sequence having at least 70% amino acid sequence identity, e.g. one of ≥75%, ≥80%, ≥85%, ≥90%, ≥91%, ≥92%, ≥93%, ≥94%, ≥95%, ≥96%, ≥97%, ≥98% or 99% amino acid sequence identity to SEQ ID NO:1, 4, 5, 6 or 7. In some embodiments, a SERPINB9 according to the present disclosure comprises, or consists of an amino acid sequence having the amino acid sequence of SEQ ID NO:4, 5, 6 or 7, or an amino acid sequence having at least 70% amino acid sequence identity, e.g. one of ≥75%, ≥80%, ≥85%, ≥90%, ≥91%, ≥92%, ≥93%, ≥94%, ≥95%, ≥96%, ≥97%, ≥98% or ≥99% amino acid sequence identity to SEQ ID NO:4, 5, 6 or 7. In some embodiments, a SERPINB9 according to the present disclosure comprises, or consists of an amino acid sequence having the amino acid sequence of SEQ ID NO:4, 5 or 7, or an amino acid sequence having at least 70% amino acid sequence identity, e.g. one of ≥75%, ≥80%, ≥85%, ≥90%, ≥91%, ≥92%, ≥93%, ≥94%, ≥95%, ≥96%, ≥97%, ≥98% or ≥99% amino acid sequence identity to SEQ ID NO:4, 5 or 7.

In some embodiments, a SERPINB9 (or a SERPINB9 variant) comprises, or consists of, an amino acid sequence having the amino acid sequence of SEQ ID NO:1, or an amino acid sequence having at least 70% amino acid sequence identity, e.g. one of ≥75%, ≥80%, ≥85%, ≥90%, ≥91%, ≥92%, ≥93%, ≥94%, ≥95%, ≥96%, ≥97%, ≥98% or ≥99% sequence identity to SEQ ID NO:1.

In some embodiments, a SERPINB9 (or a SERPINB9 variant) comprises, or consists of, an amino acid sequence having the amino acid sequence of SEQ ID NO:4, or an amino acid sequence having at least 70% amino acid sequence identity, e.g. one of ≥75%, ≥80%, ≥85%, ≥90%, ≥91%, ≥92%, ≥93%, ≥94%, ≥95%, ≥96%, ≥97%, ≥98% or ≥99% sequence identity to SEQ ID NO:4.

In some embodiments, a SERPINB9 (or a SERPINB9 variant) comprises, or consists of, an amino acid sequence having the amino acid sequence of SEQ ID NO:5, or an amino acid sequence having at least 70% amino acid sequence identity, e.g. one of ≥75%, ≥80%, ≥85%, ≥90%, ≥91%, ≥92%, ≥93%, ≥94%, ≥95%, ≥96%, ≥97%, ≥98% or ≥99% sequence identity to SEQ ID NO:5.

In some embodiments, a SERPINB9 (or a SERPINB9 variant) comprises, or consists of, an amino acid sequence having the amino acid sequence of SEQ ID NO:6, or an amino acid sequence having at least 70% amino acid sequence identity, e.g. one of ≥75%, ≥80%, ≥85%, ≥90%, ≥91%, ≥92%, ≥93%, ≥94%, ≥95%, ≥96%, ≥97%, ≥98% or ≥99% sequence identity to SEQ ID NO:6.

In some embodiments, a SERPINB9 (or a SERPINB9 variant) comprises, or consists of, an amino acid sequence having the amino acid sequence of SEQ ID NO:7, or an amino acid sequence having at least 70% amino acid sequence identity, e.g. one of ≥75%, ≥80%, ≥85%, ≥90%, ≥91%, ≥92%, ≥93%, ≥94%, ≥95%, ≥96%, ≥97%, ≥98% or ≥99% sequence identity to SEQ ID NO:7.

In some embodiments, a SERPINB9 (or a SERPINB9 variant) according to the present disclosure does not comprise, or does not consist of, the amino acid sequence of SEQ ID NO:1. In some embodiments, a SERPINB9 comprises or consists of an amino acid sequence which is non-identical to the amino acid sequence of SEQ ID NO:1.

In some embodiments, a SERPINB9 (or a SERPINB9 variant) according to the present disclosure does not comprise, or does not consist of, the amino acid sequence of SEQ ID NO:5. In some embodiments, a SERPINB9 comprises or consists of an amino acid sequence which is non-identical to the amino acid sequence of SEQ ID NO:5.

In some embodiments, a SERPINB9 (or a SERPINB9 variant) according to the present disclosure does not comprise, or does not consist of, the amino acid sequence of SEQ ID NO:6. In some embodiments, a SERPINB9 comprises or consists of an amino acid sequence which is non-identical to the amino acid sequence of SEQ ID NO:6.

In some embodiments, a SERPINB9 (or a SERPINB9 variant) according to the present disclosure does not comprise, or does not consist of, the amino acid sequence of SEQ ID NO:7. In some embodiments, a SERPINB9 comprises or consists of an amino acid sequence which is non-identical to the amino acid sequence of SEQ ID NO:7.

In some embodiments, a SERPINB9 (or a SERPINB9 variant) according to the present disclosure does not comprise, or does not consist of, the amino acid sequence of SEQ ID NO:47. In some embodiments, a SERPINB9 comprises or consists of an amino acid sequence which is non-identical to the amino acid sequence of SEQ ID NO:47.

In some embodiments, one or more amino acids of an amino acid sequence referred to herein (e.g. SERPINB9 or a SERPINB9 variant) are substituted with another amino acid. A substitution comprises substitution of an amino acid residue with a non-identical ‘replacement’ amino acid residue. A replacement amino acid residue of a substitution according to the present disclosure may be a naturally-occurring amino acid residue (i.e. encoded by the genetic code) which is non-identical to the amino acid residue at the relevant position of the equivalent, unsubstituted amino acid sequence, selected from: alanine (Ala), arginine (Arg), asparagine (Asn), aspartic acid (Asp), cysteine (Cys), glutamine (Gin), glutamic acid (Glu), glycine (Gly), histidine (His), isoleucine (Ile): leucine (Leu), lysine (Lys), methionine (Met), phenylalanine (Phe), proline (Pro), serine (Ser), threonine (Thr), tryptophan (Trp), tyrosine (Tyr), and valine (Val). In some embodiments, a replacement amino acid may be a non-naturally occurring amino acid residue—i.e. an amino acid residue other than those recited in the preceding sentence.

Examples of non-naturally occurring amino acid residues include norleucine, ornithine, norvaline, homoserine, aib, and other amino acid residue analogues such as those described in Ellman et al., Meth. Enzym. (1991) 202:301-336.

In some embodiments, a substitution may be biochemically conservative. In some embodiments, where an amino acid to be substituted is provided in one of rows 1 to 5 of the table below, the replacement amino acid of the substitution is another, non-identical amino acid provided in the same row:

| Row | Shared property | Amino acids |
|---|---|---|
| 1 | Hydrophobic | Met, Ala, Val, Leu, Ile, Trp, Tyr, |
| | | Phe, Norleucine |
| 2 | Neutral hydrophilic | Cys, Ser, Thr, Asn, Gln |
| 3 | Acidic or negatively-charged | Asp, Glu |
| 4 | Basic or positively-charged | His, Lys, Arg |
| 5 | Orientation influencing | Gly, Pro |

By way of illustration, in some embodiments wherein substitution is of a Met residue, the replacement amino acid may be selected from Ala, Val, Leu, Ile, Trp, Tyr, Phe and Norleucine.

In some embodiments, a replacement amino acid in a substitution may have the same side chain polarity as the amino acid residue it replaces. In some embodiments, a replacement amino acid in a substitution may have the same side chain charge (at pH 7.4) as the amino acid residue it replaces:

| | | Side-chain charge |
|---|---|---|
| Amino Acid | Side-chain polarity | (pH 7.4) |
| Alanine | nonpolar | neutral |
| Arginine | basic polar | positive |
| Asparagine | polar | neutral |
| Aspartic acid | acidic polar | negative |
| Cysteine | nonpolar | neutral |
| Glutamic acid | acidic polar | negative |
| Glutamine | polar | neutral |
| Glycine | nonpolar | neutral |
| Histidine | basic polar | positive (10%) |
| | | neutral (90%) |
| Isoleucine | nonpolar | neutral |
| Leucine | nonpolar | neutral |
| Lysine | basic polar | positive |
| Methionine | nonpolar | neutral |
| Phenylalanine | nonpolar | neutral |
| Proline | nonpolar | neutral |
| Serine | polar | neutral |
| Threonine | polar | neutral |
| Tryptophan | nonpolar | neutral |
| Tyrosine | polar | neutral |
| Valine | nonpolar | neutral |

That is, in some embodiments, a nonpolar amino acid is substituted with another, non-identical nonpolar amino acid. In some embodiments, a polar amino acid is substituted with another, non-identical polar amino acid. In some embodiments, an acidic polar amino acid is substituted with another, non-identical acidic polar amino acid. In some embodiments, a basic polar amino acid is substituted with another, non-identical basic polar amino acid. In some embodiments, a neutral amino acid is substituted with another, non-identical neutral amino acid. In some embodiments, a positive amino acid is substituted with another, non-identical positive amino acid. In some embodiments, a negative amino acid is substituted with another, non-identical negative amino acid.

In some embodiments, substitution(s) may be functionally conservative. That is, in some embodiments, the substitution may not affect (or may not substantially affect) one or more functional properties (e.g. target binding) of the antigen-binding molecule comprising the substitution as compared to the equivalent unsubstituted molecule.

Aspects and embodiments of the present disclosure pertain to SERPINB9 polypeptides. A SERPINB9 polypeptide according to the present disclosure may be a SERPINB9 or a SERPINB9 variant according to any embodiment described hereinabove.

The present disclosure also provides a fusion polypeptide comprising a SERPINB9 polypeptide as described herein, and another polypeptide of interest. The amino acid sequence of the SERPINB9 polypeptide and the amino acid sequence of the other polypeptide of interest may be provided

In accordance with such aspects and embodiments, the polypeptide of interest may be a molecule for directing activity of an immune cell against a cell expressing a given target antigen. In some embodiments, a molecule for directing activity of an immune cell against a cell expressing a given target antigen may be a chimeric antigen receptor (CAR) or a T cell receptor (TCR).

In accordance with such embodiments, the SERPINB9 polypeptide and other polypeptide of interest may be joined by a linker sequence. In some embodiments, the linker sequence may be a cleavable linker. That is, the linker sequence may comprise a sequence of amino acids which is capable of being cleaved. For example, the linker sequence may comprise a sequence capable of acting as a substrate for an enzyme capable of cleaving peptide bonds—i.e. a cleavage site. Many such cleavage sites are known to and can be employed by the person skilled in the art of molecular biology. In some embodiments, the cleavable linker may comprise an autocleavage site. Autocleavage sites are automatically cleaved without the need for treatment with enzymes. 2A cleavage sites comprise the canonical ‘NPGP’ motif, which is cleaved at ‘G/P’. 2A cleavage sites include the P2A T2A, E2A, and F2A autocleavage sites. A linker sequence comprising a 2A autocleavage site is herein referred to as a 2A linker. Certain constructs described in the experimental examples employ a P2A autocleavage site (i.e. in a P2A linker). Certain constructs described in the experimental examples employ a T2A autocleavage site (i.e. in a T2A linker) In some embodiments, the fusion polypeptide comprises the structure: N term-[ . . . ]-[SERPINB9 polypeptide]-[other polypeptide of interest]-[ . . . ]-C term. In some embodiments, the fusion polypeptide comprises the structure: N term-[ . . . ]-[SERPINB9 polypeptide]-[cleavable linker]-[other polypeptide of interest]-[ . . . ]-C term. In some embodiments, the fusion polypeptide comprises the structure: N term-[ . . . ]-[SERPINB9 polypeptide]-[cleavable linker]-[CAR]-[ . . . ]-C term. In some embodiments, the fusion polypeptide comprises the structure: N term-[ . . . ]-[SERPINB9 polypeptide]-[2A linker]-[CAR]-[ . . . ]-C term. In some embodiments, the fusion polypeptide comprises the structure: N term-[ . . . ]-[SERPINB9 polypeptide]-[2A linker]-[CAR]-[2A linker]-[other polypeptide of interest]-[ . . . ]-C term. In some embodiments, the fusion polypeptide comprises the structure: N term-[ . . . ]-[SERPINB9 polypeptide]-[2A linker]-[other polypeptide of interest]-[2A linker]-[CAR]-[ . . . ]-C term. In some embodiments, the fusion polypeptide comprises the structure: N term-[ . . . ]-[other polypeptide of interest]-[SERPINB9 polypeptide]-[ . . . ]-C term. In some embodiments, the fusion polypeptide comprises the structure: N term-[ . . . ]-[other polypeptide of interest]-[cleavable linker]-[SERPINB9 polypeptide]-[ . . . ]-C term. In some embodiments, the fusion polypeptide comprises the structure: N term-[ . . . ]-[CAR]-[cleavable linker]-[SERPINB9 polypeptide]-[ . . . ]-C term. In some embodiments, the fusion polypeptide comprises the structure: N term-[ . . . ]-[CAR]-[2A linker]-[SERPINB9 polypeptide]-[ . . . ]-C term. In some embodiments, the fusion polypeptide comprises the structure: N term-[ . . . ]-[CAR]-[2A linker]-[SERPINB9 polypeptide]-[2A linker]-[other polypeptide of interest]-[ . . . ]-C term. In some embodiments, the fusion polypeptide comprises the structure: N term-[ . . . ]-[CAR]-[2A linker]-[other polypeptide of interest]-[2A linker]-[SERPINB9 polypeptide]-[ . . . ]-C term. In some embodiments, the fusion polypeptide comprises the structure: N term-[ . . . ]-[other polypeptide of interest]-[2A linker]-[SERPINB9 polypeptide]-[2A linker]-[CAR]-[ . . . ]-C term. In some embodiments, the fusion polypeptide comprises the structure: N term-[ . . . ]-[other polypeptide of interest]-[2A linker]-[CAR]-[2A linker]-[SERPINB9 polypeptide]-[ . . . ]-C term.

As used in representations of polypeptide structures herein, ‘[ . . . ]’ indicates the optional presence of further polypeptide(s) of interest/protein domain(s)/region(s). For example, in the structures of the preceding paragraph, further polypeptide(s) of interest/protein domain(s)/region(s) may optionally be present upstream of the SERPINB9 polypeptide, before the N terminus of the fusion polypeptide. Furthermore, as used in representations of polypeptide structures herein ‘-’ indicates an optional linker sequence. For example, in the final structure of the preceding paragraph, a linker sequence may optionally be provided between the SERPINB9 polypeptide and the 2A linker.

In some embodiments, a polypeptide according to the present disclosure comprises, or consists of, an amino acid sequence having at least 70% amino acid sequence identity, e.g. one of ≥75%, ≥80%, ≥85%, ≥90%, ≥91%, ≥92%, ≥93%, ≥94%, ≥95%, ≥96%, ≥97%, ≥98%, ≥99% or 100% amino acid sequence identity to SEQ ID NO:43, 44, 45 or 46.

### Agents for Increasing the Expression or Activity of SERPINB9

Aspects and embodiments of the present disclosure concern agents for increasing the expression or activity of SERPINB9. As used herein, an ‘agent for increasing the expression or activity of SERPINB9’ refers to an agent for increasing the expression (i.e. gene and/or protein expression) or an activity of SERPINB9. Agents that upregulate expression and/or activity of SERPINB9 may alternatively be referred to as agents for increasing/enhancing the expression or activity of SERPINB9. Such agents may also be referred to as ‘SERPINB9 agonists’ or ‘SERPINB9 enhancers’.

As used herein, ‘expression’ may be gene or protein expression. Gene expression encompasses transcription of DNA to RNA, and can be measured by various means known to those skilled in the art, for example by measuring levels of mRNA by quantitative real-time PCR (qRT-PCR), or by reporter-based methods. Similarly, protein expression can be measured by various methods well known in the art, e.g. by antibody-based methods, for example by western blot, immunohistochemistry, immunocytochemistry, flow cytometry, ELISA, ELISPOT, or reporter-based methods.

Upregulation of expression of SERPINB9 may be characterised by an increase in the level of RNA encoding SERPINB9, and/or an increase in the level of SERPINB9 protein, relative to the level in the absence of such upregulation. Similarly, upregulation of an activity of SERPINB9 may be characterised by an increase in the level of the relevant activity relative to the level in the absence of such upregulation.

Accordingly, agents for increasing the expression or activity of SERPINB9 include agents that increase the level of gene and/or protein expression of SERPINB9, and agents that increase the level of an activity of SERPINB9. It will be appreciated that the increase in the preceding sentence refers to the level of expression of SERPINB9, or the level of the relevant SERPINB9 activity, observed in the absence of the agent.

As used herein, an ‘activity’ of SERPINB9 may refer to inhibition of a protease. An activity of SERPINB9 may be inhibition of a serine protease (e.g. granzyme B) and/or a cysteine protease (e.g. a caspase).

Inhibition of a serine protease (e.g. granzyme B) may comprise binding to and/or inactivating the serine protease.

In some embodiments, an agent for increasing the expression or activity of SERPINB9 (i.e. a SERPINB9 agonist) according to the present disclosure displays one or more of the following properties:
Increases expression (e.g. gene and/or protein expression) of SERPINB9;
Increases the level of RNA encoding SERPINB9;
Increases transcription of nucleic acid encoding SERPINB9;
Reduces degradation of RNA encoding SERPINB9;
Increases the level of SERPINB9 protein;
Reduces degradation of SERPINB9 protein; or
Increases the level of a correlate of SERPINB9 activity.

It will be appreciated that a given SERPINB9 agonist may display more than one of the properties recited in the preceding paragraph. A given SERPINB9 agonist may be evaluated for the properties recited in the preceding paragraph using suitable assays. For example, the assays may be e.g. in vitro assays, optionally cell-based assays or cell-free assays. In some embodiments, the assays may be e.g. in vivo assays, i.e. performed in non-human animals. In some embodiments, the assays may be e.g. ex vivo assays, i.e. performed using cells/tissue/an organ obtains from a subject.

Where assays are cell-based assays, they may comprise treating cells with a given agent in order to determine whether the agent displays one or more of the recited properties. Assays may employ species labelled with detectable entities in order to facilitate their detection. Assays may comprise evaluating the recited properties following treatment of cells separately with a range of quantities/concentrations of a given agent (e.g. a dilution series). The cells employed in such cell-based assays may express SERPINB9.

Agents for increasing the expression or activity of SERPINB9 capable of increasing gene expression of SERPINB9 and/or increasing the level of RNA encoding SERPINB9 and/or increasing transcription of nucleic acid encoding SERPINB9 and/or reducing degradation of RNA encoding SERPINB9 may be identified using assays comprising detecting and/or quantifying the level of RNA encoding SERPINB9. Such assays may comprise quantifying RNA encoding SERPINB9 by RT-qPCR, northern blot, etc., which are techniques well known to the skilled person. The methods may employ primers and/or probes for the detection and/or quantification of RNA encoding SERPINB9. Such assays may comprise contacting cells in in vitro culture with a putative SERPINB9 agonist, and subsequently (e.g. after an appropriate period of time, i.e. a period of time sufficient for a change in the level of RNA encoding SERPINB9 to be observed) measuring the level of RNA encoding SERPINB9. Such assays may further comprise comparing the level of RNA encoding SERPINB9 in cells treated with the putative SERPINB9 agonist to the level of RNA encoding SERPINB9 detected in a control condition in which cells of the same type are subjected to the same conditions, except that instead of being treated with the putative SERPINB9 agonist they are untreated, or otherwise treated with a negative control agent known not to affect the level of RNA encoding SERPINB9.

Increased transcription of nucleic acid encoding SERPINB9 may be a consequence of promotion of assembly and/or activity of factors required for transcription of the DNA encoding SERPINB9. Reduced degradation of RNA encoding SERPINB9 may be a consequence of reduced enzymatic degradation of RNA encoding SERPINB9, e.g. as a consequence of RNA interference (RNAi), and/or increased stability of RNA encoding SERPINB9.

Herein, ‘contacting’ cells with a given agent (e.g. a putative SERPINB9 agonist) may comprise applying the agent to, and/or mixing the agent with, the cells. In some embodiments, the SERPINB9 agonist is provided to the cells in combination with one or more further agents for facilitating introduction of the SERPINB9 agonist into the cells, and/or for facilitating uptake of the SERPINB9 agonist by the cells. For example, in embodiments wherein an SERPINB9 agonist is, or is encoded by, one or more nucleic acids, the cells may be contacted with the nucleic acid(s) and an agent for facilitating introduction of the nucleic acid(s) into the cells, e.g. by transfection or transduction.

Agents for increasing the expression or activity of SERPINB9 capable of increasing the level of SERPINB9 protein and/or reducing degradation of SERPINB9 protein and/or increasing translation of mRNA encoding SERPINB9 may be identified using assays comprising detecting the level of SERPINB9 protein, e.g. using techniques well known to the skilled person, such as antibody or reporter-based methods (western blot, ELISA, immunohisto/cytochemistry, etc.). The methods may employ antibodies specific for SERPINB9. Such assays may comprise contacting cells in in vitro culture with a putative SERPINB9 agonist, and subsequently (e.g. after an appropriate period of time, i.e. a period of time sufficient for a change in the level of SERPINB9 protein to be observed) measuring the level of SERPINB9 protein. Such assays may further comprise comparing the level of SERPINB9 protein in cells treated with the putative SERPINB9 agonist to the level of SERPINB9 protein detected in a control condition in which cells of the same type are subjected to the same conditions, except that instead of being treated with the putative SERPINB9 agonist they are untreated, or otherwise treated with a negative control agent known not to affect the level of SERPINB9 protein.

An increase in the level of SERPINB9 protein may e.g. be the result of an increase in the level of RNA encoding SERPINB9, increased post-transcriptional processing of RNA encoding SERPINB9, or reduced degradation of SERPINB9 protein.

Agents for increasing the expression or activity of SERPINB9 capable of increasing the level of a function of SERPINB9 (e.g. a function of SERPINB9 as described hereinabove) may be identified using assays comprising detecting the level of the relevant function. Detecting the level of a given function may comprise detecting and/or quantifying a correlate of the function. Such assays may comprise contacting cells in in vitro culture with a putative SERPINB9 agonist, and subsequently (e.g. after an appropriate period of time, i.e. a period of time sufficient for a change in the level of the relevant function and/or a correlate thereof to be observed) measuring the level of the relevant function and/or a correlate thereof. Such assays may further comprise comparing the level of a function of SERPINB9 in cells treated with the putative SERPINB9 agonist to the level of the function of SERPINB9 detected in a control condition in which cells of the same type are subjected to the same conditions, except that instead of being treated with the putative SERPINB9 agonist they are untreated, or otherwise treated with a negative control agent known not to affect the level of the relevant function of SERPINB9.

In some embodiments, an SERPINB9 agonist according to the present disclosure is capable of increasing the expression (e.g. gene and/or protein expression) of SERPINB9/increasing the level of RNA encoding SERPINB9/increasing transcription of nucleic acid encoding SERPINB9/increasing the level of SERPINB9 protein/increasing the level of a correlate of SERPINB9 activity to more than 1 times, e.g. one of ≥1.01 times, ≥1.02 times, ≥1.03 times, ≥1.04 times, ≥1.05 times, ≥1.1 times, ≥1.2 times, ≥1.3 times, ≥21.4 times, ≥1.5 times, ≥1.6 times, ≥1.7 times, ≥1.8 times, ≥1.9 times, ≥2 times, ≥3 times, ≥4 times, ≥5 times, ≥6 times, ≥7 times, ≥8 times, ≥9 times or ≥10 times the level observed in the absence of the SERPINB9 agonist, or in the presence of the same quantity of a control agent known not to possess such agonist activity, in a given assay.

In some embodiments, an SERPINB9 agonist according to the present disclosure is capable of reducing degradation of SERPINB9 protein/reducing degradation of RNA encoding SERPINB9 to less than 1 times, e.g. one of ≤0.99 times, ≤0.95 times, ≤0.9 times, ≤0.85 times, ≤0.8 times, ≤0.75 times, ≤0.7 times, ≤0.65 times, ≤0.6 times, ≤0.55 times, ≤0.5 times, ≤0.45 times, ≤0.4 times, ≤0.35 times, ≤0.3 times, ≤0.25 times, ≤0.2 times, ≤0.15 times, ≤0.1 times, ≤0.05 times, or ≤0.01 times the level observed in the absence of the SERPINB9 agonist, or in the presence of the same quantity of a control agent known not to possess such agonist activity, in a given assay.

In some embodiments, an agent for increasing the expression or activity of SERPINB9 according to the present disclosure is or comprises nucleic acid encoding SERPINB9, e.g. as described herein.

### Nucleic Acids and Vectors

The present disclosure provides a nucleic acid, or a plurality of nucleic acids, encoding polypeptide(s) of the present disclosure. For example, the present disclosure provides a nucleic acid, or a plurality of nucleic acids encoding a SERPINB9 polypeptide. Also provided is a nucleic acid, or a plurality of nucleic acids encoding an agent for increasing the expression or activity of SERPINB9.

In some embodiments, the nucleic acid(s) comprise or consist of DNA and/or RNA. In some embodiments, the nucleic acid(s) may be, or may be comprised in, a vector, or a plurality of vectors. That is, the nucleotide sequence(s) of the nucleic acid(s) may be contained in vector(s). The SERPINB9/agent for increasing the expression or activity of SERPINB9 may be produced within a cell by transcription from a vector encoding the peptide/polypeptide, and subsequent translation of the transcribed RNA.

Accordingly, the present disclosure also provides a vector, or plurality of vectors, comprising the nucleic acid or plurality of nucleic acids according to the present disclosure. The vector may facilitate delivery of the nucleic acid(s) comprising/encoding a SERPINB9 polypeptide. The vector may be an expression vector comprising elements required for expressing nucleic acid(s) comprising/encoding a SERPINB9 polypeptide.

A ‘vector’ as used herein is a nucleic acid molecule used as a vehicle to transfer exogenous nucleic acid into a cell. The vector may be a vector for expression of the nucleic acid in the cell. Such vectors may include a promoter sequence operably linked to the nucleotide sequence encoding the sequence to be expressed. A vector may also include a termination codon and expression enhancers. Any suitable vectors, promoters, enhancers and termination codons known in the art may be used to express a peptide or polypeptide from a vector according to the present disclosure.

The term ‘operably linked’ may include the situation where a selected nucleic acid sequence and regulatory nucleic acid sequence (e.g. promoter and/or enhancer) are covalently linked in such a way as to place the expression of nucleic acid sequence under the influence or control of the regulatory sequence (thereby forming an expression cassette). Thus, a regulatory sequence is operably linked to the selected nucleic acid sequence if the regulatory sequence is capable of effecting transcription of the nucleic acid sequence. The resulting transcript(s) may then be translated into a desired peptide(s)/polypeptide(s).

Suitable vectors include plasmids, binary vectors, DNA vectors, mRNA vectors, viral vectors (e.g. retroviral vectors, e.g. gammaretroviral vectors (e.g. murine Leukemia virus (MLV)-derived vectors, e.g. SFG vector), lentiviral vectors, adenovirus vectors, adeno-associated virus vectors, vaccinia virus vectors and herpesvirus vectors), transposon-based vectors, and artificial chromosomes (e.g. yeast artificial chromosomes), e.g. as described in Maus et al., Annu Rev Immunol (2014) 32:189-225 or Morgan and Boyerinas, Biomedicines (2016) 4:9, which are both hereby incorporated by reference in their entirety.

In some embodiments, the vector is a viral vector. In some embodiments, the vector is a retroviral vector. In some embodiments, the vector is an MLV-derived vector. In some embodiments, the vector is an SFG vector.

In some embodiments, the vector may be a eukaryotic vector, e.g. a vector comprising the elements necessary for expression of nucleic acid from the vector in a eukaryotic cell. In some embodiments, the vector may be a mammalian vector, e.g. comprising a cytomegalovirus (CMV) or SV40 promoter to drive expression. In some embodiments, the vector comprises a cell- or tissue-specific promoter, e.g. an immune cell-specific promoter.

In some embodiments, a vector is selected based on tropism for a cell type/tissue/organ to which it is desired to deliver the nucleic acid. In some embodiments, a vector is selected based on tropism for a cell type/tissue/organ in which it is desired to express SERPINB9. For example, it may be desired to deliver the nucleic acid encoding a SERPINB9 polypeptide to an immune cell (e.g. an effector immune cell, e.g. a T cell or an NK cell).

In some embodiments, nucleic acids according to the present disclosure comprise modification to incorporate one or more moieties facilitating delivery to, and/or uptake by, a cell type or tissue of interest (e.g. an immune cell). In some embodiments, nucleic acids according to the present disclosure are linked (e.g. chemically conjugated to) one or more moieties facilitating delivery to, and/or uptake by, a cell type or tissue of interest.

Modification to, and formulation of, nucleic acids to facilitate targeted delivery to cell types and/or tissues of interest is described e.g. in Lorenzer et al., J Control Release (2015) 203:1-15, which is hereby incorporated by reference in its entirety. The moiety facilitating delivery to, and/or uptake by, a cell type or tissue of interest may bind selectively to the target cell type/tissue of interest. The moiety may facilitate traversal of the cell membrane of the target cell type and/or of cells of the tissue of interest. The moiety may bind to a molecule expressed at the cell surface of the target cell type/tissue of interest. The moiety may facilitate internalisation of the nucleic acid by the target cell type/tissue of interest (e.g. by endocytosis).

In some embodiments the nucleic acid further encodes another polypeptide of interest, e.g. a molecule for directing activity of an immune cell against a cell expressing a given target antigen (e.g. a chimeric antigen receptor (CAR) or a T cell receptor (TCR)).

The nucleic acid may comprise a nucleotide sequence encoding a SERPINB9 polypeptide as described herein, and a nucleotide sequence encoding another polypeptide of interest. The nucleotide sequences encoding the SERPINB9 polypeptide and the other polypeptide of interest may be in the same reading frame, and may not comprise a stop codon provided in between the nucleotide sequences.

In some embodiments, a nucleic acid/vector according to the present disclosure is multicistronic (e.g. bicistronic, tricistronic, etc.); that is, in some embodiments the vector encodes mRNA with multiple protein-coding regions. In some embodiments the vector is bicistronic. In some embodiments the vector comprises nucleic acid encoding an internal ribosome entry site (IRES). In some embodiments the vector comprises nucleic acid permitting a SERPINB9 polypeptide and another polypeptide of interest (e.g. a CAR) to be translated separately from the same RNA transcript.

In some embodiments the nucleic acid encodes a fusion polypeptide as described herein, e.g. a fusion polypeptide comprising a SERPINB9 polypeptide as described herein and a CAR as described herein.

Nucleic acids and vectors according to the present disclosure may be provided in purified or isolated form, i.e. from other nucleic acid, or naturally-occurring biological material.

### Applications of SERPINB9 and Agents for Increasing the Expression/Activity of SERPINB9

SERPINB9 polypeptides according to the present disclosure, nucleic acid(s)/vector(s) encoding SERPINB9 polypeptides according to the present disclosure and agents for increasing the expression or activity of SERPINB9 according to the present disclosure find use in various applications.

Example 2.3 herein demonstrates that immune cells modified to upregulate SERPINB9 expression/activity are less susceptible to elimination by alloreactive immune cells, as compared to equivalent cells lacking such modification. Increased expression/activity of SERPINB9 appears to protect immune cells against cytolytic activity of allogeneic effector immune cells, particularly granzyme B-mediated cytolysis.

SERPINB9 polypeptides according to the present disclosure, nucleic acid(s)/vector(s) encoding SERPINB9 polypeptides according to the present disclosure and agents for increasing the expression or activity of SERPINB9 according to the present disclosure find use in methods for, and/or use in methods comprising:
Increasing the level of expression (i.e. gene or protein expression) of SERPINB9 in a cell;
Reducing the activity of a serine protease (e.g. granzyme B) in a cell;
Reducing the activity of a caspase (e.g. caspase-1, -4, -5, -2, -3, -6, -7, -8, or -10) in a cell;
Increasing the resistance of a cell (and/or reducing the susceptibility of a cell) to cell killing by a serine protease (e.g. granzyme B);
Increasing the resistance of a cell (and/or reducing the susceptibility of a cell) to cell killing by cells expressing a serine protease (e.g. cells expressing granzyme B; e.g. effector immune cells);
Reducing the rate of cell killing of a cell by cells expressing a serine protease (e.g. cells expressing granzyme B; e.g. effector immune cells);
Increasing the resistance of a cell (and/or reducing the susceptibility of a cell) to apoptosis mediated by a death receptor (e.g. Fas-mediated apoptosis);
Increasing the persistence/survival/proliferation of a cell in the presence of an allogeneic effector immune cell (e.g. when co-cultured with allogeneic effector immune cells);
Increasing the persistence/survival/proliferation of a cell in an allogeneic subject;
Reducing allograft rejection in a subject;
Increasing the anticancer activity of a cell in the presence of an allogeneic effector immune cell (e.g. when co-cultured with allogeneic effector immune cells);
Increasing the anticancer activity of a cell in an allogeneic subject; or
Increasing persistence/survival/proliferation of a cell under conditions of chronic antigen exposure.

A ‘reduction’ or ‘increase’ in accordance with the preceding paragraph may be relative to the level of the relevant property ordinarily displayed by cells of that type (e.g. a reduction or increase relative to a reference value for the level of the relevant property for cells of that type), e.g. in the absence of treatment with an article of the present disclosure. The properties identified in the preceding paragraph may be evaluated using appropriate methods known to the skilled person.

The level of expression of gene expression of SERPINB9 in a cell, and the level of SERPINB9 protein in a cell, may be evaluated e.g. as described hereinabove.

The level of activity of a given enzyme (e.g. a protease, serine protease (e.g. granzyme B), cysteine protease (e.g. a caspase, e.g. caspase-1, -4, -5, -2, -3, -6, -7, -8, or -10) in a cell can be evaluated using an appropriate assay for detecting and/or quantifying the level of its activity. Such assays may comprise measuring the level of a substrate for the relevant enzyme and/or measuring the level of a product of the activity of the enzyme over time. The methods may comprise applying a substrate for the enzyme to cells, and detecting and/or quantifying the level of substrate and/or a product of enzyme-mediated processing of the substrate, after a given period of time. Such assays may employ labelled species for the detection and/or quantification of the substrate and/or a product of the activity of the enzyme, or may employ reagents for the detection and/or quantification of the substrate and/or a product of the activity of the enzyme. Such assays may also employ colorimetric substrates or reaction products.

By way of illustration, the activity of granzyme B can be evaluated using the Granzyme B Assay Kit from Enzo Life Sciences, Inc. (Cat. No. BML-AK711-0001), which employs the colorimetric substrate IEPD-pNA. Cleavage of the p-nitroanilide (pNA) group from IEPD-pNA increases absorption at 405 nm, enabling granzyme B activity to be detected and quantified. Other suitable assays for the detection and quantification of granzyme B activity are well known in the art, and include e.g. Granzyme B Activity Assay Kit from Sigma-Aldrich (Cat No. MAK176). Granzyme B activity can also be evaluated described e.g. in Bird et al., Mol Cell Biol. (1998), 18(11):6387-98, which is incorporated by reference hereinabove.

By way of further illustration, the activity of caspases can be evaluated e.g. using Caspase-Glo assays from Promega Corporation, which employ luminogenic caspase substrates and luciferase. Cleavage of the substrate liberates aminoluciferin, a substrate for luciferase. The activity of luciferase on aminoluciferin results in the production of light, enabling the detection and quantification of caspase activity.

Herein, a ‘serine protease’ is an enzyme that cleaves a peptide bond in a polypeptide, in which serine serves as the nucleophilic amino acid at the enzymes active site. ‘Serine protease activity’ refers to catalysis of cleavage of a peptide bond in a polypeptide by a serine protease. Serine proteases are reviewed e.g. in Patel, Allergol. Immunopathol. (Madr). (2017) 45(6): 579-591, which is hereby incorporated by reference in its entirety.

In preferred embodiments, a serine protease is a granzyme. Granzymes are serine proteases contained within cytoplasmic granules of effector immune cells such as cytotoxic T lymphocytes (CTLs) and natural killer (NK) cells. Granzymes are reviewed e.g. in Chowdhury and Lieberman, Annu. Rev. Immunol. (2008) 26: 389-420 (hereby incorporated by reference in its entirety), and include granzymes A, B, H, K and M. In preferred embodiments, a granzyme according to the present disclosure is granzyme B. Accordingly, a in some embodiments, a serine protease is granzyme B. Granzyme B biology is reviewed e.g. in Lord et al. Immunol. Rev. (2003) 193:31-38 and Trapani et al., Curr. Opin. Immunol. (2003) 15:533-43. Granzyme B cleaves polypeptides after aspartic acid residues, and induces apoptosis by activating the caspases (e.g. the executioner caspase-3). Human granzyme B also activates cell death by directly cleaving bid and ICAD, respectively activating the same mitochondrial and DNA damage pathways.

Herein, a ‘cysteine protease’ is an enzyme that cleaves a peptide bond in a polypeptide, in which cysteine serves as the nucleophilic amino acid at the enzymes active site. ‘Cysteine protease activity’ refers to catalysis of cleavage of a peptide bond in a polypeptide by a cysteine protease. Cysteine proteases are reviewed e.g. in Verma et al., Front. Pharmacol. (2016) 7:107, which is hereby incorporated by reference in its entirety.

In preferred embodiments, a cysteine protease is a caspase. Caspases are cysteine-dependent aspartate-directed proteases with roles in programmed cell death including apoptosis and pyroptosis. Caspases are reviewed e.g. in Julien and Wells, Cell Death & Differentiation (2017) 24: 1380-1389 (hereby incorporated by reference in its entirety), and include caspases-1, -2, -3, -4, -5, -6, -7, -8, -9, -10, -12 and -14. In some embodiments, a caspase according to the present disclosure is a caspase involved in apoptosis, e.g. selected from caspase-2, -8, -9, -10, -3, -6 and -7. In some embodiments, a caspase is an initiator caspase, e.g. selected from caspase-2, -8, -9 and -10. In some embodiments, a caspase is an executioner caspase, e.g. selected from caspase-3, -6 and -7. In some embodiments, a caspase according to the present disclosure is a caspase involved in pyroptosis, e.g. selected from caspase-1, -4, -5, and -12.

The resistance/susceptibility of a cell to cell killing by a serine protease (e.g. granzyme B), the resistance/susceptibility of a cell to cell killing by cells expressing a serine protease, (e.g. granzyme B; e.g. effector immune cells) and the rate of cell killing of a cell by cells expressing a serine protease, (e.g. granzyme B-expressing cells; e.g. effector immune cells) can be evaluated using assays comprising detecting and/or quantifying cytolysis/cell killing of cells.

In some embodiments, the serine protease (e.g. granzyme B) may be expressed by the cell whose resistance/susceptibility to cell killing by a serine protease (e.g. granzyme B) is being evaluated. In some embodiments, the serine protease (e.g. granzyme B) may be expressed by a different cell to the cell whose resistance/susceptibility to cell killing by a serine protease (e.g. granzyme B) is being evaluated. In some embodiments, the cells expressing a serine protease (e.g. granzyme B) are allogeneic with respect to the cell. That is, the granzyme B-expressing cell may be obtained/derived from a subject other than the subject from which the test cell is obtained/derived (e.g. a genetically non-identical subject). In some embodiments, the cells expressing a serine protease (e.g. granzyme B) may be autogeneic or autologous with respect to the cell. That is, the granzyme B-expressing cell may be obtained/derived from a genetically identical subject, or the same subject, as the subject from which the test cell is obtained/derived.

Herein, ‘apoptosis mediated by a death receptor’ refers to programmed cell death (i.e. apoptosis) of cells expressing the death receptor, induced by death receptor activation. Apoptosis mediated by death receptors is reviewed e.g. in Green, Cold Spring Harb Perspect Biol. (2022) 14:a041053, which is hereby incorporated by reference in its entirety.

Death receptors belong to the tumour necrosis factor/nerve growth factor superfamily. They are type I transmembrane proteins with a conserved cytoplasmic death domain (DD). Death receptors are activated upon ligation with their cognate ligands. Following activation, the DD facilitates homotypic interactions with adaptor proteins via their death domain motifs, activating the caspase cascade and ultimately leading to apoptosis. In some embodiments, the death receptor is selected from: Fas (also called CD95 and APO-1), tumour necrosis factor receptor-1 (TNFR1), TRAIL receptor-1 (also called DR4), TRAIL receptor-2 (also called DR5), death receptor 3 (DR3), death receptor 6 (DR6), nerve growth factor receptor (NGFR) and Ectodysplasin-A receptor (EDAR). In some embodiments, the death receptor is Fas.

Herein, ‘Fas-mediated apoptosis’ refers to programmed cell death (i.e. apoptosis) of cells expressing the Fas receptor, induced by its activation. Fas-mediated apoptosis is reviewed e.g. in Timmer et al., J. Pathol. (2002) 196(2):125-34, which is hereby incorporated by reference in its entirety. Fas-mediated apoptosis typically involves cross-linking of Fas by its ligand FasL (e.g. expressed at the surface of effector immune cells), activating the caspase cascade and ultimately leading to apoptosis.

The resistance/susceptibility of a cell to apoptosis mediated by a death receptor (e.g. Fas-mediated apoptosis) can be evaluated using assays comprising detecting and/or quantifying apoptosis mediated by a death receptor. Such assays may comprise contacting the cells with an agent for activating apoptosis mediated by a death receptor (e.g. the ligand of a given death receptor or cells expressing the ligand of a given death receptor) and detecting and/or quantifying apoptosis of the cells. For example, such assays may comprise contacting the cells with an agent for activating Fas-mediated apoptosis (e.g. FasL or cells expressing FasL), and detecting and/or quantifying apoptosis of the cells. Detecting/quantifying apoptosis may comprise analysing the level of expression or activity of one or more caspases, and/or may comprise detecting and/or quantifying live, dead and/or apoptotic cells. Detecting/quantifying apoptosis may comprise detecting and/or quantifying one or markers of apoptosis (e.g. phosphatidylserine (PS) exposure, Bcl-2 family protein (e.g. Bax, Bak, Bid) activation, ROS production, caspase activation, mitochondrial membrane permeabilization or DNA fragmentation).

Cell killing can be investigated, for example, using any of the methods reviewed in Zaritskaya et al., Expert Rev Vaccines (2011), 9(6):601-616, hereby incorporated by reference in its entirety. Examples of in vitro assays of cytotoxicity/cell killing assays include release assays such as the ⁵¹Cr release assay, the lactate dehydrogenase (LDH) release assay, the 3-(4,5-dimethylthiazol-2-yl)-2,5-diphenyl tetrazolium bromide (MTT) release assay, and the calcein-acetoxymethyl (calcein-AM) release assay. These assays measure cell killing based on the detection of factors released from lysed cells. Cell killing of a given test cell type by a given effector immune cell type can be analysed e.g. by co-culturing the test cells with the effector immune cells, and measuring the number/proportion of viable/dead (e.g. lysed) test cells after a suitable period of time. Other suitable assays include the xCELLigence real-time cytolytic in vitro potency assay described in Cerignoli et al., PLoS One. (2018) 13(3): e0193498 (hereby incorporated by reference in its entirety). An increase in resistance to cell killing by granzyme B-expressing cells (e.g. effector immune cells), and/or a reduction in susceptibility to cell killing by such cells, relative to a reference level of cell killing (e.g. for that cell type) can be determined by detection of a reduction in the number/proportion of dead (e.g. lysed) test cells, and/or an increase in the number/proportion of live (e.g. viable, non-lysed) test cells, after a given period of time.

The rate of cell killing of a given test cell type can be determined by analysing cytolysis over time, e.g. by determining the number/proportion of lysed and/or non-lysed test cells at different time points. For example, such analysis could employ the xCELLigence system as described in Example 1.6 herein. A reduction in the rate of cell killing relative to a reference rate of cell killing (e.g. for that cell type) can be determined by detection of a reduction in the number/proportion of dead (e.g. lysed) test cells, and/or an increase in the number/proportion of live (e.g. viable, non-lysed) test cells, per unit of time.

Persistence/survival/proliferation/expansion of a given cell/population thereof can be evaluated in vitro by measuring or monitoring the number or proportion of such cells overtime.

Cell proliferation/expansion can be investigated by analysing cell division or the number of cells over a period of time. Cell division can be analysed, for example, by in vitro analysis of incorporation of 3H-thymidine or by CFSE dilution assay, e.g. as described in Fulcher and Wong, Immunol Cell Biol (1999) 77(6): 559-564, hereby incorporated by reference in entirety. Proliferating cells can also be identified by analysis of incorporation of 5-ethynyl-2′-deoxyuridine (EdU) by an appropriate assay, as described e.g. in Buck et al., Biotechniques. 2008 June; 44(7):927-9, and Sali and Mitchison, PNAS USA 2008 Feb. 19; 105(7): 2415-2420, both hereby incorporated by reference in their entirety.

The level of cell proliferation of, or population expansion for, a given cell type can also be evaluated by counting the number of the relevant cell type at one or more defined time points, e.g. following culture in certain conditions.

An increase in persistence/survival can be determined by detection of a greater number/proportion of live (e.g. viable, non-lysed) cells, after a given period of time.

The cell killing assays described above can also be employed for the evaluation of the persistence/survival of cells in the presence of allogeneic effector immune cells. For example, persistence/survival of a given test cell type in the presence of allogeneic effector immune cells can be analysed by co-culturing the test cells with the allogeneic effector immune cells, and measuring the number/proportion of viable/dead (e.g. lysed) test cells after a suitable period of time. An increase in persistence/survival relative to a reference level (e.g. for that cell type) can be determined by detection of a reduction in the number/proportion of dead (e.g. lysed) test cells, and/or an increase in the number/proportion of live (e.g. viable, non-lysed) test cells, after a given period of time.

Suitable assays for evaluating proliferation/expansion/persistence/survival in the presence of allogeneic effector immune cells include e.g. mixed lymphocyte reaction (MLR) assays, such as the assay described in Example 1.5 herein.

In accordance with the assays described herein, the given/defined period of time after which the level of the relevant property is evaluated may be any suitable period of time providing for meaningful comparative analysis of the level of the relevant property between the test cell(s) and controls, in the relevant assay. In some embodiments, the level of the relevant property is evaluated at or after a sufficient period of time that the maximal level of the relevant property is or has been attained, in the relevant assay. In some embodiments, the given/defined period of time is one of 1, 2, 3, 4, 5, 6, 7, 8, 9, 10, 11, 12, 13, 14, 15, 16, 17, 18, 19 or 20 days. In some embodiments, the given/defined period of time is one of 3, 4, 5, 6, 7, 8, 9, 10, 11, 12, 13, 14 or 15 days.

As used herein, where a given cell is referred to herein as being ‘allogeneic’ with respect to a reference cell, it is obtained/derived from a subject other than the subject from which the reference cell is obtained/derived. Thus, the allogeneic effector immune cells referred to in the preceding paragraph are obtained/derived from a subject other than the subject from which the test cells are obtained/derived. In some embodiments, an allogeneic cell comprises MHC/HLA genes encoding MHC/HLA molecules (e.g. MHC class I a and/or MHC class II molecules) that are non-identical to the MHC/HLA molecules (e.g. MHC class I a and/or MHC class II molecules) encoded by the MHC/HLA genes of the reference cell.

Survival/persistence of a given cell or population of cells in a subject (e.g. an allogeneic subject) in vivo can be evaluated e.g. using methods in which cells are labelled with a detectable marker or reporter, introduced into the subject, and their survival is monitored over time. Such methods include e.g. labelling cells with firefly luciferase, and measuring firefly luciferase activity at different time points by bioluminescence imaging, e.g. following administration of D-Luciferin (e.g. as described in Prescher and Contag, Curr. Opin. Chem. Biol. (2010) 14(1):80-9, which is hereby incorporated by reference in its entirety). An increase in persistence/survival relative to a reference level (e.g. for that cell type) can be determined by detection of a greater number/proportion of the test cells in the subject, after a given period of time.

As used herein, where a subject is referred to herein as being ‘allogeneic’ with respect to a reference cell, the subject is a subject other than the subject from which the reference cell is obtained/derived. Thus, the allogeneic subject referred to in the preceding paragraph is a subject other than the subject from which the test cells are obtained/derived. In some embodiments, an allogeneic subject comprises MHC/HLA genes encoding MHC/HLA molecules (e.g. MHC class I a and/or MHC class II molecules) that are non-identical to the MHC/HLA molecules (e.g. MHC class I a and/or MHC class II molecules) encoded by the MHC/HLA genes of the reference cell.

Suitable assays for evaluating the survival/persistence of a given cell or population of cells in an allogeneic subject (i.e. allogeneic with respect to the test cell(s)) in vivo may be performed in a proxy allogeneic subject. A proxy allogeneic subject may be established by injecting allogeneic immune cells (i.e. allogeneic with respect to the test cell(s)) into a non-allogeneic subject, e.g. by injecting allogeneic effector immune cells into an MHC knockout mouse. Accordingly, a suitable assay for evaluating the survival/persistence of a given cell or population of cells in an allogeneic subject in vivo may comprise co-infusion of the given cell or population of cells and allogeneic immune cells (i.e. allogeneic with respect to the test cell(s)) into an MHC knockout mouse. In some embodiments, the survival/persistence of a given cell or population of cells in an allogeneic subject (e.g. a proxy allogeneic subject) in vivo may be evaluated essentially as described in Example 1.10 and Example 4.

The methods for evaluating persistence/survival/proliferation of cells in an allogeneic subject described above can also be employed for evaluation of allograft rejection in a subject. The SERPINB9 polypeptides and other agents for increasing the expression or activity of SERPINB9 according to the present disclosure are also useful for reducing/preventing of graft rejection. Graft rejection refers to the destruction of transplanted cells/tissue/organs by a recipient's immune system following transplantation.

Where graft rejection is of an allotransplant, it may be referred to as allograft rejection. Increasing expression or activity of SERPINB9 in a cell confers resistance to serine protease (e.g. granzyme B)-mediated depletion by granzyme B-expressing cells (e.g. effector immune cells) in the recipient subject.

Anticancer activity of a given cell type, or population of such cells, can be evaluated e.g. by analysing cell killing of cancer cells by such cells, and/or a correlate thereof.

Anticancer activity can be analysed e.g. in vitro, using assays to detect and/or quantify cell killing of cancer cells. The cell killing assays described hereinabove above can be employed for the evaluation of cell killing of cancer cells.

For example, the anticancer activity of a given test cell type can be evaluated in the presence of allogeneic effector immune cells, by culturing the test cells with allogeneic effector immune cells and cancer cells, and monitoring the number/proportion of live and/or dead (e.g. lysed) cancer cells over time. An increase in anticancer activity (e.g. relative to level anticancer activity displayed by cells of that type in the absence of treatment with an article of the present disclosure) can be determined by detection of a reduction in the number/proportion of live cancer cells and/or an increase in the number/proportion of dead (e.g. lysed) cancer cells, after a given period of time.

Suitable assays for evaluating the anticancer activity of a cell, or population of cells, include e.g. mixed lymphocyte reaction (MLR) assays. In some embodiments, the anticancer activity of a cell, or population of cells may be evaluated essentially as described in Example 1.5 herein.

Anticancer activity can also be analysed e.g. in vivo, using assays to detect and/or quantify cell killing of cancer cells in a subject.

For example, the anticancer activity of a given test cell type can be evaluated using assays in which test cells are administered to a subject having a cancer, and the number/proportion of cancer cells, cancer burden and/or tumor volume are monitored over time. An increase in anticancer activity (e.g. relative to level anticancer activity displayed by cells of that type, e.g. in the absence of treatment with an article of the present disclosure) can be determined by detection of a reduction in the number/proportion of cancer cells, a reduction in cancer burden and/or a decrease in tumor volume in the subject, after a given period of time. In some embodiments, such assays may employ cancer cells labelled with a detectable marker or reporter, and anticancer activity in vivo can be evaluated by monitoring the number/proportion of the cells overtime. Such methods include e.g. labelling cancer cells with firefly luciferase, and measuring firefly luciferase activity at different time points by bioluminescence imaging, e.g. following administration of D-Luciferin (e.g. as described in Prescher and Contag, Curr. Opin. Chem. Biol. (2010) 14(1):80-9, which is hereby incorporated by reference in its entirety).

In some embodiments, the anticancer activity of a cell, or population of cells, may be evaluated essentially as described in Example 1.10 herein.

Persistence/survival/proliferation of a cell, or population of cells, under conditions of chronic antigen exposure can be evaluated in vitro by exposing cells to serial antigen challenge, and measuring or monitoring the number or proportion of such cells over time or after a given period of time. For example, persistence/survival of a given test cell type can be analysed by performing serial co-culture of the test cells with cancer cells (e.g. multiple rounds of tumour challenge by re-plating test cells with fresh cancer cells), and monitoring the number/proportion of live and/or dead (e.g. lysed) test cells after a suitable period of time. An increase in persistence/survival of a given test cell type (e.g. relative to level displayed by cells of that type, e.g. in the absence of treatment with an article of the present disclosure) can be determined by detection of a reduction in the number/proportion of dead (e.g. lysed) test cells, and/or an increase in the number/proportion of live (e.g. viable, non-lysed) test cells, after a given period of time.

In some embodiments, ‘conditions of chronic antigen exposure’ may refer to multiple rounds (e.g. at least 2, 3, 4, 5, 6, 7, 8, 9, or 10 rounds) of stimulation of the immune cell with an antigen, or a cell comprising/expressing an antigen, for which the immune cell comprises a specific receptor (e.g. a CAR having an antigen-binding domain that binds to the antigen, and/or a TCR that binds to an MHC:peptide complex comprising a peptide of the antigen). In some embodiments, ‘conditions of chronic antigen exposure’ may refer to multiple separate instances (e.g. at least 2, 3, 4, 5, 6, 7, 8, 9, or 10 instances) of adding an antigen, or a cell comprising/expressing an antigen, for which the immune cell comprises a specific receptor (e.g. a CAR having an antigen-binding domain that binds to the antigen, and/or a TCR that binds to an MHC:peptide complex comprising a peptide of the antigen) to the immune cells in culture. In some embodiments, the rounds of stimulation with an antigen/addition of antigen are performed at regular intervals e.g. every 1, 2, 3, 4, or 5 days. In some embodiments, the rounds of stimulation with an antigen/addition of an antigen are performed at regular intervals, e.g. every 2-3 days. In some embodiments, ‘conditions of antigen exposure’ may refer to stimulation of the immune cells in an in vivo model engrafted with a tumour/tumour cells comprising/expressing an antigen for which the immune cell comprises a specific receptor (e.g. a CAR having an antigen-binding domain that binds to the antigen, and/or a TCR that binds to an MHC:peptide complex comprising a peptide of the antigen).

In some embodiments, persistence/survival/proliferation a cell, or population of cells, under conditions of chronic antigen exposure may be evaluated essentially as described in Example 1.9 and/or Example 1.10 ‘Activation Induced Cell Death (AICD) model’ herein.

In some embodiments, in methods and uses according to the present disclosure, a SERPINB9 polypeptide, nucleic acid(s), vector(s) or agent for increasing the expression or activity of SERPINB9 according to the present disclosure may increase the level of expression (i.e. gene or protein expression) of SERPINB9 in a cell/increase the resistance of a cell to cell killing by a serine protease (e.g. granzyme B)/increase the resistance of a cell to cell killing by cells expressing a serine protease (e.g. granzyme B; e.g. effector immune cells)/increase the resistance of a cell to apoptosis mediated by a death receptor (e.g. Fas-mediated apoptosis)/increase the persistence, survival or proliferation of a cell in the presence of an allogeneic effector immune cell/increase the persistence, survival or proliferation of a cell in an allogeneic subject/increase the anticancer activity of a cell in the presence of an allogeneic effector immune cell/increase the anticancer activity of a cell in an allogeneic subject/increasing persistence, survival or proliferation of a cell under conditions of chronic antigen exposure to greater than 1 times, e.g. one of ≥1.01 times, ≥1.02 times, ≥1.03 times, ≥1.04 times, ≥1.05 times, ≥1.1 times, ≥1.2 times, ≥1.3 times, ≥1.4 times, ≥1.5 times, ≥1.6 times, ≥1.7 times, ≥1.8 times, ≥1.9 times, ≥2 times, ≥3 times, ≥4 times, ≥25 times, ≥6 times, ≥7 times, ≥8 times, ≥9 times or ≥10 times the level of the relevant property ordinarily displayed cells of that type (e.g. a reference value for the level of the relevant property, for cells of that type, i.e. in the absence of treatment with an article of the present disclosure).

In some embodiments, in methods and uses according to the present disclosure, a SERPINB9 polypeptide, nucleic acid(s), vector(s) or agent for increasing the expression or activity of SERPINB9 according to the present disclosure may reduce the activity of a serine protease (e.g. granzyme B) in a cell/reduce the activity of a caspase (e.g. caspase-1, -4, -5, -2, -3, -6, -7, -8, or -10) in a cell/reduce the susceptibility of a cell to cell killing by a serine protease (e.g. granzyme B)/reduce the susceptibility of a cell to cell killing by cells expressing a serine protease (e.g. granzyme B; e.g. effector immune cells)/reduce the susceptibility of a cell to apoptosis mediated by a death receptor (e.g. Fas-mediated apoptosis)/reduce the rate of cell killing of a cell by cells expressing a serine protease (e.g. granzyme B; e.g. effector immune cells)/reduce allograft rejection in a subject to less than 1 times, e.g. one of ≤0.99 times, ≤0.95 times, ≤0.9 times, ≤0.85 times, ≤0.8 times, ≤0.75 times, ≤0.7 times, ≤0.65 times, ≤0.6 times, ≤0.55 times, ≤0.5 times, ≤0.45 times, ≤0.4 times, ≤0.35 times, ≤0.3 times, ≤0.25 times, ≤0.2 times, ≤0.15 times, ≤0.1 times, ≤0.05 times, or ≤0.01 times the level of the relevant property ordinarily displayed cells of that type (e.g. a reference value for the level of the relevant property, for cells of that type).

### Cells Modified to Increase the Expression or Activity of SERPINB9

The present disclosure provides a cell modified to increase the expression (i.e. gene and/or protein expression) or activity of SERPINB9. It will be appreciated that where cells are referred to herein in the singular (i.e. ‘a/the cell’), pluralities/populations of such cells are also contemplated.

A cell having increased expression (i.e. gene and/or protein expression) or activity of SERPINB9 may be characterised by a level of expression of SERPINB9, or in the level of an activity of SERPINB9, which is greater than the level of expression/the activity ordinarily displayed by cells of that type. A cell having increased expression or activity of SERPINB9 may be characterised by a level of expression of SERPINB9, or in the level of an activity of SERPINB9, which is greater than a reference value for the level of expression/the activity for cells of that type.

A cell having increased expression (i.e. gene and/or protein expression) or activity of SERPINB9 may do so as a consequence of treatment/modification as described herein.

In some embodiments, the cell is a cell that has been treated/modified to increase the level or activity of SERPINB9 protein in the cell. In some embodiments, the cell is a cell that has been treated/modified to increase gene and/or protein expression of SERPINB9.

The present disclosure provides a cell comprising or expressing a SERPINB9 polypeptide as described herein. The present disclosure also provides a cell comprising or expressing nucleic acid(s) (e.g. exogenous nucleic acid) encoding a SERPINB9 polypeptide as described herein. The present disclosure also provides a cell comprising or expressing vector(s) encoding a SERPINB9 polypeptide as described herein.

As used herein, ‘exogenous’ nucleic acid refers to nucleic acid which is non-endogenous to the cell comprising the exogenous nucleic acid. The exogenous nucleic acid may not be encoded by the genome of the subject from which the cell is obtained/derived. The cell comprising exogenous nucleic acid may do so as a consequence of having been modified as described herein, e.g. to introduce nucleic acid(s)/vector(s) encoding a SERPINB9 polypeptide into the cell.

In some embodiments, the cell is a cell into which nucleic acid(s)/vector(s) encoding a SERPINB9 polypeptide according to the present disclosure have been introduced. Such cells may be characterised by a level of expression of SERPINB9, or a level of an activity of SERPINB9, which is greater than the level of expression/the activity displayed by equivalent cells into which the nucleic acid(s)/vector(s) have not been introduced.

In some embodiments, the cell is a cell that has been treated with an agent for increasing the expression or activity of SERPINB9 according to the present disclosure. Such cells may be characterised by a level of expression of SERPINB9, or a level of an activity of SERPINB9, which is greater than the level of expression/the activity displayed by equivalent cells that have not been treated with the agent.

The level of expression of SERPINB9 can be measured by suitable means well known to the skilled person. The level of SERPINB9 gene expression can be analysed using assays comprising detecting and/or quantifying the level of RNA encoding SERPINB9. Such assays may comprise quantifying RNA encoding SERPINB9 by RT-qPCR, northern blot, etc. The methods may employ primers and/or probes for the detection and/or quantification of RNA encoding SERPINB9. The level of SERPINB9 protein can be analysed using assays comprising detecting and/or quantifying the level of SERPINB9 protein. Such assays include e.g. antibody/reporter-based methods (western blot, ELISA, immunohisto/cytochemistry, etc.), and may e.g. employ antibodies specific for SERPINB9.

The level of an activity of SERPINB9 can be measured using an appropriate assay for the activity. Such assays include assays analysing inhibition of the activity of a serine protease (e.g. granzyme B). Such assay may comprise evaluating the activity of a serine protease, e.g. as described hereinabove. In some embodiments, SERPINB9 activity can be evaluated as described e.g. in Bird et al., Mol Cell Biol. (1998), 18(11):6387-98, which is incorporated by reference hereinabove.

In some embodiments, a cell having increased expression (i.e. gene and/or protein expression) or activity of SERPINB9 according to the present disclosure may display a level of expression, or a level of an activity of SERPINB9, that is greater than 1 times, e.g. one of ≥1.01 times, ≥1.02 times, ≥1.03 times, ≥21.04 times, ≥1.05 times, ≥1.1 times, ≥1.2 times, ≥1.3 times, ≥1.4 times, ≥1.5 times, ≥1.6 times, ≥1.7 times, ≥1.8 times, ≥1.9 times, ≥2 times, ≥3 times, ≥4 times, ≥5 times, ≥6 times, ≥7 times, ≥8 times, ≥9 times or ≥10 times the level of expression, or the level of the activity, ordinarily displayed cells of that type (e.g. a reference value for the level of expression, or for the level of the activity, for cells of that type).

In some embodiments, a cell comprising or expressing nucleic acid(s) (e.g. exogenous nucleic acid) or vectors(s) encoding a SERPINB9 polypeptide according to the present disclosure may display a level of expression of SERPINB9, or a level of an activity of SERPINB9, that is greater than 1 times, e.g. one of ≥1.01 times, ≥1.02 times, ≥1.03 times, ≥1.04 times, ≥1.05 times, ≥1.1 times, ≥1.2 times, ≥1.3 times, ≥1.4 times, ≥1.5 times, ≥1.6 times, ≥1.7 times, ≥1.8 times, ≥1.9 times, ≥2 times, ≥3 times, ≥4 times, ≥5 times, ≥26 times, ≥7 times, ≥8 times, ≥9 times or ≥10 times the level of expression, or the level of the activity, displayed by equivalent cells not comprising the nucleic acid(s)/vector(s).

In some embodiments, a cell that has been treated with an agent for increasing the expression or activity of SERPINB9 according to the present disclosure may display a level of expression of SERPINB9, or a level of an activity of SERPINB9, that is greater than 1 times, e.g. one of ≥1.01 times, ≥1.02 times, ≥1.03 times, ≥1.04 times, ≥1.05 times, ≥1.1 times, ≥1.2 times, ≥1.3 times, ≥1.4 times, ≥1.5 times, ≥1.6 times, ≥1.7 times, ≥1.8 times, ≥1.9 times, ≥2 times, ≥3 times, ≥4 times, ≥5 times, ≥6 times, ≥7 times, ≥8 times, ≥9 times or ≥10 times the level of expression, or the level of the activity, displayed by equivalent cells that have not been treated with the agent. In some embodiments, a cell that has been treated with an agent for increasing the expression or activity of SERPINB9 according to the present disclosure may display a level of expression of SERPINB9 that is greater than 1.3 times, e.g. one of ≥1.3 times, ≥1.4 times, ≥1.5 times, ≥1.6 times, ≥1.7 times, ≥1.8 times, ≥1.9 times, ≥2 times, ≥3 times, ≥4 times, ≥5 times, ≥6 times, ≥7 times, ≥8 times, ≥9 times or ≥10 times the level of expression displayed by equivalent cells not comprising the nucleic acid(s)/vector(s) (e.g. as determined by analysis essentially as described in Example 1.7).

The cell may be a eukaryotic cell, e.g. a mammalian cell. The mammal may be a primate (rhesus, cynomolgous, non-human primate or human) or a non-human mammal (e.g. rabbit, guinea pig, rat, mouse or other rodent (including any animal in the order Rodentia), cat, dog, pig, sheep, goat, cattle (including cows, e.g. dairy cows, or any animal in the order Bos), horse (including any animal in the order Equidae), donkey, and non-human primate). In preferred embodiments, the cell is a human cell.

In preferred embodiments, the cell is an immune cell. The immune cell may be a cell of hematopoietic origin, e.g. a neutrophil, eosinophil, basophil, dendritic cell, lymphocyte, or monocyte. A lymphocyte may be e.g. a T cell, B cell, NK cell, NKT cell or innate lymphoid cell (ILC), or a precursor thereof (e.g. a thymocyte or pre-B cell). The immune cell may express a CD3 polypeptide (e.g. CD3γ CD3ε CD3ζ or CD3δ), a TCR polypeptide (TCRα or TCRβ), CD27, CD28, CD4 or CD8. In some embodiments, the immune cell is a T cell, e.g. a CD3+ T cell. In some embodiments, the T cell is a CD3+, CD4+ T cell. In some embodiments, the T cell is a CD3+, CD8+ T cell. In some embodiments, the T cell is a T helper cell (TH cell). In some embodiments, the T cell is a cytotoxic T cell (e.g. a cytotoxic T lymphocyte (CTL)). In some embodiments, the immune cell is a T cell or an NK cell.

In preferred embodiments, the cell is an effector immune cell. As used herein, an ‘effector immune cell’ may be an immune cell displaying an effector function. An effector immune cell may be a CD8+ T cell, CD8+ cytotoxic T lymphocyte (CD8+ CTL), CD4+ T cell, CD4+T helper cell, NK cell, IFNγ-producing cell, memory T cell, central memory T cell, antigen-experienced T cell or CD45RO+ T cell. An effector immune cell may be characterised by one or more of the following properties: granzyme B expression, IFNγ expression, CD107a expression, IL-2 expression, TNFα expression, perforin expression, granulysin expression, and/or FAS ligand (FASL) expression. In some embodiments, an effector immune cell according to the present disclosure is a granzyme B-expressing cell.

An immune cell may be from any suitable source. An immune cell may have been obtained/isolated from a subject, e.g. a subject as described herein. An immune cell may be suitable for administration to a subject in accordance with a therapeutic/prophylactic intervention according to the present disclosure. The cell may have been obtained/isolated from the subject to be treated in accordance with therapeutic/prophylactic intervention according to the present disclosure. The cell may have been obtained/isolated from a subject other than the subject to be treated in accordance with therapeutic/prophylactic intervention according to the present disclosure. The cell may have been obtained/isolated from a healthy subject, e.g. a subject that is not known to be suffering from a disease/condition.

The present disclosure also provides a method for producing a cell according to the present disclosure, comprising introducing nucleic acid(s) or vector(s) according to the present disclosure into a cell. In some embodiments, introducing nucleic acid(s) or vector(s) according to the present disclosure into a cell comprises transformation, transfection, electroporation or transduction (e.g. retroviral transduction).

Transfection relates to the process of introducing nucleic acids into cells using means other than viral infection and is hence a non-viral method. Transfection may be performed by physical/mechanical methods (including electroporation, sonoporation, magnetofection, gene microinjection and laser irradiation) or chemical methods (liposomal-based or non-liposomal based). Liposomal-based transfection reagents are chemicals which enable the formation of positively charged lipid aggregates, which can then merge with the phospholipid bilayer of the cell to facilitate the entry of foreign genetic material. Examples of liposomal-based transfection reagents include, but are not limited to Oligofectamine®, Lipofectamine® and DharmaFECT®. Non-liposomal transfection reagents include, but are not limited to, calcium phosphate, nanoparticles, polymers, dendrimers and non-liposomal lipids. One example of a non-liposomal transfection reagent is polyethylenimine (PEI).

Electroporation may be performed e.g. as described in Koh et al., Molecular Therapy—Nucleic Acids (2013) 2, e114, which is hereby incorporated by reference in its entirety.

Transduction is a process by which nucleic acids may be introduced into a cell by a virus or a viral vector. Accordingly, in some embodiments the nucleic acid(s) is/are comprised in a viral vector(s), or the vector(s) is/are a viral vector(s). Transduction of immune cells with viral vectors is described e.g. in Simmons and Alberola-IIa, Methods Mol Biol. (2016) 1323:99-108, which is hereby incorporated by reference in its entirety. Agents may be employed in the methods of the present disclosure to enhance the efficiency of transduction. Hexadimethrine bromide (polybrene) is a cationic polymer which is commonly used to improve transduction, through neutralising charge repulsion between virions and sialic acid residues expressed on the cell surface. Other agents commonly used to enhance transduction include e.g. the poloxamer-based agents such as LentiBOOST (Sirion Biotech), Retronectin (Takara), Vectofusin (Miltenyi Biotech) and also SureENTRY (Qiagen) and ViraDuctin (Cell Biolabs).

In some embodiments the methods comprise centrifuging the cells into which it is desired to introduce nucleic acid(s) according to the present disclosure in the presence of cell culture medium comprising viral vector(s) comprising the nucleic acid(s) (referred to in the art as ‘spinfection’).

In some embodiments, the methods additionally comprise culturing the cell under conditions suitable for expression of the nucleic acid(s)/vector(s) by the cell.

Methods for culturing (including generating and/or expanding) populations of immune cells in vitrolex vivo are well known to the skilled person. Suitable culture conditions (i.e. cell culture media, additives, stimulations, temperature, gaseous atmosphere), cell numbers, culture periods and methods for introducing nucleic acid encoding a polypeptide of interest into cells, etc. can be determined by reference e.g. to Hombach et al. J Immunol (2001) 167:6123-6131, Ramos et al. J. Clin. Invest. (2017) 127(9):3462-3471, WO 2021/245249 A1, WO 2021/222927 A1, WO 2021/222928 A1, WO 2021/222929 A1, WO 2015/028444 A1 or WO 2016/008973 A1, all of which are hereby incorporated by reference in their entirety.

Conveniently, cultures of cells according to the present disclosure may be maintained at 37° C. in a humidified atmosphere containing 5% CO₂. The cells of cell cultures can be established and/or maintained at any suitable density, as can readily be determined by the skilled person.

Cell cultures can be performed in any vessel suitable for the volume of the culture, e.g. in wells of a cell culture plate, cell culture flasks, a bioreactor, etc. In some embodiments cells are cultured in a bioreactor, e.g. a bioreactor described in Somerville and Dudley, Oncoimmunology (2012) 1(8):1435-1437, which is hereby incorporated by reference in its entirety. In some embodiments cells are cultured in a GRex cell culture vessel, e.g. a GRex flask or a GRex 100 bioreactor.

In some embodiments, the methods are performed in vitro. The present disclosure also provides cells obtained or obtainable by the methods according to the present disclosure, and of course populations of such cells.

In some embodiments, the cell is a cell for use in a method of medical treatment or prophylaxis. A cell ‘for use in a method of medical treatment or prophylaxis’ refers to a cell that is suitable for use in a method of medical treatment or prophylaxis. Such cells may be free of certain agents/contaminants that would render them unsuitable for such use.

In some embodiments, the cell is a cell for use in a method of medical treatment or prophylaxis by adoptive cell transfer (ACT). Adoptive cell transfer comprises administering a cell/population of cells to a subject in order to treat/prevent a disease/condition. In particular, adoptive cell transfer typically involves administering immune cells (e.g. T cells) to a subject, in order to provide the subject with a population of immune cells for treatment/preventing the disease/condition, or to increase the number of such cells in the subject. In some embodiments, the adoptively-transferred cells comprise a molecule for directing an activity of the immune cells against cells comprising/expressing a given target antigen, e.g. a disease-associated antigen.

Adoptive cell transfer may involve isolating/obtaining cells (e.g. immune cells) from a subject, e.g. by drawing a blood sample from which the cells are isolated. The cells are then typically modified and/or expanded, and then administered either to the same subject (in the case of adoptive transfer of autologous/autogeneic cells) or to a different subject (in the case of adoptive transfer of allogeneic cells). The treatment is typically aimed at providing a population of cells with certain desired characteristics to a subject, or increasing the frequency of such cells with such characteristics in that subject.

A cell for use in a method of medical treatment or prophylaxis by adoptive cell transfer may comprise/express a molecule for directing activity of the cell against a cell expressing a given target antigen.

In some embodiments, a cell comprises a T cell receptor (TCR) for directing activity of the cell against a cell presenting the MHC-peptide complex for which the TCR is specific. In some embodiments, the TCR is encoded by the genome of the subject from which the cell is obtained/derived. In some embodiments, the TCR is encoded by nucleic acid which has been introduced into the cell.

In some embodiments, a cell comprises a chimeric antigen receptor (CAR) for directing activity of the cell against a cell expressing the antigen for which the CAR is specific. In some embodiments, the CAR is encoded by nucleic acid which has been introduced into the cell.

In some embodiments, the immune cell is an immune cell engineered to express a molecule for directing activity of the immune cell against a cell expressing a given target antigen (e.g. a CAR-engineered immune cell or a TCR-engineered immune cell), and/or an immune cell specific for a disease-associated antigen (e.g. an immune cell specific for a pathogen, e.g. a virus-specific immune cell). In some embodiments, the immune cell is an immune cell specific for a disease-associated antigen, that has been engineered to express a molecule for directing activity of the immune cell against a cell comprising/expressing a given target antigen. In some embodiments, the immune cell is a virus-specific, CAR-engineered immune cell.

In some embodiments, the immune cell is engineered to express a chimeric antigen receptor (CAR; i.e., the immune cell is CAR-engineered immune cell), or is engineered to express a T cell receptor (TCR; i.e., the immune cell is TCR-engineered immune cell).

TCR-engineered immune cells are described e.g. in Zhao et al., Front. Immunol. (2021) 30; 12:658753, which is hereby incorporated by reference in its entirety. TCR-engineered immune cells may be modified to express a TCR specific for a given MHC-peptide complex, e.g. through introduction of nucleic acid into the cell encoding constituent polypeptide(s) of the TCR.

In some embodiments, the immune cell comprises/expresses a TCR encoded by non-endogenous nucleic acid (i.e. nucleic acid which is not encoded by the genome of the cell prior to the introduction of nucleic acid encoding constituent polypeptide(s) of the TCR into the cell).

In some embodiments, methods according to the present disclosure comprise introducing into an immune cell (i) nucleic acid encoding a molecule for directing activity of the immune cell against a cell comprising/expressing a given target antigen (e.g. a CAR or a TCR), and (ii) nucleic acid a SERPINB9 polypeptide. In accordance with such embodiments, the nucleic acids of (i) and (ii) may be introduced into the cell simultaneously or sequentially. Where the nucleic acids of (i) and (ii) are introduced simultaneously, they may be introduced in the form of a nucleic acid (e.g. a vector) comprising both of the nucleic acids of (i) and (ii). Alternatively, the nucleic acids of (i) and (ii) may be comprised in separate nucleic acids (e.g. in separate vectors). Where the nucleic acids of (i) and (ii) are introduced sequentially, the methods may comprise (a) introducing into an immune cell a nucleic acid of (i) or (ii), and (b) subsequently (e.g. after a defined period of time, e.g. after 12 hours to 14 days, e.g. one of 1 to 7 days, 2 to 5 days, or 3 to 4 days), introducing into the immune cell the other nucleic acid (i.e. the nucleic acid not introduced into the cell at (a)).

A TCR-engineered immune cell may comprise an TCR specific for any MHC-peptide complex of interest. In some embodiments, the TCR of a TCR-engineered immune cell is specific for an MHC-peptide complex comprising the peptide of a disease-associated antigen.

Through engineering to express a TCR specific for a particular MHC-peptide complex, immune cells T cells can be directed to kill cells expressing the MHC-peptide complex. Binding of a TCR-engineered T cell to its cognate MHC-peptide complex triggers intracellular signalling, and consequently activation of the T cell. The activated TCR-engineered T cell is stimulated to divide and produce factors resulting in killing of the cell expressing the MHC-peptide complex.

In some embodiments, a cell according to the present disclosure (i.e. a cell comprising/expressing a SERPINB9 as described herein, or a cell comprising/expressing nucleic acid(s)/vector(s) a SERPINB9 as described herein) does not comprise a chimeric HLA Accessory Receptor (CHAR) for directing activity of the cell against alloreactive T cells. CHARs and CHAR-expressing cells are described e.g. in US 2021/0238255 A1, which is hereby incorporated by reference in its entirety.

In some embodiments, a cell according to the present disclosure (i.e. a cell comprising/expressing a SERPINB9 as described herein, or a cell comprising/expressing nucleic acid(s)/vector(s) a SERPINB9 as described herein) has not been modified to comprise/express a CHAR. In some embodiments, a cell does not comprise nucleic acid(s) (e.g. exogenous nucleic acid(s)) encoding a CHAR. In some embodiments, methods according to the present disclosure do not comprise introducing into a cell a nucleic acid(s) (e.g. exogenous nucleic acid(s)) encoding a CHAR.

In some embodiments, a cell according to the present disclosure (i.e. a cell comprising/expressing a SERPINB9 as described herein, or a cell comprising/expressing nucleic acid(s)/vector(s) a SERPINB9 as described herein) has not been modified to express or overexpress cellular FLICE-inhibitory protein (cFLIP), or a variant thereof.

Human cFLIP is the protein identified by UniProt 015519-1. Variants of cFLIP include polypeptides having at least 70% (e.g. 80%, 85%, 90%, 91%, 92%, 93%, 94%, 95%, 96%, 97%, 98%, 99% or greater) amino acid sequence identity to the amino acid sequence of human cFLIP. In some embodiments, a variant of cFLIP may comprise the substitution K167R relative to UniProt 015519-1.

In some embodiments, a cell does not comprise nucleic acid(s) (e.g. exogenous nucleic acid(s)) encoding cFLIP or a variant thereof. In some embodiments, methods according to the present disclosure do not comprise introducing into a cell a nucleic acid(s) (e.g. exogenous nucleic acid(s)) encoding cFLIP or a variant thereof.

As used herein, a ‘disease-associated antigen’ refers to an antigen whose presence is indicative of a given disease/disease state, or an antigen for which an elevated level of the antigen is positively-correlated with a given disease/disease state. The disease-associated antigen may be an antigen whose expression is associated with the development, progression or severity of symptoms of a given disease.

The disease-associated antigen may be associated with the cause or pathology of the disease, or may be expressed abnormally as a consequence of the disease. A disease-associated antigen may be an antigen of an infectious agent or pathogen, a cancer-associated antigen or an autoimmune disease-associated antigen.

In some embodiments, the disease-associated antigen is an antigen of a pathogen. The pathogen may be prokaryotic (bacteria), eukaryotic (e.g. protozoan, helminth, fungus), virus or prion. In some embodiments, the pathogen is an intracellular pathogen. In some embodiments the pathogen is a virus, e.g. a virus as described hereinabove. In some embodiments the pathogen is a bacterium. The bacterium may be gram positive or gram negative. In particular, the present disclosure contemplates bacteria of the genera Bacillus, Bartonella, Bordetella, Borrelia, Brucella, Campylobacter, Chlamydia, and, Chlamydophila, Clostridium, Corynebacterium, Enterococcus, Escherichia, Francisella, Haemophilus, Helicobacter, Legionella, Leptospira, Listeria, Mycobacterium, Mycoplasma, Neisseria, Pseudomonas, Rickettsia, Salmonella, Shigella, Staphylococcus, Streptococcus, Treponema, Ureaplasma, Vibrio and Yersinia. In some embodiments the pathogen is protozoan. In particular, the present disclosure contemplates protozoa of the genera Entamoeba, Plasmodium, Giardia, Trypanosoma, Leishmania, Besnoitia and Toxoplasma. In some embodiments the pathogen is a fungus. In particular, the present disclosure contemplates fungi of the genera Candida, Aspergillus, Blastomyces, Coccidioides, Sporothrix, Cryptococcus, Histoplasma, Pneumocystis, Stachybotrys, Rhizopus, Mucor, Cunninghamella, Apophysomyces, Trichophyton, Microsporum, Epidermophyton, Fusarium, and Lichtheimia.

In some embodiments, the disease-associated antigen is a cancer-associated antigen. In some embodiments the cancer-associated antigen is an antigen whose expression is associated with the development, progression or severity of symptoms of a cancer. The cancer-associated antigen may be associated with the cause or pathology of the cancer, or may be expressed abnormally as a consequence of the cancer. In some embodiments, the cancer-associated antigen is an antigen whose expression is upregulated (e.g. at the RNA and/or protein level) by cells of a cancer, e.g. as compared to the level of expression of by comparable non-cancerous cells (e.g. non-cancerous cells derived from the same tissue/cell type).

In some embodiments, the cancer-associated antigen may be preferentially expressed by cancerous cells, and not expressed by comparable non-cancerous cells (e.g. non-cancerous cells derived from the same tissue/cell type). In some embodiments, the cancer-associated antigen may be the product of a mutated oncogene or mutated tumor suppressor gene. In some embodiments, the cancer-associated antigen may be the product of an overexpressed cellular protein, a cancer antigen produced by an oncogenic virus, an oncofetal antigen, or a cell surface glycolipid or glycoprotein.

Cancer-associated antigens are reviewed by Zarour H M, DeLeo A, Finn O J, et al. Categories of Tumor Antigens. In: Kufe D W, Pollock R E, Weichselbaum R R, et al., editors. Holland-Frei Cancer Medicine. 6th edition. Hamilton (ON): BC Decker; 2003. Cancer-associated antigens include oncofetal antigens: CEA, Immature laminin receptor, TAG-72; oncoviral antigens such as HPV E6 and E7; overexpressed proteins: BING-4, calcium-activated chloride channel 2, cyclin-B1, 9D7, Ep-CAM, EphA3, HER2/neu, telomerase, mesothelin, SAP-1, survivin; cancer-testis antigens: BAGE, CAGE, GAGE, MAGE, SAGE, XAGE, CT9, CT10, NY-ESO-1, PRAME, SSX-2; lineage restricted antigens: MART1, Gp100, tyrosinase, TRP-1/2, MC1R, prostate specific antigen; mutated antigens: β-catenin, BRCA1/2, CDK4, CML66, Fibronectin, MART-2, p53, Ras, TGF-βRII; post-translationally altered antigens: MUC1, idiotypic antigens: Ig, TCR. Other cancer-associated antigens include heat-shock protein 70 (HSP70), heat-shock protein 90 (HSP90), glucose-regulated protein 78 (GRP78), vimentin, nucleolin, feto-acinar pancreatic protein (FAPP), alkaline phosphatase placental-like 2 (ALPPL-2), siglec-5, stress-induced phosphoprotein 1 (STIP1), protein tyrosine kinase 7 (PTK7), and cyclophilin B. In some embodiments the cancer-associated antigen is a cancer-associated antigen described in Zhao and Cao, Front Immunol. 2019; 10: 2250, which is hereby incorporated by reference in its entirety. In some embodiments, a cancer-associated antigen is selected from CD30, CD19, CD20, CD22, B7H3, c-Met, ROR1R, CD4, CD7, CD38, BCMA, Mesothelin, EGFR, GPC3, MUC1, HER2, GD2, CEA, EpCAM, LeY and PSCA. In some embodiments, a cancer-associated antigen is an antigen expressed by cells of a hematological malignancy. In some embodiments, a cancer-associated antigen is selected from CD30, CD19, CD20, CD22, B7H3, c-Met, ROR1R, CD4, CD7, CD38 and BCMA. In some embodiments, a cancer-associated antigen is an antigen expressed by cells of a solid tumor. In some embodiments, a cancer-associated antigen is selected from Mesothelin, EGFR, GPC3, MUC1, HER2, GD2, CEA, EpCAM, LeY and PSCA.

In some embodiments, the immune cell is a chimeric antigen receptor (CAR)-engineered immune cell. In some embodiments, the immune cell comprises/expresses a CAR. Chimeric Antigen Receptors (CARs) are recombinant receptor molecules which provide both antigen-binding and T cell activating functions. CAR structure and engineering is reviewed, for example, in Dotti et al., Immunol Rev (2014) 257(1), which is hereby incorporated by reference in its entirety.

CAR-expressing immune cells may comprise or express nucleic acid encoding a CAR according to the present disclosure. It will be appreciated that a CAR-expressing cell comprises the CAR it expresses. It will also be appreciated that a cell expressing nucleic acid encoding a CAR also expresses and comprises the CAR encoded by the nucleic acid.

CARs comprise an antigen-binding domain linked via a transmembrane domain to a signalling domain. An optional hinge or spacer domain may provide separation between the antigen-binding domain and transmembrane domain, and may act as a flexible linker. When expressed by a cell, the antigen-binding domain is provided in the extracellular space, and the signalling domain is intracellular.

Through engineering to express a CAR specific for a particular target antigen, immune cells (typically T cells, but also other immune cells such as NK cells) can be directed to kill cells expressing the target antigen. Binding of a CAR-expressing T cell (CAR-T cell) to the target antigen for which it is specific triggers intracellular signalling, and consequently activation of the T cell. The activated CAR-T cell is stimulated to divide and produce factors resulting in killing of the cell expressing the target antigen.

The antigen-binding domain mediates binding to the target antigen for which the CAR is specific. An ‘antigen-binding domain’ refers to a domain which is capable of binding to a target antigen. The antigen-binding domain of a CAR may be based on the antigen-binding region of an antibody which is specific for the antigen to which the CAR is targeted. For example, the antigen-binding domain of a CAR may comprise amino acid sequences for the complementarity-determining regions (CDRs) of an antibody which binds specifically to the target antigen. The antigen-binding domain of a CAR may comprise or consist of the light chain and heavy chain variable region amino acid sequences of an antibody which binds specifically to the target antigen. The antigen-binding domain may be provided as a single chain variable fragment (scFv) comprising the sequences of the light chain and heavy chain variable region amino acid sequences of an antibody. Antigen-binding domains of CARs may target antigen based on other protein:protein interaction, such as ligand:receptor binding; for example an IL-13Ra2-targeted CAR has been developed using an antigen-binding domain based on IL-13 (see e.g. Kahlon et al. 2004 Cancer Res 64(24): 9160-9166).

The antigen-binding domain of a CAR according to the present disclosure may be specific for any antigen, e.g. a disease-associated antigen as described herein. It will be appreciated that the antigen-binding domain of a CAR is specific for an antigen expressed by cells against which it is desired to direct the activity of the CAR-expressing cell. In the example of a CAR-expressing T cell, the CAR may comprise an antigen-binding domain that binds to an antigen expressed by a target cell to against which it is desired to direct T cell effector activity. In preferred embodiments, the antigen-binding domain of a CAR according to the present disclosure binds to a cancer-associated antigen. In further preferred embodiments, the antigen-binding domain of a CAR according to the present disclosure binds to CD30.

Antigen-binding domains according to the present disclosure may be derived from an antibody/antibody fragment (e.g. Fv, scFv, Fab, single chain Fab (scFab), single domain antibodies (e.g. VhH), etc.) directed against a disease-associated antigen, or another disease-associated antigen-binding molecule (e.g. a target antigen-binding peptide or nucleic acid aptamer, ligand or other molecule). In some embodiments, the antigen-binding domain comprises an antibody heavy chain variable region (VH) and an antibody light chain variable region (VL) of an antibody capable of specific binding to a disease-associated antigen. In some embodiments, the domain capable of binding to a target antigen comprises or consists of a disease-associated antigen-binding peptide/polypeptide, e.g. a peptide aptamer, thioredoxin, monobody, anticalin, Kunitz domain, avimer, knottin, fynomer, atrimer, DARPin, affibody, nanobody (i.e. a single-domain antibody (sdAb)) affilin, armadillo repeat protein (ArmRP), OBody or fibronectin—reviewed e.g. in Reverdatto et al., Curr Top Med Chem. 2015; 15(12): 1082-1101, which is hereby incorporated by reference in its entirety (see also e.g. Boersma et al., J Biol Chem (2011) 286:41273-85 and Emanuel et al., Mabs (2011) 3:38-48).

The antigen-binding domain of a CAR of the present disclosure may be derived from the VH and a VL of an antibody capable of specific binding to a disease-associated antigen. Antibodies generally comprise six complementarity-determining regions CDRs; three in the heavy chain variable region (VH): HC-CDR1, HC-CDR2 and HC-CDR3, and three in the light chain variable region (VL): LC-CDR1, LC-CDR2, and LC-CDR3. The six CDRs together define the paratope of the antibody, which is the part of the antibody that binds to the target antigen. The VH region and VL region comprise framework regions (FRs) either side of each CDR, which provide a scaffold for the CDRs. From N-terminus to C-terminus, VHs comprise the following structure: N term-[HC-FR1]-[HC-CDR1]-[HC-FR2]-[HC-CDR2]-[HC-FR3]-[HC-CDR3]-[HC-FR4]-C term; and VLs comprise the following structure: N term-[LC-FR1]-[LC-CDR1]-[LC-FR2]-[LC-CDR2]-[LC-FR3]-[LC-CDR3]-[LC-FR4]-C term.

VH and VL sequences may be provided in any suitable format provided that the antigen-binding domain can be linked to the other domains of the CAR. Formats contemplated in connection with the antigen-binding domain of the present disclosure include those described in Carter, Nat. Rev. Immunol 2006, 6: 343-357, such as scFv, dsFV, (scFv)2 diabody, triabody, tetrabody, Fab, minibody, and F(ab)2 formats.

In some embodiments, the antigen-binding domain comprises the CDRs of an antibody/antibody fragment which is capable of binding to a disease-associated antigen. In some embodiments, the antigen-binding domain comprises the VH region and the VL region of an antibody/antibody fragment which is capable of binding to a disease-associated antigen. A moiety comprised of the VH and a VL of an antibody may also be referred to herein as a variable fragment (Fv). The VH and VL may be provided on the same polypeptide chain, and joined via a linker sequence; such moieties are referred to as single-chain variable fragments (scFvs). Suitable linker sequences for the preparation of scFv are known to the skilled person, and may comprise serine and glycine residues. In some embodiments, the antigen-binding domain comprises, or consists of, Fv capable of binding to a disease-associated antigen. In some embodiments, the antigen-binding domain comprises, or consists of, a scFv capable of binding to a disease-associated antigen. In some embodiments, the antigen-binding domain is derived from a ligand for the disease-associated antigen.

In some embodiments, the antigen-binding domain of a CAR according to the present disclosure binds to CD30 (i.e. is a CD30-binding domain). In some embodiments, the antigen-binding domain of a CAR according to the present disclosure binds to CD19 (i.e. is a CD19-binding domain).

In some embodiments, the CD30-binding domain is derived from the antigen-binding moiety of an anti-CD30 antibody. In some embodiments a CD30-binding domain according to the present disclosure comprises the CDRs of an anti-CD30 antibody. In some embodiments a CD30-binding domain according to the present disclosure comprises the VH and VL regions of an anti-CD30 antibody. In some embodiments a CD30-binding domain according to the present disclosure comprises an scFv comprising the VH and VL regions of an anti-CD30 antibody. Anti-CD30 antibodies include HRS3 and HRS4 (described e.g. in Hombach et al., Scand J Immunol. (1998) 48(5):497-501), HRS3 derivatives described in Schlapschy et al., Protein Engineering, Design and Selection (2004) 17(12): 847-860, BerH2 (MBL International Cat #K0145-3, RRID:AB 590975), SGN-30 (also known as cAC10, described e.g. in Forero-Torres et al., Br J Haematol (2009) 146:171-9), MDX-060 (described e.g. in Ansell et al., J Clin Oncol (2007) 25:2764-9; also known as 5F11, iratumumab), and MDX-1401 (described e.g. in Cardarelli et al., Clin Cancer Res. (2009) 15(10):3376-83), and anti-CD30 antibodies described in WO 2020/068764 A1, WO 2003/059282 A2, WO 2006/089232 A2, WO 2007/084672 A2, WO 2007/044616 A2, WO 2005/001038 A2, US 2007/166309 A1, US 2007/258987 A1, WO 2004/010957 A2 and US 2005/009769 A1.

In some embodiments, the CD19-binding domain is derived from the antigen-binding moiety of an anti-CD19 antibody. In some embodiments a CD19-binding domain according to the present disclosure comprises the CDRs of an anti-CD19 antibody. In some embodiments a CD19-binding domain according to the present disclosure comprises the VH and VL regions of an anti-CD19 antibody. In some embodiments a CD19-binding domain according to the present disclosure comprises an scFv comprising the VH and VL regions of an anti-CD19 antibody. Anti-CD19 antibodies include FMC63 (described e.g. in Zola et al., Immunol Cell Biol (1991) 69:411-422), HD37 (described in e.g. Pezzutto et al., J Immunol (1987) 138:2793-2799), tafasitamab (also known as MOR208 or XmAB5574) and anti-CD19 antibodies described in WO 2009/054863 A2, WO 2010/053716 A1, WO 2006/089133 A2 and WO 2021/173471 A1. There are several different conventions for defining antibody CDRs and FRs, such as those described in Kabat et al., Sequences of Proteins of Immunological Interest, 5th Ed. Public Health Service, National Institutes of Health, Bethesda, MD (1991), Chothia et al., J. Mol. Biol. 196:901-917 (1987), and VBASE2, as described in Retter et al., Nucl. Acids Res. (2005) 33 (suppl 1): D671-D674. The CDRs and FRs of the VH regions and VL regions of the antibodies described herein are defined according to VBASE2.

In some embodiments the CD30-binding domain comprises:
a VH incorporating the following CDRs:
   HC-CDR1 having the amino acid sequence of SEQ ID NO:15
   HC-CDR2 having the amino acid sequence of SEQ ID NO:16
   HC-CDR3 having the amino acid sequence of SEQ ID NO:17,
or a variant thereof in which one or two or three amino acids in one or more of HC-CDR1, HC-CDR2, or HC-CDR3 are substituted with another amino acid;
and
a VL incorporating the following CDRs:
   LC-CDR1 having the amino acid sequence of SEQ ID NO:18
   LC-CDR2 having the amino acid sequence of SEQ ID NO:19
   LC-CDR3 having the amino acid sequence of SEQ ID NO:20,
or a variant thereof in which one or two or three amino acids in one or more of LC-CDR1, LC-CDR2, or LC-CDR3 are substituted with another amino acid.

In some embodiments the CD30-binding domain comprises:
a VH comprising, or consisting of, an amino acid sequence having at least 80% sequence identity (e.g. at least 85%, 86%, 87%, 88%, 89%, 90%, 91%, 92%, 93%, 94%, 95%, 96%, 97%, 98%, 99% or 100%) to the amino acid sequence of SEQ ID NO:21;
   and
a VL comprising, or consisting of, an amino acid sequence having at least 80% sequence identity (e.g. at least 85%, 86%, 87%, 88%, 89%, 90%, 91%, 92%, 93%, 94%, 95%, 96%, 97%, 98%, 99% or 100%) to the amino acid sequence of SEQ ID NO:22.

In some embodiments, the CD30-binding domain may comprise or consist of a single chain variable fragment (scFv) comprising a VH sequence and a VL sequence as described herein. The VH sequence and VL sequence may be covalently linked. In some embodiments, the VH and the VL sequences are linked by a flexible linker sequence, e.g. a flexible linker sequence as described herein. The flexible linker sequence may be joined to ends of the VH sequence and VL sequence, thereby linking the VH and VL sequences. In some embodiments the VH and VL are joined via a linker sequence comprising, or consisting of, the amino acid sequence of SEQ ID NO:23.

In some embodiments the CD30-binding domain comprises, or consists of, an amino acid sequence having at least 80%, 85% 86%, 87%, 88%, 89%, 90%, 91%, 92%, 93%, 94%, 95%, 96%, 97%, 98%, 99%, or 100% sequence identity to the amino acid sequence of SEQ ID NO:24.

In some embodiments the CD30-binding domain is capable of binding to CD30, e.g. in the extracellular domain of CD30. In some embodiments, the CD30-binding domain is capable of binding to the epitope of CD30 which is bound by antibody HRS3, e.g. within the region of amino acid positions 185-335 of human CD30 numbered according to SEQ ID NO:10, shown in SEQ ID NO:12 (Schlapschy et al., Protein Engineering, Design and Selection (2004) 17(12): 847-860, hereby incorporated by reference in its entirety).

In some embodiments the CD19-binding domain comprises:
a VH incorporating the following CDRs:
   HC-CDR1 having the amino acid sequence of SEQ ID NO:58
   HC-CDR2 having the amino acid sequence of SEQ ID NO:59
   HC-CDR3 having the amino acid sequence of SEQ ID NO:60,
or a variant thereof in which one or two or three amino acids in one or more of HC-CDR1, HC-CDR2, or HC-CDR3 are substituted with another amino acid;
and
a VL incorporating the following CDRs:
   LC-CDR1 having the amino acid sequence of SEQ ID NO:61
   LC-CDR2 having the amino acid sequence of SEQ ID NO:62
   LC-CDR3 having the amino acid sequence of SEQ ID NO:63,
or a variant thereof in which one or two or three amino acids in one or more of LC-CDR1, LC-CDR2, or LC-CDR3 are substituted with another amino acid.

In some embodiments the CD19-binding domain comprises:
a VH comprising, or consisting of, an amino acid sequence having at least 80% sequence identity (e.g. at least 85%, 86%, 87%, 88%, 89%, 90%, 91%, 92%, 93%, 94%, 95%, 96%, 97%, 98%, 99% or 100%) to the amino acid sequence of SEQ ID NO:64:
   and
a VL comprising, or consisting of, an amino acid sequence having at least 80% sequence identity (e.g. at least 85%, 86%, 87%, 88%, 89%, 90%, 91%, 92%, 93%, 94%, 95%, 96%, 97%, 98%, 99% or 100%) to the amino acid sequence of SEQ ID NO:65.

In some embodiments, the CD19-binding domain may comprise or consist of a single chain variable fragment (scFv) comprising a VH sequence and a VL sequence as described herein. The VH sequence and VL sequence may be covalently linked. In some embodiments, the VH and the VL sequences are linked by a flexible linker sequence, e.g. a flexible linker sequence as described herein. The flexible linker sequence may be joined to ends of the VH sequence and VL sequence, thereby linking the VH and VL sequences. In some embodiments the VH and VL are joined via a linker sequence comprising, or consisting of, the amino acid sequence of SEQ ID NO:66.

In some embodiments the CD19-binding domain comprises, or consists of, an amino acid sequence having at least 80%, 85% 86%, 87%, 88%, 89%, 90%, 91%, 92%, 93%, 94%, 95%, 96%, 97%, 98%, 99%, or 100% sequence identity to the amino acid sequence of SEQ ID NO:67.

In some embodiments the CD19-binding domain is capable of binding to CD19, e.g. in the extracellular domain of CD19.

In some embodiments, the antigen-binding domain (and thus the CAR) is multispecific. By ‘multispecific’ it is meant that the antigen-binding domain displays specific binding to more than one target. In some embodiments the antigen-binding domain is a bispecific antigen-binding domain. In some embodiments the antigen-binding molecule comprises at least two different antigen-binding moieties (i.e. at least two antigen-binding moieties, e.g. comprising non-identical VHs and VLs). Individual antigen-binding moieties of multispecific antigen-binding domains may be connected, e.g. via linker sequences. The antigen-binding domain may bind to at least two, non-identical target antigens, and so is at least bispecific. The term ‘bispecific’ means that the antigen-binding domain is able to bind specifically to at least two distinct antigenic determinants. At least one of the target antigens for the multispecific antigen-binding domain/CAR may be CD30. At least one of the target antigens for the multispecific antigen-binding domain/CAR may be CD19. It will be appreciated that an antigen-binding domain according to the present disclosure (e.g. a multispecific antigen-binding domain) comprises antigen-binding moieties capable of binding to the target(s) for which the antigen-binding domain is specific. For example, an antigen-binding domain which is capable of binding to CD30 and an antigen other than CD30 may comprise: (i) an antigen-binding moiety which is capable of binding to CD30, and (ii) an antigen-binding moiety which is capable of binding to a target antigen other than CD30. In a further example, an antigen-binding domain which is capable of binding to CD19 and an antigen other than CD19 may comprise: (i) an antigen-binding moiety which is capable of binding to CD19, and (ii) an antigen-binding moiety which is capable of binding to a target antigen other than CD19.

CARs according to the present disclosure comprise a transmembrane domain. A transmembrane domain refers to any three-dimensional structure formed by a sequence of amino acids which is thermodynamically stable in a biological membrane, e.g. a cell membrane. In connection with the present disclosure, the transmembrane domain may be an amino acid sequence which spans the cell membrane of a cell expressing the CAR. The transmembrane domain of a CAR is provided between the antigen-binding domain and the signalling domain of the CAR. The transmembrane domain provides for anchoring the CAR to the cell membrane of a cell expressing a CAR, with the antigen-binding domain in the extracellular space, and signalling domain inside the cell. Transmembrane domains of CARs may be derived from transmembrane region sequences for cell membrane-bound proteins (e.g. CD28, CD8, etc.).

Throughout this specification, polypeptides, domains and amino acid sequences which are ‘derived from’ a reference polypeptide/domain/amino acid sequence have at least 60%, preferably one of 70%, 75%, 80%, 85%, 90%, 91%, 92%, 93%, 94%, 95%, 96%, 97%, 98%, 99% or 100% amino acid sequence identity to the amino acid sequence of the reference polypeptide/domain/amino acid sequence. Polypeptides, domains and amino acid sequences which are ‘derived from’ a reference polypeptide/domain/amino acid sequence preferably retain the functional and/or structural properties of the reference polypeptide/domain/amino acid sequence.

By way of illustration, an amino acid sequence derived from the intracellular domain of CD28 may comprise an amino acid sequence having 60%, preferably one of 70%, 75%, 80%, 85%, 90%, 91%, 92%, 93%, 94%, 95%, 96%, 97%, 98%, 99% or 100% amino acid sequence identity to the intracellular domain of CD28, e.g. as shown in SEQ ID NO:32. Furthermore, an amino acid sequence derived from the intracellular domain of CD28 preferably retains the functional properties of the amino acid sequence of SEQ ID NO:32, i.e. the ability activate CD28-mediated signalling.

The amino acid sequence of a given polypeptide or domain thereof can be retrieved from, or determined from a nucleic acid sequence retrieved from, databases known to the person skilled in the art. Such databases include GenBank, EMBL and UniProt.

The transmembrane domain may comprise or consist of a sequence of amino acids which forms a hydrophobic alpha helix or beta-barrel. The amino acid sequence of the transmembrane domain of the CAR of the present disclosure may be, or may be derived from, the amino acid sequence of a transmembrane domain of a protein comprising a transmembrane domain. Transmembrane domains are recorded in databases such as GenBank, UniProt, Swiss-Prot, TrEMBL, Protein Information Resource, Protein Data Bank, Ensembl, and InterPro, and/or can be identified/predicted e.g. using amino acid sequence analysis tools such as TMHMM (Krogh et al., 2001 J Mol Biol 305: 567-580).

In some embodiments, the amino acid sequence of the transmembrane domain of the CAR of the present disclosure may be, or may be derived from, the amino acid sequence of the transmembrane domain of a protein expressed at the cell surface. In some embodiments the protein expressed at the cell surface is a receptor or ligand, e.g. an immune receptor or ligand. In some embodiments the amino acid sequence of the transmembrane domain may be, or may be derived from, the amino acid sequence of the transmembrane domain of one of ICOS, ICOSL, CD86, CTLA-4, CD28, CD80, MHC class I a, MHC class II α, MHC class II β, CD3ε, CD3δ, CD3γ, CD3-ζ, TCRα TCRβ, CD4, CD8α, CD8β, CD40, CD40L, PD-1, PD-L1, PD-L2, 4-1BB, 4-1BBL, OX40, OX40L, GITR, GITRL, TIM-3, Galectin 9, LAG3, CD27, CD70, LIGHT, HVEM, TIM-4, TIM-1, ICAM1, LFA-1, LFA-3, CD2, BTLA, CD160, LILRB4, LILRB2, VTCN1, CD2, CD48, 2B4, SLAM, CD30, CD30L, DR3, TL1A, CD226, CD155, CD112 and CD276. In some embodiments, the transmembrane is, or is derived from, the amino acid sequence of the transmembrane domain of CD28, CD3-ζ, CD8α, CD8β or CD4. In some embodiments, the transmembrane is, or is derived from, the amino acid sequence of the transmembrane domain of CD28.

In some embodiments, the transmembrane domain comprises, or consists of, an amino acid sequence having at least 80%, 85% 86%, 87%, 88%, 89%, 90%, 91%, 92%, 93%, 94%, 95%, 96%, 97%, 98%, 99%, or 100% sequence identity to the amino acid sequence of SEQ ID NO:26.

In some embodiments, the transmembrane domain comprises, or consists of, an amino acid sequence having at least 80%, 85% 86%, 87%, 88%, 89%, 90%, 91%, 92%, 93%, 94%, 95%, 96%, 97%, 98%, 99%, or 100% sequence identity to the amino acid sequence of SEQ ID NO:27.

In some embodiments, the transmembrane domain comprises, or consists of, an amino acid sequence having at least 80%, 85% 86%, 87%, 88%, 89%, 90%, 91%, 92%, 93%, 94%, 95%, 96%, 97%, 98%, 99%, or 100% sequence identity to the amino acid sequence of SEQ ID NO:28.

The chimeric antigen receptor of the present disclosure comprises a signalling domain. The signalling domain provides sequences for initiating intracellular signalling in cells expressing the CAR. The signalling domain comprises amino acid sequences required activation of immune cell function. The CAR signalling domains may comprise the amino acid sequence of the intracellular domain of CD3-ζ, which provides immunoreceptor tyrosine-based activation motifs (ITAMs) for phosphorylation and activation of the CAR-expressing cell. Signalling domains comprising sequences of other ITAM-containing proteins have also been employed in CARs, such as domains comprising the ITAM containing region of FcγRI (Haynes et al., 2001 J Immunol 166(1):182-187). CARs comprising a signalling domain derived from the intracellular domain of CD3-ζ are often referred to as first generation CARs.

The signalling domains of CARs typically also comprise the signalling domain of a costimulatory protein (e.g. CD28, 4-1BB etc.), for providing the costimulation signal necessary for enhancing immune cell activation and effector function. CARs having a signalling domain including additional co-stimulatory sequences are often referred to as second generation CARs. In some cases CARs are engineered to provide for co-stimulation of different intracellular signalling pathways. For example, CD28 costimulation preferentially activates the phosphatidylinositol 3-kinase (P13K) pathway, whereas 4-1BB costimulation triggers signalling is through TNF receptor associated factor (TRAF) adaptor proteins. Signalling domains of CARs therefore sometimes contain co-stimulatory sequences derived from signalling domains of more than one co-stimulatory molecule. CARs comprising a signalling domain with multiple co-stimulatory sequences are often referred to as third generation CARs.

The signalling domain comprises an ITAM-containing sequence. An ITAM-containing sequence comprises one or more immunoreceptor tyrosine-based activation motifs (ITAMs). ITAMs comprise the amino acid sequence YXXL/I (SEQ ID NO:29), wherein ‘X’ denotes any amino acid. In ITAM-containing proteins, sequences according to SEQ ID NO:29 are often separated by 6 to 8 amino acids; YXXL/I(X)₅₋ₐYXXL/I (SEQ ID NO:30). When phosphate groups are added to the tyrosine residue of an ITAM by tyrosine kinases, a signalling cascade is initiated within the cell. In some embodiments, the signalling domain comprises one or more copies of an amino acid sequence according to SEQ ID NO:29 or SEQ ID NO:30. In some embodiments, the signalling domain comprises at least 1, 2, 3, 4, 5 or 6 copies of an amino acid sequence according to SEQ ID NO:30. In some embodiments, the signalling domain comprises at least 1, 2, or 3 copies of an amino acid sequence according to SEQ ID NO:30.

In some embodiments, the signalling domain comprises an amino acid sequence which is, or which is derived from, the amino acid sequence of an ITAM-containing sequence of a protein having an ITAM-containing amino acid sequence. In some embodiments the signalling domain comprises an amino acid sequence which is, or which is derived from, the amino acid sequence of the intracellular domain of one of CD3-ζ, FcγRI, CD3ε, CD3δ, CD3γ, CD79α, CD79β, FcγRIIA, FcγRIIC, FcγRIIIA, FcγRIV or DAP12. In some embodiments the signalling domain comprises an amino acid sequence which is, or which is derived from, the intracellular domain of CD3-ζ. In some embodiments, the signalling domain comprises an amino acid sequence which comprises, or consists of, an amino acid sequence having at least 80%, 85% 86%, 87%, 88%, 89%, 90%, 91%, 92%, 93%, 94%, 95%, 96%, 97%, 98%, 99%, or 100% sequence identity to the amino acid sequence of SEQ ID NO:31.

The signalling domain may additionally comprise one or more costimulatory sequences. A costimulatory sequence is an amino acid sequence which provides for costimulation of the cell expressing the CAR of the present disclosure. Costimulation promotes proliferation and survival of a CAR-expressing cell upon binding to the target antigen, and may also promote cytokine production, differentiation, cytotoxic function and memory formation by the CAR-expressing cell. Molecular mechanisms of T cell costimulation are reviewed in Chen and Flies, 2013 Nat Rev Immunol 13(4):227-242.

A costimulatory sequence may be, or may be derived from, the amino acid sequence of a costimulatory protein. In some embodiments the costimulatory sequence is an amino acid sequence which is, or which is derived from, the amino acid sequence of the intracellular domain of a costimulatory protein. Upon binding of the CAR to the target antigen, the costimulatory sequence provides costimulation to the cell expressing the CAR costimulation of the kind which would be provided by the costimulatory protein from which the costimulatory sequence is derived upon ligation by its cognate ligand. By way of example in the case of a CAR comprising a signalling domain comprising a costimulatory sequence derived from CD28, binding to the target antigen triggers signalling in the cell expressing the CAR of the kind that would be triggered by binding of CD80 and/or CD86 to CD28. Thus a costimulatory sequence is capable of delivering the costimulation signal of the costimulatory protein from which the costimulatory sequence is derived.

In some embodiments, the costimulatory protein may be a member of the B7-CD28 superfamily (e.g. CD28, ICOS), or a member of the TNF receptor superfamily (e.g. 4-1BB, OX40, CD27, DR3, GITR, CD30, HVEM). In some embodiments, the costimulatory sequence is, or is derived from, the intracellular domain of one of CD28, 4-1BB, ICOS, CD27, OX40, HVEM, CD2, SLAM, TIM-1, CD30, GITR, DR3, CD226 and LIGHT. In some embodiments, the costimulatory sequence is, or is derived from, the intracellular domain of CD28.

In some embodiments the signalling domain comprises more than one non-overlapping costimulatory sequences. In some embodiments the signalling domain comprises 1, 2, 3, 4, 5 or 6 costimulatory sequences. Plural costimulatory sequences may be provided in tandem.

Whether a given amino acid sequence is capable of initiating signalling mediated by a given costimulatory protein can be investigated e.g. by analysing a correlate of signalling mediated by the costimulatory protein (e.g. expression/activity of a factor whose expression/activity is upregulated or downregulated as a consequence of signalling mediated by the costimulatory protein).

Costimulatory proteins upregulate expression of genes promoting cell growth, effector function and survival through several transduction pathways. For example, CD28 and ICOS signal through phosphatidylinositol 3 kinase (PI3K) and AKT to upregulate expression of genes promoting cell growth, effector function and survival through NF-κB, mTOR, NFAT and AP1/2. CD28 also activates AP1/2 via CDC42/RAC1 and ERK1/2 via RAS, and ICOS activates C-MAF. 4-1BB, OX40, and CD27 recruit TNF receptor associated factor (TRAF) and signal through MAPK pathways, as well as through PI3K.

In some embodiments the signalling domain comprises a costimulatory sequence which is, or which is derived from CD28. In some embodiments, the signalling domain comprises a costimulatory sequence which comprises, or consists of, an amino acid sequence having at least 80%, 85% 86%, 87%, 88%, 89%, 90%, 91%, 92%, 93%, 94%, 95%, 96%, 97%, 98%, 99%, or 100% sequence identity to the amino acid sequence of SEQ ID NO:32.

Kofler et al. Mol. Ther. (2011) 19: 760-767 describes a variant CD28 intracellular domain in which the Ick kinase binding site is mutated in order to reduce induction of IL-2 production on CAR ligation, in order to minimise regulatory T cell-mediated suppression of CAR-T cell activity. The amino acid sequence of the variant CD28 intracellular domain is shown in SEQ ID NO:33. In some embodiments, the signalling domain comprises a costimulatory sequence which comprises, or consists of, an amino acid sequence having at least 80%, 85% 86%, 87%, 88%, 89%, 90%, 91%, 92%, 93%, 94%, 95%, 96%, 97%, 98%, 99%, or 100% sequence identity to the amino acid sequence of SEQ ID NO:33.

In some embodiments, the signalling domain comprises, or consists of, an amino acid sequence having at least 80%, 85% 86%, 87%, 88%, 89%, 90%, 91%, 92%, 93%, 94%, 95%, 96%, 97%, 98%, 99%, or 100% sequence identity to the amino acid sequence of SEQ ID NO:34.

The chimeric antigen receptor of the present disclosure may comprise a hinge or spacer region. The hinge/spacer region may provide separation between the antigen-binding domain and the transmembrane domain, and may act as a flexible linker. Such regions may be or comprise flexible domains allowing the binding moiety to orient in different directions, and may e.g. be derived from the CH1-CH2 hinge region of IgG. The presence, absence and length of hinge regions has been shown to influence CAR function (reviewed e.g. in Dotti et al., Immunol Rev (2014) 257(1) supra).

In some embodiments, the CAR comprises a hinge region which comprises, or consists of, an amino acid sequence which is, or which is derived from, the CH1-CH2 hinge region of human IgG1, a hinge region derived from CD8α, e.g. as described in WO 2012/031744 A1, or a hinge region derived from CD28, e.g. as described in WO 2011/041093 A1. In some embodiments, the CAR comprises a hinge region derived from the CH1-CH2 hinge region of human IgG1. In some embodiments, the hinge region comprises, or consists of, an amino acid sequence having at least 80%, 85% 86%, 87%, 88%, 89%, 90%, 91%, 92%, 93%, 94%, 95%, 96%, 97%, 98%, 99%, or 100% sequence identity to the amino acid sequence of SEQ ID NO:35 or 36.

In some embodiments, the CAR comprises a hinge region which comprises, or consists of: an amino acid sequence which is, or which is derived from, the CH2-CH3 region (i.e. the Fc region) of human IgG1. In some embodiments, the hinge region comprises, or consists of, an amino acid sequence having at least 80%, 85% 86%, 87%, 88%, 89%, 90%, 91%, 92%, 93%, 94%, 95%, 96%, 97%, 98%, 99%, or 100% sequence identity to the amino acid sequence of SEQ ID NO:37.

Hombach et al., Gene Therapy (2010) 17:1206-1213 describes a variant CH2-CH3 region for reduced activation of FcγR-expressing cells such as monocytes and NK cells. The amino acid sequence of the variant CH2-CH3 region is shown in SEQ ID NO:38. In some embodiments, the hinge region comprises, or consists of, an amino acid sequence having at least 80%, 85% 86%, 87%, 88%, 89%, 90%, 91%, 92%, 93%, 94%, 95%, 96%, 97%, 98%, 99%, or 100% sequence identity to the amino acid sequence of SEQ ID NO:38.

In some embodiments, the hinge region comprises, or consists of: an amino acid sequence which is, or which is derived from, the CH1-CH2 hinge region of human IgG1, and an amino acid sequence which is, or which is derived from, the CH2-CH3 region (i.e. the Fc region) of human IgG1. In some embodiments, the hinge region comprises, or consists of, an amino acid sequence having at least 80%, 85% 86%, 87%, 88%, 89%, 90%, 91%, 92%, 93%, 94%, 95%, 96%, 97%, 98%, 99%, or 100% sequence identity to the amino acid sequence of SEQ ID NO:39.

CARs according to the present disclosure may comprise additional amino acid sequence(s), i.e. in addition to those amino acid sequences forming the antigen-binding domain, the optional hinge/spacer region, the transmembrane domain and the signalling domain. The CAR may additionally comprise a signal peptide (also known as a leader sequence or signal sequence). Signal peptides normally consist of a sequence of 5-30 hydrophobic amino acids, which form a single alpha helix. Secreted proteins and proteins expressed at the cell surface often comprise signal peptides. Signal peptides are known for many proteins, and are recorded in databases such as GenBank, UniProt and Ensembl, and/or can be identified/predicted e.g. using amino acid sequence analysis tools such as SignalP (Petersen et al., 2011 Nature Methods 8: 785-786) or Signal-BLAST (Frank and Sippl, 2008 Bioinformatics 24: 2172-2176).

The signal peptide may be present at the N-terminus of the CAR, and may be present in the newly synthesised CAR. The signal peptide provides for efficient trafficking of the CAR to the cell surface. Signal peptides are removed by cleavage, and thus are not comprised in the mature CAR expressed by the cell surface.

In some embodiments, the signal peptide comprises, or consists of, an amino acid sequence having at least 80%, 85% 86%, 87%, 88%, 89%, 90%, 91%, 92%, 93%, 94%, 95%, 96%, 97%, 98%, 99%, or 100% sequence identity to the amino acid sequence of SEQ ID NO:40.

In some embodiments the CAR comprises one or more linker sequences between the different domains (i.e. the antigen-binding domain, hinge region, transmembrane domain, signalling domain). In some embodiments the CAR comprises one or more linker sequences between subsequences of the domains (e.g. between VH and VL of an antigen-binding domain).

Linker sequences are known to the skilled person, and are described, for example in Chen et al., Adv Drug Deliv Rev (2013) 65(10): 1357-1369, which is hereby incorporated by reference in its entirety. In some embodiments, a linker sequence may be a flexible linker sequence. Flexible linker sequences allow for relative movement of the amino acid sequences which are linked by the linker sequence. Flexible linkers are known to the skilled person, and several are identified in Chen et al., Adv Drug Deliv Rev (2013) 65(10): 1357-1369. Flexible linker sequences often comprise high proportions of glycine and/or serine residues. In some embodiments, the linker sequence comprises at least one glycine residue and/or at least one serine residue. In some embodiments the linker sequence consists of glycine and/or serine residues. In some embodiments, the linker sequence comprises at least one glycine residue and/or at least one serine residue. In some embodiments, the linker sequence comprises or consists of glycine and serine residues. In some embodiments, the linker sequence comprises one or more (e.g. 1, 2, 3, 4, 5 or 6) copies (e.g. in tandem) of the sequence motif G₃S. In some embodiments, the linker sequence comprises one or more (e.g. 1, 2, 3, 4, 5 or 6) copies (e.g. in tandem) of the sequence motif G₄S. In some embodiments, the linker sequence has a length of 1-2, 1-3, 1-4, 1-5, 1-10, 1-15, 1-20, 1-25, or 1-30 amino acids. In some embodiments, the signal peptide comprises, or consists of, an amino acid sequence having at least 80%, 85% 86%, 87%, 88%, 89%, 90%, 91%, 92%, 93%, 94%, 95%, 96%, 97%, 98%, 99%, or 100% sequence identity to the amino acid sequence of SEQ ID NO:23.

In some embodiments, a CAR of the present disclosure further comprises an epitope tag, e.g. for facilitating identification. Suitable tags, are well known in the art, and include e.g. His, (e.g. (His)6), c-Myc, GST, MBP, CBP, FLAG, HA, E and C tags. Such tags may be provided at the N- and/or C-terminus of the CAR.

In some embodiments of the present disclosure, the CAR of the present disclosure comprises, or consists of: an extracellular moiety of the anti-CD30 HRS3 scFv domain, connected to spacer/hinge domains derived from the CH2-CH3 of human IgG1, the transmembrane and intracellular domains of CD28, and the and the intracellular domain of CD3ζ.

In some embodiments, the CAR is selected from an embodiment of a CD30-specific CAR described in Hombach et al. Cancer Res. (1998) 58(6):1116-9, Hombach et al. Gene Therapy (2000) 7:1067-1075, Hombach et al. J Immunother. (1999) 22(6):473-80, Hombach et al. Cancer Res. (2001) 61:1976-1982, Hombach et al. J Immunol (2001) 167:6123-6131, Savoldo et al. Blood (2007) 110(7):2620-30, Koehler et al. Cancer Res. (2007) 67(5):2265-2273, Di Stasi et al. Blood (2009) 113(25):6392-402, Hombach et al. Gene Therapy (2010) 17:1206-1213, Chmielewski et al. Gene Therapy (2011) 18:62-72, Kofler et al. Mol. Ther. (2011) 19(4):760-767, Gilham, Abken and Pule. Trends in Mol. Med. (2012) 18(7):377-384, Chmielewski et al. Gene Therapy (2013) 20:177-186, Hombach et al. Mol. Ther. (2016) 24(8):1423-1434, Ramos et al. J. Clin. Invest. (2017) 127(9):3462-3471, WO 2021/245249 A1, WO 2021/222927 A1, WO 2021/222928 A1, WO 2021/222929 A1, WO 2015/028444 A1 or WO 2016/008973 A1, all of which are hereby incorporated by reference in their entirety.

In some embodiments of the present disclosure, the CAR comprises, or consists of:
an antigen-binding domain comprising or consisting of an amino acid sequence having at least 60%, 65%, 70%, 75%, 80%, 85%, 80%, 85% 86%, 87%, 88%, 89%, 90%, 91%, 92%, 93%, 94%, 95%, 96%, 97%, 98%, 99%, or 100% sequence identity to the amino acid sequence of SEQ ID NO:24;
a hinge region comprising or consisting of an amino acid sequence having at least 60%, 65%, 70%, 75%, 80%, 85%, 80%, 85% 86%, 87%, 88%, 89%, 90%, 91%, 92%, 93%, 94%, 95%, 96%, 97%, 98%, 99%, or 100% sequence identity to the amino acid sequence of SEQ ID NO:39 or 49;
a transmembrane domain comprising or consisting of an amino acid sequence having at least 60%, 65%, 70%, 75%, 80%, 85%, 80%, 85% 86%, 87%, 88%, 89%, 90%, 91%, 92%, 93%, 94%, 95%, 96%, 97%, 98%, 99%, or 100% sequence identity to the amino acid sequence of SEQ ID NO:26; and
a signalling domain comprising or consisting of an amino acid sequence having at least 60%, 65%, 70%, 75%, 80%, 85%, 80%, 85% 86%, 87%, 88%, 89%, 90%, 91%, 92%, 93%, 94%, 95%, 96%, 97%, 98%, 99%, or 100% sequence identity to the amino acid sequence of SEQ ID NO:34.

In some embodiments of the present disclosure, the CAR comprises, or consists of an amino acid sequence having at least 60%, 65%, 70%, 75%, 80%, 85%, 80%, 85% 86%, 87%, 88%, 89%, 90%, 91%, 92%, 93%, 94%, 95%, 96%, 97%, 98%, 99%, or 100% sequence identity to the amino acid sequence of SEQ ID NO:41, 42, 50 or 51.

In some embodiments, the CAR is a CD19-specific CAR.

In some embodiments of the present disclosure, the CAR comprises, or consists of:
an antigen-binding domain comprising or consisting of an amino acid sequence having at least 60%, 65%, 70%, 75%, 80%, 85%, 80%, 85% 86%, 87%, 88%, 89%, 90%, 91%, 92%, 93%, 94%, 95%, 96%, 97%, 98%, 99%, or 100% sequence identity to the amino acid sequence of SEQ ID NO:67;
a hinge region comprising or consisting of an amino acid sequence having at least 60%, 65%, 70%, 75%, 80%, 85%, 80%, 85% 86%, 87%, 88%, 89%, 90%, 91%, 92%, 93%, 94%, 95%, 96%, 97%, 98%, 99%, or 100% sequence identity to the amino acid sequence of SEQ ID NO:68;
a transmembrane domain comprising or consisting of an amino acid sequence having at least 60%, 65%, 70%, 75%, 80%, 85%, 80%, 85% 86%, 87%, 88%, 89%, 90%, 91%, 92%, 93%, 94%, 95%, 96%, 97%, 98%, 99%, or 100% sequence identity to the amino acid sequence of SEQ ID NO:26; and
a signalling domain comprising or consisting of an amino acid sequence having at least 60%, 65%, 70%, 75%, 80%, 85%, 80%, 85% 86%, 87%, 88%, 89%, 90%, 91%, 92%, 93%, 94%, 95%, 96%, 97%, 98%, 99%, or 100% sequence identity to the amino acid sequence of SEQ ID NO:34.

In some embodiments of the present disclosure, the CAR comprises, or consists of an amino acid sequence having at least 60%, 65%, 70%, 75%, 80%, 85%, 80%, 85% 86%, 87%, 88%, 89%, 90%, 91%, 92%, 93%, 94%, 95%, 96%, 97%, 98%, 99%, or 100% sequence identity to the amino acid sequence of SEQ ID NO:69 or 70.

Methods for producing CAR-expressing immune cells are well known to the skilled person. They generally involve modifying immune cells to express/comprise a CAR, e.g. by introducing nucleic acid encoding a CAR into the immune cells. Immune cells may be modified to comprise/express a CAR or nucleic acid encoding a CAR described herein according to methods that are well known to the skilled person. The methods generally comprise nucleic acid transfer for permanent (stable) or transient expression of the transferred nucleic acid. The methods may further comprise culturing the cell under conditions suitable for expression of the CAR by the cell. In some embodiments, the methods culturing cells into which nucleic acid encoding a CAR has been introduced in order to expand their number.

Suitable methods for modifying a cell include the use of genetic engineering platforms such as gammaretroviral vectors, lentiviral vectors, adenovirus vectors, DNA transfection, transposon-based gene delivery and RNA transfection, for example as described in Maus et al., Annu Rev Immunol (2014) 32:189-225, hereby incorporated by reference in its entirety. Methods also include those described e.g. in Wang and Rivibre Mol Ther Oncolytics. (2016) 3:16015, which is hereby incorporated by reference in its entirety. Suitable methods for introducing nucleic acid(s)/vector(s) into cells include introducing nucleic acid(s) or vector(s) encoding a CAR into a cell by transformation, transfection, electroporation or transduction (e.g. retroviral transduction), e.g. as described herein. In some embodiments, a CAR-expressing immune cell according to the present disclosure is produced essentially as described in Example 1.4 herein.

In particular, the present disclosure contemplates the production of CAR-expressing immune cells according to the methods described in WO 2021/245249 A1, WO 2021/222927 A1, WO 2021/222928 A1 and WO 2021/222929 A1, all of which are incorporated by reference herein.

In some embodiments, the immune cell according to the present disclosure is a virus-specific immune cell. A ‘virus-specific immune cell’ as used herein refers to an immune cell which is specific for a virus. A virus-specific immune cell expresses/comprises a receptor (preferably a T cell receptor) capable of recognising a peptide of an antigen of a virus (e.g. when presented by an MHC molecule). The virus-specific immune cell may express/comprise such a receptor as a result of expression of endogenous nucleic acid encoding such antigen receptor, or as a result of having been engineered to express such a receptor. The virus-specific immune cell preferably expresses/comprises a TCR specific for a peptide of an antigen of a virus.

In some embodiments, the immune cell is a virus-specific T cell. A virus-specific T cell may display certain functional properties of a T cell in response to the viral antigen for which the T cell is specific, or in response to a cell comprising/expressing the virus/antigen. In some embodiments, the properties are functional properties associated with effector T cells, e.g. cytotoxic T cells.

In some embodiments, a virus-specific T cell may display one or more of the following properties: cytotoxicity to a cell comprising/expressing the virus/the viral antigen for which the T cell is specific; proliferation, IFNγ expression, CD107a expression, IL-2 expression, TNFα expression, perforin expression, granzyme expression, granulysin expression, and/or FAS ligand (FASL) expression in response to stimulation with the virus/the viral antigen for which the T cell is specific, or in response to exposure to a cell comprising/expressing the virus/the viral antigen for which the T cell is specific.

Virus-specific T cells express/comprise a TCR capable of recognising a peptide of the viral antigen for which the T cell is specific when presented by the appropriate MHC molecule. Virus-specific T cells may be CD4+ T cells and/or CD8+ T cells.

The virus for which the virus-specific immune cell is specific may be any virus. For example, the virus may be a dsDNA virus (e.g. adenovirus, herpesvirus, poxvirus), ssRNA virus (e.g. parvovirus), dsRNA virus (e.g. reovirus), (+)ssRNA virus (e.g. picornavirus, togavirus), (−)ssRNA virus (e.g. orthomyxovirus, rhabdovirus), ssRNA-RT virus (e.g. retrovirus) or dsDNA-RT virus (e.g. hepadnavirus). In particular, the present disclosure contemplates viruses of the families adenoviridae, herpesviridae, papillomaviridae, polyomaviridae, poxviridae, hepadnaviridae, parvoviridae, astroviridae, caliciviridae, picornaviridae, coronaviridae, flaviviridae, togaviridae, hepeviridae, retroviridae, orthomyxoviridae, arenaviridae, bunyaviridae, filoviridae, paramyxoviridae, rhabdoviridae and reoviridae. In some embodiments the virus is selected from Epstein-Barr virus, adenovirus, Herpes simplex type 1 virus, Herpes simplex type 2 virus, Varicella-zoster virus, Human cytomegalovirus, Human herpesvirus type 8, Human papillomavirus, BK virus, JC virus, Smallpox, Hepatitis B virus, Parvovirus B19, Human Astrovirus, Norwalk virus, coxsackievirus, hepatitis A virus, poliovirus, rhinovirus, severe acute respiratory syndrome virus, Hepatitis C virus, yellow fever virus, dengue virus, West Nile virus, TBE virus, Rubella virus, Hepatitis E virus, Human immunodeficiency virus, influenza virus, lassa virus, Crimean-Congo hemorrhagic fever virus, Hantaan virus, ebola virus, Marburg virus, measles virus, mumps virus, parainfluenza virus, picornavirus, respiratory syncytial virus, rabies virus, hepatitis D virus, rotavirus, orbivirus, coltivirus, and banna virus.

In some embodiments, the virus is selected from Epstein-Barr virus (EBV), adenovirus, cytomegalovius (CMV), human papilloma virus (HPV), influenza virus, measles virus, hepatitis B virus (HBV), hepatitis C virus (HCV), human immunodeficiency virus (HIV), lymphocytic choriomeningitis virus (LCMV), herpes simplex virus (HSV), BK virus (BKV) or varicella zoster virus (VZV).

In some embodiments, the virus-specific immune cell may be specific for a peptide/polypeptide of a virus e.g. selected from Epstein-Barr virus (EBV), adenovirus, cytomegalovius (CMV), human papilloma virus (HPV), influenza virus, measles virus, hepatitis B virus (HBV), hepatitis C virus (HCV), human immunodeficiency virus (HIV), lymphocytic choriomeningitis virus (LCMV), or herpes simplex virus (HSV).

A T cell which is specific for an antigen of a virus may be referred to herein as a virus-specific T cell (VST). A T cell which is specific for an antigen of a particular virus may be described as being specific for the relevant virus; for example, a T cell which is specific for an antigen of EBV may be referred to as an EBV-specific T cell, or “EBVST”.

Accordingly, in some embodiments the virus-specific immune cell is an Epstein-Barr virus-specific T cell (EBVST), adenovirus-specific T cell (AdVST), cytomegalovius-specific T cell (CMVST), human papilloma virus (HPVST), influenza virus-specific T cell, measles virus-specific T cell, hepatitis B virus-specific T cell (HBVST), hepatitis C virus-specific T cell (HCVST), human immunodeficiency virus-specific T cell (HIVST), lymphocytic choriomeningitis virus-specific T cell (LCMVST), herpes simplex virus-specific T cell (HSVST) BK virus-specific T cell (BKVST) or varicella zoster virus-specific T cell (VZVST).

In some preferred embodiments, the virus-specific immune cell is specific for a peptide/polypeptide of an EBV antigen. In preferred embodiments the virus-specific immune cell is an Epstein-Barr virus-specific T cell (EBVST). An ‘EBV-specific immune cell’ as used herein refers to an immune cell which is specific for Epstein-Barr virus (EBV). An EBV-specific immune cell expresses/comprises a receptor (preferably a T cell receptor) capable of recognising a peptide of an antigen of EBV (e.g. when presented by an MHC molecule). The EBV-specific immune cell preferably expresses/comprises a TCR specific for a peptide of an EBV antigen presented by MHC class I.

An immune cell specific for EBV may be specific for any EBV antigen, e.g. an EBV antigen described herein. A population of immune cell specific for EBV, or a composition comprising a plurality of immune cells specific for EBV, may comprise immune cells specific for one or more EBV antigens.

In some embodiments, an EBV antigen is an EBV latent antigen, e.g. a type Ill latency antigen (e.g. EBNA1, EBNA-LP, LMP1, LMP2A, LMP2B, BARF1, EBNA2, EBNA3A, EBNA3B or EBNA3C), a type II latency antigen (e.g. EBNA1, EBNA-LP, LMP1, LMP2A, LMP2B or BARF1), or a type I latency antigen, (e.g. EBNA1 or BARF1). In some embodiments, an EBV antigen is an EBV lytic antigen, e.g. an immediate-early lytic antigen (e.g. BZLF1, BRLF1 or BMRF1), an early lytic antigen (e.g. BMLF1, BMRF1, BXLF1, BALF1, BALF2, BARF1, BGLF5, BHRF1, BNLF2A, BNLF2B, BHLF1, BLLF2, BKRF4, BMRF2, FU or EBNA1-FUK), or a late lytic antigen (e.g. BALF4, BILF1, BILF2, BNFR1, BVRF2, BALF3, BALF5, BDLF3 or gp350).

Virus-specific immune cells may be produced by methods that are well known to the skilled person. The methods may comprise culturing populations of immune cells (e.g. heterogeneous populations of immune cells, e.g. peripheral blood mononuclear cells; PBMCs) comprising cells having antigen-specific receptors (TCRs) in the presence of antigen-presenting cells (APCs) presenting viral antigen peptide:MHC complexes, under conditions providing appropriate costimulation and signal amplification so as to cause activation and expansion. The APCs may be infected with virus encoding, or may comprise/express, the viral antigen/peptide(s), and present the viral antigen peptide in the context of an MHC molecule.

Stimulation causes T cell activation, and promotes cell division (proliferation), resulting in generation and/or expansion of a population of T cells specific for the viral antigen. The process of T cell activation is well known to the skilled person and described in detail, for example, in Immunobiology, 5th Edn. Janeway C A Jr, Travers P, Walport M, et al. New York: Garland Science (2001), Chapter 8, which is incorporated by reference in its entirety.

The population of cells obtained following stimulation is enriched for T cells specific for the virus as compared to the population prior to stimulation (i.e. the virus-specific T cells are present at an increased frequency in the population following stimulation). In this way, a population of T cells specific for the virus is expanded/generated out of a heterogeneous population of T cells having different specificities. A population of T cells specific for a virus may be generated from a single T cell by stimulation and consequent cell division. An existing population of T cells specific for a virus may be expanded by stimulation and consequent cell division of cells of the population of virus-specific T cells.

In some embodiments, a virus-specific immune cell according to the present disclosure is produced essentially as described in Example 1.4 herein.

Aspects and embodiments of the present disclosure relate particularly to EBV-specific immune cells. Methods for generating/expanding populations of EBV-specific immune cells are described e.g. in WO 2013/088114 A1, Lapteva and Vera, Stem Cells Int. (2011): 434392, Straathof et al., Blood (2005) 105(5): 1898-1904, WO 2017/202478 A1, WO 2018/052947 A1 and WO 2020/214479 A1, all of which are hereby incorporated by reference in their entirety.

In particular, the present disclosure contemplates the production of virus-specific immune cells (e.g. EBV-specific immune cells) according to the methods described in WO 2021/222927 A1, WO 2021/222928 A1 and WO 2021/222929 A1, all of which are incorporated by reference herein.

Aspects and embodiments of the present disclosure relate to virus-specific, CAR-expressing, immune cells. In some embodiments, the immune cells are EBV-specific, CAR-expressing, immune cells. In some embodiments, the immune cells are virus-specific, CD30-specific CAR-expressing, immune cells. In some embodiments, the immune cells are EBV-specific, CD30-specific CAR-expressing, immune cells. In some embodiments, the immune cells are virus-specific, CD19-specific CAR-expressing, immune cells. In some embodiments, the immune cells are EBV-specific, CD19-specific CAR-expressing, immune cells. Methods for producing such cells are known in the art, and described in WO 2021/222927 A1, WO 2021/222928 A1 and WO 2021/222929 A1, all of which are incorporated by reference herein.

In some embodiments, the immune cell according to the present disclosure is an activated T cell (ATC). An ‘activated T cell’ or ‘ATC’ as used herein refers to a T cell in which CD3-TCR complex-mediated signalling is activated. In some embodiments, activated T cells have broad specificity (i.e. the activated T cells are reactive to a plurality of distinct antigens).

In some embodiments, an activated T cell may display certain functional properties of a T cell. In some embodiments, the properties are functional properties associated with effector T cells, e.g. cytotoxic T cells. In some embodiments, an activated T cell may display one or more of the following properties: proliferation, expression of one or more surface markers characteristic of T cell activation (e.g. CD25, CD71, CD26, CD27, CD28, CD30, CD154, CD40L, CD134), growth factor (e.g. IL-2) expression, IFNγ expression, CD107a expression, TNFα expression, GM-CSF expression, perforin expression, granzyme expression, granulysin expression, and/or FAS ligand (FASL) expression.

Activated T cells may be CD4+ T cells and/or CD8+ T cells.

Activated T cells may be produced by methods that are well known to the skilled person. The methods may comprise non-specifically activating T cells in vitro by stimulating PBMCs with agonist anti-CD3 and agonist anti-CD28 antibodies in the presence of one or more cytokines (e.g. IL-2, IL-7, IL-15). In some embodiments, an activated T cell can be generated as described in Example 1.4 herein.

In some embodiments, cells modified to increase the expression or activity of SERPINB9 display no toxicity (i.e. display substantially no toxicity) to allogeneic cells (e.g. allogeneic T cells). In some embodiments, cells modified to increase the expression or activity of SERPINB9 display no toxicity to allogeneic T cells. In some embodiments, cells modified to increase the expression or activity of SERPINB9 display no toxicity to allogeneic NK cells.

In some embodiments, “allogeneic cells” are cells which are HLA-mismatched to the test cells (e.g. cells modified to increase the expression or activity of SERPINB9). In some embodiments, “allogeneic cells” are PBMCs which are HLA-mismatched to the test cells. In some embodiments, “allogeneic cells” are alloreactive T cells which are HLA-mismatched to the test cells, e.g. alloreactive T cells generated by activating PBMCs from a HLA-mismatched donor with CD3 and CD28. In some embodiments, “allogeneic cells” do not comprise tumour/disease cells.

As used herein, cells that are “HLA-mismatched” with respect to a reference cell/subject may be: (i) a <8/8 (i.e. 0/8, 1/8, 2/8, 3/8, 4/8, 5/8, 6/8 or 7/8) match across HLA-A, -B, -C, and -DRB1; or (ii) a <10/10 (i.e. 0/10, 1/10, 2/10, 3/10, 4/10, 5/10, 6/10, 7/10, 8/10 or 9/10) match across HLA-A, -B, -C, -DRB1 and -DQB1; or (iii) a <12/12 (i.e. 0/12, 1/12, 2/12, 3/12, 4/12, 5/12, 6/12, 7/12, 8/12, 9/12, 10/12 or 11/12) match across HLA-A, -B, -C, -DRB1, -DQB1 and -DPB1. As used herein, cells that are ‘HLA-matched’ with respect to a reference cell/subject may be: (i) an 8/8 match across HLA-A, -B, -C, and -DRB1; or (ii) a 10/10 match across HLA-A, -B, -C, -DRB1 and -DQB1; or (iii) a 12/12 match across HLA-A, -B, -C, -DRB1, -DQB1 and -DPB1.

In some embodiments, cells that “display no toxicity” or “display substantially no toxicity” to cells of a given type (e.g. allogeneic T cells/NK cells) may display a level of cell killing of/cytolysis of/cytotoxicity to cells of the given type which is similar to (e.g. ≥0.5 times and ≤2 times. e.g. one of ≥0.55 times and ≤1.9 times, ≥0.6 times and ≤1.8 times, ≥0.65 times and ≤1.7 times, ≥0.7 times and ≤1.6 times, ≥0.75 times and ≤1.5 times, ≥0.8 times and ≤1.4 times, ≥0.85 times and ≥1.3 times, ≥0.9 times and ≤1.2 times or ≥0.95 times and ≤1.1 times) or less than (i.e. <1 times) the level displayed by a reference cell type known not to kill/cause cytolysis of/display cytotoxicity of cells of the given type, in the same assay.

The level of killing of allogeneic cells may be evaluated using suitable assays. Suitable assays for evaluating the level of killing of allogeneic cells include e.g. mixed lymphocyte reaction (MLR) assays, such as the assay described in Example 1.5. Suitable assays for evaluating the level of killing of allogeneic cells include e.g. in vivo assays, such as the assay described in Example 1.10. In some embodiments, allogeneic cells or allogeneic cell subtypes are identified by expression or lack of expression of one or more of cell markers CD3, CD4, CD8, HLA-A2 and/or HLA-A3, TCRαβ, CD45, CD19, CD30.

### Properties of Cells Comprising/Expressing Polypeptides, Nucleic Acids and Vectors of the Present Disclosure

Cells having increased expression or activity of SERPINB9 (e.g. cells comprising or expressing nucleic acid(s)/vector(s) according to the present disclosure, or cells treated with an agent for increasing the expression or activity of SERPINB9 according to the present disclosure) may possess one or more of the following:
Reduced activity of a serine protease (e.g. granzyme B);
Reduced activity of a caspase (e.g. caspase-1, -4, -5, -2, -3, -6, -7, -8, or -10);
Increased resistance (and/or reduced susceptibility) to cell killing by a serine protease (e.g. granzyme B);
Increased resistance (and/or reduced susceptibility) to cell killing by cells expressing a serine protease (e.g. granzyme B; e.g. effector immune cells);
Reduced rate of cell killing by cells expressing a serine protease (e.g. cells expressing granzyme B; e.g. effector immune cells);
Increased resistance (and/or reduced susceptibility) to apoptosis mediated by a death receptor (e.g. Fas-mediated apoptosis);
Similar proliferation/expansion in vitro (e.g. when cultured alone, i.e. as a monoculture);
Similar effector activity (i.e. in embodiments wherein the cell is an effector immune cell);
Increased persistence/survival/proliferation in the presence of an allogeneic effector immune cell (e.g. when co-cultured with allogeneic effector immune cells);
Increased persistence/survival/proliferation in an allogeneic subject;
Reduced rejection when administered as an allograft;
Increased anticancer activity in the presence of an allogeneic effector immune cell (e.g. when co-cultured with allogeneic effector immune cells);
Increased anticancer activity in an allogeneic subject;
Increased persistence/survival/proliferation under conditions of chronic antigen exposure; or
Similar expression of one or more markers of immune cell exhaustion (i.e. in embodiments wherein the cell is an effector immune cell).

It will be appreciated that a given cell may display more than one of the properties recited in the preceding paragraph. A ‘reduced’ or ‘increased’ or ‘similar’ level of a given property in accordance with the preceding paragraph may be relative to the level of the relevant property ordinarily displayed by cells of that type (e.g. a reduction or increase relative to a reference value for the level of the relevant property for cells of that type). For cells comprising or expressing nucleic acid(s)/vector(s) according to the present disclosure, a ‘reduced’, ‘increased’ or ‘similar’ level of a given property in accordance with the preceding paragraph may be relative to the level of the relevant property displayed by equivalent cells not comprising the nucleic acid(s)/vector(s). For cells treated with an agent for increasing the expression or activity of SERPINB9 according to the present disclosure, a ‘reduced’, ‘increased’ or ‘similar’ level in accordance with the preceding paragraph may be relative to the level of the relevant property displayed by equivalent cells that have not been treated with the agent. The properties identified in the preceding paragraph can be evaluated e.g. as described hereinabove.

A level of a given property which is ‘increased’ relative to a reference level may be greater than 1 times, e.g. one of ≥1.01 times, ≥1.02 times, ≥1.03 times, ≥1.04 times, ≥1.05 times, ≥1.1 times, ≥1.2 times, ≥1.3 times, ≥1.4 times, ≥1.5 times, ≥1.6 times, ≥1.7 times, ≥1.8 times, ≥1.9 times, ≥2 times, ≥3 times, ≥4 times, ≥25 times, ≥6 times, ≥7 times, ≥8 times, ≥9 times or ≥10 times the reference level. A level of a given property which is ‘reduced’ relative to a reference level may be less than 1 times, e.g. one of ≤0.99 times, ≤0.95 times, ≤0.9 times, ≤0.85 times, ≤0.8 times, ≤0.75 times, ≤0.7 times, ≤0.65 times, ≤0.6 times, ≤0.55 times, ≤0.5 times, ≤0.45 times, ≤0.4 times, ≤0.35 times, ≤0.3 times, ≤0.25 times, ≤0.2 times, ≤0.15 times, ≤0.1 times, ≤0.05 times, or 0.01 times the reference level. A level of a given property which is ‘similar’ to a reference level may be ≤0.5 times and ≤2 times, e.g. one of ≥0.55 times and ≤1.9 times, ≥0.6 times and ≤1.8 times, ≥0.65 times and ≤1.7 times, ≥0.7 times and ≤1.6 times, ≥0.75 times and ≤1.5 times, ≥0.8 times and ≤1.4 times, ≥0.85 times and ≤1.3 times, ≥0.9 times and ≤1.2 times or ≥0.95 times and ≤1.1 times the reference level.

Effector activity of an effector immune cell may be evaluated using an appropriate assay for an effector activity. In some embodiments, an effector activity is selected from: cytotoxicity to a cell comprising/expressing an antigen for which the effector immune cell comprises a molecule (e.g. a TCR, CAR) for directing activity of the effector immune cell against cells comprising/expressing such antigen; proliferation, IFNγ expression, CD107a expression, IL-2 expression, TNFα expression, perforin expression, granzyme expression, granulysin expression, and/or FAS ligand (FASL) expression in response to stimulation with the antigen for which the effector immune cell comprises a molecule (e.g. a TCR, CAR) for directing activity of the effector immune cell against cells comprising/expressing such antigen, or cells comprising/expressing such antigen. By way of illustration, where the cell is an effector immune cell comprising a CAR specific for CD30, an effector activity may be selected from: cytotoxicity to a cell comprising/expressing CD30; proliferation, IFNγ expression, CD107a expression, IL-2 expression, TNFα expression, perforin expression, granzyme expression, granulysin expression, and/or FAS ligand (FASL) expression in response to stimulation with CD30, or cells comprising/expressing CD30. By way of further illustration, where the cell is an effector immune cell comprising a TCR specific for an MHC-peptide complex comprising a peptide of an EBV antigen, an effector activity may be selected from: cytotoxicity to a cell comprising/expressing the MHC-peptide complex; proliferation, IFNγ expression, CD107a expression, IL-2 expression, TNFα expression, perforin expression, granzyme expression, granulysin expression, and/or FAS ligand (FASL) expression in response to stimulation with the MHC-peptide complex, or cells comprising/expressing the MHC-peptide complex.

Gene and/or protein expression of markers of immune cell exhaustion by a given cell (e.g. an effector immune cell) may be evaluated as described herein (by measuring levels of mRNA by quantitative real-time PCR (qRT-PCR), by antibody-based methods, for example by western blot, immunohistochemistry, immunocytohistochemistry, flow cytometry, ELISA, or ELISAPOT). Markers of immune cell exhaustion include e.g. immune checkpoint molecules (e.g. PD-1, CTLA-4, LAG-3, TIM-3, VISTA, TIGIT and BTLA), CD160 and CD244. In preferred embodiments, a marker of immune cell exhaustion is selected from PD-1, LAG-3 and TIM-3. In some embodiments, the cell surface expression of one or more markers of immune cell exhaustion may be evaluated, e.g. by flow cytometry.

In some embodiments, a cell having increased expression or activity of SERPINB9 (e.g. a cell comprising or expressing nucleic acid(s)/vector(s) according to the present disclosure, or a cell treated with an agent for increasing the expression or activity of SERPINB9 according to the present disclosure) has a level of activity of a serine protease (e.g. granzyme B)/activity of a caspase (e.g. caspase-1, -4, -5, -2, -3, -6, -7, -8, or -10)/susceptibility to cell killing by a serine protease (e.g. granzyme B)/susceptibility to cell killing by cells expressing a serine protease (e.g. granzyme B; e.g. effector immune cells)/susceptibility to apoptosis mediated by a death receptor (e.g. Fas-mediated apoptosis)/rate of cell killing by cells expressing a serine protease (e.g. granzyme B; e.g. effector immune cells)/rejection when administered as an allograft that is less than 1 times, e.g. one of ≤0.99 times, ≤0.95 times, ≤0.9 times, ≤0.85 times, ≤0.8 times, ≤0.75 times, ≤0.7 times, ≤0.65 times, ≤0.6 times, ≤0.55 times, ≤0.5 times, ≤0.45 times, ≤0.4 times, ≤0.35 times, ≤0.3 times, ≤0.25 times, ≤0.2 times, ≤0.15 times, ≤0.1 times, ≤0.05 times, or 50.01 times the level of the relevant property ordinarily displayed by cells of that type (e.g. a reference value for the level of the relevant property, for cells of that type), in the same assay.

In some embodiments, a cell comprising or expressing a nucleic acid(s)/vector(s) according to the present disclosure has a level of activity of a serine protease (e.g. granzyme B)/activity of a caspase (e.g. caspase-1, -4, -5, -2, -3, -6, -7, -8, or -10)/susceptibility to cell killing by a serine protease (e.g. granzyme B)/susceptibility to cell killing by cells expressing a serine protease (e.g. granzyme B; e.g. effector immune cells)/susceptibility to apoptosis mediated by a death receptor (e.g. Fas-mediated apoptosis)/rate of cell killing by cells expressing a serine protease (e.g. granzyme B; e.g. effector immune cells)/rejection when administered as an allograft that is less than 1 times, e.g. one of ≤0.99 times, ≤0.95 times, ≤0.9 times, ≤0.85 times, ≤0.8 times, ≤0.75 times, ≤0.7 times, ≤0.65 times, ≤0.6 times, ≤0.55 times, ≤0.5 times, ≤0.45 times, ≤0.4 times, ≤0.35 times, ≤0.3 times, ≤0.25 times, ≤0.2 times, ≤0.15 times, ≤0.1 times, ≤0.05 times, or ≤0.01 times the level of the relevant property displayed by equivalent cells not comprising the nucleic acid(s)/vector(s), in the same assay.

In some embodiments, a cell treated with an agent for increasing the expression or activity of SERPINB9 according to the present disclosure has a level of activity of a serine protease (e.g. granzyme B)/activity of a caspase (e.g. caspase-1, -4, -5, -2, -3, -6, -7, -8, or -10)/susceptibility to cell killing by a serine protease (e.g. granzyme B)/susceptibility to cell killing by cells expressing a serine protease (e.g. granzyme B; e.g. effector immune cells)/susceptibility to apoptosis mediated by a death receptor (e.g. Fas-mediated apoptosis)/rate of cell killing by cells expressing a serine protease (e.g. granzyme B; e.g. effector immune cells)/rejection when administered as an allograft that is less than 1 times, e.g. one of ≥0.99 times, ≤0.95 times, ≤0.9 times, ≤0.85 times, ≤0.8 times, ≤0.75 times, ≤0.7 times, ≤0.65 times, ≤0.6 times, ≤0.55 times, ≤0.5 times, ≤0.45 times, ≤0.4 times, ≤0.35 times, ≤0.3 times, ≤0.25 times, ≤0.2 times, ≤0.15 times, ≤0.1 times, ≤0.05 times, or ≤0.01 times the level of the relevant property displayed by equivalent cells that have not been treated with the agent, in the same assay.

In some embodiments, a cell having increased expression or activity of SERPINB9 (e.g. a cell comprising or expressing nucleic acid(s)/vector(s) according to the present disclosure, or a cell treated with an agent for increasing the expression or activity of SERPINB9 according to the present disclosure) has a level of resistance to cell killing by a serine protease (e.g. granzyme B)/resistance to cell killing by cells expressing a serine protease (e.g. granzyme B; e.g. effector immune cells)/resistance to apoptosis mediated by a death receptor (e.g. Fas-mediated apoptosis)/persistence, survival or proliferation in the presence of an allogeneic effector immune cell/persistence, survival or proliferation in an allogeneic subject/anticancer activity in the presence of an allogeneic effector immune cell/anticancer activity in an allogeneic subject/persistence, survival or proliferation under conditions of chronic antigen exposure that is greater than 1 times, e.g. one of ≥1.01 times, ≥1.02 times, ≥1.03 times, ≥1.04 times, ≥1.05 times, ≥1.1 times, ≥1.2 times, ≥1.3 times, ≥1.4 times, ≥1.5 times, ≥1.6 times, ≥1.7 times, ≥1.8 times, ≥1.9 times, ≥2 times, ≥3 times, ≥4 times, ≥5 times, ≥6 times, ≥7 times, ≥8 times, ≥9 times or ≥10 times the level of the relevant property ordinarily displayed by cells of that type (e.g. a reference value for the level of the relevant property, for cells of that type), in the same assay.

In some embodiments, a cell comprising or expressing a nucleic acid(s)/vector(s) according to the present disclosure has a level of resistance to cell killing by a serine protease (e.g. granzyme B)/resistance to cell killing by cells expressing a serine protease (e.g. granzyme B; e.g. effector immune cells)/persistence, survival or proliferation in the presence of an allogeneic effector immune cell/resistance to apoptosis mediated by a death receptor (e.g. Fas-mediated apoptosis)/persistence, survival or proliferation in an allogeneic subject/anticancer activity in the presence of an allogeneic effector immune cell/anticancer activity in an allogeneic subject/persistence, survival or proliferation under conditions of chronic antigen exposure that is greater than 1 times, e.g. one of ≥1.01 times, ≥1.02 times, ≥1.03 times, ≥1.04 times, ≥1.05 times, ≥1.1 times, ≥1.2 times, ≥1.3 times, ≥1.4 times, ≥1.5 times, ≥1.6 times, ≥1.7 times, ≥1.8 times, ≥1.9 times, ≥2 times, ≥3 times, ≥4 times, ≥5 times, ≥6 times, ≥7 times, ≥8 times, ≥9 times or ≥10 times the level of the relevant property displayed by equivalent cells not comprising the nucleic acid(s)/vector(s), in the same assay.

In some embodiments, a cell treated with an agent for increasing the expression or activity of SERPINB9 according to the present disclosure has a level of resistance to cell killing by a serine protease (e.g. granzyme B)/resistance to cell killing by cells expressing a serine protease (e.g. granzyme B; e.g. effector immune cells)/resistance to apoptosis mediated by a death receptor (e.g. Fas-mediated apoptosis)/persistence, survival or proliferation in an allogeneic subject/anticancer activity in the presence of an allogeneic effector immune cell/anticancer activity in an allogeneic subject/persistence, survival or proliferation under conditions of chronic antigen exposure that is greater than 1 times, e.g. one of ≥1.01 times, ≥1.02 times, ≥1.03 times, ≥1.04 times, ≥1.05 times, ≥11 times, ≥1.2 times, ≥1.3 times, ≥1.4 times, ≥21.5 times, ≥1.6 times, ≥1.7 times, ≥1.8 times, ≥1.9 times, ≥2 times, ≥3 times, ≥4 times, ≥5 times, ≥6 times, ≥7 times, ≥8 times, ≥9 times or ≥10 times the level of the relevant property displayed by equivalent cells that have not been treated with the agent, in the same assay.

In some embodiments, a cell having increased expression or activity of SERPINB9 (e.g. a cell comprising or expressing nucleic acid(s)/vector(s) according to the present disclosure, or a cell treated with an agent for increasing the expression or activity of SERPINB9 according to the present disclosure) displays increased resistance (and/or reduced susceptibility) to cell killing by a serine protease (e.g. granzyme B) and increased resistance (and/or reduced susceptibility) to apoptosis mediated by a death receptor (e.g. Fas-mediated apoptosis). In some embodiments, a cell having increased expression or activity of SERPINB9 (e.g. a cell comprising or expressing nucleic acid(s)/vector(s) according to the present disclosure, or a cell treated with an agent for increasing the expression or activity of SERPINB9 according to the present disclosure) does not display increased resistance (and/or reduced susceptibility) to apoptosis mediated by a death receptor (e.g. Fas-mediated apoptosis).

In some embodiments, a cell treated with an agent for increasing the expression or activity of SERPINB9 according to the present disclosure survives/persists for at least 3 days (e.g. at least 3, 4, 5, 6, 7, 8, 9, 10, 11, 12, 13, 14, or 15 days) in the presence of an allogeneic effector immune cell (e.g. when co-cultured with allogeneic effector immune cells). In some embodiments, a cell treated with an agent for increasing the expression or activity of SERPINB9 according to the present disclosure survives/persists for at least 11 days (e.g. at least 11, 12, 13, 14, or 15 days) in an allogeneic subject.

In some embodiments, a cell having increased expression or activity of SERPINB9 (e.g. a cell comprising or expressing nucleic acid(s)/vector(s) according to the present disclosure, or a cell treated with an agent for increasing the expression or activity of SERPINB9 according to the present disclosure) has a level of proliferation or expansion/a level of an effector activity/expression of one or more markers of immune cell exhaustion that is ≥0.5 times and ≤2 times, e.g. one of ≥0.55 times and ≤1.9 times, ≥0.6 times and ≤1.8 times, ≥0.65 times and ≤1.7 times, ≥0.7 times and ≤1.6 times, ≥0.75 times and ≤1.5 times, ≥0.8 times and ≤1.4 times, ≥0.85 times and ≤1.3 times, ≥0.9 times and ≤1.2 times, ≥0.95 times and ≤1.1 times the level of the relevant property ordinarily displayed by cells of that type (e.g. a reference value for the level of the relevant property, for cells of that type), in the same assay.

In some embodiments, a cell comprising or expressing a nucleic acid(s)/vector(s) according to the present disclosure has a level of proliferation or expansion/a level of an effector activity/expression of one or more markers of immune cell exhaustion that is ≥0.5 times and ≤2 times, e.g. one of ≥0.55 times and ≤1.9 times, ≥0.6 times and ≤1.8 times, ≥0.65 times and ≤1.7 times, ≥0.7 times and ≤1.6 times, ≥0.75 times and ≤1.5 times, ≥0.8 times and ≤1.4 times, ≥0.85 times and ≤1.3 times, ≥0.9 times and ≤1.2 times, ≥0.95 times and ≤1.1 times the level of the relevant property displayed by equivalent cells not comprising the nucleic acid(s)/vector(s), in the same assay.

In some embodiments, a cell treated with an agent for increasing the expression or activity of SERPINB9 according to the present disclosure has a level of proliferation or expansion/a level of an effector activity/expression of one or more markers of immune cell exhaustion that is ≥0.5 times and ≤2 times, e.g. one of ≥0.55 times and ≤1.9 times, ≥0.6 times and ≤1.8 times, ≥0.65 times and ≤1.7 times, ≥0.7 times and ≤1.6 times, ≥0.75 times and ≤1.5 times, ≥0.8 times and ≤1.4 times, ≥0.85 times and ≤1.3 times, ≥0.9 times and ≤1.2 times, ≥0.95 times and ≤1.1 times the level of the relevant property displayed by equivalent cells that have not been treated with the agent, in the same assay.

The present disclosure is concerned in particular with SERPINB9 variants, nucleic acid(s)/vector(s) encoding such SERPINB9 variants, and cells comprising such SERPINB9 variants/nucleic acid(s)/vector(s). In some embodiments, such cells may possess one or more of the following, relative to the level of the relevant property displayed by equivalent cells instead comprising wildtype human SERPINB9 polypeptide (e.g. having the amino acid sequence of SEQ ID NO:1) or by equivalent cells instead comprising comprising/expressing nucleic acid(s)/vector(s) encoding wildtype human SERPINB9 polypeptide (e.g. having the amino acid sequence of SEQ ID NO:1): Reduced activity of a serine protease (e.g. granzyme B);
Reduced activity of a caspase (e.g. caspase-1, -4, -5, -2, -3, -6, -7, -8, or -10);
Increased resistance (and/or reduced susceptibility) to cell killing by a serine protease (e.g. granzyme B);
Increased resistance (and/or reduced susceptibility) to cell killing by cells expressing a serine protease (e.g. granzyme B; e.g. effector immune cells);
Reduced rate of cell killing by cells expressing a serine protease (e.g. cells expressing granzyme B; e.g. effector immune cells);
Increased resistance (and/or reduced susceptibility) to apoptosis mediated by a death receptor (e.g. Fas-mediated apoptosis);
Similar proliferation/expansion in vitro (e.g. when cultured alone, i.e. as a monoculture);
Similar effector activity (i.e. in embodiments wherein the cell is an effector immune cell);
Increased persistence/survival/proliferation in the presence of an allogeneic effector immune cell (e.g. when co-cultured with allogeneic effector immune cells);
Increased persistence/survival/proliferation in an allogeneic subject;
Reduced rejection when administered as an allograft;
Increased anticancer activity in the presence of an allogeneic effector immune cell (e.g. when co-cultured with allogeneic effector immune cells);
Increased anticancer activity in an allogeneic subject;
Increased persistence/survival/proliferation under conditions of chronic antigen exposure; or
Similar expression of one or more markers of immune cell exhaustion (i.e. in embodiments wherein the cell is an effector immune cell).

It will be appreciated that a given cell may display more than one of the properties recited in the preceding paragraph. The properties identified in the preceding paragraph can be evaluated e.g. as described hereinabove.

In some embodiments, a cell comprising a SERPINB9 variant according to the present disclosure (e.g. having the amino acid sequence of SEQ ID NO:5, 6 or 7) has a level of activity of a serine protease (e.g. granzyme B)/activity of a caspase (e.g. caspase-1, -4, -5, -2, -3, -6, -7, -8, or -10)/susceptibility to cell killing by a serine protease (e.g. granzyme B)/susceptibility to cell killing by cells expressing a serine protease (e.g. granzyme B; e.g. effector immune cells)/susceptibility to apoptosis mediated by a death receptor (e.g. Fas-mediated apoptosis)/rate of cell killing by cells expressing a serine protease (e.g. granzyme B; e.g. effector immune cells)/rejection when administered as an allograft that is less than 1 times, e.g. one of ≤0.99 times, ≤0.95 times, ≤0.9 times, ≤0.85 times, ≤0.8 times, ≤0.75 times, ≤0.7 times, ≤0.65 times, ≤0.6 times, ≤0.55 times, ≤0.5 times, ≤0.45 times, ≤0.4 times, ≤0.35 times, ≤0.3 times, ≤0.25 times, ≤0.2 times, ≤0.15 times, ≤0.1 times, ≤0.05 times, or ≤0.01 times the level of the relevant property displayed by cells of the same type instead comprising wildtype human SERPINB9 (e.g. having the amino acid sequence of SEQ ID NO:1), in the same assay. In some embodiments, a cell comprising/expressing nucleic acid(s)/vector(s) encoding a SERPINB9 variant according to the present disclosure (e.g. having the amino acid sequence of SEQ ID NO:5, 6 or 7) has a level of activity of a serine protease (e.g. granzyme B)/activity of a caspase (e.g. caspase-1, -4, -5, -2, -3, -6, -7, -8, or -10)/susceptibility to cell killing by a serine protease (e.g. granzyme B)/susceptibility to cell killing by cells expressing a serine protease (e.g. granzyme B; e.g. effector immune cells)/susceptibility to apoptosis mediated by a death receptor (e.g. Fas-mediated apoptosis)/rate of cell killing by cells expressing a serine protease (e.g. granzyme B; e.g. effector immune cells)/rejection when administered as an allograft that is less than 1 times, e.g. one of ≤0.99 times, ≤0.95 times, ≤0.9 times, ≤0.85 times, ≤0.8 times, ≤0.75 times, ≤0.7 times, ≤0.65 times, ≤0.6 times, ≤0.55 times, ≤0.5 times, ≤0.45 times, ≤0.4 times, ≤0.35 times, ≤0.3 times, ≤0.25 times, ≤0.2 times, ≤0.15 times, ≤0.1 times, ≤0.05 times, or ≤0.01 times the level of the relevant property displayed by cells of the same type instead comprising/expressing nucleic acid(s)/vector(s) encoding wildtype human SERPINB9 (e.g. having the amino acid sequence of SEQ ID NO:1), in the same assay.

In some embodiments, a cell comprising a SERPINB9 variant according to the present disclosure (e.g. having the amino acid sequence of SEQ ID NO:5, 6 or 7) has a level of resistance to cell killing by a serine protease (e.g. granzyme B)/resistance to cell killing by cells expressing a serine protease (e.g. granzyme B; e.g. effector immune cells)/resistance to apoptosis mediated by a death receptor (e.g. Fas-mediated apoptosis)/persistence, survival or proliferation in an allogeneic subject/anticancer activity in the presence of an allogeneic effector immune cell/anticancer activity in an allogeneic subject/persistence, survival or proliferation under conditions of chronic antigen exposure that is greater than 1 times, e.g. one of ≥21.01 times, ≥1.02 times, ≥1.03 times, ≥1.04 times, ≥1.05 times, ≥1.1 times, ≥1.2 times, ≥1.3 times, ≥1.4 times, ≥1.5 times, ≥1.6 times, ≥1.7 times, ≥1.8 times, ≥1.9 times, ≥2 times, ≥3 times, ≥4 times, ≥5 times, ≥6 times, ≥7 times, ≥8 times, ≥9 times or ≥10 times the level of the relevant property displayed by cells of the same type instead comprising wildtype human SERPINB9 (e.g. having the amino acid sequence of SEQ ID NO:1), in the same assay. In some embodiments, a cell comprising/expressing nucleic acid(s)/vector(s) encoding a SERPINB9 variant according to the present disclosure (e.g. having the amino acid sequence of SEQ ID NO:5, 6 or 7) has a level of resistance to cell killing by a serine protease (e.g. granzyme B)/resistance to cell killing by cells expressing a serine protease (e.g. granzyme B; e.g. effector immune cells)/resistance to apoptosis mediated by a death receptor (e.g. Fas-mediated apoptosis)/persistence, survival or proliferation in an allogeneic subject/anticancer activity in the presence of an allogeneic effector immune cell/anticancer activity in an allogeneic subject/persistence, survival or proliferation under conditions of chronic antigen exposure that is greater than 1 times, e.g. one of ≥1.01 times, ≥1.02 times, ≥1.03 times, ≥1.04 times, ≥1.05 times, ≥1.1 times, ≥1.2 times, ≥1.3 times, ≥1.4 times, ≥1.5 times, ≥1.6 times, ≥1.7 times, ≥1.8 times, ≥1.9 times, ≥2 times, ≥3 times, ≥4 times, ≥5 times, ≥6 times, ≥7 times, ≥8 times, ≥9 times or ≥10 times the level of the relevant property displayed by cells of the same type instead comprising/expressing nucleic acid(s)/vector(s) encoding wildtype human SERPINB9 (e.g. having the amino acid sequence of SEQ ID NO:1), in the same assay.

In some embodiments, a cell comprising a SERPINB9 variant according to the present disclosure (e.g. having the amino acid sequence of SEQ ID NO:5, 6 or 7) has a level of proliferation or expansion/a level of an effector activity/expression of one or more markers of immune cell exhaustion that is ≥0.5 times and ≤2 times, e.g. one of ≥0.55 times and ≤1.9 times, ≥0.6 times and ≤1.8 times, ≥0.65 times and ≤1.7 times, ≥0.7 times and ≤1.6 times, ≥0.75 times and ≤1.5 times, ≥0.8 times and ≤1.4 times, ≥0.85 times and s 1.3 times, ≥0.9 times and ≤1.2 times, ≥0.95 times and ≤1.1 times the level of the relevant property displayed by cells of the same type instead comprising wildtype human SERPINB9 (e.g. having the amino acid sequence of SEQ ID NO:1), in the same assay. In some embodiments, a cell comprising/expressing nucleic acid(s)/vector(s) encoding a SERPINB9 variant according to the present disclosure (e.g. having the amino acid sequence of SEQ ID NO:5, 6 or 7) has a level of proliferation or expansion/a level of an effector activity/expression of one or more markers of immune cell exhaustion that is ≥0.5 times and ≤2 times, e.g. one of ≥0.55 times and ≤1.9 times, ≥0.6 times and ≤1.8 times, ≥0.65 times and ≤1.7 times, ≥0.7 times and ≤1.6 times, ≥0.75 times and ≤1.5 times, ≥0.8 times and ≤1.4 times, ≥0.85 times and ≤1.3 times, ≥0.9 times and ≤1.2 times, ≥0.95 times and ≤1.1 times the level of the relevant property displayed by cells of the same type instead comprising/expressing nucleic acid(s)/vector(s) encoding wildtype human SERPINB9 (e.g. having the amino acid sequence of SEQ ID NO:1), in the same assay.

### Particular Exemplary Cells

The following particular exemplary cells are contemplated in connection with the present disclosure:
(1) An effector immune cell (e.g. a T cell or an NK cell) comprising exogenous nucleic acid encoding a SERPINB9 polypeptide.
(2) An effector immune cell (e.g. a T cell or an NK cell) comprising:
   (i) exogenous nucleic acid encoding a SERPINB9 polypeptide; and
   (ii) nucleic acid encoding a chimeric antigen receptor (CAR) comprising an antigen-binding domain that binds to CD30.
(3) An effector immune cell (e.g. a T cell or an NK cell) comprising:
   (i) exogenous nucleic acid encoding a SERPINB9 polypeptide; and
   (ii) nucleic acid encoding a chimeric antigen receptor (CAR) comprising an antigen-binding domain that binds to CD19.
(4) A virus-specific T cell (e.g. an Epstein Barr Virus (EBV)-specific T cell) comprising exogenous nucleic acid encoding a SERPINB9 polypeptide.
(5) A virus-specific T cell (e.g. an Epstein Barr Virus (EBV)-specific T cell) comprising:
   (i) exogenous nucleic acid encoding a SERPINB9 polypeptide; and
   (ii) nucleic acid encoding a chimeric antigen receptor (CAR), comprising an antigen-binding domain that binds to CD30.
(6) A virus-specific T cell (e.g. an Epstein Barr Virus (EBV)-specific T cell) comprising:
   (i) exogenous nucleic acid encoding a SERPINB9 polypeptide; and
   (ii) nucleic acid encoding a chimeric antigen receptor (CAR), comprising an antigen-binding domain that binds to CD19.

### Compositions

The present disclosure also provides compositions comprising the polypeptides, nucleic acids, expression vectors and cells described herein.

The polypeptides, nucleic acids, expression vectors and cells described herein may be formulated as pharmaceutical compositions or medicaments for use in therapeutic and/or prophylactic methods and may comprise a pharmaceutically-acceptable carrier, diluent, excipient or adjuvant. The polypeptides, nucleic acids, expression vectors and cells described herein may be formulated for use in diagnostic and/or prognostic applications.

The compositions of the present disclosure may comprise one or more pharmaceutically-acceptable carriers (e.g. liposomes, micelles, microspheres, nanoparticles), diluents/excipients (e.g. starch, cellulose, a cellulose derivative, a polyol, dextrose, maltodextrin, magnesium stearate), adjuvants, fillers, buffers, preservatives (e.g. vitamin A, vitamin E, vitamin C, retinyl palmitate, selenium, cysteine, methionine, citric acid, sodium citrate, methyl paraben, propyl paraben), anti-oxidants (e.g. vitamin A, vitamin E, vitamin C, retinyl palmitate, selenium), lubricants (e.g. magnesium stearate, talc, silica, stearic acid, vegetable stearin), binders (e.g. sucrose, lactose, starch, cellulose, gelatin, polyethylene glycol (PEG), polyvinylpyrrolidone (PVP), xylitol, sorbitol, mannitol), stabilisers, solubilisers, surfactants (e.g., wetting agents), masking agents or colouring agents (e.g. titanium oxide).

The term ‘pharmaceutically-acceptable’ as used herein pertains to compounds, ingredients, materials, compositions, dosage forms, etc., which are, within the scope of sound medical judgment, suitable for use in contact with the tissues of the subject in question (e.g. a human subject) without excessive toxicity, irritation, allergic response, or other problem or complication, commensurate with a reasonable benefit/risk ratio. Each carrier, diluent, excipient, adjuvant, filler, buffer, preservative, anti-oxidant, lubricant, binder, stabiliser, solubiliser, surfactant, masking agent, colouring agent, flavouring agent or sweetening agent of a composition according to the present disclosure must also be ‘acceptable’ in the sense of being compatible with the other ingredients of the formulation. Suitable carriers, diluents, excipients, adjuvants, fillers, buffers, preservatives, anti-oxidants, lubricants, binders, stabilisers, solubilisers, surfactants, masking agents, colouring agents, flavouring agents or sweetening agents can be found in standard pharmaceutical texts, for example, Remington's ‘The Science and Practice of Pharmacy’ (Ed. A. Adejare), 23rd Edition (2020), Academic Press.

Compositions may be formulated for topical, parenteral, systemic, intracavitary, intravenous, intra-arterial, intramuscular, intrathecal, intraocular, intraconjunctival, intratumoral, subcutaneous, intradermal, intrathecal, oral or transdermal routes of administration. In some embodiments, a pharmaceutical composition/medicament may be formulated for administration by injection or infusion, or administration by ingestion.

Suitable formulations may comprise the relevant article in a sterile or isotonic medium. Medicaments and pharmaceutical compositions may be formulated in fluid, including gel, form. Fluid formulations may be formulated for administration by injection or infusion (e.g. via catheter) to a selected region of the human or animal body.

In some embodiments, the composition is formulated for injection or infusion, e.g. into a blood vessel or tissue/organ of interest.

The present disclosure also provides methods for the production of pharmaceutically useful compositions, such methods of production may comprise one or more steps selected from: producing a polypeptide, nucleic acid, expression vector or cell described herein; isolating polypeptide, nucleic acid, expression vector or cell described herein; and/or mixing polypeptide, nucleic acid, expression vector or cell described herein with a pharmaceutically-acceptable carrier, adjuvant, excipient or diluent.

### Therapeutic and Prophylactic Applications According to the Present Disclosure

The immune cells modified to increase the expression or activity of SERPINB9 described herein, and pharmaceutical compositions comprising such cells, find use in therapeutic and/or prophylactic methods.

A method for treating/preventing a disease/condition in a subject is provided, comprising administering to a subject a therapeutically- or prophylactically-effective quantity of an immune cell or pharmaceutical composition according to the present disclosure.

Also provided is an immune cell or pharmaceutical composition according to the present disclosure for use in a method of medical treatment/prophylaxis. Also provided is the use of an immune cell or pharmaceutical composition according to the present disclosure in the manufacture of a medicament for use in a method for treating/preventing a disease/condition.

It will be appreciated that the methods generally comprise administering a population of immune cells according to the present disclosure to the present disclosure to a subject. In some embodiments, the immune cells may be administered in the form of a pharmaceutical composition comprising such cells.

In particular, use of immune cells according to the present disclosure according to the present disclosure in methods to treat/prevent diseases/conditions by adoptive cell transfer (ACT) is contemplated. The disease/condition to be treated/prevented can be any disease/condition which would derive therapeutic or prophylactic benefit from adoptive transfer of the immune cells. In some embodiments, the disease/condition to be treated/prevented by adoptive cell transfer may e.g. be a T cell dysfunctional disorder, a cancer, an infectious disease or an autoimmune disease.

The immune cells and compositions of the present disclosure can be used in methods involving allotransplantation, e.g. to treat/prevent a disease/condition in a subject.

As used herein, “allotransplantation” refers to the transplantation to a recipient subject of cells, tissues or organs which are genetically non-identical to the recipient subject. The cells, tissues or organs may be from, or may be derived from, cells, tissues or organs of a donor subject that is genetically non-identical to the recipient subject. Allotransplantation is distinct from autotransplantation, which refers to the transplantation of cells, tissues or organs which are from/derived from a donor subject genetically identical to the recipient subject.

The immune cells and compositions of the present disclosure are useful in methods to reduce/prevent the deleterious consequences of alloreactive immune responses (particularly T and/or NK cell-mediated alloreactive immune responses) on allografts. The immune cells of the present disclosure are less susceptible to/more resistant to T and/or NK cell-mediated alloreactive immune responses of the recipient following adoptive transfer, and thus exhibit enhanced persistence/survival in the recipient subject after transfer, and superior therapeutic/prophylactic effects.

In particular, the immune cells and compositions of the present disclosure are contemplated for use in the production and administration of “off-the-shelf” materials for use in therapeutic and prophylactic methods comprising administration of allogeneic material.

It will be appreciated that adoptive transfer of allogeneic immune cells is a form of allotransplantation. In some embodiments, the immune cells are used as therapeutic/prophylactic agents in methods for treating/preventing diseases/conditions by allotransplantation.

Administration of the immune cells and compositions of present disclosure is preferably in a “therapeutically effective” or “prophylactically effective” amount, this being sufficient to show therapeutic or prophylactic benefit to the subject. The actual amount administered, and rate and time-course of administration, will depend on the nature and severity of the disease/condition and the particular article administered. Prescription of treatment, e.g. decisions on dosage etc., is within the responsibility of general practitioners and other medical doctors, and typically takes account of the disease/disorder to be treated, the condition of the individual subject, the site of delivery, the method of administration and other factors known to practitioners. Examples of the techniques and protocols mentioned above can be found in Remington's ‘The Science and Practice of Pharmacy’ (ed. A. Adejare), 23rd Edition (2020), Academic Press.

Multiple doses may be provided. Multiple doses may be separated by a predetermined time interval, which may be selected to be one of 1, 2, 3, 4, 5, 6, 7, 8, 9, 10, 11, 12, 13, 14, 15, 16, 17, 18, 19, 20, 21, 22, 23, or more hours or 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, or 31 days, or 1, 2, 3, 4, 5, or 6 months. By way of example, doses may be given once every 7, 14, 21 or 28 days (plus or minus 3, 2, or 1 days).

In some embodiments, the treatment may further comprise other therapeutic or prophylactic intervention, e.g. chemotherapy, immunotherapy, radiotherapy, surgery, vaccination and/or hormone therapy. Such other therapeutic or prophylactic intervention may occur before, during and/or after the therapies encompassed by the disclosure, and the deliveries of the other therapeutic or prophylactic interventions may occur via different administration routes as the therapies of the disclosure.

Administration may be alone or in combination with other treatments, either simultaneously or sequentially dependent upon the condition to be treated. The immune cells and compositions described herein may be administered simultaneously or sequentially with another therapeutic intervention.

Simultaneous administration refers to administration of two or more therapeutic interventions together, for example as a pharmaceutical composition containing both active agents (i.e. in a combined preparation), or immediately after one another and optionally via the same route of administration, e.g. to the same artery, vein or other blood vessel.

Sequential administration refers to administration of one therapeutic intervention followed after a given time interval by separate administration of one or more further therapeutic interventions. It is not required that the therapies are administered by the same route, although this is the case in some embodiments. The time interval may be any time interval.

Adoptive transfer of immune cells is described, for example, in Kalos and June (2013), Immunity 39(1): 49-60, and Davis et al. (2015), Cancer J. 21(6): 486-491, both of which are hereby incorporated by reference in their entirety. The skilled person is able to determine appropriate reagents and procedures for adoptive transfer of immune cells according to the present disclosure, for example by reference to Dai et al., 2016 J Nat Cancer Inst 108(7): djv439, which is incorporated by reference in its entirety.

Aspects and embodiments of the present disclosure comprise modifying an immune cell to increase the expression or activity of SERPINB9.

In some embodiments, the methods comprise:
(a) modifying an immune cell to increase the expression or activity of SERPINB9, and
(b) administering the modified immune cell to a subject.

As explained hereinabove, such modification may comprise treatment of a cell with an agent for increasing the expression or activity of SERPINB9. In some embodiments, modifying an immune cell to increase the expression or activity of SERPINB9 comprises introducing into an immune cell nucleic acid encoding a SERPINB9 polypeptide.

In some embodiments, the immune cell of (a) comprises a molecule for directing an activity of the immune cells against cells comprising/expressing a given target antigen, e.g. a disease-associated antigen.

In some embodiments, the immune cell of (a) comprises a TCR specific for an MHC-peptide complex comprising the peptide of a disease-associated antigen. In some embodiments, the immune cell of (a) is a virus-specific immune cell, e.g. as described herein.

In some embodiments, the immune cell of (a) comprises a CAR specific for a disease-associated antigen. In some embodiments, the immune cell of (a) is a CAR-expressing immune cell, e.g. as described herein.

In some embodiments, the methods comprise:
(a) modifying an immune cell to comprise/express a molecule for directing an activity of the immune cells against cells comprising/expressing a given target antigen, e.g. a disease-associated antigen (e.g. a TCR or a CAR);
(b) modifying the immune cell to increase the expression or activity of SERPINB9, and
(c) administering the modified immune cell to a subject.

In some embodiments, the methods comprise:
(a) isolating or obtaining an immune cell specific for a virus;
(b) modifying the immune cell to comprise/express a molecule for directing an activity of the immune cells against cells comprising/expressing a given target antigen, e.g. a disease-associated antigen (e.g. a TCR or a CAR);
(c) modifying the immune cell to increase the expression or activity of SERPINB9, and
(d) administering the modified immune cell to a subject.

In some embodiments, the methods comprise:
(a) isolating immune cells (e.g. PBMCs) from a subject;
(b) generating/expanding a population of immune cells specific for a virus;
(b) modifying an immune cell specific for a virus to comprise/express a molecule for directing an activity of the immune cells against cells comprising/expressing a given target antigen, e.g. a disease-associated antigen (e.g. a TCR or a CAR);
(c) modifying the immune cell specific for a virus to increase the expression or activity of SERPINB9, and
(d) administering the modified immune cell specific for a virus to a subject.

In preferred embodiments, the subject from which the immune cell(s) is/are derived is a different subject to the subject to which cells are administered (i.e., adoptive transfer may be of allogeneic cells). In some embodiments, the subject from which the immune cell(s) is/are derived is the same subject to which cells are administered (i.e., adoptive transfer may be of autologous/autogeneic cells).

In some embodiments, modifying the immune cell specific for a virus to increase the expression or activity of SERPINB9 and modifying an immune cell to comprise/express a molecule for directing an activity of the immune cells against cells comprising/expressing a given target antigen, e.g. a disease-associated antigen (e.g. a TCR or a CAR) is performed simultaneously, e.g. in embodiments wherein the immune cell is modified with nucleic acid comprising a nucleotide sequence encoding a SERPINB9 polypeptide, and a nucleotide sequence encoding a molecule for directing an activity of the immune cells against cells comprising/expressing a given target antigen, e.g. a disease-associated antigen (e.g. a TCR or a CAR).

In some embodiments, the methods may comprise one or more of:
obtaining a blood sample from a subject;
isolating immune cells (e.g. PBMCs) from a blood sample which has been obtained from a subject;
generating/expanding a population of immune cells specific for a virus (e.g. by culturing PBMCs in the presence of cells (e.g. APCs) comprising/expressing antigen(s)/peptide(s) of the virus, or by culturing PBMCs in the presence of cells (e.g. APCs) infected with the virus);
culturing the immune cells specific for a virus in in vitro or ex vivo cell culture;
modifying an immune cell specific for a virus to express or comprise a CAR according to the present disclosure, or to express or comprise a nucleic acid encoding a CAR according to the present disclosure (e.g. by transduction with a viral vector encoding such CAR, or a viral vector comprising such nucleic acid);
culturing immune cells specific for a virus expressing/comprising a CAR according to the present disclosure, or expressing/comprising a nucleic acid encoding a CAR according to the present disclosure in in vitro or ex vivo cell culture;
collecting/isolating immune cells specific for a virus expressing/comprising a CAR according to the present disclosure, or expressing/comprising a nucleic acid encoding a CAR according to the present disclosure;
formulating immune cells specific for a virus expressing/comprising a CAR according to the present disclosure, or a nucleic acid encoding a CAR according to the present disclosure to a pharmaceutical composition, e.g. by mixing the cells with a pharmaceutically acceptable adjuvant, diluent, or carrier;
administering immune cells specific for a virus expressing/comprising a CAR according to the present disclosure, or expressing/comprising a nucleic acid encoding a CAR according to the present disclosure, or a pharmaceutical composition comprising such cells, to a subject.

The therapeutic and/or prophylactic methods may be effective to reduce the development/progression of a disease/condition, alleviate the symptoms of a disease/condition, or reduce the pathology of a disease/condition. The methods may be effective to prevent progression of the disease/condition, e.g. to prevent worsening of, or to slow the rate of development of, the disease/condition. In some embodiments the methods may lead to an improvement in the disease/condition, e.g. a reduction in the severity of symptoms of the disease/condition, or a reduction in some other correlate of the severity/activity of the disease/condition. In some embodiments the methods may prevent development of the disease/condition to a later stage (e.g. a chronic stage or metastasis).

It will be appreciated that the therapeutic and prophylactic utility of the CAR-expressing immune cells according to the present disclosure extends to the treatment/prevention of any disease/condition that would derive therapeutic or prophylactic benefit from a reduction in the number/activity of cells expressing/overexpressing the target antigen of the CAR. Similarly, it will be appreciated that the therapeutic and prophylactic utility of the virus-specific immune cells according to the present disclosure extends to the treatment/prevention of any disease/condition that would derive therapeutic or prophylactic benefit from a reduction in the number/activity of cells infected with the virus (or comprising/expressing an antigen of the virus).

In some embodiments, the disease/condition to be treated/prevented in accordance with the present disclosure is a disease/condition in which the virus for which the immune cells are specific is pathologically implicated. That is, in some embodiments the disease/condition is a disease/condition which is caused or exacerbated by infection with the virus, a disease/condition for which infection with the virus is a risk factor and/or a disease/condition for which infection with the virus is positively associated with onset, development, progression, and/or severity of the disease/condition.

In some embodiments, the disease/condition to be treated/prevented in accordance with the present disclosure in which the target antigen for the CAR is pathologically implicated. That is, in some embodiments the disease/condition is a disease/condition which is caused or exacerbated by the expression/overexpression of the target antigen, a disease/condition for which expression/overexpression of the target antigen is a risk factor and/or a disease/condition for which expression/overexpression of the target antigen is positively associated with onset, development, progression, severity of the disease/condition.

The disease/condition may be a disease/condition in which CD30 or cells expressing/overexpressing CD30 are pathologically implicated, e.g. a disease/condition in which cells expressing/overexpressing CD30 are positively associated with the onset, development or progression of the disease/condition, and/or severity of one or more symptoms of the disease/condition, or for which CD30 expression/overexpression is a risk factor for the onset, development or progression of the disease/condition.

The disease/condition may be a disease/condition in which CD19 or cells expressing/overexpressing CD19 are pathologically implicated, e.g. a disease/condition in which cells expressing/overexpressing CD19 are positively associated with the onset, development or progression of the disease/condition, and/or severity of one or more symptoms of the disease/condition, or for which CD19 expression/overexpression is a risk factor for the onset, development or progression of the disease/condition.

The disease/condition to be treated/prevented in accordance with the present disclosure may be a disease/condition characterised by EBV infection. For example, the disease/condition may be a disease/condition in which EBV or cells infected with EBV are pathologically implicated, e.g. a disease/condition in which EBV infection is positively associated with the onset, development or progression of the disease/condition, and/or severity of one or more symptoms of the disease/condition, or for which EBV infection is a risk factor for the onset, development or progression of the disease/condition.

The treatment may be aimed at one or more of: reducing the viral load, reducing the number/proportion of virus-positive cells (e.g. EBV-positive cells), reducing the number/proportion of cells expressing/overexpressing the target antigen of the CAR (e.g. CD30-expressing cells, CD19-expressing cells), reducing the activity of virus-positive cells (e.g. EBV-positive cells), reducing the activity of cells expressing/overexpressing the target antigen of the CAR (e.g. CD30-expressing cells, CD19-expressing cells), delaying/preventing the onset/progression of symptoms of the disease/condition, reducing the severity of symptoms of the disease/condition, reducing the survival/growth of virus-positive cells (e.g. EBV-positive cells), reducing the survival/growth of cells expressing/overexpressing the target antigen of the CAR (e.g. CD30-expressing cells, CD19-expressing cells), or increasing survival of the subject.

In some embodiments, a subject may be selected for treatment described herein based on the detection of the virus (e.g. EBV), cells infected with the virus (e.g. EBV), or cells expressing/overexpressing the target antigen of the CAR (e.g. CD30, CD19) e.g. in the periphery, or in an organ/tissue which is affected by the disease/condition (e.g. an organ/tissue in which the symptoms of the disease/condition manifest), or by the detection of virus-positive cancer cells (e.g. EBV-positive cancer cells) or the detection of cancer cells expressing/overexpressing the target antigen of the CAR (e.g. CD30, CD19). The disease/condition may affect any tissue or organ or organ system. In some embodiments the disease/condition may affect several tissues/organs/organ systems.

In some embodiments a subject may be selected for therapy/prophylaxis in accordance with the present disclosure based on determination that the subject is infected with EBV or comprises cells infected with EBV. In some embodiments a subject may be selected for therapy/prophylaxis in accordance with the present disclosure based on determination that the subject comprises cells expressing/overexpressing CD30, e.g. CD30-expressing/overexpressing cancer cells. In some embodiments a subject may be selected for therapy/prophylaxis in accordance with the present disclosure based on determination that the subject comprises cells expressing/overexpressing CD19, e.g. CD19-expressing/overexpressing cancer cells.

In some embodiments, a subject is administered lymphodepleting chemotherapy prior to administration of immune cells specific for a virus expressing/comprising a CAR described herein (or expressing/comprising nucleic acid encoding such a CAR).

That is, in some embodiments, methods of treating/preventing a disease/condition in accordance with the present disclosure comprise: (i) administering a lymphodepleting chemotherapy to a subject, and (ii) subsequently administering an immune cell specific for a virus expressing/comprising a CAR according to the present disclosure, or expressing/comprising a nucleic acid encoding a CAR according to the present disclosure.

As used herein, “lymphodepleting chemotherapy” refers to treatment with a chemotherapeutic agent which results in depletion of lymphocytes (e.g. T cells, B cells, NK cells, NKT cells or innate lymphoid cell (ILCs), or precursors thereof) within the subject to which the treatment is administered. A “lymphodepleting chemotherapeutic agent” refers to a chemotherapeutic agent which results in depletion of lymphocytes.

Lymphodepleting chemotherapy and its use in methods of treatment by adoptive cell transfer are described e.g. in Klebanoff et al., Trends Immunol. (2005) 26(2):111-7 and Muranski et al., Nat Clin Pract Oncol. (2006) (12):668-81, both of which are hereby incorporated by reference in their entirety. The aim of lymphodepleting chemotherapy is to deplete the recipient subject's endogenous lymphocyte population.

In the context of treatment of disease by adoptive transfer of immune cells, lymphodepleting chemotherapy is typically administered prior to adoptive cell transfer, to condition the recipient subject to receive the adoptively transferred cells. Lymphodepleting chemotherapy is thought to promote the persistence and activity of adoptively transferred cells by creating a permissive environment, e.g. through elimination of cells expressing immunosuppressive cytokines, and creating the ‘lymphoid space’ required for expansion and activity of adoptively transferred lymphoid cells.

Chemotherapeutic agents commonly used in lymphodepleting chemotherapy include e.g. fludarabine, cyclophosphamide, bendamustine and pentostatin.

In some embodiments, the disease to be treated/prevented in accordance with the present disclosure is a cancer. Cancer may refer to any unwanted cell proliferation (or any disease manifesting itself by unwanted cell proliferation), neoplasm or tumor. The cancer may be benign or malignant and may be primary or secondary (metastatic). A neoplasm or tumor may be any abnormal growth or proliferation of cells and may be located in any tissue. The cancer may be of tissues/cells derived from e.g. the adrenal gland, adrenal medulla, anus, appendix, bladder, blood, bone, bone marrow, brain, breast, cecum, central nervous system (including or excluding the brain) cerebellum, cervix, colon, duodenum, endometrium, epithelial cells (e.g. renal epithelia), gallbladder, oesophagus, glial cells, heart, ileum, jejunum, kidney, lacrimal glad, larynx, liver, lung, lymph, lymph node, lymphoblast, maxilla, mediastinum, mesentery, myometrium, nasopharynx, omentum, oral cavity, ovary, pancreas, parotid gland, peripheral nervous system, peritoneum, pleura, prostate, salivary gland, sigmoid colon, skin, small intestine, soft tissues, spleen, stomach, testis, thymus, thyroid gland, tongue, tonsil, trachea, uterus, vulva, and/or white blood cells. Tumors may be nervous or non-nervous system tumors. Nervous system tumors may originate either in the central or peripheral nervous system, e.g. glioma, medulloblastoma, meningioma, neurofibroma, ependymoma, Schwannoma, neurofibrosarcoma, astrocytoma and oligodendroglioma. Non-nervous system cancers/tumors may originate in any other non-nervous tissue, examples include melanoma, mesothelioma, lymphoma, myeloma, leukemia, Non-Hodgkin's lymphoma (NHL), Hodgkin's lymphoma, chronic myelogenous leukemia (CML), acute myeloid leukemia (AML), myelodysplastic syndrome (MDS), cutaneous T cell lymphoma (CTCL), chronic lymphocytic leukemia (CLL), hepatoma, epidermoid carcinoma, prostate carcinoma, breast cancer, lung cancer, colon cancer, ovarian cancer, pancreatic cancer, thymic carcinoma, NSCLC, hematologic cancer and sarcoma.

In some embodiments the cancer is selected from the group consisting of: a solid cancer, a hematological cancer, gastric cancer (e.g. gastric carcinoma, gastric adenocarcinoma, gastrointestinal adenocarcinoma), liver cancer (hepatocellular carcinoma, cholangiocarcinoma), head and neck cancer (e.g. head and neck squamous cell carcinoma), oral cavity cancer (e.g. oropharyngeal cancer (e.g. oropharyngeal carcinoma), oral cancer, laryngeal cancer, nasopharyngeal carcinoma, oesophageal cancer), colorectal cancer (e.g. colorectal carcinoma), colon cancer, colon carcinoma, cervical carcinoma, prostate cancer, lung cancer (e.g. NSCLC, small cell lung cancer, lung adenocarcinoma, squamous lung cell carcinoma), bladder cancer, urothelial carcinoma, skin cancer (e.g. melanoma, advanced melanoma), renal cell cancer (e.g. renal cell carcinoma), ovarian cancer (e.g. ovarian carcinoma), mesothelioma, breast cancer, brain cancer (e.g. glioblastoma), prostate cancer, pancreatic cancer, a myeloid hematologic malignancy, a lymphoblastic hematologic malignancy, myelodysplastic syndrome (MDS), acute myeloid leukemia (AML), chronic myeloid leukemia (CML), acute lymphoblastic leukemia (ALL), lymphoma, non-Hodgkin's lymphoma (NHL), thymoma or multiple myeloma (MM).

In some embodiments the cancer is a cancer in which the virus for which the immune cells are specific is pathologically implicated. That is, in some embodiments the cancer is a cancer which is caused or exacerbated by infection with the virus, a cancer for which infection with the virus is a risk factor and/or a cancer for which infection with the virus is positively associated with onset, development, progression, severity or metastasis of the cancer.

EBV infection is implicated in several cancers, as reviewed e.g. in Jha et al., Front Microbiol. (2016) 7:1602, which is hereby incorporated by reference in its entirety.

In some embodiments, the cancer to be treated/prevented is an EBV-associated cancer. In some embodiments, the cancer is a cancer which is caused or exacerbated by infection with EBV, a cancer for which infection with EBV is a risk factor and/or a cancer for which infection with EBV is positively associated with onset, development, progression, severity or metastasis of the cancer. The cancer may be characterised by EBV infection, e.g. the cancer may comprise cells infected with EBV. Such cancers may be referred to as EBV-positive cancers.

EBV-associated cancers which may be treated/prevented in accordance with the present disclosure include B cell-associated cancers such as Burkitt's lymphoma, post-transplant lymphoproliferative disease (PTLD), central nervous system lymphoma (CNS lymphoma), Hodgkin's lymphoma, non-Hodgkin's lymphoma, and EBV-associated lymphomas associated with immunodeficiency (including e.g. EBV-positive lymphoma associated with X-linked lymphoproliferative disorder, EBV-positive lymphoma associated with HIV infection/AIDS, and oral hairy leukoplakia), and epithelial cell-related cancers such as nasopharyngeal carcinoma (NPC) and gastric carcinoma (GC).

In some embodiments, the cancer is selected from lymphoma (e.g. EBV-positive lymphoma), head and neck squamous cell carcinoma (HNSCC; e.g. EBV-positive HNSCC), nasopharyngeal carcinoma (NPC; e.g. EBV-positive NPC), and gastric carcinoma (GC; e.g. EBV-positive GC).

In some embodiments the cancer is a cancer in which the target antigen for the CAR is pathologically implicated. That is, in some embodiments the cancer is a cancer which is caused or exacerbated by the expression of the target antigen, a cancer for which expression of the target antigen is a risk factor and/or a cancer for which expression of the target antigen is positively associated with onset, development, progression, severity or metastasis of the cancer. The cancer may be characterised by expression of the target antigen, e.g. the cancer may comprise cells expressing the target antigen. Such cancers may be referred to as being positive for the target antigen.

A cancer which is ‘positive’ for the target antigen may be a cancer comprising cells expressing the target antigen (e.g. at the cell surface). A cancer which is ‘positive’ for the target antigen may overexpress the target antigen. Overexpression of the target antigen may be determined by detection of a level of gene or protein expression of the target antigen which is greater than the level of expression by equivalent non-cancerous cells/non-tumor tissue.

In some embodiments the target antigen is a cancer cell antigen as described herein. In some embodiments the target antigen is CD30. In some embodiments the cancer is a cancer in which CD30 is pathologically implicated. That is, in some embodiments the cancer is a cancer which is caused or exacerbated CD30 expression, a cancer for which expression of CD30 is a risk factor and/or a cancer for which expression of CD30 is positively associated with onset, development, progression, severity or metastasis of the cancer. The cancer may be characterised by CD30 expression, e.g. the cancer may comprise cells expressing CD30. Such cancers may be referred to as CD30-positive cancers.

A CD30-positive cancer may be a cancer comprising cells expressing CD30 (e.g. cells expressing CD30 protein at the cell surface). A CD30-positive cancer may overexpress CD30. Overexpression of CD30 can be determined by detection of a level of gene or protein expression of CD30 which is greater than the level of expression by equivalent non-cancerous cells/non-tumor tissue.

CD30-positive cancers are described e.g. in van der Weyden et al., Blood Cancer Journal (2017) 7:e603 and Muta and Podack, Immunol Res (2013), 57(1-3):151-8, both of which are hereby incorporated by reference in their entirety. CD30 is expressed on small subsets of activated T and B lymphocytes, and by various lymphoid neoplasms including classical Hodgkin's lymphoma and anaplastic large cell lymphoma. Variable expression of CD30 has also been shown for peripheral T cell lymphoma, not otherwise specified (PTCL-NOS), adult T cell leukemia/lymphoma, cutaneous T cell lymphoma (CTCL), extra-nodal NK-T cell lymphoma, various B cell non-Hodgkin's lymphomas (including diffuse large B cell lymphoma, particularly EBV-positive diffuse large B cell lymphoma), and advanced systemic mastocytosis. CD30 expression has also been observed in some non-hematopoietic malignancies, including germ cell tumors and testicular embryonal carcinomas.

The transmembrane glycoprotein CD30, is a member of the tumor necrosis factor receptor superfamily (Falini et al, Blood (1995) 85(1):1-14). Members of the TNF/TNF-receptor (TNF-R) superfamily coordinate the immune response at multiple levels and CD30 plays a role in regulating the function or proliferation of normal lymphoid cells. CD30 was originally described as an antigen recognized by a monoclonal antibody, Ki-1, which was raised by immunizing mice with a HL-derived cell line, L428 (Muta and Podack, Immunol Res (2013) 57: 151-158). CD30 antigen expression has been used to identify ALCL and Reed-Sternberg cells in Hodgkin's disease (Falini et al., Blood (1995) 85(1):1-14). With the wide expression in the lymphoma malignant cells, CD30 is therefore a potential target for developing both antibody-based immunotherapy and cellular therapies. Importantly, CD30 is not typically expressed on normal tissues under physiologic conditions, thus is notably absent on resting mature or precursor B or T cells (Younes and Ansell, Semin Hematol (2016) 53: 186-189). Brentuximab vedotin, an antibody-drug conjugate that targets CD30 was initially approved for the treatment of CD30-positive HL (Adcetris® US Package Insert 2018). Data from brentuximab vedotin trials support CD30 as a therapeutic target for the treatment of CD30-positive lymphoma, although toxicities associated with its use are of concern.

Hodgkin lymphoma (HL) is an uncommon malignancy involving lymph nodes and the lymphatic system. The incidence of HL is bimodal with most patients diagnosed between 15 and 30 years of age, followed by another peak in adults aged 55 years or older. In 2019 it is estimated there will be 8,110 new cases (3,540 in females and 4570 in males) in the United States and 1,000 deaths (410 females and 590 males) from this disease (American Cancer Society 2019). Based on 2012-2016 cases in National Cancer Institute's SEER database, the incidence rate for HL for the pediatric HL patients in US is as follows: Age 1-4: 0.1; Age 5-9: 0.3; Age 10-14: 1.3; Age 15-19: 3.3 per 100,000 (SEER Cancer Statistics Review, 1975-2016]). The World Health Organization (WHO) classification divides HL into 2 main types: classical Hodgkin lymphoma (cHL) and nodular lymphocyte-predominant Hodgkin lymphoma (NLPHL). In Western countries, cHL accounts for 95% and NLPHL accounts for 5% of all HL (National Comprehensive Cancer Network Guidelines 2019).

First-line chemotherapy for cHL patients with advanced disease is associated with cure rates between 70% and 75% (Karantanos et al., Blood Lymphat Cancer (2017) 7:37-52). Salvage chemotherapy followed by Autologous Stem Cell Transplant (ASCT) is commonly used in patients who relapse after primary therapy. Unfortunately, up to 50% of the cHL patients experience disease recurrence after ASCT. The median overall survival of patients who relapse after ASCT is approximately two years (Alinari Blood (2016) 127:287-295). Despite aggressive combination chemotherapy, between 10% and 40% of patients do not achieve a response to salvage chemotherapy and there are no randomized clinical trial data supporting ASCT in non-responders. For patients who do not respond to salvage chemotherapy, relapse after ASCT or who are not candidates for this approach, the prognosis continues to be grave and new treatment approaches are urgently needed (Keudell British Journal of Haematology (2019) 184:105-112). While a majority of the pediatric population (children, adolescents, and young adults) will be cured with currently available therapy, a small fraction of patients may have refractory or relapsed disease and require novel therapies that have an acceptable safety profile with improved efficacy benefit (Flerlage et al., Blood (2018) 132: 376-384; Kelly, Blood (2015) 126: 2452-2458; McClain and Kamdar, in UpToDate 2019; Moskowitz, ASCO Educational Book (2019) 477-486). HL patients treated with high dose chemotherapy during childhood commonly experience treatment-related long-term sequelae, such as cardiac, pulmonary, gonadal, and endocrine toxicity as well as second malignant neoplasms (Castellino et al, Blood (2011) 117(6): 1806-1816).

In some embodiments, a CD30-positive cancer may be selected from: a solid cancer, a hematological cancer, a hematopoietic malignancy, Hodgkin's lymphoma (HL), anaplastic large cell lymphoma (ALCL), ALK-positive anaplastic T cell lymphoma, ALK-negative anaplastic T cell lymphoma, peripheral T cell lymphoma (e.g. PTCL-NOS), T cell leukemia, T cell lymphoma, cutaneous T cell lymphoma (CTCL), NK-T cell lymphoma (e.g. extra-nodal NK-T cell lymphoma), non-Hodgkin's lymphoma (NHL), B cell non-Hodgkin's lymphoma, diffuse large B cell lymphoma (e.g. diffuse large B cell lymphoma-NOS), primary mediastinal B cell lymphoma, EBV-positive B cell lymphoma, EBV-positive diffuse large B cell lymphoma, advanced systemic mastocytosis, a germ cell tumor and testicular embryonal carcinoma. In some embodiments, the target antigen is a cancer cell antigen as described herein. In some embodiments the target antigen is CD19. In some embodiments the cancer is a cancer in which CD19 is pathologically implicated. That is, in some embodiments the cancer is a cancer which is caused or exacerbated CD19 expression, a cancer for which expression of CD19 is a risk factor and/or a cancer for which expression of CD19 is positively associated with onset, development, progression, severity or metastasis of the cancer. The cancer may be characterised by CD19 expression, e.g. the cancer may comprise cells expressing CD19. Such cancers may be referred to as CD19-positive cancers.

A CD19-positive cancer may be a cancer comprising cells expressing CD19 (e.g. cells expressing CD19 protein at the cell surface). A CD19-positive cancer may overexpress CD19. Overexpression of CD19 can be determined by detection of a level of gene or protein expression of CD19 which is greater than the level of expression by equivalent non-cancerous cells/non-tumor tissue.

CD19 is a transmembrane glycoprotein belonging to the immunoglobulin superfamily. CD19 is classified as a type I transmembrane protein, with a single transmembrane domain, a cytoplasmic C-terminus, and extracellular N-terminus. CD19 was first identified as the B4 antigen of human B lymphocytes through the use of anti-B4 monoclonal antibody (mAb) against CD19. CD19 is expressed throughout B-cell development until terminal plasma cell differentiation. Expression is not seen in stem cells or most other normal cell types. CD19 enhances B-cell antigen receptor signalling by amplification of phosphoinositide-3-kinase and Bruton's tyrosine kinase activity (Wang et al., Exp Hematol Oncol (2012) 1(1): 36, Fujimoto et al., Semin Immunol (1998) 10:267-277), which plays a crucial role in tumor cell proliferation and survival (Seda et al., Eur J Haematol (2015) 94(3):193-205). CD19 is broadly and homogeneously expressed across different B-cell malignancies including diffuse large B-cell lymphoma (DLBCL), follicular lymphoma (FL) and chronic lymphocytic leukemia (CLL) (Olejniczak et al., Immunol Invest (2006) 35(1):93-114, Schuurman et al., Am J Pathol (1988) 131:102-111).

Non-Hodgkin lymphoma (NHL) is one of the most common cancers in the United States, accounting for about 4% of all cancers. In 2023 it is estimated that in the United States about 80,550 people (44,880 males and 35,670 females) will be diagnosed with NHL and about 20,180 people (11,780 males and 8,400 females) will die from this cancer (American Cancer Society 2023). The World Health Organisation (WHO) classification divides NHL into main two groups: those of B-cell origin and those of T-cell/natural killer (NK) cell origin. The two main groups are then further classified into subtypes. In relation to those of B-cell origin, the WHO provides the further classification subtypes: precursor B-lymphoblastic leukemia/lymphoma, and peripheral B-cell neoplasms (which includes the further classification subtypes B-cell chronic lymphocytic leukemia/small lymphocytic lymphoma, B-cell prolymphocytic leukemia, lymphoplasmacytic lymphoma/immunocytoma, mantle cell lymphoma, follicular lymphoma, extranodal marginal zone B-cell lymphoma of mucosa-associated lymphatic tissue (MALT) type, nodal marginal zone B-cell lymphoma (± monocytoid B cells), splenic marginal zone lymphoma (± villous lymphocytes), hairy cell leukemia, plasmacytoma/plasma cell myeloma, diffuse large B-cell lymphoma (DLBCL), and Burkitt lymphoma)

In real life practice and in the vast majority of clinical trials histological subtypes of NHL have been roughly segregated into indolent, aggressive and very aggressive groups, according to their usual clinical behaviour. Indolent B-cell lymphomas represent 35 to 40 percent of the non-Hodgkin lymphomas (NHL), and survival is generally measured in years. The most common subtypes include follicular lymphoma (FL), chronic lymphocytic leukemia/small lymphocytic lymphoma (CLL/SLL), a fraction of mantle cell lymphoma (MCL) cases, extramedullary, nodal and splenic marginal zone lymphoma (MZL), and lymphoplasmacytic lymphoma (LPL) (Swerdlow et al., Blood (2016) 127:2375-90, Rizvi et al., Blood (2006) 107:1255-64). Aggressive subtypes if left untreated survive a few months but if adequately treated may achieve definitive remissions and cure in a significant fraction of patients. The most common subtypes are large B-cell lymphomas, including anaplastic and primary mediastinal lymphoma, and various kinds of diffuse large B cell lymphoma (DLBCL). The highly aggressive subtypes represent about 5 percent of NHL cases and survival may be measured in only a few weeks if left untreated. However, curing is possible if vigorously treated with high-intensity chemotherapy protocols.

Chemotherapy, radiotherapy, and immunotherapy have been used, alone or in combination, in the last decades to treat B-cell NHL. Therapeutic outcomes may vary according to clinical behaviour, whether indolent or aggressive, and patients may suffer various patterns of recurrence requiring subsequent lines of rescue therapies. Dismal prognosis still affects a significant fraction of patients with mature B-cell lymphomas, and new treatment strategies should be conceived to improve both objective response and survival (Jiang et al. Expert Rev Hematol (2017) 10:405-15, Gisselbrecht et al., Br J Haematol (2018) 182:633-43, El-Mallawany et al., Clin Adv Hematol Oncol (2015) 13:113-23, Mei et al., Clin Lymphoma Myeloma Leuk (2018) 18:26-33, Shanbhag et al., CA Cancer J Clin (2018) 68:116-32, Biccler et al., Leuk Lymphoma (2018) doi:10.1080/10428194.2018.1540044, Barth et al., Br J Haematol (2019) doi:10.1111/bjh.15783).

In some embodiments, a CD19-positive cancer may be selected from: non-Hodgkin's Lymphoma, diffuse large B-cell lymphoma (DLBCL), follicular lymphoma, (FL), Mantle Cell lymphoma (MCL), chronic lymphatic lymphoma (CLL), Marginal Zone B-cell lymphoma (MZBL), extranodal marginal zone B-cell lymphoma of mucosa-associated lymphatic tissue (MALT) type, nodal marginal zone B-cell lymphoma (monocytoid B cells), splenic marginal zone lymphoma (±villous lymphocytes), leukemia, Hairy cell leukemia (HCL), Hairy cell leukemia variant (HCL-v), Acute Lymphoblastic Leukaemia (ALL), Philadelphia chromosome-positive ALL (Ph+ALL) and Philadelphia chromosome-negative ALL (Ph−ALL), B-cell chronic lymphocytic leukemia/small lymphocytic lymphoma, B-cell prolymphocytic leukemia, lymphoplasmacytic lymphoma/immunocytoma, plasmacytoma/plasma cell myeloma, and Burkitt lymphoma.

In some embodiments, the cancer is selected from: a CD30-positive cancer, a CD19-positive cancer, an EBV-associated cancer, a hematological cancer, a myeloid hematologic malignancy, a hematopoietic malignancy a lymphoblastic hematologic malignancy, myelodysplastic syndrome, leukemia, T cell leukemia, acute myeloid leukemia, chronic myeloid leukemia, acute lymphoblastic leukemia, lymphoma, Hodgkin's lymphoma, non-Hodgkin's lymphoma, B cell non-Hodgkin's lymphoma, diffuse large B cell lymphoma, primary mediastinal B cell lymphoma, EBV-associated lymphoma, EBV-positive B cell lymphoma, EBV-positive diffuse large B cell lymphoma, EBV-positive lymphoma associated with X-linked lymphoproliferative disorder, EBV-positive lymphoma associated with HIV infection/AIDS, oral hairy leukoplakia, Burkitt's lymphoma, post-transplant lymphoproliferative disease, central nervous system lymphoma, anaplastic large cell lymphoma, T cell lymphoma, ALK-positive anaplastic T cell lymphoma, ALK-negative anaplastic T cell lymphoma, peripheral T cell lymphoma, cutaneous T cell lymphoma, NK-T cell lymphoma, extra-nodal NK-T cell lymphoma, thymoma, multiple myeloma, a solid cancer, epithelial cell cancer, gastric cancer, gastric carcinoma, gastric adenocarcinoma, gastrointestinal adenocarcinoma, liver cancer, hepatocellular carcinoma, cholangiocarcinoma, head and neck cancer, head and neck squamous cell carcinoma, oral cavity cancer, oropharyngeal cancer, oropharyngeal carcinoma, oral cancer, laryngeal cancer, nasopharyngeal carcinoma, oesophageal cancer, colorectal cancer, colorectal carcinoma, colon cancer, colon carcinoma, cervical carcinoma, prostate cancer, lung cancer, non-small cell lung cancer, small cell lung cancer, lung adenocarcinoma, squamous lung cell carcinoma, bladder cancer, urothelial carcinoma, skin cancer, melanoma, advanced melanoma, renal cell cancer, renal cell carcinoma, ovarian cancer, ovarian carcinoma, mesothelioma, breast cancer, brain cancer, glioblastoma, prostate cancer, pancreatic cancer, mastocytosis, advanced systemic mastocytosis, germ cell tumor or testicular embryonal carcinoma.

In some embodiments, the cancer may be a relapsed cancer. As used herein, a “relapsed” cancer refers to a cancer which responded to a treatment (e.g. a first line therapy for the cancer), but which has subsequently re-emerged/progressed, e.g. after a period of remission. For example, a relapsed cancer may be a cancer whose growth/progression was inhibited by a treatment (e.g. a first line therapy for the cancer), and which has subsequently grown/progressed.

In some embodiments, the cancer may be a refractory cancer. As used herein, a “refractory” cancer refers to a cancer which has not responded to a treatment (e.g. a first line therapy for the cancer). For example, a refractory cancer may be a cancer whose growth/progression was not inhibited by a treatment (e.g. a first line therapy for the cancer). In some embodiments a refractory cancer may be a cancer for which a subject receiving treatment for the cancer did not display a partial or complete response to the treatment.

In embodiments where the cancer is anaplastic large cell lymphoma, the cancer may be relapsed or refractory with respect to treatment with chemotherapy, brentuximab vedotin, or crizotinib. In embodiments where the cancer is peripheral T cell lymphoma, the cancer may be relapsed or refractory with respect to treatment with chemotherapy or brentuximab vedotin. In embodiments where the cancer is extranodal NK-T cell lymphoma, the cancer may be relapsed or refractory with respect to treatment with chemotherapy (with or without asparaginase) or brentuximab vedotin. In embodiments where the cancer is diffuse large B cell lymphoma, the cancer may be relapsed or refractory with respect to treatment with chemotherapy (with or without rituximab) or CD19 CAR-T therapy. In embodiments where the cancer is primary mediastinal B cell lymphoma, the cancer may be relapsed or refractory with respect to treatment with chemotherapy, immune checkpoint inhibitor (e.g. PD-1 inhibitor) or CD19 CAR-T therapy.

Treatment of a cancer in accordance with the methods of the present disclosure achieves one or more of the following treatment effects: reduces the number of cancer cells in the subject, reduces the size of a cancerous tumor/lesion in the subject, inhibits (e.g. prevents or slows) growth of cancer cells in the subject, inhibits (e.g. prevents or slows) growth of a cancerous tumor/lesion in the subject, inhibits (e.g. prevents or slows) the development/progression of a cancer (e.g. to a later stage, or metastasis), reduces the severity of symptoms of a cancer in the subject, increases survival of the subject (e.g. progression free survival or overall survival), reduces a correlate of the number or activity of cancer cells in the subject, and/or reduces cancer burden in the subject.

Subjects may be evaluated in accordance with the Revised Criteria for Response Assessment: The Lugano Classification (described e.g. in Cheson et al., J Clin Oncol (2014) 32: 3059-3068, incorporated by reference hereinabove) in order to determine their response to treatment. In some embodiments, treatment of a subject in accordance with the methods of the present disclosure achieves one of the following: complete response, partial response, or stable disease.

In some embodiments, treatment of cancer further comprises chemotherapy and/or radiotherapy.

Chemotherapy and radiotherapy respectively refer to treatment of a cancer with a drug or with ionising radiation (e.g. radiotherapy using X-rays or γ-rays). The drug may be a chemical entity, e.g. small molecule pharmaceutical, antibiotic, DNA intercalator, protein inhibitor (e.g. kinase inhibitor), or a biological agent, e.g. antibody, antibody fragment, aptamer, nucleic acid (e.g. DNA, RNA), peptide, polypeptide, or protein. The drug may be formulated as a pharmaceutical composition or medicament. The formulation may comprise one or more drugs (e.g. one or more active agents) together with one or more pharmaceutically acceptable diluents, excipients or carriers.

Chemotherapy may involve administration of more than one drug. A drug may be administered alone or in combination with other treatments, either simultaneously or sequentially dependent upon the condition to be treated.

The chemotherapy may be administered by one or more routes of administration, e.g. parenteral, intravenous injection, oral, subcutaneous, intradermal or intratumoral.

The chemotherapy may be administered according to a treatment regime. The treatment regime may be a pre-determined timetable, plan, scheme or schedule of chemotherapy administration which may be prepared by a physician or medical practitioner and may be tailored to suit the patient requiring treatment. The treatment regime may indicate one or more of: the type of chemotherapy to administer to the patient; the dose of each drug or radiation; the time interval between administrations; the length of each treatment; the number and nature of any treatment holidays, if any etc. For a co-therapy a single treatment regime may be provided which indicates how each drug is to be administered.

Chemotherapeutic drugs may be selected from: Abemaciclib, Abiraterone Acetate, Abitrexate (Methotrexate), Abraxane (Paclitaxel Albumin-stabilized Nanoparticle Formulation), ABVD, ABVE, ABVE-PC, AC, Acalabrutinib, AC-T, Adcetris (Brentuximab Vedotin), ADE, Ado-Trastuzumab Emtansine, Adriamycin (Doxorubicin Hydrochloride), Afatinib Dimaleate, Afinitor (Everolimus), Akynzeo (Netupitant and Palonosetron Hydrochloride), Aldara (Imiquimod), Aldesleukin, Alecensa (Alectinib), Alectinib, Alemtuzumab, Alimta (Pemetrexed Disodium), Aliqopa (Copanlisib Hydrochloride), Alkeran for Injection (Melphalan Hydrochloride), Alkeran Tablets (Melphalan), Aloxi (Palonosetron Hydrochloride), Alunbrig (Brigatinib), Ambochlorin (Chlorambucil), Amboclorin (Chlorambucil), Amifostine, Aminolevulinic Acid, Anastrozole, Aprepitant, Aredia (Pamidronate Disodium), Arimidex (Anastrozole), Aromasin (Exemestane), Arranon (Nelarabine), Arsenic Trioxide, Arzerra (Ofatumumab), Asparaginase Erwinia chrysanthemi, Atezolizumab, Avastin (Bevacizumab), Avelumab, Axicabtagene Ciloleucel, Axitinib, Azacitidine, Bavencio (Avelumab), BEACOPP, Becenum (Carmustine), Beleodaq (Belinostat), Belinostat, Bendamustine Hydrochloride, BEP, Besponsa (Inotuzumab Ozogamicin), Bevacizumab, Bexarotene, Bexxar (Tositumomab and Iodine I 131 Tositumomab), Bicalutamide, BiCNU (Carmustine), Bleomycin, Blinatumomab, Blincyto (Blinatumomab), Bortezomib, Bosulif (Bosutinib), Bosutinib, Brentuximab Vedotin, Brigatinib, BuMel, Busulfan, Busulfex (Busulfan), Cabazitaxel, Cabometyx (Cabozantinib-S-Malate), Cabozantinib-S-Malate, CAF, Calquence (Acalabrutinib), Campath (Alemtuzumab), Camptosar (Irinotecan Hydrochloride), Capecitabine, CAPOX, Carac (Fluorouracil-Topical), Carboplatin, CARBOPLATIN-TAXOL, Carfilzomib, Carmubris (Carmustine), Carmustine, Carmustine Implant, Casodex (Bicalutamide), CEM, Ceritinib, Cerubidine (Daunorubicin Hydrochloride), Cervarix (Recombinant HPV Bivalent Vaccine), Cetuximab, CEV, Chlorambucil, CHLORAMBUCIL-PREDNISONE, CHOP, Cisplatin, Cladribine, Clafen (Cyclophosphamide), Clofarabine, Clofarex (Clofarabine), Clolar (Clofarabine), CMF, Cobimetinib, Cometriq (Cabozantinib-S-Malate), Copanlisib Hydrochloride, COPDAC, COPP, COPP-ABV, Cosmegen (Dactinomycin), Cotellic (Cobimetinib), Crizotinib, CVP, Cyclophosphamide, Cyfos (Ifosfamide), Cyramza (Ramucirumab), Cytarabine, Cytarabine Liposome, Cytosar-U (Cytarabine), Cytoxan (Cyclophosphamide), Dabrafenib, Dacarbazine, Dacogen (Decitabine), Dactinomycin, Daratumumab, Darzalex (Daratumumab), Dasatinib, Daunorubicin Hydrochloride, Daunorubicin Hydrochloride and Cytarabine Liposome, Decitabine, Defibrotide Sodium, Defitelio (Defibrotide Sodium), Degarelix, Denileukin Diftitox, Denosumab, DepoCyt (Cytarabine Liposome), Dexamethasone, Dexrazoxane Hydrochloride, Dinutuximab, Docetaxel, Doxil (Doxorubicin Hydrochloride Liposome), Doxorubicin Hydrochloride, Doxorubicin Hydrochloride Liposome, Dox-SL (Doxorubicin Hydrochloride Liposome), DTIC-Dome (Dacarbazine), Durvalumab, Efudex (Fluorouracil-Topical), Elitek (Rasburicase), Ellence (Epirubicin Hydrochloride), Elotuzumab, Eloxatin (Oxaliplatin), Eltrombopag Olamine, Emend (Aprepitant), Empliciti (Elotuzumab), Enasidenib Mesylate, Enzalutamide, Epirubicin Hydrochloride, EPOCH, Erbitux (Cetuximab), Eribulin Mesylate, Erivedge (Vismodegib), Erlotinib Hydrochloride, Erwinaze (Asparaginase Erwinia chrysanthemi), Ethyol (Amifostine), Etopophos (Etoposide Phosphate), Etoposide, Etoposide Phosphate, Evacet (Doxorubicin Hydrochloride Liposome), Everolimus, Evista (Raloxifene Hydrochloride), Evomela (Melphalan Hydrochloride), Exemestane, 5-FU (Fluorouracil Injection), 5-FU (Fluorouracil-Topical), Fareston (Toremifene), Farydak (Panobinostat), Faslodex (Fulvestrant), FEC, Femara (Letrozole), Filgrastim, Fludara (Fludarabine Phosphate), Fludarabine Phosphate, Fluoroplex (Fluorouracil-Topical), Fluorouracil Injection, Fluorouracil-Topical, Flutamide, Folex (Methotrexate), Folex PFS (Methotrexate), FOLFIRI, FOLFIRI-BEVACIZUMAB, FOLFIRI-CETUXIMAB, FOLFIRINOX, FOLFOX, Folotyn (Pralatrexate), FU-LV, Fulvestrant, Gardasil (Recombinant HPV Quadrivalent Vaccine), Gardasil 9 (Recombinant HPV Nonavalent Vaccine), Gazyva (Obinutuzumab), Gefitinib, Gemcitabine Hydrochloride, GEMCITABINE-CISPLATIN, GEMCITABINE-OXALIPLATIN, Gemtuzumab Ozogamicin, Gemzar (Gemcitabine Hydrochloride), Gilotrif (Afatinib Dimaleate), Gleevec (Imatinib Mesylate), Gliadel (Carmustine Implant), Gliadel wafer (Carmustine Implant), Glucarpidase, Goserelin Acetate, Halaven (Eribulin Mesylate), Hemangeol (Propranolol Hydrochloride), Herceptin (Trastuzumab), HPV Bivalent Vaccine, Recombinant, HPV Nonavalent Vaccine, Recombinant, HPV Quadrivalent Vaccine, Recombinant, Hycamtin (Topotecan Hydrochloride), Hydrea (Hydroxyurea), Hydroxyurea, Hyper-CVAD, Ibrance (Palbociclib), Ibritumomab Tiuxetan, Ibrutinib, ICE, Iclusig (Ponatinib Hydrochloride), Idamycin (Idarubicin Hydrochloride), Idarubicin Hydrochloride, Idelalisib, Idhifa (Enasidenib Mesylate), Ifex (Ifosfamide), Ifosfamide, Ifosfamidum (Ifosfamide), IL-2 (Aldesleukin), Imatinib Mesylate, Imbruvica (Ibrutinib), Imfinzi (Durvalumab), Imiquimod, Imlygic (Talimogene Laherparepvec), Inlyta (Axitinib), notuzumab Ozogamicin, Interferon Alfa-2b, Recombinant, Interleukin-2 (Aldesleukin), Intron A (Recombinant Interferon Alfa-2b), Iodine I 131 Tositumomab and Tositumomab, Ipilimumab, Iressa (Gefitinib), Irinotecan Hydrochloride, Irinotecan Hydrochloride Liposome, Istodax (Romidepsin), Ixabepilone, Ixazomib Citrate, Ixempra (Ixabepilone), Jakafi (Ruxolitinib Phosphate), JEB, Jevtana (Cabazitaxel), Kadcyla (Ado-Trastuzumab Emtansine), Keoxifene (Raloxifene Hydrochloride), Kepivance (Palifermin), Keytruda (Pembrolizumab), Kisqali (Ribociclib), Kymriah (Tisagenlecleucel), Kyprolis (Carfilzomib), Lanreotide Acetate, Lapatinib Ditosylate, Lartruvo (Olaratumab), Lenalidomide, Lenvatinib Mesylate, Lenvima (Lenvatinib Mesylate), Letrozole, Leucovorin Calcium, Leukeran (Chlorambucil), Leuprolide Acetate, Leustatin (Cladribine), Levulan (Aminolevulinic Acid), Linfolizin (Chlorambucil), LipoDox (Doxorubicin Hydrochloride Liposome), Lomustine, Lonsurf (Trifluridine and Tipiracil Hydrochloride), Lupron (Leuprolide Acetate), Lupron Depot (Leuprolide Acetate), Lupron Depot-Ped (Leuprolide Acetate), Lynparza (Olaparib), Marqibo (Vincristine Sulfate Liposome), Matulane (Procarbazine Hydrochloride), Mechlorethamine Hydrochloride, Megestrol Acetate, Mekinist (Trametinib), Melphalan, Melphalan Hydrochloride, Mercaptopurine, Mesna, Mesnex (Mesna), Methazolastone (Temozolomide), Methotrexate, Methotrexate LPF (Methotrexate), Methylnaltrexone Bromide, Mexate (Methotrexate), Mexate-AQ (Methotrexate), Midostaurin, Mitomycin C, Mitoxantrone Hydrochloride, Mitozytrex (Mitomycin C), MOPP, Mozobil (Plerixafor), Mustargen (Mechlorethamine Hydrochloride), Mutamycin (Mitomycin C), Myleran (Busulfan), Mylosar (Azacitidine), Mylotarg (Gemtuzumab Ozogamicin), Nanoparticle Paclitaxel (Paclitaxel Albumin-stabilized Nanoparticle Formulation), Navelbine (Vinorelbine Tartrate), Necitumumab, Nelarabine, Neosar (Cyclophosphamide), Neratinib Maleate, Nerlynx (Neratinib Maleate), Netupitant and Palonosetron Hydrochloride, Neulasta (Pegfilgrastim), Neupogen (Filgrastim), Nexavar (Sorafenib Tosylate), Nilandron (Nilutamide), Nilotinib, Nilutamide, Ninlaro (Ixazomib Citrate), Niraparib Tosylate Monohydrate, Nivolumab, Nolvadex (Tamoxifen Citrate), Nplate (Romiplostim), Obinutuzumab, Odomzo (Sonidegib), OEPA, Ofatumumab, OFF, Olaparib, Olaratumab, Omacetaxine Mepesuccinate, Oncaspar (Pegaspargase), Ondansetron Hydrochloride, Onivyde (Irinotecan Hydrochloride Liposome), Ontak (Denileukin Diftitox), Opdivo (Nivolumab), OPPA, Osimertinib, Oxaliplatin, Paclitaxel, Paclitaxel Albumin-stabilized Nanoparticle Formulation, PAD, Palbociclib, Palifermin, Palonosetron Hydrochloride, Palonosetron Hydrochloride and Netupitant, Pamidronate Disodium, Panitumumab, Panobinostat, Paraplat (Carboplatin), Paraplatin (Carboplatin), Pazopanib Hydrochloride, PCV, PEB, Pegaspargase, Pegfilgrastim, Peginterferon Alfa-2b, PEG-Intron (Peginterferon Alfa-2b), Pembrolizumab, Pemetrexed Disodium, Perjeta (Pertuzumab), Pertuzumab, Platinol (Cisplatin), Platinol-AQ (Cisplatin), Plerixafor, Pomalidomide, Pomalyst (Pomalidomide), Ponatinib Hydrochloride, Portrazza (Necitumumab), Pralatrexate, Prednisone, Procarbazine Hydrochloride, Proleukin (Aldesleukin), Prolia (Denosumab), Promacta (Eltrombopag Olamine), Propranolol Hydrochloride, Provenge (Sipuleucel-T), Purinethol (Mercaptopurine), Purixan (Mercaptopurine), Radium 223 Dichloride, Raloxifene Hydrochloride, Ramucirumab, Rasburicase, R-CHOP, R-CVP, Recombinant Human Papillomavirus (HPV) Bivalent Vaccine, Recombinant Human Papillomavirus (HPV) Nonavalent Vaccine, Recombinant Human Papillomavirus (HPV) Quadrivalent Vaccine, Recombinant Interferon Alfa-2b, Regorafenib, Relistor (Methylnaltrexone Bromide), R-EPOCH, Revlimid (Lenalidomide), Rheumatrex (Methotrexate), Ribociclib, R-ICE, Rituxan (Rituximab), Rituxan Hycela (Rituximab and Hyaluronidase Human), Rituximab, Rituximab and Hyaluronidase Human, Rolapitant Hydrochloride, Romidepsin, Romiplostim, Rubidomycin (Daunorubicin Hydrochloride), Rubraca (Rucaparib Camsylate), Rucaparib Camsylate, Ruxolitinib Phosphate, Rydapt (Midostaurin), Sclerosol Intrapleural Aerosol (Talc), Siltuximab, Sipuleucel-T, Somatuline Depot (Lanreotide Acetate), Sonidegib, Sorafenib Tosylate, Sprycel (Dasatinib), STANFORD V, Sterile Talc Powder (Talc), Steritalc (Talc), Stivarga (Regorafenib), Sunitinib Malate, Sutent (Sunitinib Malate), Sylatron (Peginterferon Alfa-2b), Sylvant (Siltuximab), Synribo (Omacetaxine Mepesuccinate), Tabloid (Thioguanine), TAC, Tafinlar (Dabrafenib), Tagrisso (Osimertinib), Talc, Talimogene Laherparepvec, Tamoxifen Citrate, Tarabine PFS (Cytarabine), Tarceva (Erlotinib Hydrochloride), Targretin (Bexarotene), Tasigna (Nilotinib), Taxol (Paclitaxel), Taxotere (Docetaxel), Tecentriq (Atezolizumab), Temodar (Temozolomide), Temozolomide, Temsirolimus, Thalidomide, Thalomid (Thalidomide), Thioguanine, Thiotepa, Tisagenlecleucel, Tolak (Fluorouracil-Topical), Topotecan Hydrochloride, Toremifene, Torisel (Temsirolimus), Tositumomab and Iodine I 131 Tositumomab, Totect (Dexrazoxane Hydrochloride), TPF, Trabectedin, Trametinib, Trastuzumab, Treanda (Bendamustine Hydrochloride), Trifluridine and Tipiracil Hydrochloride, Trisenox (Arsenic Trioxide), Tykerb (Lapatinib Ditosylate), Unituxin (Dinutuximab), Uridine Triacetate, VAC, Valrubicin, Valstar (Valrubicin), Vandetanib, VAMP, Varubi (Rolapitant Hydrochloride), Vectibix (Panitumumab), VelP, Velban (Vinblastine Sulfate), Velcade (Bortezomib), Velsar (Vinblastine Sulfate), Vemurafenib, Venclexta (Venetoclax), Venetoclax, Verzenio (Abemaciclib), Viadur (Leuprolide Acetate), Vidaza (Azacitidine), Vinblastine Sulfate, Vincasar PFS (Vincristine Sulfate), Vincristine Sulfate, Vincristine Sulfate Liposome, Vinorelbine Tartrate, VIP, Vismodegib, Vistogard (Uridine Triacetate), Voraxaze (Glucarpidase), Vorinostat, Votrient (Pazopanib Hydrochloride), Vyxeos (Daunorubicin Hydrochloride and Cytarabine Liposome), Wellcovorin (Leucovorin Calcium), Xalkori (Crizotinib), Xeloda (Capecitabine), XELIRI, XELOX, Xgeva (Denosumab), Xofigo (Radium 223 Dichloride), Xtandi (Enzalutamide), Yervoy (Ipilimumab), Yescarta (Axicabtagene Ciloleucel), Yondelis (Trabectedin), Zaltrap (Ziv-Aflibercept), Zarxio (Filgrastim), Zejula (Niraparib Tosylate Monohydrate), Zelboraf (Vemurafenib), Zevalin (lbritumomab Tiuxetan), Zinecard (Dexrazoxane Hydrochloride), Ziv-Aflibercept, Zofran (Ondansetron Hydrochloride), Zoladex (Goserelin Acetate), Zoledronic Acid, Zolinza (Vorinostat), Zometa (Zoledronic Acid), Zydelig (Idelalisib), Zykadia (Ceritinib) and Zytiga (Abiraterone Acetate).

EBV-infection is also implicated in the development/progression of a variety of autoimmune diseases, such as multiple sclerosis and systemic lupus erythematosus (SLE; see e.g. Ascherio and Munger Curr Top Microbiol Immunol. (2015); 390(Pt 1):365-85), and EBV antigen EBNA2 has recently been shown to associate with genetic regions implicated as risk factors for the development of SLE, multiple sclerosis, rheumatoid arthritis, inflammatory bowel disease, type 1 diabetes, juvenile idiopathic arthritis and celiac disease (Harley et al, Nat Genet. (2018) 50(5): 699-707).

Accordingly, in some embodiments the disease/condition to be treated/prevented in accordance with the present disclosure is selected from: an autoimmune disease, SLE, multiple sclerosis, rheumatoid arthritis, inflammatory bowel disease, type 1 diabetes, juvenile idiopathic arthritis and celiac disease.

### Subjects

The subject in accordance with aspects of the present disclosure may be any animal or human. The subject is preferably mammalian, more preferably human. The subject may be a non-human mammal, but is more preferably human. The subject may be male or female. The subject may be a patient. A subject may have been diagnosed with a disease or condition described herein (e.g. a cancer) requiring treatment (e.g. a cancer), may be suspected of having such a disease/condition, or may be at risk of developing/contracting such a disease/condition.

In embodiments according to the present disclosure, the subject is preferably a human subject. In some embodiments, the subject to be treated according to a therapeutic or prophylactic method of the present disclosure is a subject having, or at risk of developing, a disease/condition described herein. In embodiments according to the present disclosure, a subject may be selected for treatment according to the methods based on characterisation for certain markers of such a disease/condition.

A subject may be an allogeneic subject with respect to an intervention in accordance with the present disclosure. A subject to be treated/prevented in accordance with the present disclosure may be genetically non-identical to the subject from which the immune cells are derived. A subject to be treated/prevented in accordance with the present disclosure may be HLA mismatched with respect to the subject from which the immune cells are derived. A subject to be treated/prevented in accordance with the present disclosure may be HLA matched with respect to the subject from which the immune cells are derived.

The subject to which cells are administered in accordance with the present disclosure may be allogeneic/non-autologous with respect to the source from which the cells are/were derived. The subject to which cells are administered may be a different subject to the subject from which cells are/were obtained for the production of the cells to be administered. The subject to which the cells are administered may be genetically non-identical to the subject from which cells are/were obtained for the production of the cells to be administered.

The subject to which cells are administered may comprise MHC/HLA genes encoding MHC/HLA molecules which are non-identical to the MHC/HLA molecules encoded by the MHC/HLA genes of the subject from which cells are/were obtained for the production of the cells to be administered. The subject to which cells are administered may comprise MHC/HLA genes encoding MHC/HLA molecules which are identical to the MHC/HLA molecules encoded by the MHC/HLA genes of the subject from which cells are/were obtained for the production of the cells to be administered.

In some embodiments, the subject to which cells are administered is HLA matched with respect to the subject from which cells are/were obtained for the production of the cells to be administered. In some embodiments, the subject to which cells are administered is a near or complete HLA match with respect to the subject from which cells are/were obtained for the production of the cells to be administered. In some embodiments, the subject is a 24/8 (i.e. 4/8, 5/8, 6/8, 7/8 or 8/8) match across HLA-A, -B, -C, and -DRB1. In some embodiments, the subject is a ≥5/10 (i.e. 5/10, 6/10, 7/10, 8/10, 9/10 or 10/10) match across HLA-A, -B, -C, -DRB1 and -DQB1. In some embodiments, the subject is a ≥6/12 (i.e. 6/12, 7/12 8/12, 9/12, 10/12, 11/12 or 12/12) match across HLA-A, -B, -C, -DRB1, -DQB1 and -DPB1. In some embodiments, the subject is an 8/8 match across HLA-A, -B, -C, and -DRB1. In some embodiments, the subject is a 10/10 match across HLA-A, -B, -C, -DRB1 and -DQB1. In some embodiments, the subject is a 12/12 match across HLA-A, -B, -C, -DRB1, -DQB1 and -DPB1.

### Sequence Identity

As used herein, ‘sequence identity’ refers to the percent of nucleotides/amino acid residues in a subject sequence that are identical to nucleotides/amino acid residues in a reference sequence, after aligning the sequences and, if necessary, introducing gaps, to achieve the maximum percent sequence identity between the sequences. Pairwise and multiple sequence alignment for the purposes of determining percent sequence identity between two or more amino acid or nucleic acid sequences can be achieved in various ways known to a person of skill in the art, for instance, using publicly available computer software such as ClustalOmega (Söding, J., Bioinformatics (2005) 21, 951-960), T-coffee (Notredame et al., J. Mol. Biol. (2000) 302, 205-217), Kalign (Lassmann and Sonnhammer, BMC Bioinformatics (2005) 6,298) and MAFFT (Katoh and Standley, Molecular Biology and Evolution (2013) 30(4) 772-780) software. When using such software, the default parameters, e.g. for gap penalty and extension penalty, are preferably used.

Sequences

| SEQ ID | | |
|---|---|---|
| NO: | DESCRIPTION | SEQUENCE |
| 1 | Human SERPINB9 | METLSNASGTFAIRLLKILCQDNPSHNVFCSPVSISSALAMVLLGAKGNTATQM |
| | | AQALSLNTEEDIHRAFQSLLTEVNKAGTQYLLRTANRLFGEKTCQFLSTFKESC |
| | | LQFYHAELKELSFIRAAEESRKHINTWVSKKTEGKIEELLPGSSIDAETRLVLVN |
| | | AIYFKGKWNEPFDETYTREMPFKINQEEQRPVQMMYQEATFKLAHVGEVRAQ |
| | | LLELPYARKELSLLVLLPDDGVELSTVEKSLTFEKLTAWTKPDCMKSTEVEVLLP |
| | | KFKLQEDYDMESVLRHLGIVDAFQQGKADLSAMSAERDLCLSKFVHKSFVEVN |
| | | EEGTEAAAASSCFVVAECCMESGPRFCADHPFLFFIRHNRANSILFCGRFSSP |
| 2 | Human SERPINB9 | SCFWVAECCMESGPR |
| | reactive centre loop | |
| 3 | Human SERPINB9 | SCFVVAX₁X₂X₃MESGPR |
| | reactive centre loop | wherein X₁ = E or D; X₂ = C or S; X₃ = C or S |
| | variant CON | |
| 4 | SERPINB9 variant | METLSNASGTFAIRLLKILCQDNPSHNVFCSPVSISSALAMVLLGAKGNTATQM |
| | CON | AQALSLNTEEDIHRAFQSLLTEVNKAGTQYLLRTANRLFGEKTCQFLSTFKESC |
| | | LQFYHAELKELSFIRAAEESRKHINTWVSKKTEGKIEELLPGSSIDAETRLVLVN |
| | | AIYFKGKWNEPFDETYTREMPFKINQEEQRPVQMMYQEATFKLAHVGEVRAQ |
| | | LLELPYARKELSLLVLLPDDGVELSTVEKSLTFEKLTAWTKPDCMKSTEVEVLLP |
| | | KFKLQEDYDMESVLRHLGIVDAFQQGKADLSAMSAERDLCLSKFVHKSFVEVN |
| | | EEGTEAAAASSCFVVAX₁X₂X₃MESGPRFCADHPFLFFIRHNRANSILFCGRFSS |
| | | P |
| | | wherein X₁ = E or D; X₂ = C or S; X₃ = C or S |
| 5 | SERPINB9 (E340D) | METLSNASGTFAIRLLKILCQDNPSHNVFCSPVSISSALAMVLLGAKGNTATQM |
| | | AQALSLNTEEDIHRAFQSLLTEVNKAGTQYLLRTANRLFGEKTCQFLSTFKESC |
| | | LQFYHAELKELSFIRAAEESRKHINTWVSKKTEGKIEELLPGSSIDAETRLVLVN |
| | | AIYFKGKWNEPFDETYTREMPFKINQEEQRPVQMMYQEATFKLAHVGEVRAQ |
| | | LLELPYARKELSLLVLLPDDGVELSTVEKSLTFEKLTAWTKPDCMKSTEVEVLLP |
| | | KFKLQEDYDMESVLRHLGIVDAFQQGKADLSAMSAERDLCLSKFVHKSFVEVN |
| | | EEGTEAAAASSCFVVADCCMESGPRFCADHPFLFFIRHNRANSILFCGRFSSP |
| 6 | SERPINB9 (C341S; | METLSNASGTFAIRLLKILCQDNPSHNVFCSPVSISSALAMVLLGAKGNTATQM |
| | C342S) | AQALSLNTEEDIHRAFQSLLTEVNKAGTQYLLRTANRLFGEKTCQFLSTFKESC |
| | | LQFYHAELKELSFIRAAEESRKHINTWVSKKTEGKIEELLPGSSIDAETRLVLVN |
| | | AIYFKGKWNEPFDETYTREMPFKINQEEQRPVQMMYQEATFKLAHVGEVRAQ |
| | | LLELPYARKELSLLVLLPDDGVELSTVEKSLTFEKLTAWTKPDCMKSTEVEVLLP |
| | | KFKLQEDYDMESVLRHLGIVDAFQQGKADLSAMSAERDLCLSKFVHKSFVEVN |
| | | EEGTEAAAASSCFVVAESSMESGPRFCADHPFLFFIRHNRANSILFCGRFSSP |
| 7 | SERPINB9 (E340D; | METLSNASGTFAIRLLKILCQDNPSHNVFCSPVSISSALAMVLLGAKGNTATQM |
| | C341S; C342S) | AQALSLNTEEDIHRAFQSLLTEVNKAGTQYLLRTANRLFGEKTCQFLSTFKESC |
| | | LQFYHAELKELSFIRAAEESRKHINTWVSKKTEGKIEELLPGSSIDAETRLVLVN |
| | | AIYFKGKWNEPFDETYTREMPFKINQEEQRPVQMMYQEATFKLAHVGEVRAQ |
| | | LLELPYARKELSLLVLLPDDGVELSTVEKSLTFEKLTAWTKPDCMKSTEVEVLLP |
| | | KFKLQEDYDMESVLRHLGIVDAFQQGKADLSAMSAERDLCLSKFVHKSFVEVN |
| | | EEGTEAAAASSCFVVADSSMESGPRFCADHPFLFFIRHNRANSILFCGRFSSP |
| 8 | Human CD30 | MRVLLAALGLLFLGALRAFPQDRPFEDTCHGNPSHYYDKAVRRCCYRCPMGL |
| | isoform 1 (UniProt: | FPTQQCPQRPTDCRKQCEPDYYLDEADRCTACVTCSRDDLVEKTPCAWNSS |
| | P28908-1, v1) | RVCECRPGMFCSTSAVNSCARCFFHSVCPAGMIVKFPGTAQKNTVCEPASPG |
| | | VSPACASPENCKEPSSGTIPQAKPTPVSPATSSASTMPVRGGTRLAQEAASKL |
| | | TRAPDSPSSVGRPSSDPGLSPTQPCPEGSGDCRKQCEPDYYLDEAGRCTAC |
| | | VSCSRDDLVEKTPCAWNSSRTCECRPGMICATSATNSCARCVPYPICAAETVT |
| | | KPQDMAEKDTTFEAPPLGTQPDCNPTPENGEAPASTSPTQSLLVDSQASKTLP |
| | | IPTSAPVALSSTGKPVLDAGPVLFWVILVLVVVVGSSAFLLCHRRACRKRIRQKL |
| | | HLCYPVQTSQPKLELVDSRPRRSSTQLRSGASVTEPVAEERGLMSQPLMETC |
| | | HSVGAAYLESLPLQDASPAGGPSSPRDLPEPRVSTEHTNNKIEKIYIMKADTVIV |
| | | GTVKAELPEGRGLAGPAEPELEEELEADHTPHYPEQETEPPLGSCSDVMLSVE |
| | | EEGKEDPLPTAASGK |
| 9 | Human CD30 | MSQPLMETCHSVGAAYLESLPLQDASPAGGPSSPRDLPEPRVSTEHTNNKIEK |
| | isoform 2 (UniProt: | IYIMKADTVIVGTVKAELPEGRGLAGPAEPELEEELEADHTPHYPEQETEPPLG |
| | P28908-2) | SCSDVMLSVEEEGKEDPLPTAASGK |
| 10 | Human CD30 | MFCSTSAVNSCARCFFHSVCPAGMIVKFPGTAQKNTVCEPASPGVSPACASP |
| | isoform 3 (UniProt: | ENCKEPSSGTIPQAKPTPVSPATSSASTMPVRGGTRLAQEAASKLTRAPDSPS |
| | P28908-3) | SVGRPSSDPGLSPTQPCPEGSGDCRKQCEPDYYLDEAGRCTACVSCSRDDL |
| | | VEKTPCAWNSSRTCECRPGMICATSATNSCARCVPYPICAAETVTKPQDMAE |
| | | KDTTFEAPPLGTQPDCNPTPENGEAPASTSPTQSLLVDSQASKTLPIPTSAPVA |
| | | LSSTGKPVLDAGPVLFWVILVLVVVVGSSAFLLCHRRACRKRIRQKLHLCYPVQ |
| | | TSQPKLELVDSRPRRSSTLRSGASVTEPVAEERGLMSQPLMETCHSVGAAYL |
| | | ESLPLQDASPAGGPSSPRDLPEPRVSTEHTNNKIEKIYIMKADTVIVGTVKAELP |
| | | EGRGLAGPAEPELEEELEADHTPHYPEQETEPPLGSCSDVMLSVEEEGKEDP |
| | | LPTAASGK |
| 11 | Human CD30 signal | MRVLLAALGLLFLGALRA |
| | peptide | |
| 12 | Human CD30 | FPQDRPFEDTCHGNPSHYYDKAVRRCCYRCPMGLFPTQQCPQRPTDCRKQC |
| | extracellular domain | EPDYYLDEADRCTACVTCSRDDLVEKTPCAWNSSRVCECRPGMFCSTSAVNS |
| | | CARCFFHSVCPAGMIVKFPGTAQKNTVCEPASPGVSPACASPENCKEPSSGTI |
| | | PQAKPTPVSPATSSASTMPVRGGTRLAQEAASKLTRAPDSPSSVGRPSSDPG |
| | | LSPTQPCPEGSGDCRKQCEPDYYLDEAGRCTACVSCSRDDLVEKTPCAWNS |
| | | SRTCECRPGMICATSATNSCARCVPYPICAAETVTKPQDMAEKDTTFEAPPLG |
| | | TQPDCNPTPENGEAPASTSPTQSLLVDSQASKTLPIPTSAPVALSSTGKPVLDA |
| | | G |
| 13 | Human CD30 | PVLFWVILVLVVVVGSSAFLL |
| | transmembrane | |
| | domain | |
| 14 | Human CD30 | CHRRACRKRIRQKLHLCYPVQTSQPKLELVDSRPRRSSTQLRSGASVTEPVAE |
| | cytoplasmic domain | ERGLMSQPLMETCHSVGAAYLESLPLQDASPAGGPSSPRDLPEPRVSTEHTN |
| | | NKIEKIYIMKADTVIVGTVKAELPEGRGLAGPAEPELEEELEADHTPHYPEQETE |
| | | PPLGSCSDVMLSVEEEGKEDPLPTAASGK |
| 15 | HRS3 HC-CDR1 | GYTFTTYT |
| 16 | HRS3 HC-CDR2 | INPSSGCS |
| 17 | HRS3 HC-CDR3 | ARRADYGNYEYTWFAY |
| 18 | HRS3 LC-CDR1 | QNVGTN |
| 19 | HRS3 LC-CDR2 | SAS |
| 20 | HRS3 LC-CDR3 | QQYHTYPLT |
| 21 | HRS3 VH | QVQLQQSGAELARPGASVKMSCKASGYTFTTYTIHWRRRPGHDLEWIGYIN |
| | | PSSGCSDYNQNFKGKTTLTADKSSNTAYMQLNSLTSEDSAVYYCARRADYGN |
| | | YEYTWFAYWGQGTTVTVSS |
| 22 | HRS3 VL | VIELTQSPKFMSTSVGDRVNVTYKASQNVGTNVAWFQQKPGQSPKVLIYSASY |
| | | RYSGVPDRFTGSGSGTDFTLTISNVQSEDLAEYFCQQYHTYPLTFGGGTKLEIK |
| 23 | HRS3 scFv linker | SGGGSGGGGSGGGGS |
| 24 | HRS3 scFv | QVQLQQSGAELARPGASVKMSCKASGYTFTTYTIHWVRRRPGHDLEWIGYIN |
| | | PSSGCSDYNQNFKGKTTLTADKSSNTAYMQLNSLTSEDSAVYYCARRADYGN |
| | | YEYTWFAYWGQGTTVTVSSSGGGSGGGGSGGGGSVIELTQSPKFMSTSVGD |
| | | RVNVTYKASQNVGTNVAWFQQKPGQSPKVLIYSASYRYSGVPDRFTGSGSGT |
| | | DFTLTISNVQSEDLAEYFCQQYHTYPLTFGGGTKLEIK |
| 25 | HRS3 epitope | ATSSASTMPVRGGTRLAQEAASKLTRAPDSPSSVGRPSSDPGLSPTQPCPEG |
| | | SGDCRKQCEPDYYLDEAGRCTACVSCSRDDLVEKTPCAWNSSRTCECRPGM |
| | | ICATSATNSCARCVPYPICAAETVTKPQDMAEKDTTFEAPPLGTQPDC |
| 26 | Human CD28 | FWVLVVVGGVLACYSLLVTVAFIIFWV |
| | transmembrane | |
| | domain | |
| 27 | Human CD3ζ | LCYLLDGILFIYGVILTALFL |
| | transmembrane | |
| | domain | |
| 28 | Human CD8α | IYIWAPLAGTCGVLLLSLVITLYQNHRN |
| | transmembrane | |
| | domain | |
| 29 | ITAM consensus | YXXL/I |
| | | wherein X = any amino acid |
| 30 | Larger ITAM | YXXL/I(X)₆₋₈ YXXL/I |
| | consensus | wherein X = any amino acid |
| 31 | Human CD3ζ | RVKFSRSADAPAYQQGQNQLYNELNLGRREEYDVLDKRRGRDPEMGGKPRR |
| | intracellular domain | KNPQEGLYNELQKDKMAEAYSEIGMKGERRRGKGHDGLYQGLSTATKDTYDA |
| | | LHMQALPPR |
| 32 | Human CD28 | RSKRSRLLHSDYMNMTPRRPGPTRKHYQPYAPPRDFAAYRS |
| | intracellular domain | |
| 33 | Human CD28 | RSKRSRLLHSDYMNMTPRRPGPTRKHYQAYAAARDFAAYRS |
| | intracellular domain | |
| | with mutated Ick | |
| | binding site | |
| 34 | CAR signalling | RSKRSRLLHSDYMNMTPRRPGPTRKHYQPYAPPRDFAAYRSRVKFSRSADAP |
| | domain | AYQQGQNQLYNELNLGRREEYDVLDKRRGRDPEMGGKPRRKNPQEGLYNEL |
| | | QKDKMAEAYSEIGMKGERRRGKGHDGLYQGLSTATKDTYDALHMQALPPR |
| 35 | Human IgG1 CH1- | EPKSCDKTHTCP |
| | CH2 hinge region | |
| 36 | Human IgG1 CH1- | EPKSPDKTHTCP |
| | CH2 hinge region | |
| | C103P variant | |
| 37 | Human IgG1 CH2- | PCPAPELLGGPSVFLFPPKPKDTLMISRTPEVTCVVVDVSHEDPEVKFNWYVD |
| | CH3 hinge region | GVEVHNAKTKPREEQYNSTYRVVSVLTVLHQDWLNGKEYKCKVSNKALPAPIE |
| | | KTISKAKGQPREPQVYTLPPSRDELTKNQVSLTCLVKGFYPSDIAVEWESNGQ |
| | | PENNYKTTPPVLDSDGSFFLYSKLTVDKSRWQQGNVFSCSVMHEALHNHYTQ |
| | | KSLSLSPGK |
| 38 | Human IgG1 CH2- | PCPAPPVAGPSVFLFPPKPKDTLMIARTPEVTCVVVDVSHEDPEVKFNWYVDG |
| | CH3 hinge region | VEVHNAKTKPREEQYNSTYRVVSVLTVLHQDWLNGKEYKCKVSNKALPAPIEK |
| | variant | TISKAKGQPREPQVYTLPPSRDELTKNQVSLTCLVKGFYPSDIAVEWESNGQP |
| | | ENNYKTTPPVLDSDGSFFLYSKLTVDKSRWQQGNVFSCSVMHEALHNHYTQK |
| | | SLSLSPGKKDPK |
| 39 | CAR hinge region | EPKSPDKTHTCPPCPAPELLGGPSVFLFPPKPKDTLMISRTPEVTCVVVDVSHE |
| | | DPEVKFNWYVDGVEVHNAKTKPREEQYNSTYRVVSVLTVLHQDWLNGKEYK |
| | | CKVSNKALPAPIEKTISKAKGQPREPQVYTLPPSRDELTKNQVSLTCLVKGFYP |
| | | SDIAVEWESNGQPENNYKTTPPVLDSDGSFFLYSKLTVDKSRWQQGNVFSCS |
| | | VMHEALHNHYTQKSLSLSPGK |
| 40 | Signal peptide | MDFQVQIFSFLLISASVIMSRMA |
| 41 | CD30.CAR (IgG1 | QVQLQQSGAELARPGASVKMSCKASGYTFTTYTIHWRRRPGHDLEWIGYIN |
| | spacer, lacking | PSSGCSDYNQNFKGKTTLTADKSSNTAYMQLNSLTSEDSAVYYCARRADYGN |
| | signal peptide) | YEYTWFAYWGQGTTVTVSSSGGGSGGGGSGGGGSVIELTQSPKFMSTSVGD |
| | | RVNVTYKASQNVGTNVAWFQQKPGQSPKVLIYSASYRYSGVPDRFTGSGSGT |
| | | DFTLTISNVQSEDLAEYFCQQYHTYPLTFGGGTKLEIKRSDPAEPKSPDKTHTC |
| | | PPCPAPELLGGPSVFLFPPKPKDTLMISRTPEVTCVVVDVSHEDPEVKFNWYV |
| | | DGVEVHNAKTKPREEQYNSTYRVVSVLTVLHQDWLNGKEYKCKVSNKALPAPI |
| | | EKTISKAKGQPREPQVYTLPPSRDELTKNQVSLTCLVKGFYPSDIAVEWESNG |
| | | QPENNYKTTPPVLDSDGSFFLYSKLTVDKSRWQQGNVFSCSVMHEALHNHYT |
| | | QKSLSLSPGKKDPKFWVLVVVGGVLACYSLLVTVAFIIFWVRSKRSRLLHSDYM |
| | | NMTPRRPGPTRKHYQPYAPPRDFAAYRSRVKFSRSADAPAYQQGQNQLYNE |
| | | LNLGRREEYDVLDKRRGRDPEMGGKPRRKNPQEGLYNELQKDKMAEAYSEIG |
| | | MKGERRRGKGHDGLYQGLSTATKDTYDALHMQALPPR |
| 42 | CD30.CAR (IgG1 | MDFQVQIFSFLLISASVIMSRMAQVQLQQSGAELARPGASVKMSCKASGYTFT |
| | spacer, with signal | TYTIHWVRRRPGHDLEWIGYINPSSGCSDYNQNFKGKTTLTADKSSNTAYMQL |
| | peptide) | NSLTSEDSAVYYCARRADYGNYEYTWFAYWGQGTTVTVSSSGGGSGGGGS |
| | | GGGGSVIELTQSPKFMSTSVGDRVNVTYKASQNVGTNVAWFQQKPGQSPKV |
| | | LIYSASYRYSGVPDRFTGSGSGTDFTLTISNVQSEDLAEYFCQQYHTYPLTFGG |
| | | GTKLEIKRSDPAEPKSPDKTHTCPPCPAPELLGGPSVFLFPPKPKDTLMISRTP |
| | | EVTCVVVDVSHEDPEVKFNWYVDGVEVHNAKTKPREEQYNSTYRVVSVLTVL |
| | | HQDWLNGKEYKCKVSNKALPAPIEKTISKAKGQPREPQVYTLPPSRDELTKNQ |
| | | VSLTCLVKGFYPSDIAVEWESNGQPENNYKTTPPVLDSDGSFFLYSKLTVDKS |
| | | RWQQGNVFSCSVMHEALHNHYTQKSLSLSPGKKDPKFWVLVVVGGVLACYS |
| | | LLVTVAFIIFWVRSKRSRLLHSDYMNMTPRRPGPTRKHYQPYAPPRDFAAYRS |
| | | RVKFSRSADAPAYQQGQNQLYNELNLGRREEYDVLDKRRGRDPEMGGKPRR |
| | | KNPQEGLYNELQKDKMAEAYSEIGMKGERRRGKGHDGLYQGLSTATKDTYDA |
| | | LHMQALPPR |
| 43 | SERPINB9-P2A- | METLSNASGTFAIRLLKILCQDNPSHNVFCSPVSISSALAMVLLGAKGNTATQM |
| | CD30.CAR (IgG1 | AQALSLNTEEDIHRAFQSLLTEVNKAGTQYLLRTANRLFGEKTCQFLSTFKESC |
| | spacer) | LQFYHAELKELSFIRAAEESRKHINTWVSKKTEGKIEELLPGSSIDAETRLVLVN |
| | | AIYFKGKWNEPFDETYTREMPFKINQEEQRPVQMMYQEATFKLAHVGEVRAQ |
| | | LLELPYARKELSLLVLLPDDGVELSTVEKSLTFEKLTAWTKPDCMKSTEVEVLLP |
| | | KFKLQEDYDMESVLRHLGIVDAFQQGKADLSAMSAERDLCLSKFVHKSFVEVN |
| | | EEGTEAAAASSCFVVAECCMESGPRFCADHPFLFFIRHNRANSILFCGRFSSP |
| | | RKRRGSGTPDPWGSGATNFSLLKQAGDVEENPGPLELEMDFQVQIFSFLLISA |
| | | SVIMSRMAQVQLQQSGAELARPGASVKMSCKASGYTFTTYTIHWVRRRPGHD |
| | | LEWIGYINPSSGCSDYNQNFKGKTTLTADKSSNTAYMQLNSLTSEDSAVYYCA |
| | | RRADYGNYEYTWFAYWGQGTTVTVSSSGGGSGGGGSGGGGSVIELTQSPKF |
| | | MSTSVGDRVNVTYKASQNVGTNVAWFQQKPGQSPKVLIYSASYRYSGVPDRF |
| | | TGSGSGTDFTLTISNVQSEDLAEYFCQQYHTYPLTFGGGTKLEIKRSDPAEPKS |
| | | PDKTHTCPPCPAPELLGGPSVFLFPPKPKDTLMISRTPEVTCVVVDVSHEDPEV |
| | | KFNWYVDGVEVHNAKTKPREEQYNSTYRVVSVLTVLHQDWLNGKEYKCKVS |
| | | NKALPAPIEKTISKAKGQPREPQVYTLPPSRDELTKNQVSLTCLVKGFYPSDIAV |
| | | EWESNGQPENNYKTTPPVLDSDGSFFLYSKLTVDKSRWQQGNVFSCSVMHE |
| | | ALHNHYTQKSLSLSPGKKDPKFWVLVVVGGVLACYSLLVTVAFIIFWVRSKRSR |
| | | LLHSDYMNMTPRRPGPTRKHYQPYAPPRDFAAYRSRVKFSRSADAPAYQQG |
| | | QNQLYNELNLGRREEYDVLDKRRGRDPEMGGKPRRKNPQEGLYNELQKDKM |
| | | AEAYSEIGMKGERRRGKGHDGLYQGLSTATKDTYDALHMQALPPR |
| 44 | SERPINB9 | METLSNASGTFAIRLLKILCQDNPSHNVFCSPVSISSALAMVLLGAKGNTATQM |
| | (E340D)-P2A- | AQALSLNTEEDIHRAFQSLLTEVNKAGTQYLLRTANRLFGEKTCQFLSTFKESC |
| | CD30.CAR (IgG1 | LQFYHAELKELSFIRAAEESRKHINTWVSKKTEGKIEELLPGSSIDAETRLVLVN |
| | spacer) | AIYFKGKWNEPFDETYTREMPFKINQEEQRPVQMMYQEATFKLAHVGEVRAQ |
| | | LLELPYARKELSLLVLLPDDGVELSTVEKSLTFEKLTAWTKPDCMKSTEVEVLLP |
| | | KFKLQEDYDMESVLRHLGIVDAFQQGKADLSAMSAERDLCLSKFVHKSFVEVN |
| | | EEGTEAAAASSCFVVADCCMESGPRFCADHPFLFFIRHNRANSILFCGRFSSP |
| | | RKRRGSGTPDPWGSGATNFSLLKQAGDVEENPGPLELEMDFQVQIFSFLLISA |
| | | SVIMSRMAQVQLQQSGAELARPGASVKMSCKASGYTFTTYTIHWVRRRPGHD |
| | | LEWIGYINPSSGCSDYNQNFKGKTTLTADKSSNTAYMQLNSLTSEDSAVYYCA |
| | | RRADYGNYEYTWFAYWGQGTTVTVSSSGGGSGGGGSGGGGSVIELTQSPKF |
| | | MSTSVGDRVNVTYKASQNVGTNVAWFQQKPGQSPKVLIYSASYRYSGVPDRF |
| | | TGSGSGTDFTLTISNVQSEDLAEYFCQQYHTYPLTFGGGTKLEIKRSDPAEPKS |
| | | PDKTHTCPPCPAPELLGGPSVFLFPPKPKDTLMISRTPEVTCVVVDVSHEDPEV |
| | | KFNWYVDGVEVHNAKTKPREEQYNSTYRVVSVLTVLHQDWLNGKEYKCKVS |
| | | NKALPAPIEKTISKAKGQPREPQVYTLPPSRDELTKNQVSLTCLVKGFYPSDIAV |
| | | EWESNGQPENNYKTTPPVLDSDGSFFLYSKLTVDKSRWQQGNVFSCSVMHE |
| | | ALHNHYTQKSLSLSPGKKDPKFWVLVVVGGVLACYSLLVTVAFIIFWVRSKRSR |
| | | LLHSDYMNMTPRRPGPTRKHYQPYAPPRDFAAYRSRVKFSRSADAPAYQQG |
| | | QNQLYNELNLGRREEYDVLDKRRGRDPEMGGKPRRKNPQEGLYNELQKDKM |
| | | AEAYSEIGMKGERRRGKGHDGLYQGLSTATKDTYDALHMQALPPR |
| 45 | SERPINB9 (C341S; | METLSNASGTFAIRLLKILCQDNPSHNVFCSPVSISSALAMVLLGAKGNTATQM |
| | C342S)-P2A- | AQALSLNTEEDIHRAFQSLLTEVNKAGTQYLLRTANRLFGEKTCQFLSTFKESC |
| | CD30.CAR (IgG1 | LQFYHAELKELSFIRAAEESRKHINTWVSKKTEGKIEELLPGSSIDAETRLVLVN |
| | spacer) | AIYFKGKWNEPFDETYTREMPFKINQEEQRPVQMMYQEATFKLAHVGEVRAQ |
| | | LLELPYARKELSLLVLLPDDGVELSTVEKSLTFEKLTAWTKPDCMKSTEVEVLLP |
| | | KFKLQEDYDMESVLRHLGIVDAFQQGKADLSAMSAERDLCLSKFVHKSFVEVN |
| | | EEGTEAAAASSCFVVAESSMESGPRFCADHPFLFFIRHNRANSILFCGRFSSP |
| | | RKRRGSGTPDPWGSGATNFSLLKQAGDVEENPGPLELEMDFQVQIFSFLLISA |
| | | SVIMSRMAQVQLQQSGAELARPGASVKMSCKASGYTFTTYTIHWVRRRPGHD |
| | | LEWIGYINPSSGCSDYNQNFKGKTTLTADKSSNTAYMQLNSLTSEDSAVYYCA |
| | | RRADYGNYEYTWFAYWGQGTTVTVSSSGGGSGGGGSGGGGSVIELTQSPKF |
| | | MSTSVGDRVNVTYKASQNVGTNVAWFQQKPGQSPKVLIYSASYRYSGVPDRF |
| | | TGSGSGTDFTLTISNVQSEDLAEYFCQQYHTYPLTFGGGTKLEIKRSDPAEPKS |
| | | PDKTHTCPPCPAPELLGGPSVFLFPPKPKDTLMISRTPEVTCVVVDVSHEDPEV |
| | | KFNWYVDGVEVHNAKTKPREEQYNSTYRVVSVLTVLHQDWLNGKEYKCKVS |
| | | NKALPAPIEKTISKAKGQPREPQVYTLPPSRDELTKNQVSLTCLVKGFYPSDIAV |
| | | EWESNGQPENNYKTTPPVLDSDGSFFLYSKLTVDKSRWQQGNVFSCSVMHE |
| | | ALHNHYTQKSLSLSPGKKDPKFWVLVVVGGVLACYSLLVTVAFIIFWVRSKRSR |
| | | LLHSDYMNMTPRRPGPTRKHYQPYAPPRDFAAYRSRVKFSRSADAPAYQQG |
| | | QNQLYNELNLGRREEYDVLDKRRGRDPEMGGKPRRKNPQEGLYNELQKDKM |
| | | AEAYSEIGMKGERRRGKGHDGLYQGLSTATKDTYDALHMQALPPR |
| 46 | SERPINB9 (E340D; | METLSNASGTFAIRLLKILCQDNPSHNVFCSPVSISSALAMVLLGAKGNTATQM |
| | C341S; C342S)- | AQALSLNTEEDIHRAFQSLLTEVNKAGTQYLLRTANRLFGEKTCQFLSTFKESC |
| | P2A-CD30.CAR | LQFYHAELKELSFIRAAEESRKHINTWVSKKTEGKIEELLPGSSIDAETRLVLVN |
| | (IgG1 spacer) | AIYFKGKWNEPFDETYTREMPFKINQEEQRPVQMMYQEATFKLAHVGEVRAQ |
| | | LLELPYARKELSLLVLLPDDGVELSTVEKSLTFEKLTAWTKPDCMKSTEVEVLLP |
| | | KFKLQEDYDMESVLRHLGIVDAFQQGKADLSAMSAERDLCLSKFVHKSFVEVN |
| | | EEGTEAAAASSCFVVADSSMESGPRFCADHPFLFFIRHNRANSILFCGRFSSP |
| | | RKRRGSGTPDPWGSGATNFSLLKQAGDVEENPGPLELEMDFQVQIFSFLLISA |
| | | SVIMSRMAQVQLQQSGAELARPGASVKMSCKASGYTFTTYTIHWVRRRPGHD |
| | | LEWIGYINPSSGCSDYNQNFKGKTTLTADKSSNTAYMQLNSLTSEDSAVYYCA |
| | | RRADYGNYEYTWFAYWGQGTTVTVSSSGGGSGGGGSGGGGSVIELTQSPKF |
| | | MSTSVGDRVNVTYKASQNVGTNVAWFQQKPGQSPKVLIYSASYRYSGVPDRF |
| | | TGSGSGTDFTLTISNVQSEDLAEYFCQQYHTYPLTFGGGTKLEIKRSDPAEPKS |
| | | PDKTHTCPPCPAPELLGGPSVFLFPPKPKDTLMISRTPEVTCVVVDVSHEDPEV |
| | | KFNWYVDGVEVHNAKTKPREEQYNSTYRVVSVLTVLHQDWLNGKEYKCKVS |
| | | NKALPAPIEKTISKAKGQPREPQVYTLPPSRDELTKNQVSLTCLVKGFYPSDIAV |
| | | EWESNGQPENNYKTTPPVLDSDGSFFLYSKLTVDKSRWQQGNVFSCSVMHE |
| | | ALHNHYTQKSLSLSPGKKDPKFWVLVVVGGVLACYSLLVTVAFIIFWVRSKRSR |
| | | LLHSDYMNMTPRRPGPTRKHYQPYAPPRDFAAYRSRVKFSRSADAPAYQQG |
| | | QNQLYNELNLGRREEYDVLDKRRGRDPEMGGKPRRKNPQEGLYNELQKDKM |
| | | AEAYSEIGMKGERRRGKGHDGLYQGLSTATKDTYDALHMQALPPR |
| 47 | SERPINB9 (T327R; | METLSNASGTFAIRLLKILCQDNPSHNVFCSPVSISSALAMVLLGAKGNTATQM |
| | E340A) | AQALSLNTEEDIHRAFQSLLTEVNKAGTQYLLRTANRLFGEKTCQFLSTFKESC |
| | | LQFYHAELKELSFIRAAEESRKHINTWVSKKTEGKIEELLPGSSIDAETRLVLVN |
| | | AIYFKGKWNEPFDETYTREMPFKINQEEQRPVQMMYQEATFKLAHVGEVRAQ |
| | | LLELPYARKELSLLVLLPDDGVELSTVEKSLTFEKLTAWTKPDCMKSTEVEVLLP |
| | | KFKLQEDYDMESVLRHLGIVDAFQQGKADLSAMSAERDLCLSKFVHKSFVEVN |
| | | EEGREAAAASSCFVVAACCMESGPRFCADHPFLFFIRHNRANSILFCGRFSSP |
| 48 | SERPINB9 | METLSNASGTFAIRLLKILCQDNPSHNVFCSPVSISSALAMVLLGAKGNTATQM |
| | (E340D)-T2A- | AQALSLNTEEDIHRAFQSLLTEVNKAGTQYLLRTANRLFGEKTCQFLSTFKESC |
| | CD30.CAR (IgG1 | LQFYHAELKELSFIRAAEESRKHINTWVSKKTEGKIEELLPGSSIDAETRLVLVN |
| | spacer) | AIYFKGKWNEPFDETYTREMPFKINQEEQRPVQMMYQEATFKLAHVGEVRAQ |
| | | LLELPYARKELSLLVLLPDDGVELSTVEKSLTFEKLTAWTKPDCMKSTEVEVLLP |
| | | KFKLQEDYDMESVLRHLGIVDAFQQGKADLSAMSAERDLCLSKFVHKSFVEVN |
| | | EEGTEAAAASSCFVVADCCMESGPRFCADHPFLFFIRHNRANSILFCGRFSSP |
| | | TRGSGEGRGSLLTCGDVEENPGPLEMDFQVQIFSFLLISASVIMSRMAQVQLQ |
| | | QSGAELARPGASVKMSCKASGYTFTTYTIHWVRRRPGHDLEWIGYINPSSGCS |
| | | DYNQNFKGKTTLTADKSSNTAYMQLNSLTSEDSAVYYCARRADYGNYEYTWF |
| | | AYWGQGTTVTVSSSGGGSGGGGSGGGGSVIELTQSPKFMSTSVGDRVNVTY |
| | | KASQNVGTNVAWFQQKPGQSPKVLIYSASYRYSGVPDRFTGSGSGTDFTLTIS |
| | | NVQSEDLAEYFCQQYHTYPLTFGGGTKLEIKRSDPAEPKSPDKTHTCPPCPAP |
| | | ELLGGPSVFLFPPKPKDTLMISRTPEVTCVVVDVSHEDPEVKFNWYVDGVEVH |
| | | NAKTKPREEQYNSTYRVVSVLTVLHQDWLNGKEYKCKVSNKALPAPIEKTISKA |
| | | KGQPREPQVYTLPPSRDELTKNQVSLTCLVKGFYPSDIAVEWESNGQPENNY |
| | | KTTPPVLDSDGSFFLYSKLTVDKSRWQQGNVFSCSVMHEALHNHYTQKSLSL |
| | | SPGKKDPKFWVLVVVGGVLACYSLLVTVAFIIFWVRSKRSRLLHSDYMNMTPR |
| | | RPGPTRKHYQPYAPPRDFAAYRSRVKFSRSADAPAYQQGQNQLYNELNLGR |
| | | REEYDVLDKRRGRDPEMGGKPRRKNPQEGLYNELQKDKMAEAYSEIGMKGE |
| | | RRRGKGHDGLYQGLSTATKDTYDALHMQALPPR |
| 49 | 4-1BB spacer | SPADLSPGASSVTPPAPAREPGHSP |
| 50 | CD30.CAR (4-1BB | MDFQVQIFSFLLISASVIMSRMAQVQLQQSGAELARPGASVKMSCKASGYTFT |
| | spacer, with signal | TYTIHWVRRRPGHDLEWIGYINPSSGCSDYNQNFKGKTTLTADKSSNTAYMQL |
| | peptide) | NSLTSEDSAVYYCARRADYGNYEYTWFAYWGQGTTVTVSSSGGGSGGGGS |
| | | GGGGSVIELTQSPKFMSTSVGDRVNVTYKASQNVGTNVAWFQQKPGQSPKV |
| | | LIYSASYRYSGVPDRFTGSGSGTDFTLTISNVQSEDLAEYFCQQYHTYPLTFGG |
| | | GTKLEIKRSDPASPADLSPGASSVTPPAPAREPGHSPKDPKFWVLVVVGGVLA |
| | | CYSLLVTVAFIIFWVRSKRSRLLHSDYMNMTPRRPGPTRKHYQPYAPPRDFAA |
| | | YRSRVKFSRSADAPAYQQGQNQLYNELNLGRREEYDVLDKRRGRDPEMGGK |
| | | PRRKNPQEGLYNELQKDKMAEAYSEIGMKGERRRGKGHDGLYQGLSTATKDT |
| | | YDALHMQALPPR |
| 51 | CD30.CAR (4-1BB | QVQLQQSGAELARPGASVKMSCKASGYTFTTYTIHWRRRPGHDLEWIGYIN |
| | spacer, without | PSSGCSDYNQNFKGKTTLTADKSSNTAYMQLNSLTSEDSAVYYCARRADYGN |
| | signal peptide) | YEYTWFAYWGQGTTVTVSSSGGGSGGGGSGGGGSVIELTQSPKFMSTSVGD |
| | | RVNVTYKASQNVGTNVAWFQQKPGQSPKVLIYSASYRYSGVPDRFTGSGSGT |
| | | DFTLTISNVQSEDLAEYFCQQYHTYPLTFGGGTKLEIKRSDPASPADLSPGASS |
| | | VTPPAPAREPGHSPKDPKFWVLVVVGGVLACYSLLVTVAFIIFWVRSKRSRLLH |
| | | SDYMNMTPRRPGPTRKHYQPYAPPRDFAAYRSRVKFSRSADAPAYQQGQNQ |
| | | LYNELNLGRREEYDVLDKRRGRDPEMGGKPRRKNPQEGLYNELQKDKMAEA |
| | | YSEIGMKGERRRGKGHDGLYQGLSTATKDTYDALHMQALPPR |
| 52 | Human CD19 | MPPPRLLFFLLFLTPMEVRPEEPLVVKVEEGDNAVLQCLKGTSDGPTQQLTWS |
| | isoform 1 (UniProt | RESPLKPFLKLSLGLPGLGIHMRPLAIWLFIFNVSQQMGGFYLCQPGPPSEKA |
| | P15391-1, v6) | WQPGWTVNVEGSGELFRWNVSDLGGLGCGLKNRSSEGPSSPSGKLMSPKLY |
| | | VWAKDRPEIWEGEPPCLPPRDSLNQSLSQDLTMAPGSTLWLSCGVPPDSVSR |
| | | GPLSWTHVHPKGPKSLLSLELKDDRPARDMWVMETGLLLPRATAQDAGKYYC |
| | | HRGNLTMSFHLEITARPVLWHWLLRTGGWKVSAVTLAYLIFCLCSLVGILHLQR |
| | | ALVLRRKRKRMTDPTRRFFKVTPPPGSGPQNQYGNVLSLPTPTSGLGRAQRW |
| | | AAGLGGTAPSYGNPSSDVQADGALGSRSPPGVGPEEEEGEGYEEPDSEEDS |
| | | EFYENDSNLGQDQLSQDGSGYENPEDEPLGPEDEDSFSNAESYENEDEELTQ |
| | | PVARTMDFLSPHGSAWDPSREATSLGSQSYEDMRGILYAAPQLRSIRGQPGP |
| | | NHEEDADSYENMDNPDGPDPAWGGGGRMGTWSTR |
| 53 | Human CD19 | MPPPRLLFFLLFLTPMEVRPEEPLVVKVEEGDNAVLQCLKGTSDGPTQQLTWS |
| | isoform 2 (UniProt | RESPLKPFLKLSLGLPGLGIHMRPLAIWLFIFNVSQQMGGFYLCQPGPPSEKA |
| | P15391-2) | WQPGWTVNVEGSGELFRWNVSDLGGLGCGLKNRSSEGPSSPSGKLMSPKLY |
| | | VWAKDRPEIWEGEPPCLPPRDSLNQSLSQDLTMAPGSTLWLSCGVPPDSVSR |
| | | GPLSWTHVHPKGPKSLLSLELKDDRPARDMWVMETGLLLPRATAQDAGKYYC |
| | | HRGNLTMSFHLEITARPVLWHWLLRTGGWKVSAVTLAYLIFCLCSLVGILHLQR |
| | | ALVLRRKRKRMTDPTRRFFKVTPPPGSGPQNQYGNVLSLPTPTSGLGRAQRW |
| | | AAGLGGTAPSYGNPSSDVQADGALGSRSPPGVGPEEEEGEGYEEPDSEEDS |
| | | EFYENDSNLGQDQLSQDGSGYENPEDEPLGPEDEDSFSNAESYENEDEELTQ |
| | | PVARTMDFLSPHGSAWDPSREATSLAGSQSYEDMRGILYAAPQLRSIRGQPG |
| | | PNHEEDADSYENMDNPDGPDPAWGGGGRMGTWSTR |
| 54 | Human CD19 signal | MPPPRLLFFLLFLTPMEVR |
| | peptide | |
| 55 | Human CD19 | PEEPLVVKVEEGDNAVLQCLKGTSDGPTQQLTWSRESPLKPFLKLSLGLPGLG |
| | extracellular domain | IHMRPLAIWLFIFNVSQQMGGFYLCQPGPPSEKAWQPGWTVNVEGSGELFRW |
| | | NVSDLGGLGCGLKNRSSEGPSSPSGKLMSPKLYVWAKDRPEIWEGEPPCLPP |
| | | RDSLNQSLSQDLTMAPGSTLWLSCGVPPDSVSRGPLSWTHVHPKGPKSLLSL |
| | | ELKDDRPARDMWVMETGLLLPRATAQDAGKYYCHRGNLTMSFHLEITARPVL |
| | | WHWLLRTGGWK |
| 56 | Human CD19 | VSAVTLAYLIFCLCSLVGILHL |
| | transmembrane | |
| | domain | |
| 57 | Human CD19 | QRALVLRRKRKRMTDPTRRFFKVTPPPGSGPQNQYGNVLSLPTPTSGLGRAQ |
| | cytoplasmic domain | RWAAGLGGTAPSYGNPSSDVQADGALGSRSPPGVGPEEEEGEGYEEPDSEE |
| | | DSEFYENDSNLGQDQLSQDGSGYENPEDEPLGPEDEDSFSNAESYENEDEEL |
| | | TQPVARTMDFLSPHGSAWDPSREATSLGSQSYEDMRGILYAAPQLRSIRGQP |
| | | GPNHEEDADSYENMDNPDGPDPAWGGGGRMGTWSTR |
| 58 | FMC63 HC-CDR1 | GVSLPDYG |
| 59 | FMC63 HC-CDR2 | IWGSETT |
| 60 | FMC63 HC-CDR3 | AKHYYYGGSYAMDY |
| 61 | FMC63 LC-CDR1 | QDISKY |
| 62 | FMC63 LC-CDR2 | HTS |
| 63 | FMC63 LC-CDR3 | QQGNTLPYT |
| 64 | FMC63 VH | EVKLQESGPGLVAPSQSLSVTCTVSGVSLPDYGVSWIRQPPRKGLEWLGVIW |
| | | GSETTYYNSALKSRLTIIKDNSKSQVFLKMNSLQTDDTAIYYCAKHYYYGGSYA |
| | | MDYWGQGTSVTVSS |
| 65 | FMC63 VL | DIQMTQTTSSLSASLGDRVTISCRASQDISKYLNWYQQKPDGTVKLLIYHTSRL |
| | | HSGVPSRFSGSGSGTDYSLTISNLEQEDIATYFCQQGNTLPYTFGGGTKLEIT |
| 66 | FMC63 scFv linker | GSTSGSGKPGSGEGSTKG |
| 67 | FMC63 scFv | DIQMTQTTSSLSASLGDRVTISCRASQDISKYLNWYQQKPDGTVKLLIYHTSRL |
| | | HSGVPSRFSGSGSGTDYSLTISNLEQEDIATYFCQQGNTLPYTFGGGTKLEITG |
| | | STSGSGKPGSGEGSTKGEVKLQESGPGLVAPSQSLSVTCTVSGVSLPDYGVS |
| | | WIRQPPRKGLEWLGVIWGSETTYYNSALKSRLTIIKDNSKSQVFLKMNSLQTDD |
| | | TAIYYCAKHYYYGGSYAMDYWGQGTSVTVSS |
| 68 | CD28 hinge region | IEVMYPPPYLDNEKSNGTIIHVKGKHLCPSPLFPGPSKP |
| 69 | CD19.CAR | MLLLVTSLLLCELPHPAFLLIPDIQMTQTTSSLSASLGDRVTISCRASQDISKYLN |
| | sequence (with | WYQQKPDGTVKLLIYHTSRLHSGVPSRFSGSGSGTDYSLTISNLEQEDIATYFC |
| | signal peptide) | QQGNTLPYTFGGGTKLEITGSTSGSGKPGSGEGSTKGEVKLQESGPGLVAPS |
| | | QSLSVTCTVSGVSLPDYGVSWIRQPPRKGLEWLGVIWGSETTYYNSALKSRLT |
| | | IIKDNSKSQVFLKMNSLQTDDTAIYYCAKHYYYGGSYAMDYWGQGTSVTVSSA |
| | | AAIEVMYPPPYLDNEKSNGTIIHVKGKHLCPSPLFPGPSKPFWVLVVVGGVLAC |
| | | YSLLVTVAFIIFWVRSKRSRLLHSDYMNMTPRRPGPTRKHYQPYAPPRDFAAY |
| | | RSRVKFSRSADAPAYQQGQNQLYNELNLGRREEYDVLDKRRGRDPEMGGKP |
| | | RRKNPQEGLYNELQKDKMAEAYSEIGMKGERRRGKGHDGLYQGLSTATKDTY |
| | | DALHMQALPPR |
| 70 | CD19.CAR | DIQMTQTTSSLSASLGDRVTISCRASQDISKYLNWYQQKPDGTVKLLIYHTSRL |
| | sequence (without | HSGVPSRFSGSGSGTDYSLTISNLEQEDIATYFCQQGNTLPYTFGGGTKLEITG |
| | signal peptide) | STSGSGKPGSGEGSTKGEVKLQESGPGLVAPSQSLSVTCTVSGVSLPDYGVS |
| | | WIRQPPRKGLEWLGVIWGSETTYYNSALKSRLTIIKDNSKSQVFLKMNSLQTDD |
| | | TAIYYCAKHYYYGGSYAMDYWGQGTSVTVSSAAAIEVMYPPPYLDNEKSNGTII |
| | | HVKGKHLCPSPLFPGPSKPFWVLVVVGGVLACYSLLVTVAFIIFWVRSKRSRLL |
| | | HSDYMNMTPRRPGPTRKHYQPYAPPRDFAAYRSRVKFSRSADAPAYQQGQN |
| | | QLYNELNLGRREEYDVLDKRRGRDPEMGGKPRRKNPQEGLYNELQKDKMAE |
| | | AYSEIGMKGERRRGKGHDGLYQGLSTATKDTYDALHMQALPPR |

The features disclosed in the foregoing description, or in the following claims, or in the accompanying drawings, expressed in their specific forms or in terms of a means for performing the disclosed function, or a method or process for obtaining the disclosed results, as appropriate, may, separately, or in any combination of such features, be utilised for realising the invention in diverse forms thereof. The exemplary embodiments described herein are illustrative and not limiting, and equivalent modifications and variations will be apparent to those skilled in the art when given this disclosure. The present disclosure includes the combination of the aspects and preferred features described, except where such a combination is clearly impermissible or expressly avoided.

For the avoidance of any doubt, any theoretical explanations provided herein are provided for the purposes of improving the understanding of a reader. The inventors do not wish to be bound by any of these theoretical explanations.

Section headings used herein are for organisational purposes only and are not to be construed as limiting the subject matter described.

Aspects and embodiments of the present disclosure will now be illustrated, by way of example, with reference to the accompanying figures. Further aspects and embodiments will be apparent to those skilled in the art. All documents mentioned in this text are incorporated herein by reference.

Throughout this specification, including the claims which follow, unless the context requires otherwise, the word ‘comprise’ and ‘include’, and variations such as ‘comprises’ and ‘comprising’/‘includes’ and ‘including’, will be understood to imply the inclusion of a stated integer or step or group of integers or steps but not the exclusion of any other integer or step or group of integers or steps.

It must be noted that, as used in the specification and the appended claims, the singular forms ‘a,’ ‘an,’ and ‘the’ include plural referents unless the context clearly dictates otherwise. Ranges may be expressed herein as from ‘about’ one particular value, and/or to ‘about’ another particular value. When such a range is expressed, another embodiment includes from the one particular value and/or to the other particular value. Similarly, when values are expressed as approximations, by the use of the antecedent ‘about,’ it will be understood that the particular value forms another embodiment. The term ‘about’ in relation to a numerical value is optional and means for example +/−10%.

Where a nucleic acid sequence is disclosed herein, the reverse complement thereof is also expressly contemplated.

Methods described herein may preferably be performed in vitro. The term ‘in vitro’ is intended to encompass procedures performed with cells in culture whereas the term ‘in vivo’ is intended to encompass procedures with/on intact multi-cellular organisms.

### BRIEF DESCRIPTION OF THE FIGURES

Embodiments and experiments illustrating the principles of the present disclosure will now be discussed with reference to the accompanying figures.

FIG. 1. Schematic of the process for producing activated, TCR knockout (KO) cells as therapeutic graft cells.

FIG. 2. Schematic of the process for producing virus-specific cells (VSTs) as therapeutic graft cells.

FIG. 3. Schematic of the process for producing CD30 knockout (KO), allogeneic T (allo-T) cells, to serve as an example of generating allo-T cells not expressing CAR target antigens.

FIGS. 4A to 4C. Graphs and bar charts showing the expansion and expression of SERPINB9 (SB9) by T cells from two different donors transduced with either GFP or Serpin-GFP. The proliferative fold-change of T cells, over the culture period from day of transduction was determined from cell counts enumerated by Trypan Blue staining and using a hemocytometer. Transduced T cells were harvested on day 5 post-transduction, and GFP expression as a percentage of total CD3 positive cells, and intracellular SB9 expression (median fluorescence intensity) of GFP positive cells was determined by flowcytometry. (4A) Cumulative fold-change in the number of cells overtime. (4B) GFP expression as a percentage of total CD3 positive cells. (4C) Intracellular SB9 expression of GFP positive cells.

FIGS. 5A to 5C. Graphs and bar charts showing the expansion, expression of CD30.CAR (IgG1 spacer) and expression of SERPINB9 (SB9) by T cells from two different donors transduced with either GFP-CAR or Serpin-CAR. The proliferative fold-change of T cells, over the culture period from day of transduction was determined from cell counts enumerated by Trypan Blue staining and using a hemocytometer. T cells transduced with GFP served as controls. Transduced T cells were harvested on day 7 post-transduction, and CD30.CAR expression as a percentage of total CD3 positive cells, and intracellular SB9 expression (median fluorescence intensity) of CD30.CAR positive cells was determined by flowcytometry. (5A) Cumulative fold-change in the number of cells over time. (5B) CD30.CAR expression as a percentage of total CD3-positive cells. (5C) Intracellular SB9 expression of CD30.CAR-positive cells.

FIG. 6. Graphs showing cytolysis of KM-H2 cells by T cells transduced with GFP, CD30.CAR (IgG1 spacer), or SERPINB9 and CD30.CAR (IgG1 spacer) cells prepared from two different donors. CD30.CAR positive T cells were normalized to 70% of total cells in culture (for GFP-CD30.CAR and Serpin-CD30.CAR conditions) by the addition of GFP transduced T cells. Transduced T cells (effector cells) were co-cultured with CD30-expressing KM-H2 cells (target cells) at effector: target ratios of 1:1 and 2:1, and KM-H2 cytolysis was measured by the xCELLigence Real-Time Cell Analysis software (Agilent) over 48 h. T cells transduced with GFP served as negative controls.

FIGS. 7A to 7E. Schematic and graphs showing that SERPINB9 expression protects graft T cells from immune rejection in vitro. (7A) Schematic representation of the mixed lymphocyte reaction (MLR) assay, and the constructs used. GFP-transduced (negative control) or Serpin-GFP transduced TCR knockout (KO) graft T cells from two different donors were mixed with HLA-mismatched host PBMCs at a 1:10 cell ratio (PBMC mixed lymphocyte assay), and cell numbers were determined over 12 days. The MLR assay was performed in the presence of IL-7 and IL-15, both at 10 ng/mL, and the cultures were expanded when necessary. (7B) Cell counts of graft T cells when cultured alone. (7C) Cell counts of graft T cells when cultured in the PBMC MLR. (7D) Cell counts of host CD3-positive T cells. (7E) Cell counts of host CD3-negative, CD56-positive natural killer (NK) cells. All data denote mean±SD.

FIGS. 8A to 8E. Schematic, graphs and bar chart showing that SERPINB9 expression protects graft CD30.CAR (IgG1 spacer) T cells from T cell-mediated immune rejection in vitro. (8A) Schematic representation of the mixed lymphocyte reaction (MLR) assay, and the constructs used. GFP-transduced (negative control) or Serpin-GFP transduced TCR knockout (KO) graft T cells from two different donors were mixed with HLA-mismatched host's CD30 KO alloreactive T cells, primed to recognize and kill graft cells, in a 1:1-2 cell ratio (CD30 KO allo-T cell mixed lymphocyte assay), and cell numbers were determined over 3 days. The assay was performed in the presence of 10 ng/mL of both IL-7 and IL-15. (8B) Cell counts of graft T cells when cultured alone. (8C) Cell counts of graft T cells when cultured in the MLR. (8D) Cell counts of graft T cells after 3 days of culture in the MLR. (8E) Cell counts of host CD3-positive T cells when cultured in the MLR. All data denote mean±SD.

FIGS. 9A to 9K. Schematics, graphs and bar charts showing that over-expression of SB9(CAS) in CD30.CAR graft T cells does not affect CAR cytotoxic function and is superior in protecting graft cells from allogeneic rejection. (9A) Graph showing CD30.CAR (IgG1 spacer) ATC-mediated cytolysis of KM-H2 cells measured using the xCELLigence system. (9B) Schematic showing in vitro co-culture setup for 9C-9F, where graft TCRαβ^{KO} CD30.CAR (IgG1 spacer) ATCs and host CD30^{KO} ATCs, primed to recognize and kill graft cells, were mixed in a 1:1 ratio. The assay was performed in the presence of 10 ng/mL of both IL-7 and IL-15. (9C, 9D and 9E) Graphs showing cell counts, from a representative graft-host pair, of graft T cells growing in monoculture (C), graft T cells (D) and host T cells (E) in co-culture. (9F) Bar chart showing the host mediated graft killing, calculated by

$$\frac{graft cell count ( monoculture ) - graft cell count ( co - culture )}{graft cell count ( monoculture )} * 100 \% ,$$

where each dot represents a unique graft-host pair and the bar chart represents the median (n=6). P values were determined by the one-way ANOVA with Tukey post-test. *, P=0.0189; **, P=0.0034. (9G) Schematic showing in vitro co-culture setup for 9H-9K, where graft CD30.CAR (4-1 BB spacer) EBVSTs and host CD30^{KO} ATCs, primed to recognize and kill graft cells, were mixed in a 1:1-4 ratio. The assay was performed in the presence of 10 ng/mL of both IL-7 and IL-15. (9H, 9I and 9J) Graphs showing cell counts, from a representative graft-host pair, of graft T cells growing in monoculture (H), graft T cells (I) and host T cells (J) in co-culture. (9K) Bar chart showing the host mediated graft killing, where each dot represents a unique graft-host pair and the bar chart represents the median (n=4). The minimum ratio of graft:host that resulted in ≥80% host mediated graft killing of CD30.CAR (4-1BB spacer) EBVSTs on day 4 was selected. P values were determined by the one-way ANOVA with Tukey post-test. *, P=0.0150; **, P<0.01.

FIGS. 10A to 10E. Schematics, bar charts and graphs showing manufacturing and characteristics of TCRαβ^{KO} CD30.CAR (IgG1 spacer) ATCs expressing different forms of SB9. (10A) Schematic showing manufacturing timeline for the generation of TCRαβ^{KO} CD30.CAR ATCs expressing different forms of SB9 when transduced using retrovirus encoding bicistronic construct (top panel) or retroviruses separately encoding CAR and SB9 (bottom panel). (10B) Bar chart showing SB9 expression measured by intracellular staining followed by flow cytometry analysis. (10C and 10D) Bar charts showing CD30.CAR expression of CD3 positive T cells in percentage (C) and geometric mean fluorescence intensity (MFI) (D). (10E) Graphs showing expression of TCRαβ on CD3 wildtype (WT) T cells or T cells after TCRαβ^{KO} gene modification using Crispr gene editing measured by flow cytometry analysis.

FIGS. 11A and 11B. Schematic and graphs showing manufacturing of alloreactive CD30^{KO} host cells that recognize and kill graft cells (allo-T). (11A) Schematic showing manufacturing timeline for the generation of allo-T. (11B) Graphs showing CD30 expression of allo-T cells determined by the staining of CD30 with two different antibody clones, BY88 and BerH8, and flow cytometry analysis.

FIGS. 12A to 12K. Schematics, SDS-PAGE and bar charts showing manufacturing and characteristics of CD30.CAR (4-1BB spacer) EBVSTs expressing different forms of SB9. (12A) Schematic showing manufacturing timeline for the generation of CD30.CAR EBVSTs expressing different forms of SB9. (12B) SDS-PAGE analysis showing SB9 expression from a representative donor, where the top band (˜62 kDa) indicates the pre-formed GzmB and SB9 complexes in the cytosol and the bottom band (˜43 kDa) indicates the functional SB9 monomer. Transduction was carried out with either low (10 ng/mL) or high (100 ng/mL) IL-15 concentration, as denoted by L and H respectively. (12C) Bar chart showing densitometry analysis of SB9 monomer normalized to CD30.CAR EBVST control and calnexin as a loading control. (12D and 12E) Bar charts showing CD30.CAR expression of CD3 positive T cells in percentage (D) and geometric mean fluorescence intensity (MFI) (E), n=4 donors. (12F) Bar chart showing percentage of CD3 positive T cells producing IFNγ and/or TNFα in response to EBV peptides analysed by intracellular cytokine staining and flow cytometry, n=4 donors. (12G, 12H, 12I, 12J and 12K) Bar charts showing flow cytometry analysis of CD4/CD8 subsets (G), memory T cell subsets (CM: central memory, EM: effector memory, and TEMRA: T effector memory expressing CD45RA) (H), PD-1, Tim-3, Lag-3 expression (I), CD39 expression (J) and CD25 expression (K) of CD30.CAR EBVSTs from a representative donor.

FIGS. 13A to 13J. Schematics, graphs and bar charts showing over-expression of SB9(CAS) in non-CAR graft T cells (ATCs and EBVSTs) is superior in protecting graft cells from allogeneic rejection. (13A) Schematic showing in vitro co-culture setup for 13B-13E, where graft TCRαβ^{KO} ATCs and HLA-mismatched host PBMCs were mixed in a 1:10-20 ratio. The assay was performed in the presence of 10 ng/mL of both IL-7 and IL-15. (13B, 13C and 13D) Graphs showing cell counts, from a representative graft-host pair, of graft T cells growing in monoculture (B), graft T cells (C) and host T and NK cells (D) in co-culture. (13E) Bar chart showing the host mediated graft killing, calculated by

$$\frac{graft cell count ( monoculture ) - graft cell count ( co - culture )}{graft cell count ( monoculture )} * 100 \% ,$$

where each dot represents a unique graft-host pair and the bar chart represents the median (n=5). P values were determined by the one-way ANOVA with Tukey post-test. **, P=0.0016; ***, P<0.001. (13F) Schematic showing in vitro co-culture setup for 13G-13J, where graft EBVSTs and alloreactive CD30^{KO} host ATCs, primed to recognize and kill graft cells, were mixed in a 1:1-4 ratio. The assay was performed in the presence of 10 ng/mL of both IL-7 and IL-15. (13G, 13H and 13I) Graphs showing cell counts, from a representative graft-host pair, of graft T cells growing in monoculture (G), graft T cells (H) and host T cells (I) in co-culture. (13J) Bar chart showing the host mediated graft killing, calculated by

$$\frac{graft cell count ( monoculture ) - graft cell count ( co - culture )}{graft cell count ( monoculture )} * 100 \% ,$$

where each dot represents a unique graft-host pair and the bar chart represents the median (n=2). Statistics were not performed as n<3.

FIGS. 14A-14K. Schematics, images, bar charts and graphs showing overexpression of SB9(CAS) in CD30.CAR (4-1 BB spacer) EBVSTs protects CD30.CAR EBVSTs from allogeneic rejection and enhances anti-tumor efficacy both in vitro and in vivo. (14A) Schematic showing in vitro tri-culture setup for (B) and (C), where engineered NALM6 (truncated CD30-positive and HLA I and II^{KO}), graft CD30.CAR EBVSTs and host CD30^{KO} ATCs, primed to recognize and kill graft cells, were mixed in a 1:1:1-4 ratio. The assay was performed in the absence of cytokines. (14B) Bar chart showing the host mediated graft killing, calculated by

$$\frac{graft cell count ( co - culture of target and graft ) - graft cell count ( tri - culture )}{graft cell count ( co - culture of target and graft )} * 100 \% ,$$

where each dot represents a unique graft-host pair and the bar chart represents the median (n=5). The minimum ratio of graft:host that resulted in ≥60% host mediated graft killing of CD30.CAR EBVSTs on day 3 was selected. P values were determined by the one-way ANOVA with Holm-Šídák post-test post-test. *, P=0.0193. (14C) Bar chart showing the percentage of round 2 tumor cells killed in tri-culture normalized to tumor cells grown in monoculture. (14D) Schematic showing in vivo allorejection model for 14E-14F, 2.5×10⁶ engineered NALM6 (truncated CD30-positive and HLA I and II^{KO}) were injected intravenously into NSG (MHC^{KO}) mice. 18 days later, 5×10⁶ graft eGFP-ffLuc-expressing CD30.CAR EBVSTs with 5×10⁶ alloreactive host T cells (allo-T) were co-infused intravenously. (14E) Bioluminescent images showing graft CD30.CAR EBVST levels captured by IVIS Lumina S5 imaging system. (14F) Graph showing quantified bioluminescent signals from graft CD30.CAR EBVSTs over time. (14G) Schematic showing in vivo allorejection model for 14H-14K, 2.5×10⁶ engineered NALM6.eGFP-ffLuc were injected intravenously into NSG (MHC^{KO}) mice, 15 days later, 5×10⁶ graft CD30.CAR EBVSTs with 5×10⁶ alloreactive host T cells (allo-T) were co-infused intravenously. (14H and 14I) Graphs showing flow cytometry analysis of blood samples at the indicated time-points. The percentages of graft CD30.CAR EBVST cells (H) and host allo-T cells (1) in the peripheral blood are shown. (14J) Bioluminescent images showing engineered NALM6.eGFP-ffLuc tumor growth captured by IVIS Lumina S5 imaging system. (14K) Graph showing quantified bioluminescent signals from tumor cells overtime normalized to the levels of disease on day 0. All graphs for in vivo data denote mean+S.D. P values were determined using one-way ANOVA (K; day 11 post treatment) or two-way ANOVA (F and H: P values are shown for comparison between CD30.CAR+Allo-T and SB9(CAS)-CD30.CAR+Allo-T samples) with Dunnett's correction for multiple comparisons, where sample means were compared with mean of CD30.CAR+Allo-T sample.

FIGS. 15A to 15F. Graphs and bar charts showing overexpression of SB9(CAS) in CD19.CAR ATCs and CD30.CAR (4-1 BB spacer) ATCs protects them from allogeneic rejection and enhances anti-tumor efficacy in vitro. (15A) Graphs showing flow cytometry analysis of FACS-sorted engineered NALM6 cells that over-express truncated CD30 (tCD30) and were gene-edited for the knockout of HLA class I and II (HLA^{DKO}). (15B) Bar chart showing the percentage of tumor cells killed by host cells in co-culture setup of engineered NALM6 tumor cells and host allo-T cells in a ratio of 1:1. (15C) Graph showing the host mediated graft killing, calculated by

$$\frac{graft cell count ( co - culture of target and graft ) - graft cell count ( tri - culture )}{graft cell count ( co - culture of target and graft )} * 100 \% ,$$

of CD19.CAR ATC graft cells in tri-culture with host ATCs, primed to recognize and kill graft cells, and engineered NALM6 in a 1:1:1 ratio. The assay was performed in the absence of cytokines. (15D) Graph showing the host mediated graft killing of CD30.CAR ATC graft cells in tri-culture with host CD30^{KO} ATCs, primed to recognize and kill graft cells, and engineered NALM6 in a 1:4:1 ratio. The assay was performed in the absence of cytokines. (15E and 15F) Bar charts showing the tumor cell numbers counted by flow cytometry analysis on day 3 after tri-culture with CD19.CAR ATCs (E) or CD30.CAR ATCs (F).

FIGS. 16A to 16G. Schematics, images and graphs showing allogeneic rejection of graft T cells occurs with the administration of alloreactive host T cells in vivo. (16A) Schematic showing in vivo allorejection model for 16B and 16C, 2.5×10⁶ engineered NALM6 (truncated CD30-positive and HLA I and II^{KO}) were injected intravenously into NSG (MHC^{KO}) mice. 18 days later, 5×10⁶ graft eGFP-ffLuc-expressing CD30.CAR (4-1 BB spacer) EBVSTs with or without 5×10⁶ alloreactive host T cells (allo-T) were infused intravenously. (16B) Bioluminescent images showing graft CD30.CAR EBVST levels captured by IVIS Lumina S5 imaging system. (16C) Graph showing quantified bioluminescent signals from graft CD30.CAR EBVSTs overtime. (16D) Schematic showing in vivo allorejection model for 16E-16G, 2.5×10⁶ engineered NALM6.eGFP-ffLuc were injected intravenously into NSG (MHC^{KO}) mice, 15 days later, 5×10⁶ graft CD30.CAR EBVSTs with or without 5×10⁶ alloreactive host T cells (allo-T) were infused intravenously. (16E) Graph showing flow cytometry analysis of blood samples at the indicated time-points. The percentage of graft CD30.CAR EBVST cells in the peripheral blood are shown. (16F) Bioluminescent images showing engineered NALM6.eGFP-ffLuc tumor growth captured by IVIS Lumina S5 imaging system. (16G) Graph showing quantified bioluminescent signals from tumor cells over time normalized to the levels of disease on day 0. All graphs for in vivo data denote mean+S.D. P values were determined using two-way ANOVA with Sidak's correction for multiple comparisons (E) or unpaired one-tailed t-test (C and G; day 11 post treatment).

FIGS. 17A to 17Q. Schematics, graphs and bar charts showing SB9(CAS) overexpression enhances CD30.CAR (4-1BB spacer) and CD19.CAR ATC expansion following serial co-culture with tumor cells. (17A) Schematic showing in vitro co-culture of CAR ATCs with engineered NALM6 tumor cells in a fixed ratio of 1:1 or 1:5, when CD30.CAR and CD19.CAR ATCs were studied respectively. Every 2-3 days, the cells were counted by flow cytometry and re-plated in co-culture with fresh tumor cells at a fixed ratio and the process was repeated until the CAR T cells were unable to eliminate tumor cells. The experiment was performed in the absence of cytokines. (17B, 17C, 17F and 17G) Graphs showing the cumulative proliferation fold-change of CD30.CAR ATCs grown in monoculture (B and F) and in co-culture (C and G). (17D and 17H) Bar charts showing tumor cell numbers counted by flow cytometry on day 13. (17E and 17I) Bar charts showing PD-1, Tim-3 and Lag-3 expression on CD30.CAR ATCs before the start of co-culture (Day 0) and on the last day of co-culture (Day 13). (17J, 17K, 17N and 17O) Graphs showing the cumulative proliferation fold-change of CD19.CAR ATCs grown in monoculture (J and N) and in co-culture (K and O). (17L and 17P) Bar charts showing tumor cell numbers counted by flow cytometry on day 8. (17M and 17Q) Bar charts showing PD-1, Tim-3 and Lag-3 expression on CD19.CAR ATCs before the start of co-culture (Day 0) and on the last day of co-culture (Day 8).

FIGS. 18A to 18D. Schematic and bar charts showing cytokine secretion profiles of CD30.CAR EBVSTs (4-1BB spacer) expressing different forms of SB9 with and without CAR-mediated stimulation. (18A) Schematic showing the in vitro co-culture setup for 18B-18D, where effector CAR T cells were co-cultured with either clonally selected engineered NALM6 (truncated CD30-positive and HLA I and II^{KO}) or KM-H2 tumor cells in a 1:1 ratio. The assay was performed in the absence of cytokines. (18B, 18C and 18D) Bar charts showing the cytokine concentration measured using the 13-plex-Immunology Multiplex Assay kit (Merck) from the supernatant of cells cultured for 24 h in monoculture (B) or in co-culture with engineered NALM6 (C) or KM-H2 (D) tumour cells. The bars represent mean+SD.

FIGS. 19A to 19J. Schematics, graphs and bar charts showing SB9(CAS) overexpression enhances expansion and tumor killing efficacy of CD30.CAR EBVSTs in vivo. (19A) Schematic showing in vitro co-culture of CD30.CAR (4-1 BB spacer) EBVSTs with clonally selected engineered NALM6 tumor cells (expressing truncated CD30-positive with HLA I and II^{KO}) in a fixed ratio of 1:1. Every 2-3 days, the cells were counted by flow cytometry and re-plated in co-culture with fresh tumor cells at a fixed ratio and the process was repeated until the CAR T cells were unable to eliminate tumor cells. The experiment was performed in the absence of cytokines. (19B and 19C) Graphs showing the cumulative proliferation fold-change of CD30.CAR EBVSTs grown in monoculture (B) and in co-culture (C). (19D) Bar chart showing tumor cell numbers counted by flow cytometry on day 8. (19E) Bar charts showing PD-1, Tim-3 and Lag-3 expression on CD30.CAR EBVSTs before the start of co-culture (Day 0) and on the last day of co-culture (Day 8). CD30.CAR EBVSTs manufactured from 3 donors were tested in vitro and resulted in similar findings. (19F) Schematic of in vivo activation-induced cell death (AICD) model for 19G-19J, where 2.5×10⁶ clonally selected engineered NALM6 were injected intravenously into NSG (MHC^{KO}) mice. 15 days later, 10×10⁶ effector CD30.CAR (4-1BB spacer) EBVSTs were infused intravenously. (19G and 19H) Images and graphs showing tumour bioluminescence (G) captured by IVIS Lumina S5 imaging system and its quantification (H), normalized to day 0. (19I) Quantified tumor burden (normalized to day 0) on day 20 post treatment. The graph denotes mean+S.D and P values were determined using one-way ANOVA with Dunnett's correction for multiple comparisons, where means were compared with mean of SB9(CAS)-CD30.CAR sample. (19J) Flow cytometry analysis of blood samples, collected from facial veins, at the indicated time-points showing CD30.CAR EBVST levels in the peripheral blood. The graph denotes mean+S.D and P values (between CD30.CAR and SB9(CAS)-CD30.CAR samples) were determined using two-way ANOVA with Dunnett's correction for multiple comparisons.

FIG. 20. Graph showing CD30 positive B-cell acute lymphoblastic leukemia (B-ALL) burden of two in vivo models that were being used to test SB9-mediated protection of grafts against allorejection (allorejection model) and AICD (AICD model), respectively. Bioluminescence signals from engineered NALM6.eGFP-ffLuc that were prepared from either FACS sorting (allorejection model) or clonal selection (AICD model) were measured at baseline (day 0). Each point represents an individual mouse. Lines with error bars denotes mean+SD.

FIGS. 21A to 21D. Schematic, bar charts and graph showing SB9(CAS), but not SB9(WT) overexpression, protects CD30.CAR EBVSTs from Fas-mediated apoptosis. (21A) Schematic of apoptosis pathways involved in allorejection and activation-induced cell death (AICD). (21B and 21C) Bar charts showing luminescence measurements of (B) CellTiter-Glo® assay, taken 16 h post plating of CD30.CAR (4-1 BB spacer) EBVSTs or (C) Caspase-Glo® 3/7 CellTiter-Glo® assay, taken 45 min post plating of CD30.CAR EBVSTs on PBS- or anti-CD95 antibody-coated plates with/without the presence of pan-caspase inhibitor, zVAD FMK (100 μM). Each point in the bar chart represents a unique donor, and the bars represent the median (n=3-4). P values were determined by the two-way ANOVA with Tukey post-test. **, P<0.01; ****, P<0.0001. (21D) Graphs showing intracellular flow cytometry analysis of SB9(CAS)-His tag expression 16 h post plating of SB9(CAS)-His-CD30.CAR EBVSTs on PBS- or anti-CD95 antibody-coated plates with/without the presence of pan-caspase inhibitor, zVAD FMK. SB9(CAS)-His-CD30.CAR EBVSTs manufactured from 3 donors were studied and found to have similar results.

FIGS. 22A to 22D. Schematic, graphs and bar chart showing evaluation of SB9(CAS)-mediated protection against Fas-mediated apoptosis in SB9(CAS)-overexpressing NK cells. (22A) Schematic showing manufacturing timeline for the generation of NK cells that overexpress SB9(CAS). (22B) Graphs showing expansion during manufacturing of non-transduced (NT) and SB9(CAS)-overexpressing NK cells. (22C) Bar chart showing CellTiter-Glo® assay, taken 1 h post plating of NK cells on PBS- or anti-CD95 antibody-coated plates with/without the presence of pan-caspase inhibitor, zVAD FMK (100 μM). (19D) Graphs showing intracellular flow cytometry analysis of SB9(CAS)-His tag expression 4 h post plating of SB9(CAS)-His NK cells on PBS- or anti-CD95 antibody-coated plates with/without the presence of pan-caspase inhibitor, zVAD FMK. SB9(CAS)-His NK cells manufactured from 2 donors were studied and found to have similar results.

### EXAMPLES

### Example 1: Materials and Methods

### 1.1 Donors

Enriched leukapheresis products, collected from consented healthy donors by Spectra Optia® Apheresis System CMNC collection protocol and frozen in ACD-A anticoagulant, was purchased from HemaCare (Northridge, California, U.S.A.). The frozen leukopaks were thawed and PBMCs were extracted by gradient centrifugation using Ficoll-Paque PLUS (Cytiva, MA, U.S.A.). The PBMCs were either used immediately for experiments or frozen in smaller aliquots of 30-50×10⁶ cells per cryovial in CryoStor® CS10 Cell Freezing Medium (STEMCELL Technologies, Cambridge, Massachusetts, U.S.A.).

### 1.2 Tumour Cell Lines

The Hodgkin lymphoma cell line, KM-H2, was purchased from DSMZ-German Collection of Microorganisms and Cell Cultures GmbH (Braunschweig, Germany). The acute lymphoblastic leukemic cell line, NALM6 (clone G5), was purchased from American Type Culture Collection (ATCC, VA, U.S.A.). HLA class I and II were also knocked out sequentially in NALM6 tumor cells by Crispr gene editing. Two single guide RNA (sgRNA) sequences targeting Beta 2 Microglobulin (B2M) (DNA sequences: CGTGAGTAAACCTGAATCTT and AAGTCAACTTCAATGTCGGA) and three sgRNA sequences, designed using Synthego design tool, targeting class II transactivator (CIITA) (DNA sequences: AGTCGCTCACTGGTCCCACT, CCGTGGACAGTGAATCCACT and CTCTCACCGATCACTTCATC) were used. A total of 270 pmol guide RNAs (135 and 90 pmol of each sgRNA were used for B2M and CIITA^{KO} respectively) complexed with 54.9 pmol Cas9 protein (IDT, Iowa, U.S.A.) were delivered into 1×10⁶ NALM6 cells using the 4D-Nucleofector® system (Lonza, Basel, Switzerland) in 20 μL of buffer SF. NALM6 was then engineered to express truncated CD30 (tCD30) that lacks its endodomain by retrovial transduction on RetroNectin (Takara Bio, Kusatsu, Shiga, Japan)-coated plates. The tCD30 high, HLA^{DKO} cells (engineered NALM6) were purified by magnetic bead isolation (Miltenyi Biotec, Bergisch Gladbach, Germany) followed by either two to three rounds of fluorescence-activated cell sorting (FACS sorting) or clonal selection.

If tumor cells were required to express a fusion protein that consisted of enhanced green fluorescent protein-firefly luciferase (eGFP-ffLuc) driven by the CMV promoter, the cell lines were retrovirally transduced on RetroNectin (Takara Bio)-coated plates before FACS sorting or clonal selection. The eGFP-ffLuc retrovirus producer cell was a kind gift from Dr. M. Suzuki's lab (Baylor College of Medicine, TX). The cell lines were grown in RPMI medium supplemented with 10% heat inactivated fetal bovine serum (Hyclone, Cytiva, U.S.A.) and 2 mM GlutaMAX (Gibco, Thermo Fisher Scientific, U.K.). The cells were discarded after passage number 20.

### 1.3 Plasmid Constructs and Retroviral Vectors

SB9 or its derivatives, with or without a CAR, were cloned into SFG retrovirus vector. Amino acid sequences of SB9 and its derivatives are shown in SEQ ID NO: 1, 5, 6, 7 and 47. Bicistronic formats of SB9 or its derivatives with CAR, in both orientations, separated by a furin-porcine teschovirus-1 2A (P2A) linker or Thosea asigna virus 2A (T2A) linker were also generated on the SFG plasmid. SB9 with a polyhistidine tag (his tag) at the C-terminus was also generated. SB9 and/or CAR retrovirus vectors were produced by the transient transfection of RD114 packaging cell line (BioVec Pharma, Quebec, Canada) with the SFG plasmid using PEIpro transfection reagent (Polyplus, Illkirch, FRANCE). Medium containing retroviruses were harvested at 48 h and 72 h post transfection and concentrated 10-fold using RetroX Concentrator (Takara Bio, Kusatsu, Shiga, Japan). The retroviruses were either used immediately or snap frozen and stored at −80° C.

### 1.4 Generation and Transduction of Therapeutic Cells (Graft Cells)

### Activated T Cells

PBMCs were activated on CD3 and CD28 (Biolegend, CA, U.S.A.)-coated non tissue culture treated plates (JetBiofil, Alicante, Spain) and cultured in CTL medium [45% advanced RPMI (Gibco, Grand Island, New York, U.S.A.), 45% Clicks' medium (FUJIFILM Irvine Scientific, Santa Ana, California, United States), 10% heat inactivated fetal bovine serum (Hyclone, Cytiva, U.S.A.) and 2 mM GlutaMAX (Gibco, Thermo Fisher Scientific, U.K.)] supplemented with 10 ng/mL IL-7 and IL-15 (all purchased from R&D Systems). Two days later, the T cell receptor was knocked out using two single guide RNA (sgRNA) sequences (Thermo Fisher Scientific, U.K.) targeting TCRαβ (DNA sequences: AGAGTCTCTCAGCTGGTACA and GCAGTATCTGGAGTCATTGA). A total of 270 pmol guide RNAs (135 pmol of each sgRNA) together with 37 pmol Cas9 protein (IDT, Iowa, U.S.A.) were delivered into 1×10⁶ T cells using the 4D-Nucleofector® system (Lonza, Basel, Switzerland) in 20 μL of buffer P3. 5 μg of RetroNectin was coated per well of non-tissue culture treated 24-well plates (JetBiofil) overnight at 4° C.

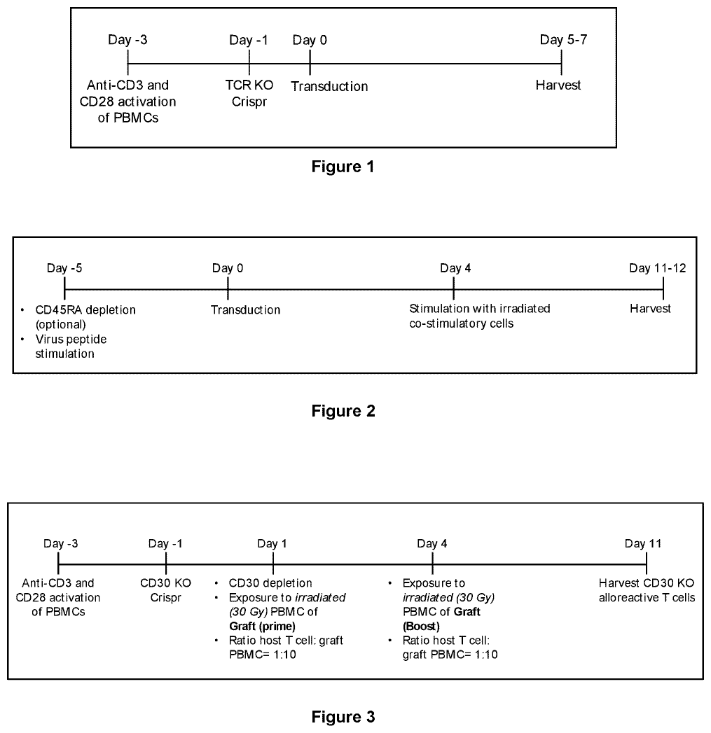

To generate SB9 expressing ATCs or CAR.ATCs (using CAR-SB9 bicistronic virus), cells were transduced with SB9 wildtype, or its derivatives, or a CAR-SB9 bicistronic virus on the RetroNectin-coated plates (Takara Bio, Kusatsu, Shiga, Japan). 100-300 μL of 10-fold concentrated retrovirus diluted with CTL medium to 1 mL was added to each RetroNectin-coated well and centrifuged at 2000×G for 1 h. After the spin, the virus supernatant was removed, and 0.2×10⁶ T cells were added to the well. The cells in the plate were centrifuged at 400×G for 5 min and placed in the 37° C. incubator and cultured for a further 5-7 days (FIG. 1, FIG. 10A).

Cell numbers were tracked by counting using Trypan Blue (ThermoFisher Scientific) and the hemocytometer or by the NC-200™ cell counter (Chemometec, Allerod Denmark). SB9 expression was analysed by intracellular staining using the BD Cytofix/Cytoperm™ (BD Biosciences, U.S.A.) or eBioscience™ Foxp3/Transcription Factor Staining Buffer (ThermoFisher Scientific). Cells were then stained for SB9 using the SB9 monoclonal antibody (7D8) (ThermoFisher Scientific).

### Virus-Specific T Cells

CD45RA depletion of PBMCs (RAD-PBMCs, optional) was performed by negative selection using CD45RA MACS Beads (Miltenyi Biotec, Bergisch Gladbach, Germany). Whole PBMCs or RAD-PBMCs were cultured 1×10⁶ cells/well with viral peptides consisting of overlapping peptide libraries (15-mers overlapping by 11 amino acids) from JPT Technologies (Berlin, Germany). Five days later, cells were transduced with SB9 wildtype or its derivatives with or without CAR using either a mixed virus cocktail (SB9 virus mixed with a CAR virus) or a SB9-CAR bicistronic virus on RetroNectin-coated 24-well plates (Takara Bio, Kusatsu, Shiga, Japan), according to the description above. T cells were then stimulated with irradiated co-stimulatory cells expressing markers such as CD80, CD86, 4-1BB four days post transduction. Seven-eight days later, VSTs were harvested, frozen or used for cell assays (FIG. 2, FIG. 12A). VSTs were grown in VST medium [47.5% advanced RPMI (Gibco), 47.5% Clicks' medium (FUJIFILM Irvine Scientific), 5% human platelet lysate (Sexton Biotechnologies, IN, U.S.A.) and 2 mM GlutaMAX (Gibco).

### NK Cells

The manufacturing protocol of NK cells was adopted from the following studies (19, 20). Briefly, NK cells were isolated from PBMCs using NK Cell Isolation Kit (Miltenyi Biotec) and co-cultured with irradiated K562 (100 Gy) for four days in NK medium (NK MACS Basal Medium with 10% heat inactivated fetal bovine serum and 1% NK MACS Supplement (Miltenyi Biotec)) supplemented with 500 IU/mL of IL-2 and 10 ng/mL of IL-15 (all purchased from R&D Systems). On the day of transduction, cells were transduced using retrovirus encoding SB9(CAS) on RetroNectin (Takara Bio)-coated 24-well plates and then cultured for three days. NK cells were then stimulated again by co-culturing with irradiated K562 cells in NK medium supplemented with 100 IU/mL of IL-2 and 10 ng/mL of IL-15. Six to eight days later, NK cells were harvested for in vitro assays.

### 1.5 Mixed-Lymphocyte Reaction (MLR) Assays

MLR with PBMC Host Cells

Graft T cells were cocultured with HLA-mismatched PBMCs (graft to PBMC ratio=1:10-20) in CTL or VST medium. The co-cultures were set up in duplicate or triplicates in 24-well G-rex (Wilson Wolf, MN, U.S.A.) in medium containing IL-7 and IL-15 (10 ng/mL each, R&D Systems). The cultures were expanded every 3-6 days in CTL medium with cytokines. At each time point, cells from each well were collected and stained with live-dead stain (ThermoFisher Scientific), anti-human CD3, CD4, CD8, CD56, HLA-A2 and/or HLA-A3 and TCRαβ (BD Biosciences). Cells were acquired using the Cytek Aurora flow (CA, U.S.A.) cytometer and analysed by FlowJo software (BD Biosciences).

MLR with Alloreactive Host T Cells

To generate alloreactive host T cells (allo-T cells), PBMCs from an HLA-mismatched donor to the graft donor were activated on CD3 and CD28 (Biolegend, CA, U.S.A.)-coated non tissue culture treated plates (JetBiofil) and cultured in CTL medium supplemented with 10 ng/mL IL-7 and IL-15 (R&D Systems). If CD30 was required to be knocked-out, Crispr gene editing was performed two days later. CD30 was knocked out using three single guide RNA (sgRNA) sequences, designed using Synthego design tool, targeting CD30 (DNA sequences: AGGTCTGGACCGGGTAGCAC, GCTGTGTCGGGAACAGCCCT and TCGACATTCGCAGACACGGG). A total of 270 pmol guide RNAs (90 pmol of each sgRNA) together with 37 pmol Cas9 protein (IDT, Iowa, U.S.A.) were delivered into 1×10⁶ T cells using the 4D-Nucleofector® system (Lonza, Basel, Switzerland) in 20 μL of buffer P3. Two days post electroporation, any remaining CD30 positive cells were depleted by negative selection using CD30 MACS Beads (Miltenyi Biotec). The efficiency of CD30 KO and depletion was assessed by staining cells with two clones of CD30− BerH8 (BD biosciences) and BY88 (Biolegend). Cells were acquired using the Cytek Aurora flow cytometer (CA, U.S.A.) and analysed by FlowJo software (BD Biosciences). The T cells were then placed in co-culture with irradiated (30 Gy) HLA-mismatched donor PBMCs in a 1:10 ratio (prime step). After 3 days, the T cells were again placed in co-culture with irradiated (30 Gy) HLA-mismatched donor PBMCs in a 1:10 ratio (boost step). Allo-T cells were harvested on day 14 (FIG. 11A).

Graft T cells were cocultured with HLA-mismatched WT or CD30 knockout (KO) allogeneic T (allo-T) cells (graft to CD30 KO allo-T cells ratio=1:1-4) in CTL medium. CD30 KO allo-T cells were used in the case of when SB9 was studied in the context of CD30.CAR expressing graft T cells. In scenarios where other CARs are studied, CAR target antigens if present on T cells, will be knocked out of the allo-T cells. The co-cultures were set up in triplicates in 96-well flat bottom tissue culture treated plates (Corning). At each time point, cells from each well were collected and stained with live-dead stain (ThermoFisher Scientific), anti-human CD3, CD4, CD8, HLA-A2 and/or HLA-A3 and TCRαβ (BD Biosciences). Cells were acquired using the Cytek Aurora flow cytometer (CA, U.S.A.) and analysed by FlowJo software (BD Biosciences).

### MLR in the Presence of Tumor Cells (Tri-Culture)

On day 0, engineered NALM6 was co-cultured with CAR T cells and HLA-mismatched allo-T cells in a ratio of 1:1:1-4 in CTL or VST medium without cytokines (round 1). The co-cultures were set up in triplicates in 96-well flat bottom tissue culture treated plates (Corning). On day 2, cells were harvested and a complete medium change for the cells was performed before adding 1×10⁵ engineered NALM6 to graft and host cells (round 2). At each time point, cells from each well were collected and stained with live-dead stain (ThermoFisher Scientific), anti-human CD3, CD4, CD8, HLA-A2 and/or HLA-A3, TCRαβ, CD45, CD19, CD30 (BD Biosciences). Cells were acquired using the Cytek Aurora flow cytometer (CA, U.S.A.) and analysed by FlowJo software (BD Biosciences).

FIG. 3 shows the manufacture of allogeneic T (allo-T) cells with CD30 knockout (KO), to serve as an example of generating allo-T cells with no expression of CAR target antigens.

### 1.6 Cytotoxicity Assay

The cytotoxicity of CAR expressing T cells was assessed by the xCELLigence assay, which uses cell impedance as a readout measured by the xCELLigence Real-Time Cell Analysis software (Agilent, CA, U.S.A.). The example of CD30.CAR cytotoxicity assay is described: 4×10⁴ target cells (CD30 positive KM-H2 cells) were seeded per well of 96-well RTCA E-Plates, which were pre-coated with CD40 antibody (Agilent). The target cells were left to adhere overnight at 37° C. The next day, T cells were added in various effector to target ratios (E:T=1:1 and 1:2) and co-cultures were left to incubate in RPMI medium supplemented with supplemented with 10% heat inactivated fetal bovine serum (Hyclone, Cytiva, U.S.A.) and 2 mM GlutaMAX (Gibco, Thermo Fisher Scientific, U.K.) for 48-72 h.

### 1.7 Detection of SB9 Over-Expression

SB9 expression was analysed either by intracellular staining and flow analysis or by western blot.

### Flow Cytometry Analysis

T cells were fixed and permeabilized by BD Cytofix/Cytoperm™ (BD Biosciences, U.S.A.) and stained for SB9 using the SB9 mouse monoclonal antibody (7D8) (ThermoFisher Scientific). For cells transduced with histidine tagged SB9, cells were stained with anti-His mouse monoclonal antibody (GG11-8F3.5.1) (Miltenyi Biotec).

### Western Blot Analysis

Cell lysates were prepared by lysing 3-5 million T cells in 60-100 μL of Nonidet P-40 (NP-40) cell lysis buffer [1% (v/v) Nonidet P-40 in 50 mM Tris, pH 8.0, 10 mM EDTA, 1× cOmplete™ Protease Inhibitor Cocktail (Merck, NJ, U.S.A.)] and boiling the samples with 4× Laemmli sample buffer (Bio-rad, CA, U.S.A.). The samples were resolved by sodium dodecyl sulfate (SDS) polyacrylamide gel electrophoresis (PAGE) and transferred to nitrocellulose membranes for immunoblotting and visualization via chemiluminescence. SB9 was probed using SB9 mouse monoclonal antibody (7D8) (ThermoFisher Scientific). Densitometry analysis was performed using the iBright Analysis Software (ThermoFisher Scientific).

### 1.8 EBV-Reactivity Assay

EBVSTs were seeded at 4×10⁵ cells/well of U-bottom 96-well plates (Corning, NY, U.S.A.), and virus-specific activity of responder cells was measured after stimulation with (1 μg/mL) EBV peptides (JPT Peptide Technologies) in the presence of 1 μg/mL co-stimulation molecules CD28 and CD49d (both from BD Biosciences). Medium alone (no peptide) and cells treated with HIV peptides (JPT Peptide Technologies) served as negative control. After overnight incubation in the presence of monensin and brefeldin A (BD Biosciences), T cells were stained with live-dead stain (ThermoFisher Scientific), CD3, CD4, CD8 antibodies, fixed and permeabilized using BD Cytofix/Cytoperm™, and stained with APC- or PE-conjugated IFN-γ and TNF-α antibodies (all reagents from BD Biosciences).

### 1.9 Serial In Vitro Tumor Cell Challenge Assay

CAR T cells were co-cultured with tumor cells (KM-H2 or engineered NALM6) in a fixed ratio that ranged from 1:1-5. The cells were cultured in CTL or VST medium, without the addition of cytokines. Every 2-3 days, the cells were harvested, and a portion of cells were stained with live/dead dye (ThermoFisher Scientific), anti-human CD3, CD4, CD8, CD45, CD30, CD19, CD45, PD-1, Tim-3 (BD Biosciences), Lag-3 (Biolegend) and in-house produced anti-HRS3 or anti-FMC63 (Acro Biosystems, DE, U.S.A.) antibodies. Cells were acquired using the Cytek Aurora flow cytometer and analysed by FlowJo software (BD Biosciences). The CAR T cells were plated in co-culture again with fresh tumor cells at a fixed ratio and the process was repeated until the CAR T cells were unable to eliminate tumor cells.

### 1.10 In Vivo Assay

Breeder pairs of NOD.Cg-Prkdcs^{cid} H2-K1^{tm1Bpe} H2-Ab1^{em1Mvw} H2-D1^{tm1Bpe} Il2rg^{tm1Wjl}/SzJ mice (NSG (MHC^{KO}), stock no. 025216) were purchased from the Jackson Laboratory (ME, U.S.A.) and bred at InVivos (Singapore) through contract breeding. All animal experiments were conducted in Biological Resource Centre (BRC, Singapore) of Agency for Science, Technology and Research (A*STAR, Singapore) in compliance with the Institutional Animal Care and Use Committee (IACUC) protocol number 201540. Both female and male mice (aged 8-12 weeks) were used for the experiments.

### Allorejection Model

To establish allorejection models, NSG (MHC^{KO}) mice were injected via tail vein with FACS sorted 2.5×10⁶ engineered NALM6 or engineered NALM6.eGFP-ffLuc. The tumor was allowed to establish for 15-18 days before 5×10⁶ allo-T cells and eGFP-ffLuc-expressing or non-expressing CAR T cells were co-infused into the mice, via tail vein injection, at a fixed ratio (1:1).

For experiments where mice were injected with engineered NALM6.eGFP-ffLuc cells, the tumor burden was measured by IVIS Lumina S5 imaging system and analysed by Living Image v.4.7 software (both from PerkinElmer, MA, U.S.A.) twice a week. Graft CAR T cell and host allo-T cell levels were evaluated from blood samples collected from facial vein, where 100-150 μL of blood was obtained at indicated time points. Red blood cells (RBC) were then lysed with RBC lysis buffer (eBioscience, CA, U.S.A.) and samples were stained with anti-mouse CD45, anti-human CD45, CD3 (BD Biosciences), HLA-A2, CD19, and CD30 (BioLegend) antibodies to determine the levels of CAR T cells, allo-T cells, and tumor cells by flow cytometry analysis. Cells were acquired using the BD FACSymphony A3 flow cytometer (CA, U.S.A.) and analysed by FlowJo software (BD Biosciences). For experiments where mice were injected with eGFP-ffLuc-expressing CAR T cells, T cell signal was quantified by IVIS Lumina S5 imaging system and analysed by Living Image v.4.7 software (Both from PerkinElmer) 3 times a week.

### Activation Induced Cell Death (AICD) Model

NSG (MHC^{KO}) mice were injected via tail vein with 2.5×10⁵ clonally selected engineered NALM6.eGFP-ffLuc. The tumor was allowed to establish for 15 days before 10×10⁶ CD30.CAR EBVSTs were infused into the mice, via tail vein injection. The tumor burden was measured by IVIS Lumina S5 imaging system and analysed by Living Image v.4.7 software twice a week. CD30.CAR EBVSTs were evaluated from blood samples collected from facial vein followed by flow analysis in a similar process as described above.

### 1.11 CD95 Cross-Linking Assay

Functional grade CD95 (APO-1/Fas) monoclonal Antibody (EOS9.1), (eBioscience, Thermo Fisher Scientific or equivalent from Biolegend) was coated on tissue-culture treated white-walled 96 well flat bottom plates at 10 μg/mL, diluted in phosphate-buffered saline (PBS, Gibco), overnight at 4° C. PBS treated wells served as controls. The next day, the plates were rinsed with PBS and T or NK cells were added at 1.5×10⁵ cells/well of 96-well plate. For conditions where cells were treated with a pan caspase inhibitor, the cells were pre-incubated at 37° C. for 30 min with 0.1 mM Z-VAD-FMK before they were plated on CD95-coated plates. Once the cells were added, the plates were centrifuged at 400×G for 5 min and placed in the 37° C. incubator for a stipulated amount of time required for different readouts. To determine caspase 3/7 activity, cells on the 96 well plate were harvested at 45 min and lysed with Caspase-Glo® 3/7 Assay System (Promega, WI, U.S.A.). To determine cell viability, cells on the 96 well plate were harvested after 1 h for NK cells and 20 h for T cells, and lysed with CellTiter-Glo® Cell Viability reagent (Promega). Luminescence was read on the Cytation 5 plate reader (Agilent BioTek, VT, U.S.A.). To determine SB9(CAS)-His tag expression by flow, T cells were fixed and permeabilized by BD Cytofix/Cytoperm™ (BD Biosciences) and stained for His using the anti-His mouse monoclonal antibody (GG11-8F3.5.1) (Miltenyi Biotec). Cells were acquired using the Cytek Aurora flow cytometer and analysed by FlowJo software (BD Biosciences).

### 1.12 Luminex Assay

CD30.CAR EBVSTs (4-1 BB spacer) were co-cultured with tumor cells (KM-H2 or engineered NALM6) in a 1:1 fixed ratio. The cells were cultured in VST medium without the addition of cytokines. The next day, clarified supernatant was harvested and processed using the MILLIPLEX MAP Human High Sensitivity T Cell Panel Premixed 13-plex-Immunology Multiplex Kit (Catalog number: HSTCMAG28SPMX13, Merck, Darmstadt, Germany). Cytokine concentration was measured using the LuminexFlexmap 3D® system and xPONENT® 4.3u1 software and analyzed using Bio-Plex Manager software.

### 1.13 Statistical Analysis

Graphs and statistics were generated using Prism 9 software for Windows (Graphpad Software Inc.). For all experiments, the number of biological replicates and statistical analysis used are described in the figure legends. For comparisons between two groups, a one-tailed T test was used. For comparisons of three or more groups, the analysis of variance (ANOVA) with Tukey or Dunnett or Holm-Šídák post-test was applied when appropriate.

### Example 2: SerpinB9 Wildtype Overexpression in Graft T Cells Protects them from Allogeneic Rejection while Having Minimal Impact on Host Immune Cells

### 2.1 T Cells Over-Expressing SB9 Wildtype can be Manufactured and Expanded

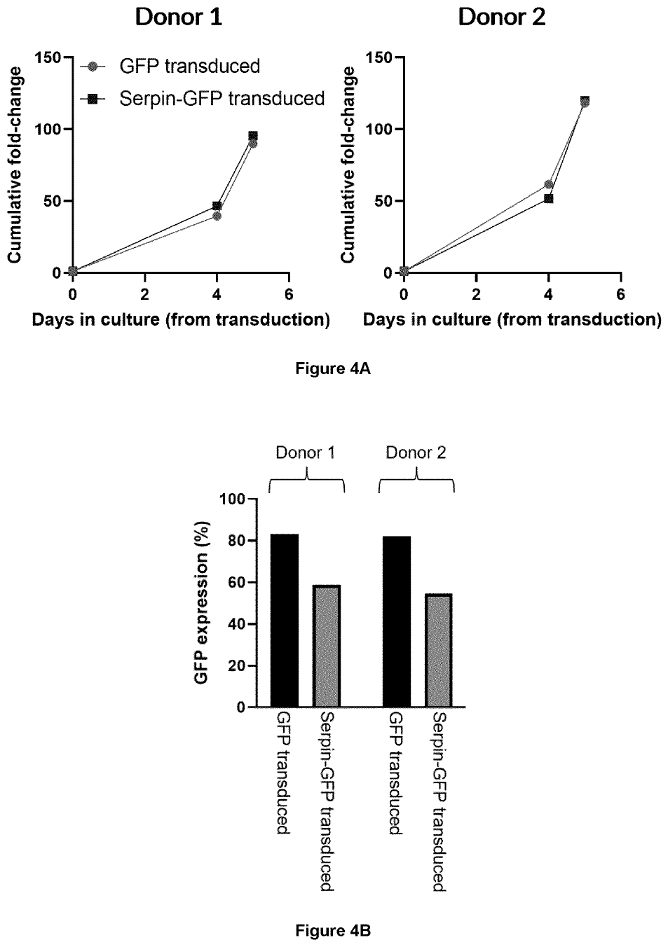

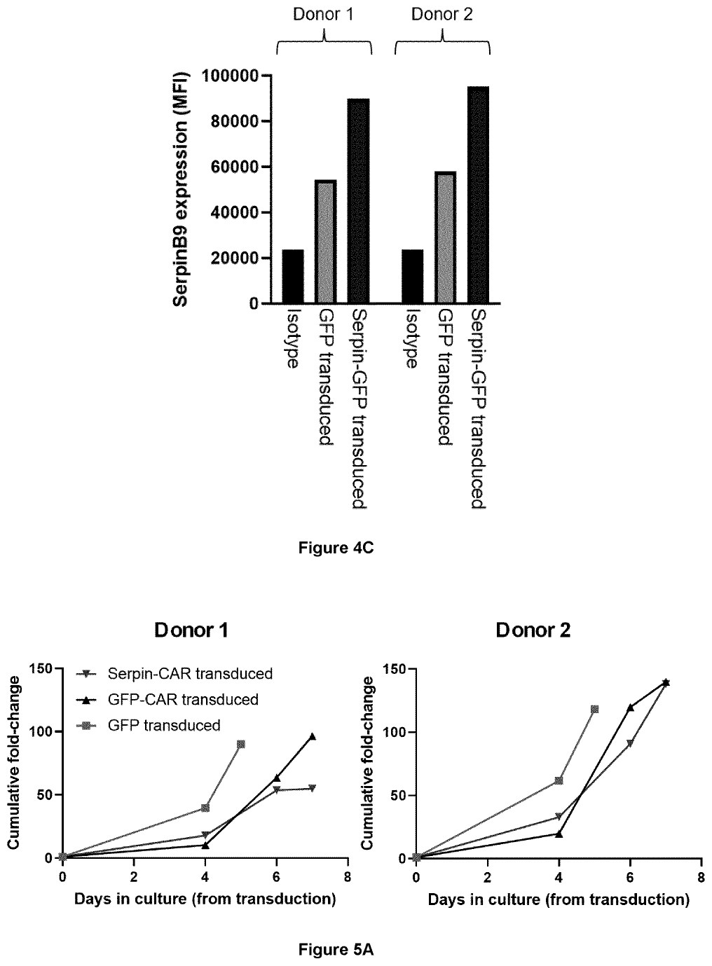

To determine whether we can manufacture T cells which over-express SB9 wildtype, we performed retroviral transduction of SB9 wildtype, as a bicistronic construct with GFP separated by a furin P2A linker (Serpin-GFP), on T cells obtained from two donors. GFP transduced T cells served as controls. The proliferation of T cells was monitored over the manufacturing period (FIG. 1) by trypan blue exclusion cell counts using the haemocytometer. The transduction efficiency and SB9 expression were determined by GFP expression and intracellular staining of SB9, respectively, by analysis by flow cytometry. Both GFP and serpin-GFP transduced T cells proliferated similarly over the manufacturing period and cell numbers increased 90-120-fold post transduction (FIG. 4A). The GFP transduction efficiency was lower for Serpin-GFP compared to GFP transduced cells (Serpin-GFP: 55-59% vs GFP: 82-83%) (FIG. 4B), in line with lower transduction efficiencies usually observed for bicistronic constructs. SB9 wildtype was over-expressed roughly 1.6-fold in GFP-positive cells, for both donors tested (FIG. 4C).

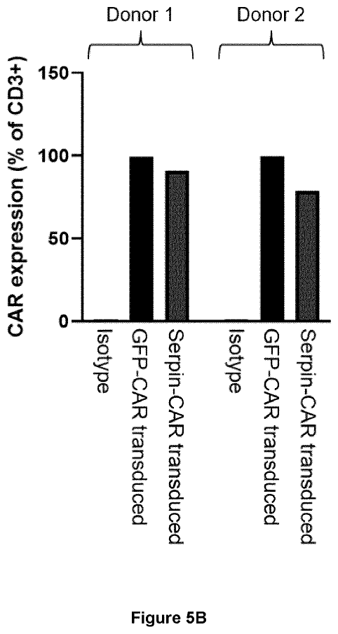

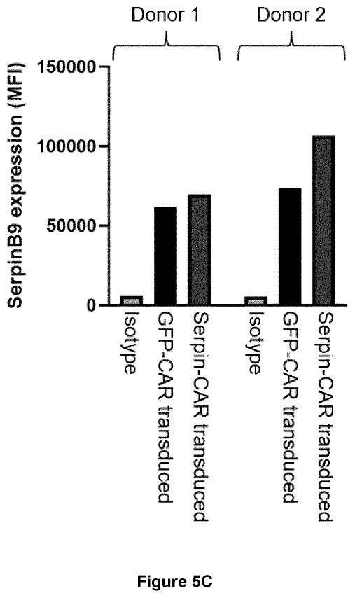

To determine whether SB9 wildtype could also be over-expressed in CAR T cells, we transduced T cells with SB9 wildtype, as a bicistronic construct with CD30.CAR separated by a furin P2A linker (Serpin-CAR). GFP-CAR transduced T cells served as controls. Serpin-CAR transduced T cells had a lower proliferation fold change compared to GFP-CAR for donor 1 (Serpin-CAR: 55-fold vs GFP-CAR: 96-fold) but had similar proliferation fold-change for donor 2 (˜140-fold) (FIG. 5A). The CD30.CAR expression was lower for both donors when Serpin-CAR construct was used compared to GFP-CAR (Serpin-CAR: 79-91% vs GFP-CAR: 99%) (FIG. 5B). SB9 wildtype was over-expressed roughly 1.2-1.5-fold in CD30.CAR-positive cells, for both donors tested (FIG. 5C).

### 2.2 CAR T Cells Over-Expressing SB9 Wildtype Maintain CAR Cytotoxicity

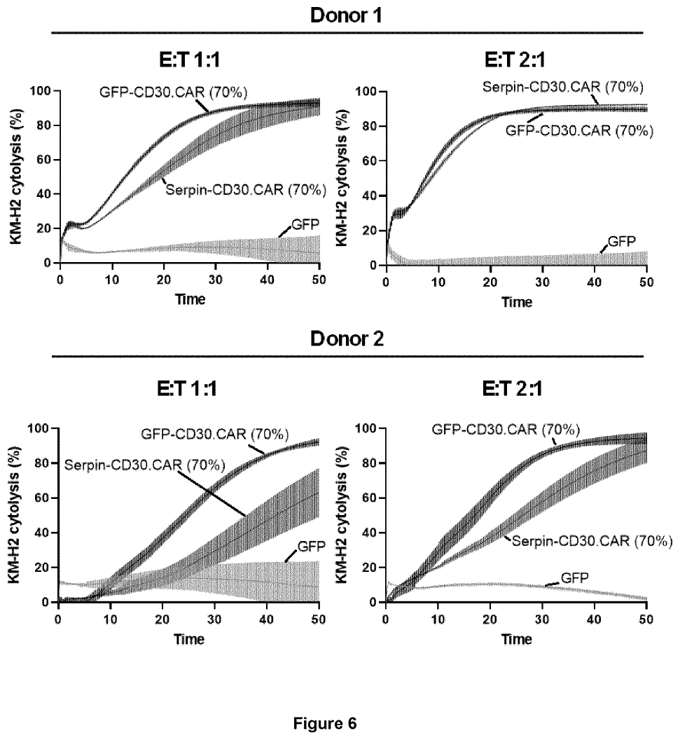

To determine whether a CAR is functional on T cells over-expressing SB9, we tested CD30.CAR cytotoxicity against CD30 positive KM-H2 targets using the xCELLigence assay platform. T cells were transduced with either GFP-CD30 CAR, Serpin-CD30.CAR or GFP, which served as a control. To ensure that the percentage of T cells expressing CD30.CAR was similar between the two conditions, we normalized the CD30.CAR T cells to 70% of total cells by the addition of GFP T cells. CD30.CAR T cells were able to kill >80% of KM-H2 targets within 48 h of the co-culture for both donors at E:T=2:1 (FIG. 6) and the CD30.CAR was functional regardless of SB9 expression.

2.3 T Cells Over-Expressing SB9 Wildtype are Protected from Allogeneic Host Rejection, with Negligible Impact on Host T and NK Cells

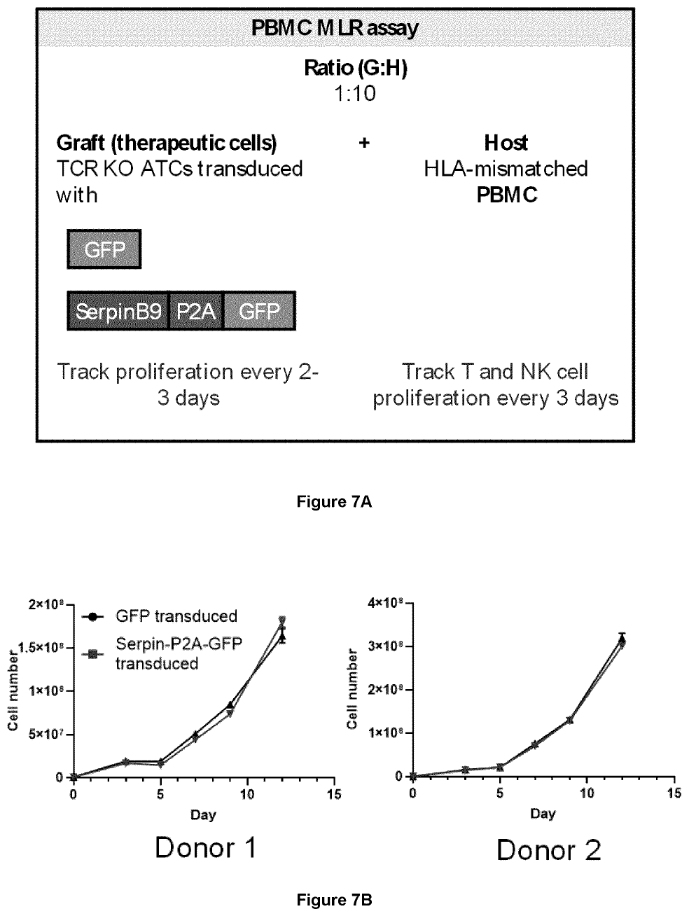

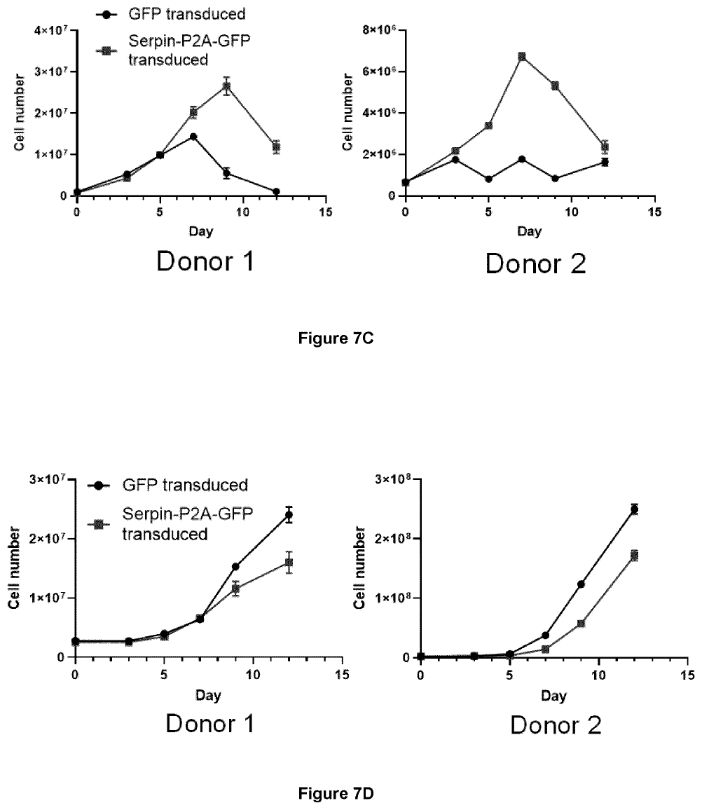

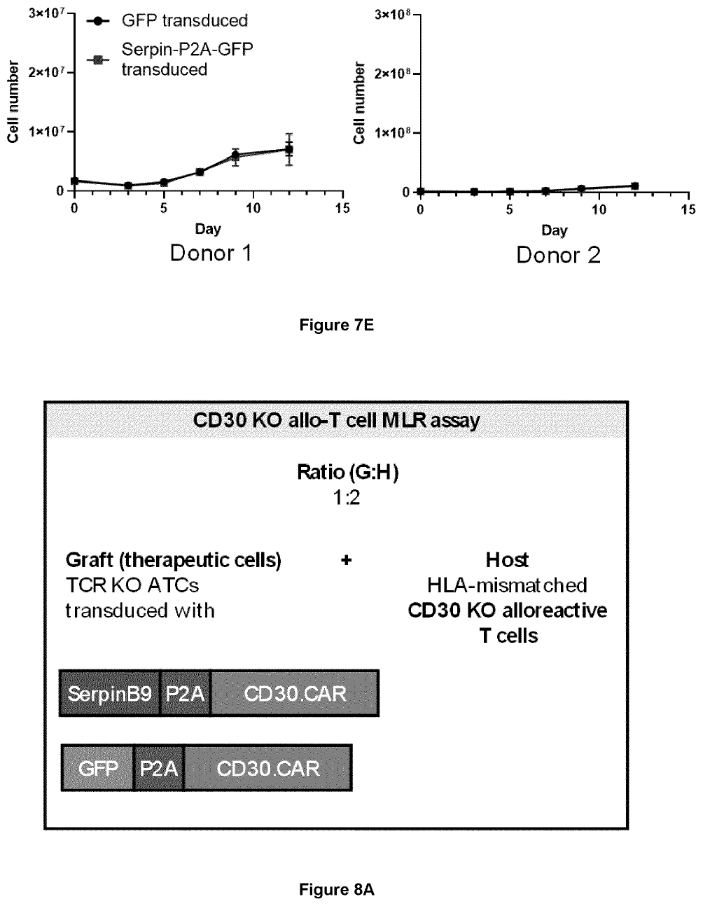

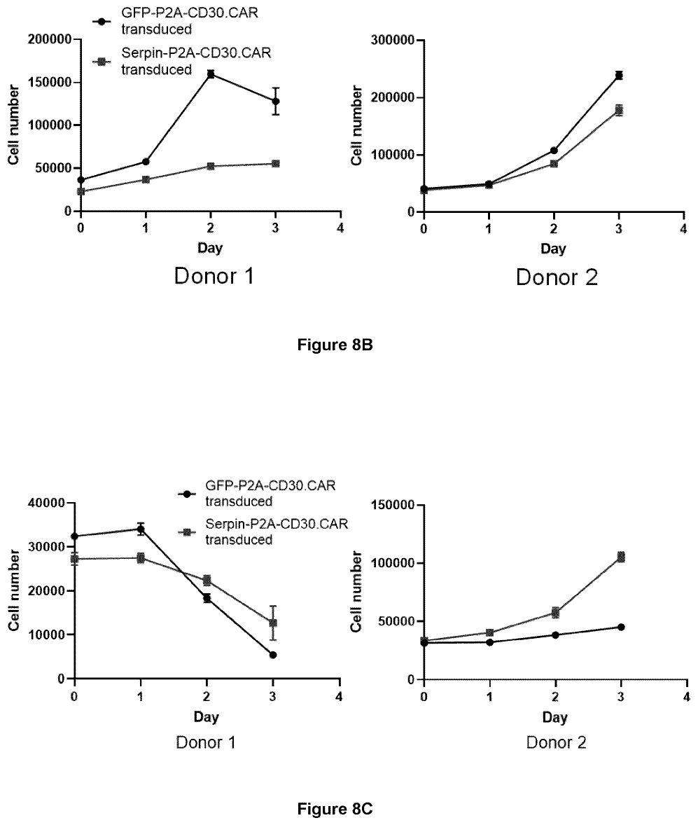

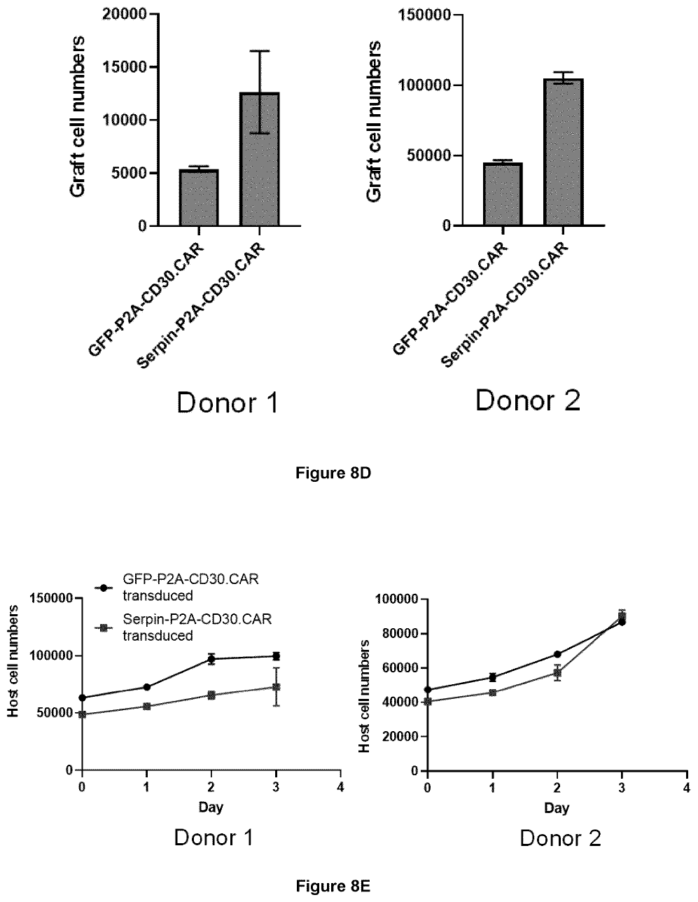

To determine whether T cells were protected from allogeneic rejection by the over-expression of SB9, mixed lymphocyte reaction (MLR) assays were set up (FIGS. 7A and 8A). Graft T cells expressing either GFP alone or Serpin-GFP were co-cultured with HLA-mismatched host PBMCs in a graft:host cell ratio of 1:10, and cell numbers were monitored by flow cytometry over 12 days (FIG. 7A). When graft T cells transduced with GFP or Serpin-GFP were cultured alone, proliferation fold-change was similar over the culture duration for two donors tested (FIG. 7B). When host PBMCs were added to the graft T cells, T cells transduced with GFP were unable to proliferate beyond day 7 for donor 1 or day 3 for donor 2 and graft cell numbers were kept low or were eventually eliminated (FIG. 7C). On the other hand, Serpin-GFP transduced graft T cells were able to proliferate further until day 9 or day 7 for donors 1 and 2 respectively, before decreasing in graft cell numbers (FIG. 7C). A maximum difference in graft cell numbers of 4 to 5-fold, between Serpin-GFP and GFP-transduced cells, was observed on day 9 or day 7 for donors 1 and 2 respectively (FIG. 7C). Host CD3 positive T cells continued to proliferate over the culture duration and NK cell numbers were similar regardless of graft co-culture (FIGS. 7D and 7E), indicating that graft T cells, over-expressing SB9, had minimal impact on host cells.

We further investigated whether CD30.CAR T cells were also protected from allogeneic rejection by the over-expression of SB9. Graft T cells transduced with either GFP-CAR or Serpin-CAR were cultured with HLA-mismatched host CD30 KO alloreactive T cells in a graft:host ratio of 1:2. Cell numbers were monitored by flow cytometry over three days (FIG. 8A). When graft T were cultured alone, GFP-CAR transduced cells had higher proliferation (1.3 to 2.3-fold) compared to Serpin-GFP transduced cells (FIG. 8B). In the MLR set up of donor 1, T cells transduced with GFP-CAR were eliminated more quickly than Serpin-CAR transduced T cells (FIG. 8C) and on day 3, Serpin-CAR transduced grafts were 2.3-fold higher in cell numbers than GFP-CAR transduced grafts (FIG. 8D). In the MLR set up for Donor 2, GFP-CAR transduced T cells did not proliferate while Serpin-CAR transduced grafts proliferated 3.2-fold over three days (FIG. 8C). On day 3, Serpin-CAR transduced grafts were 2.3-fold higher in cell numbers than GFP-CAR transduced grafts for Donor 2 (FIG. 8D). Host T cells co-cultured with either GFP-CAR or Serpin-CAR transduced graft cells proliferated similarly in MLR set ups of both donors tested, again indicating that graft T cells, over-expressing SB9, had minimal impact on host cells (FIG. 8E).

### Example 3: SerpinB9(CAS) Expression in Graft T Cells Protects them from Allogeneic Rejection while Keeping Host Immune Cells Unharmed

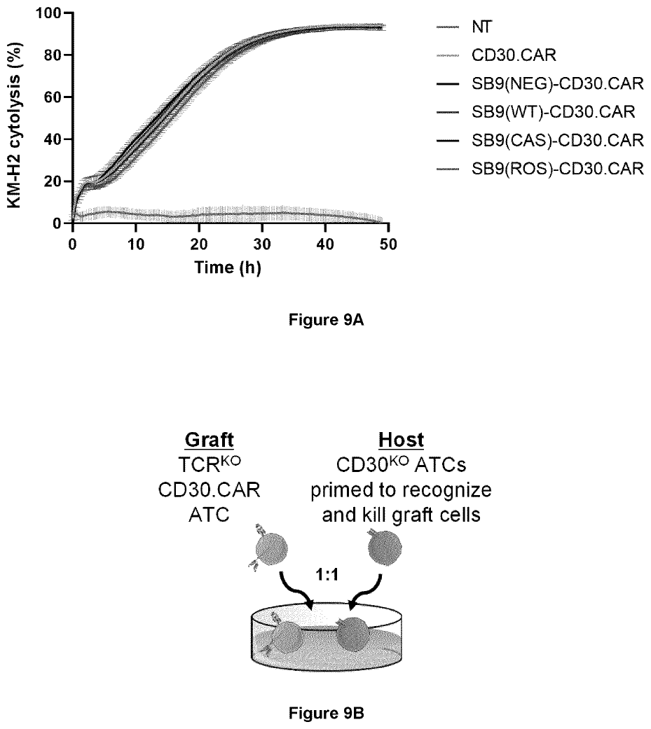

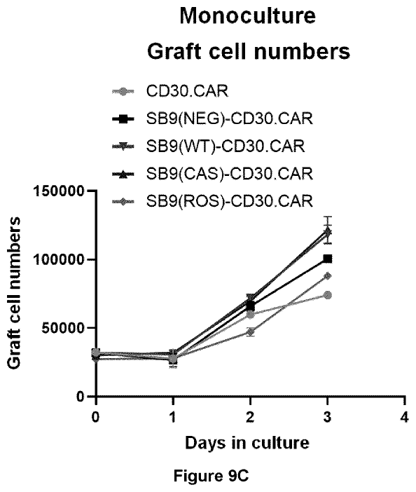

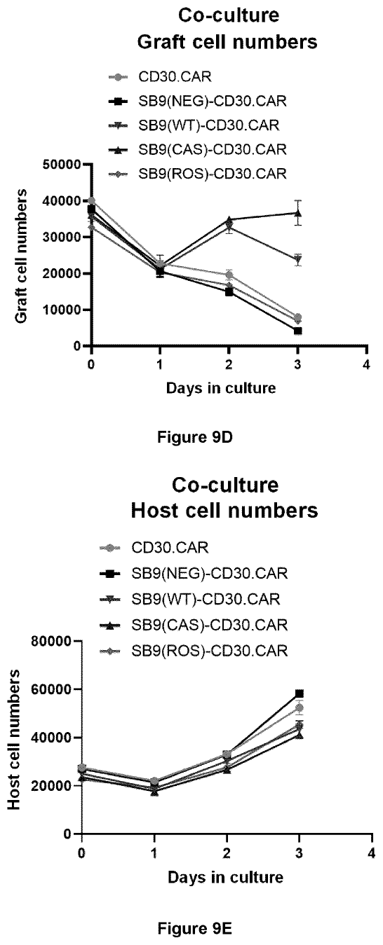

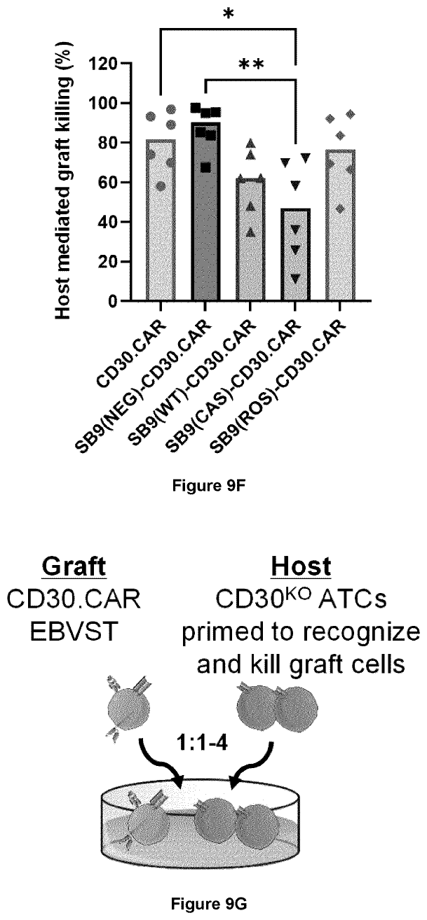

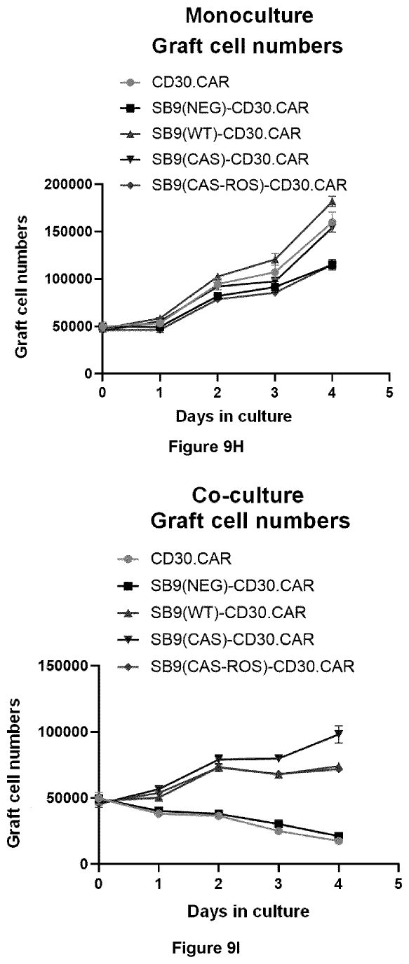

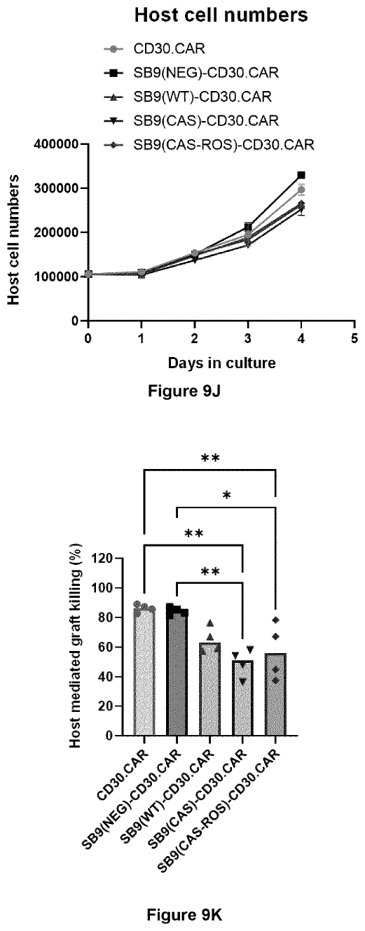

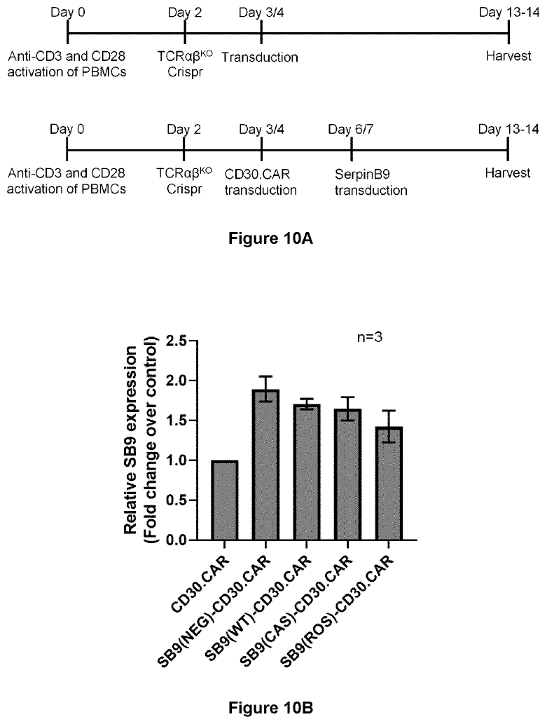

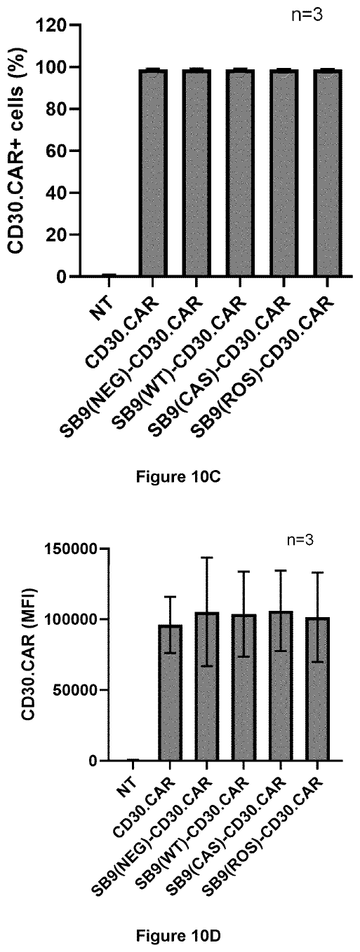

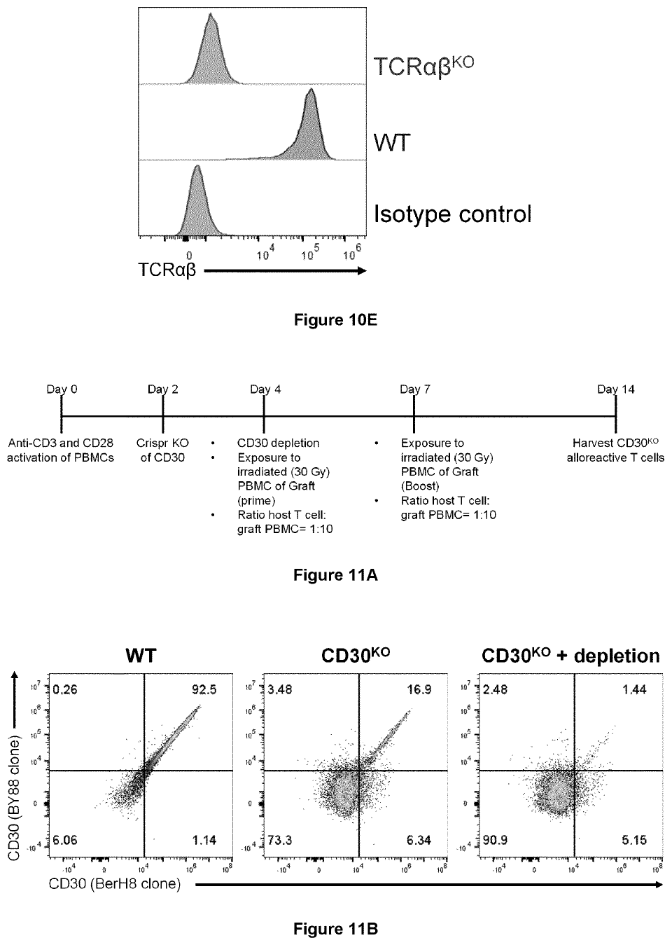

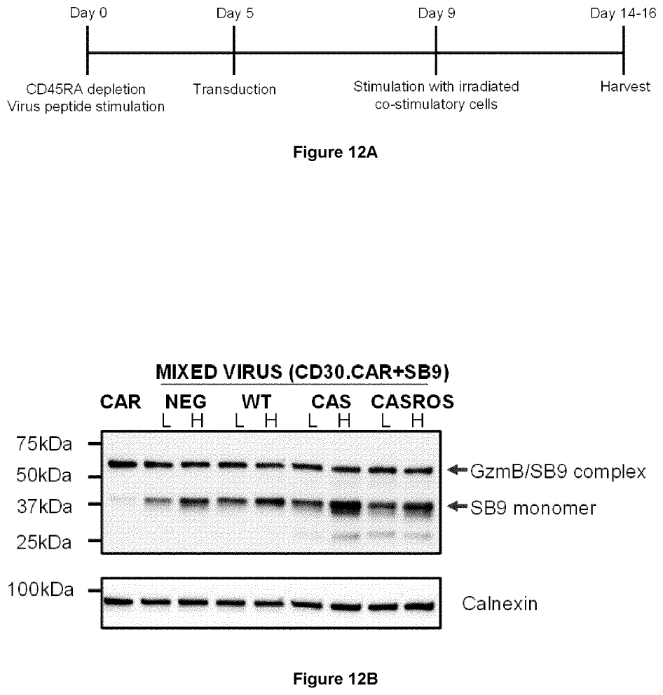

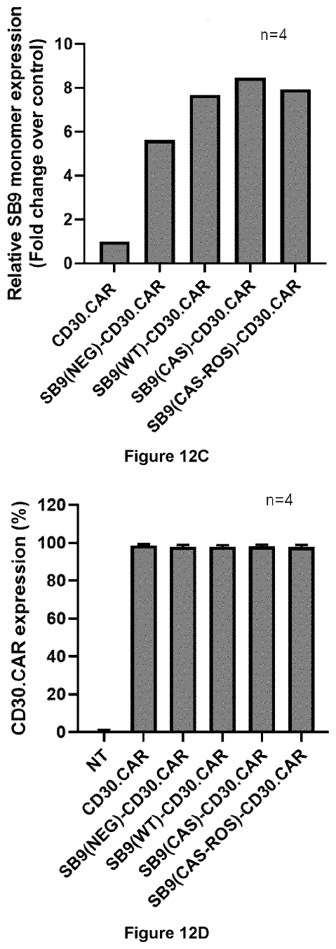

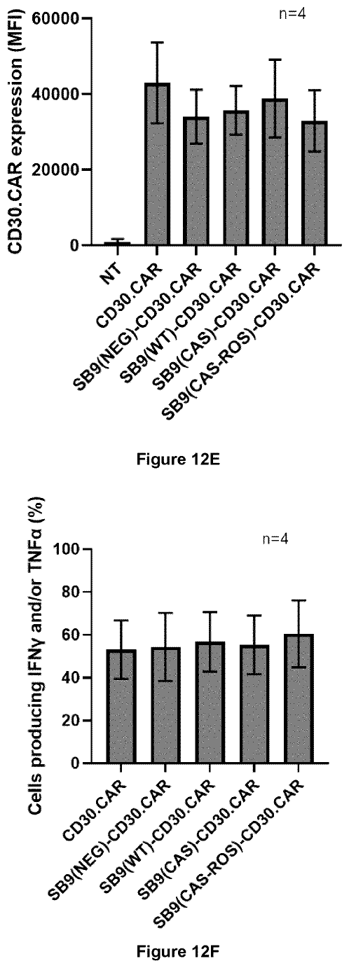

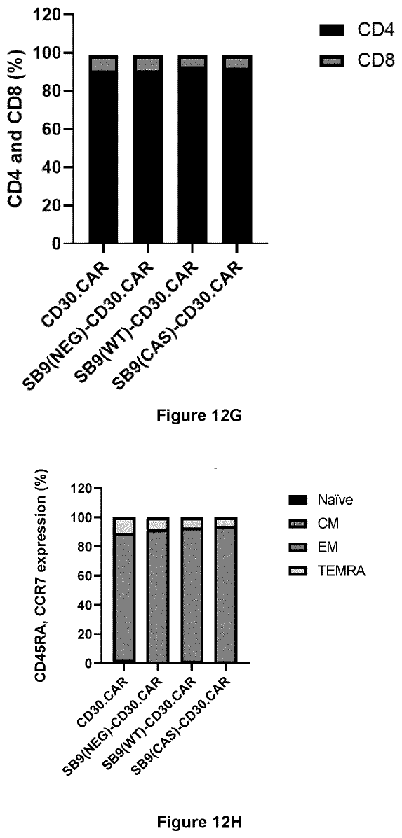

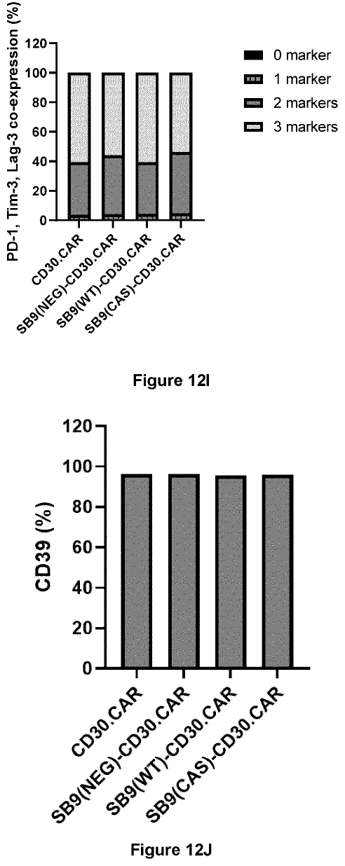

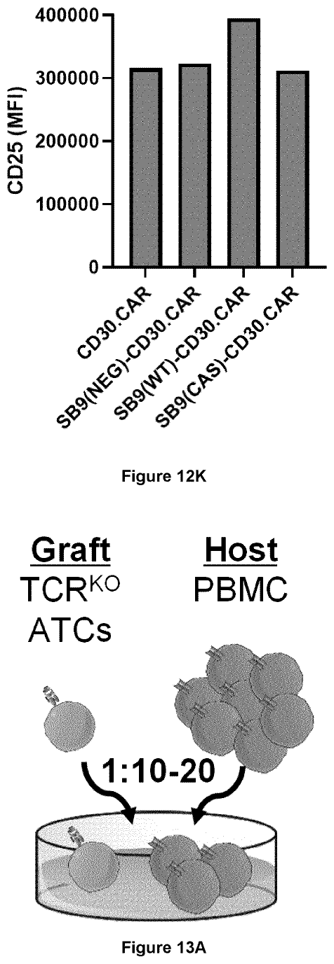

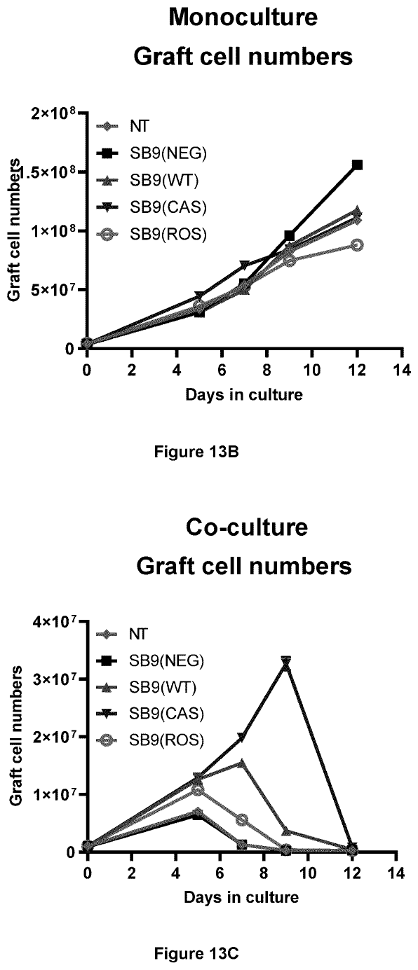

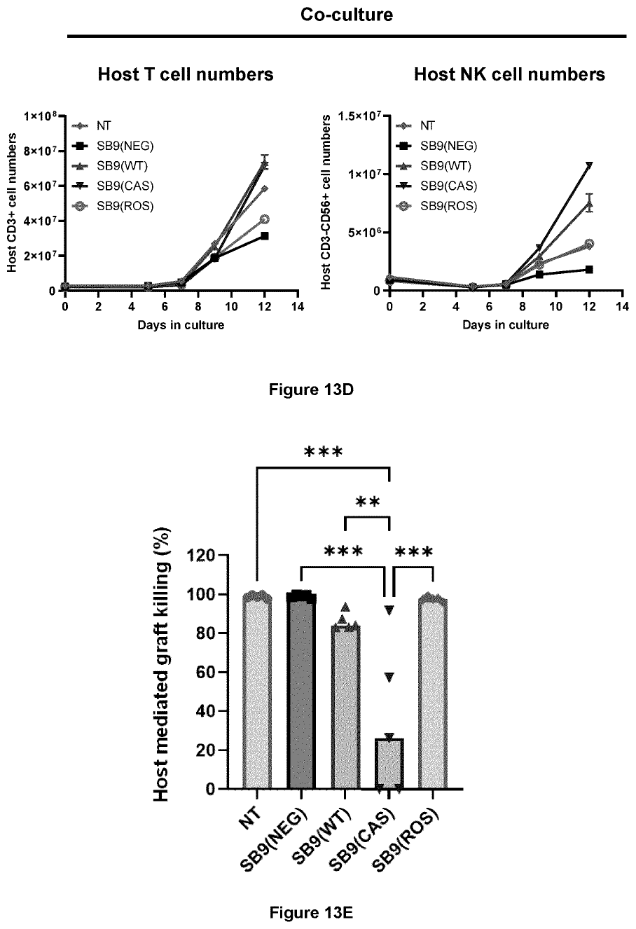

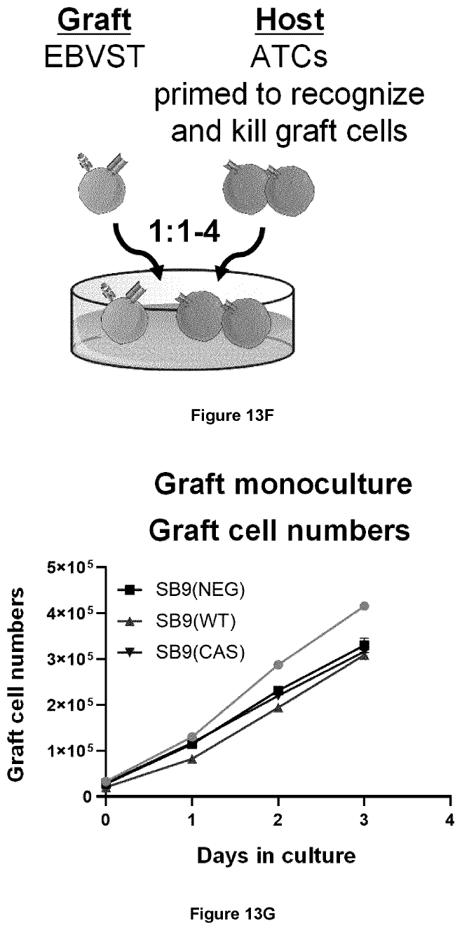

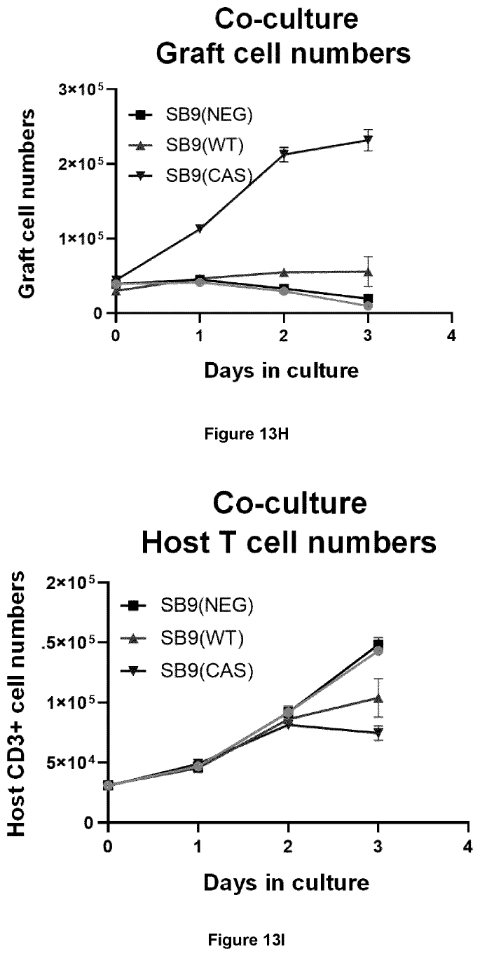

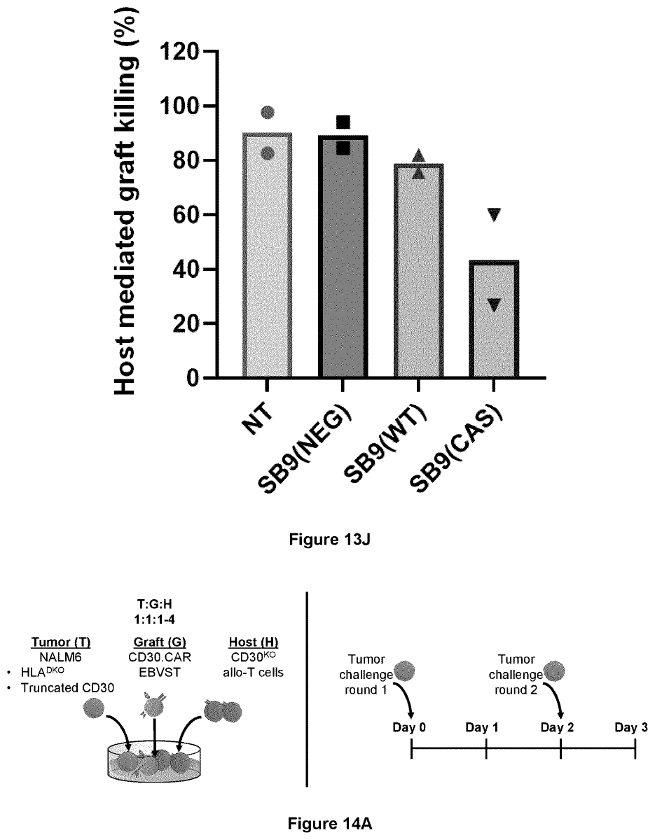

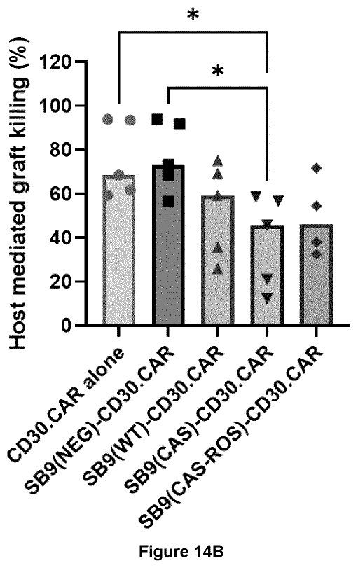

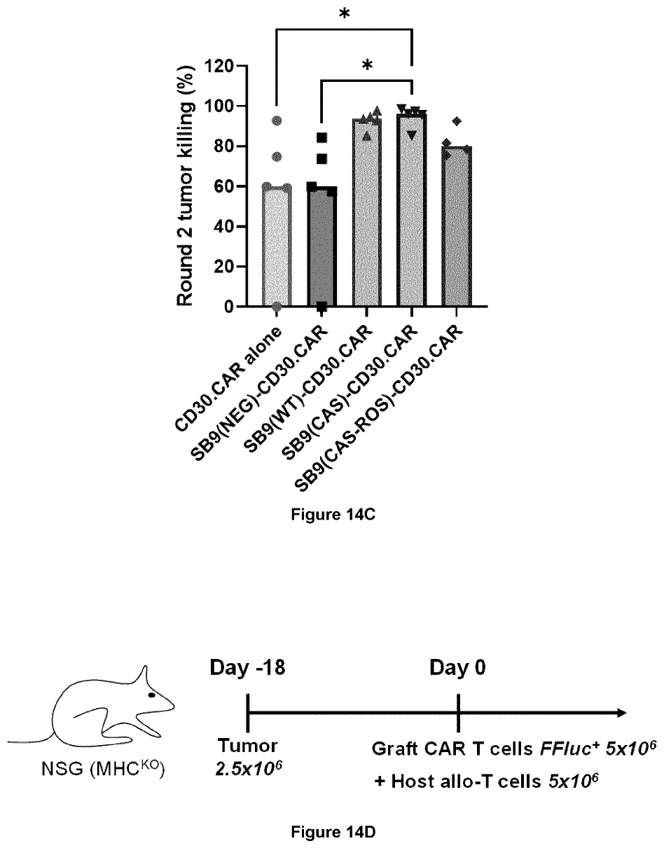

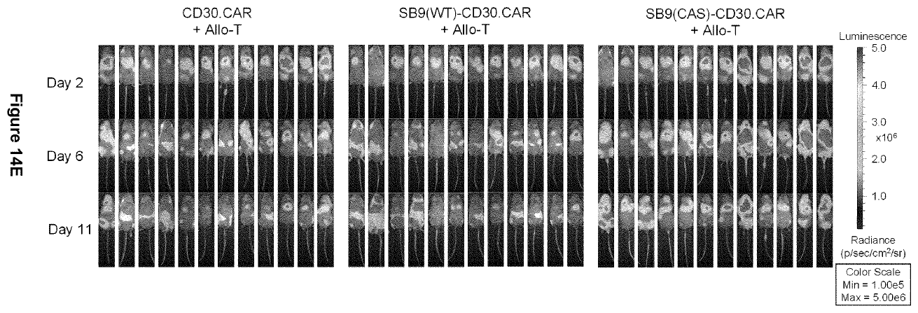

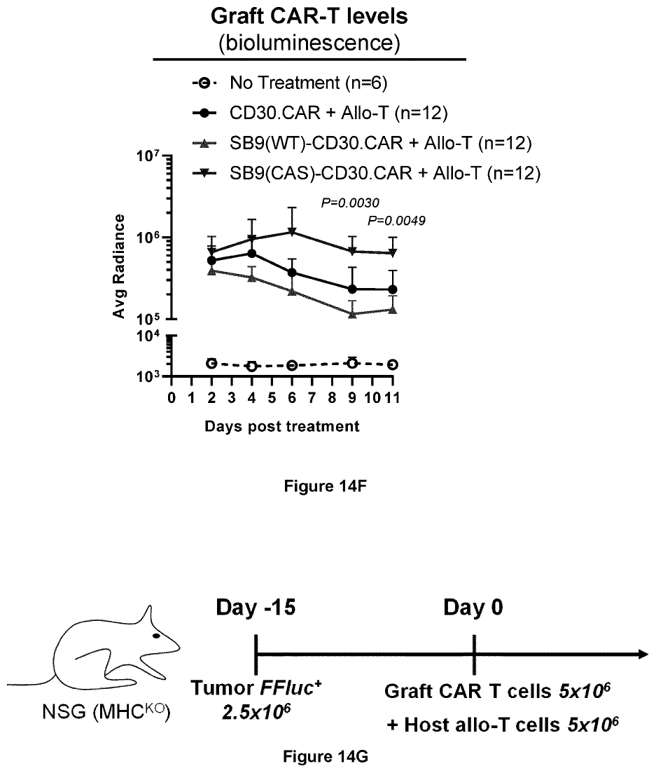

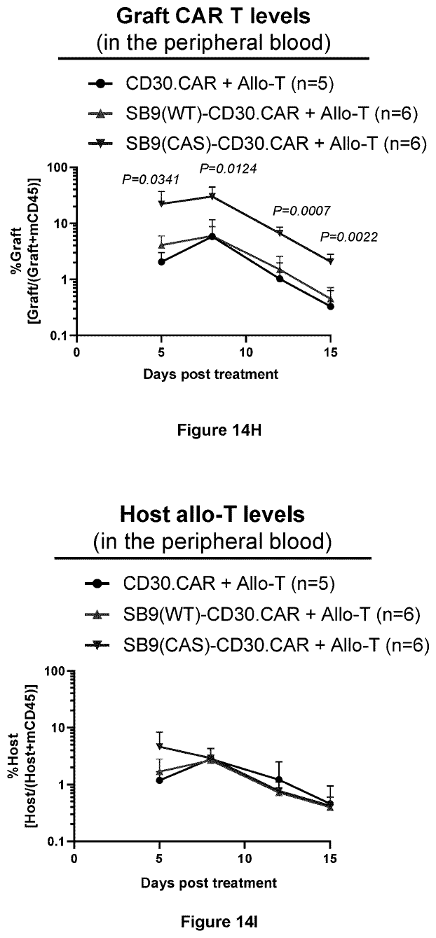

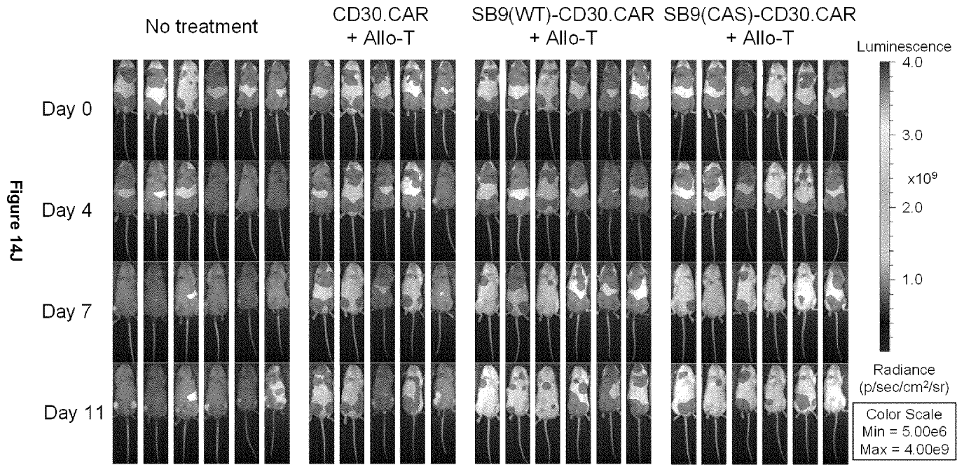

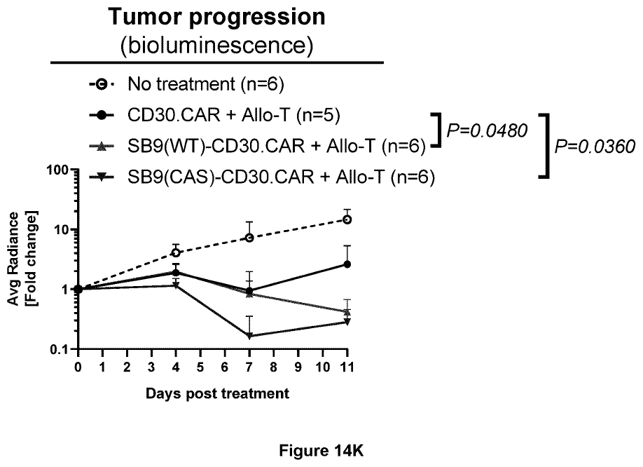

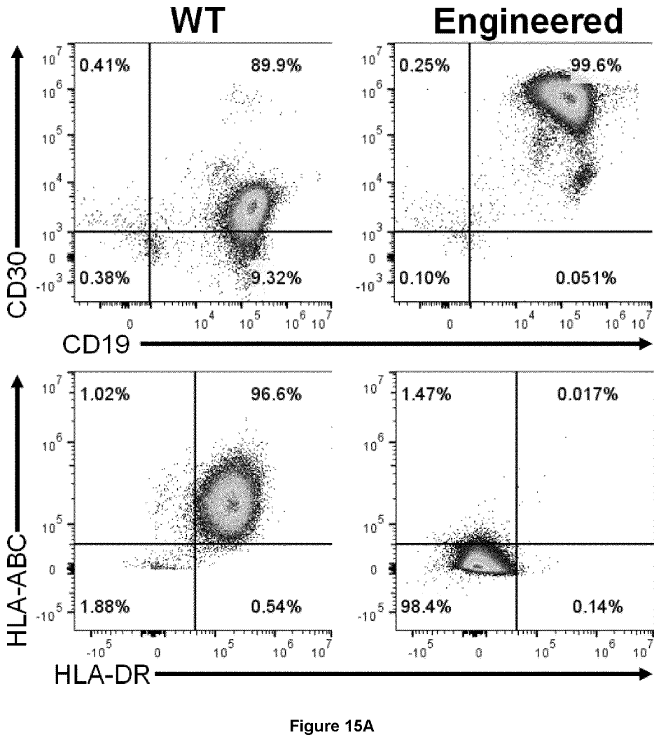

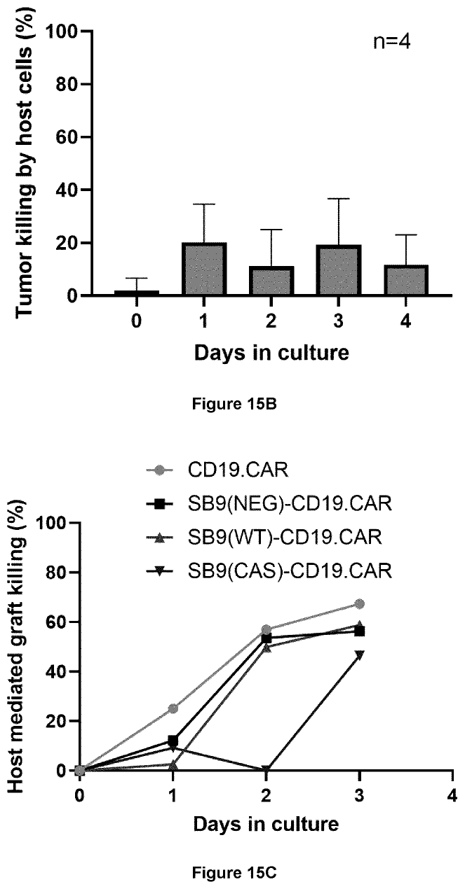

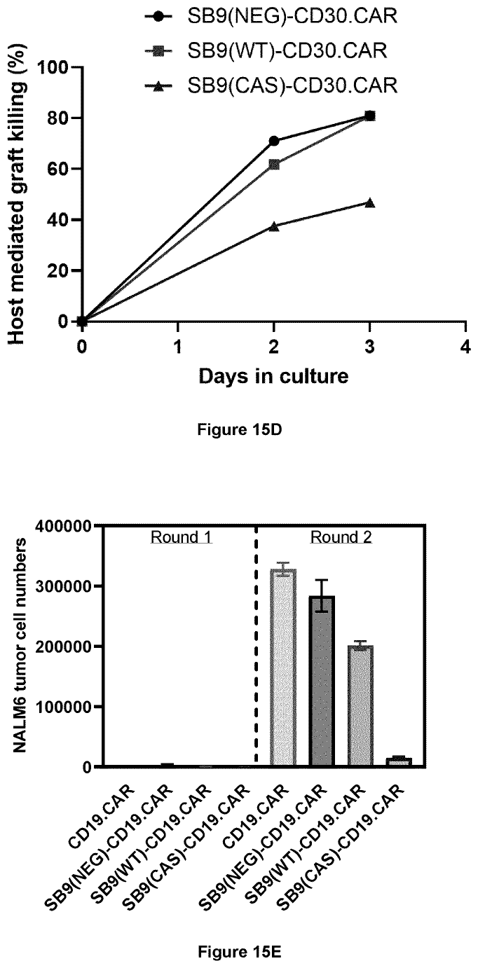

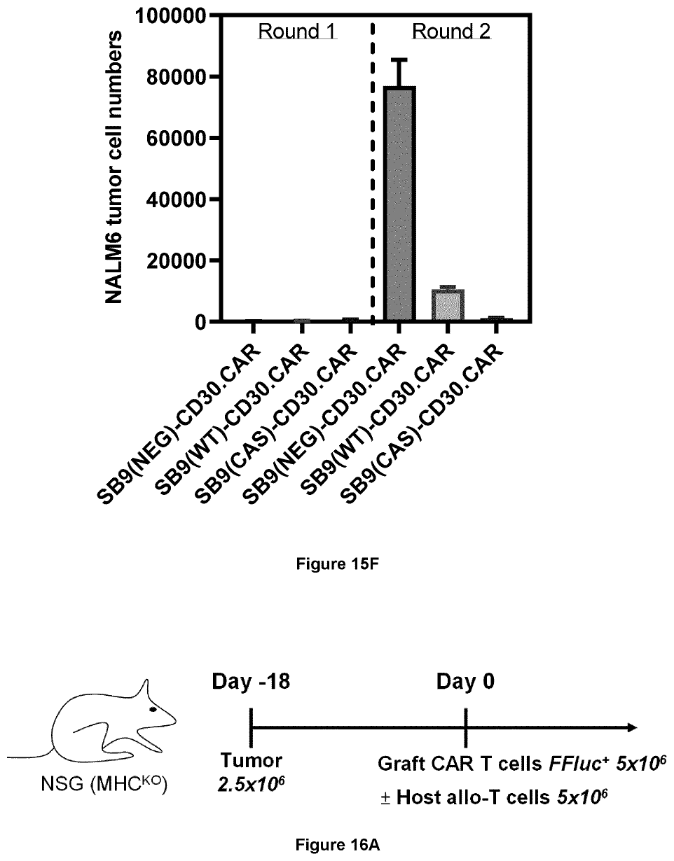

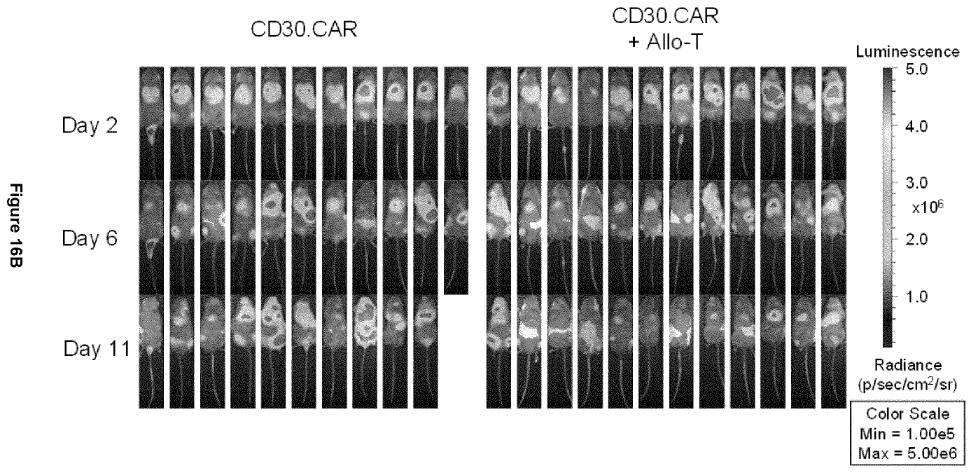

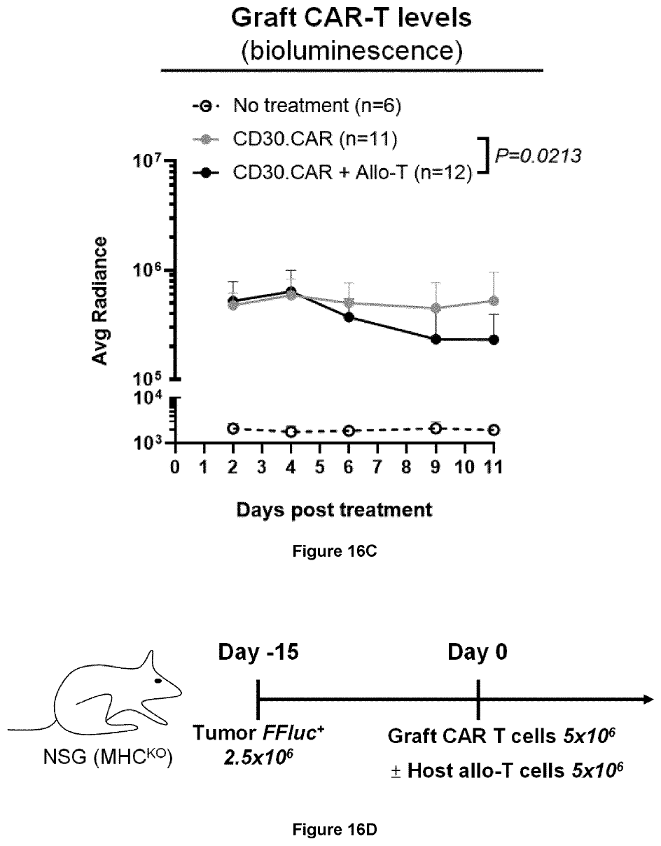

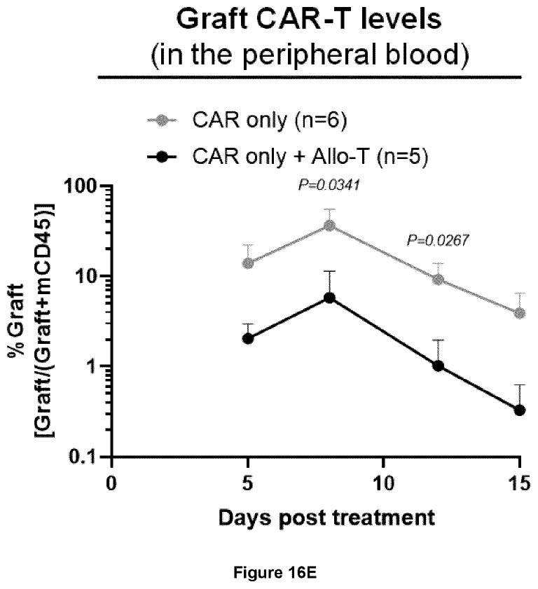

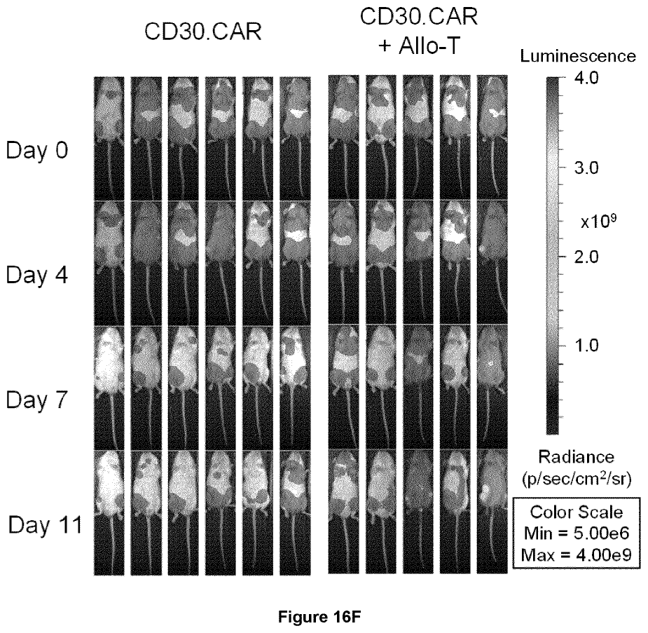

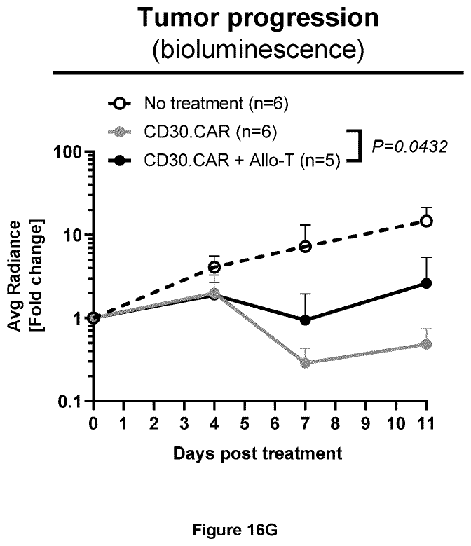

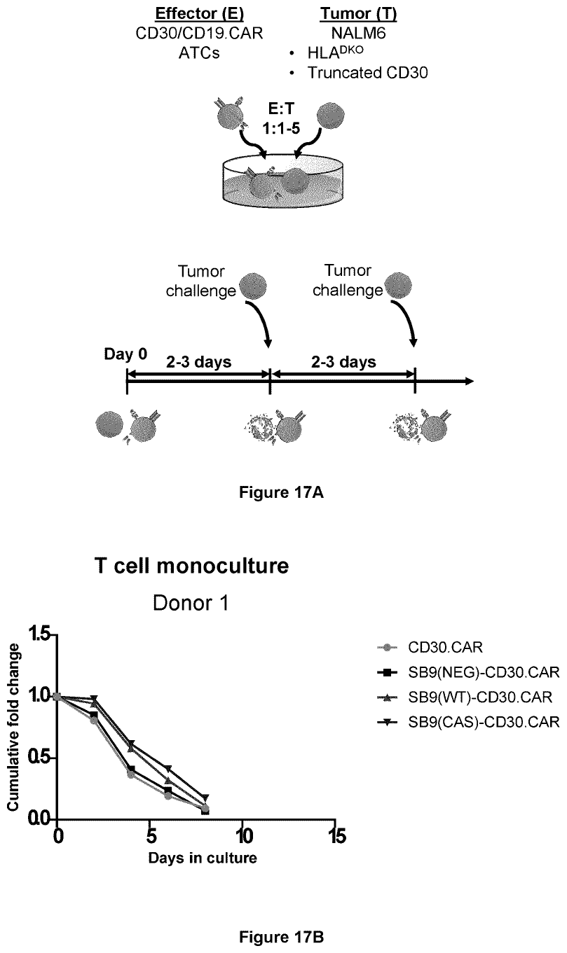

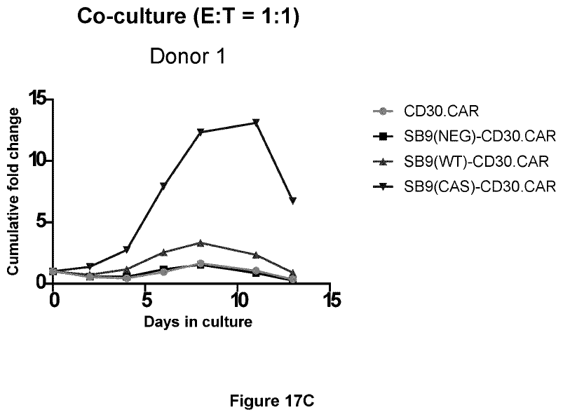

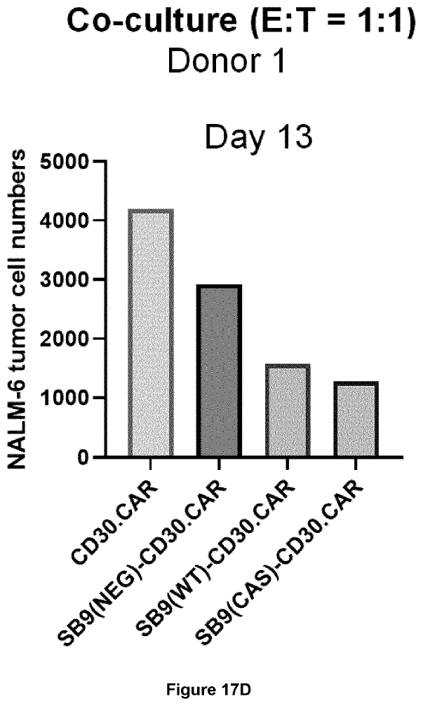

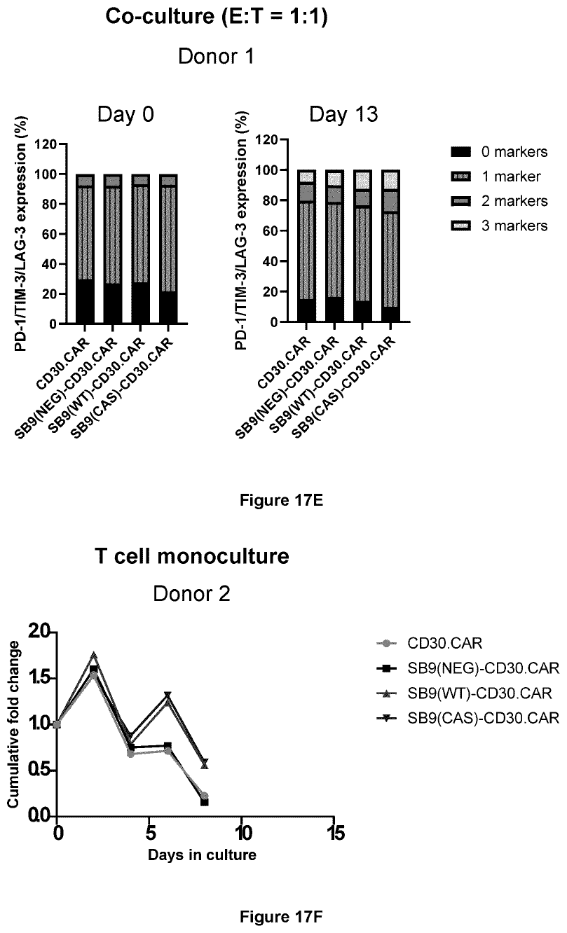

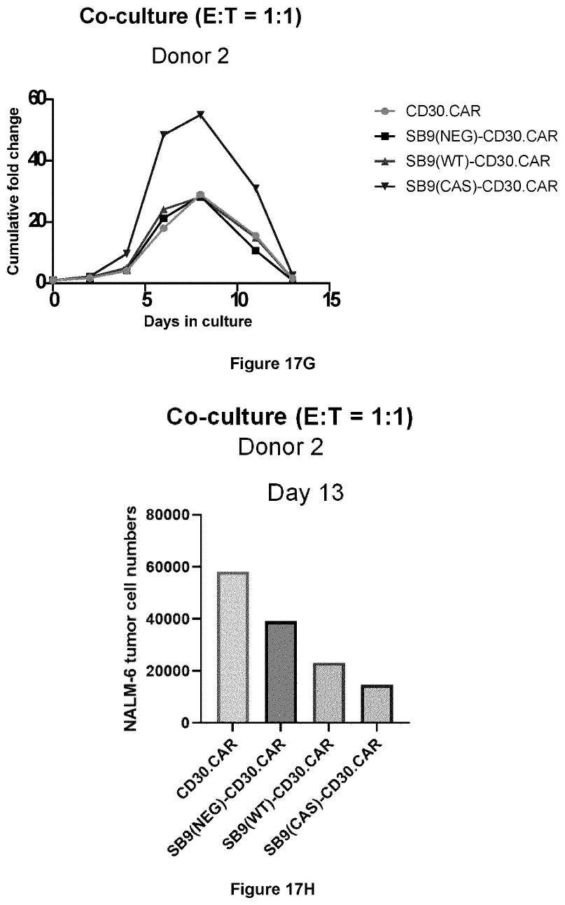

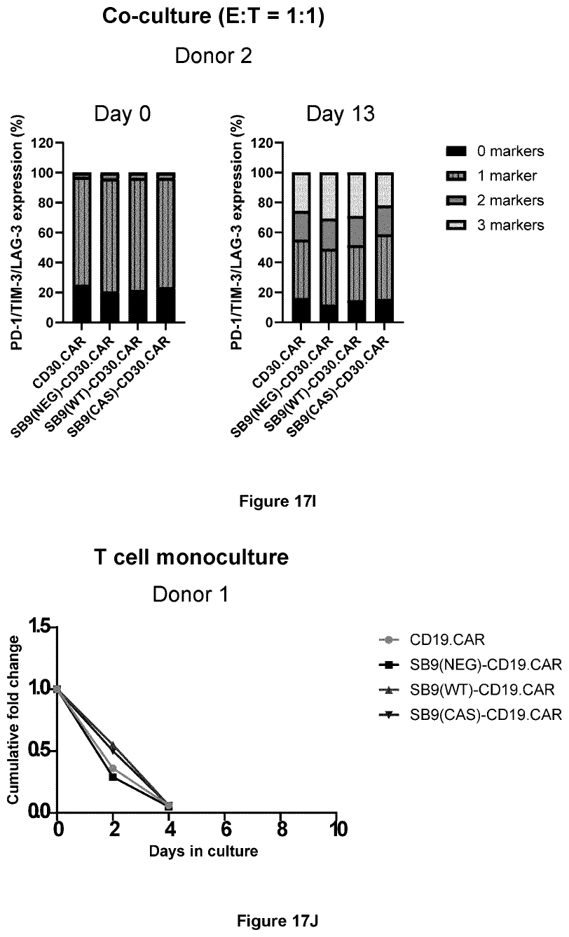

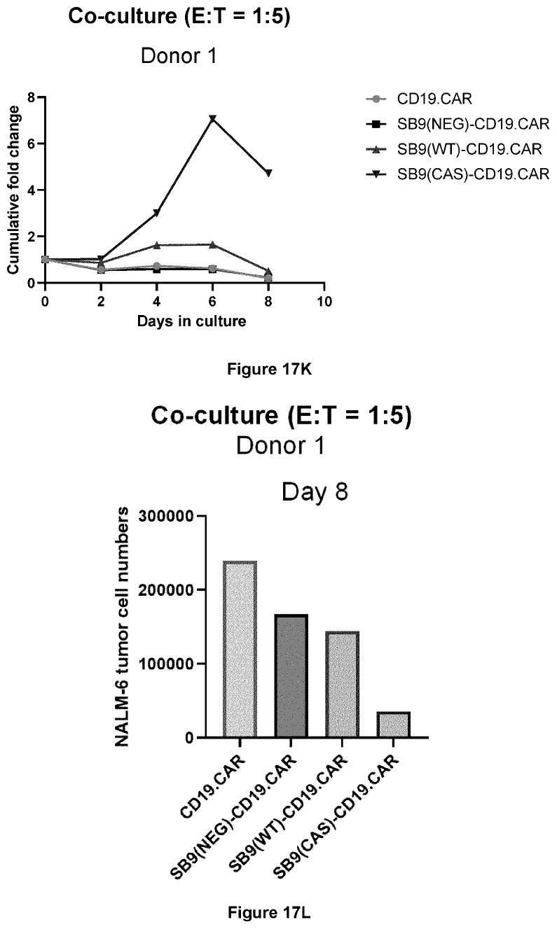

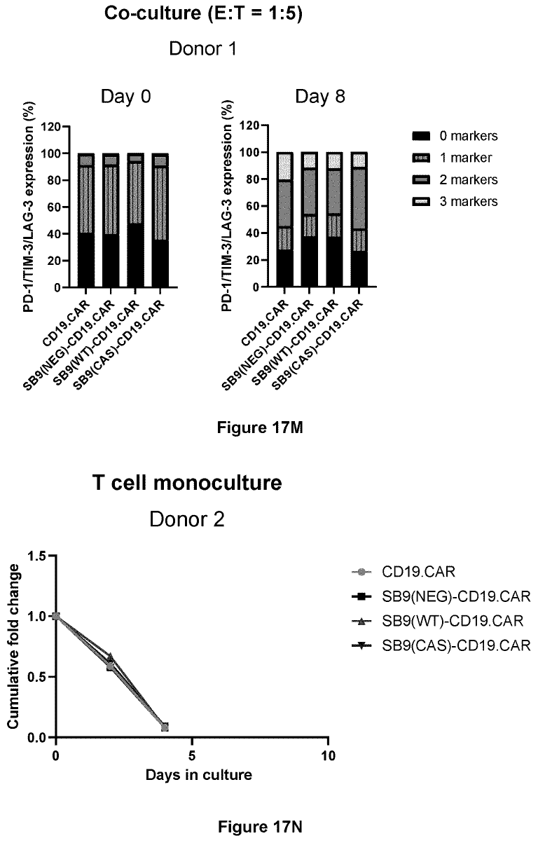

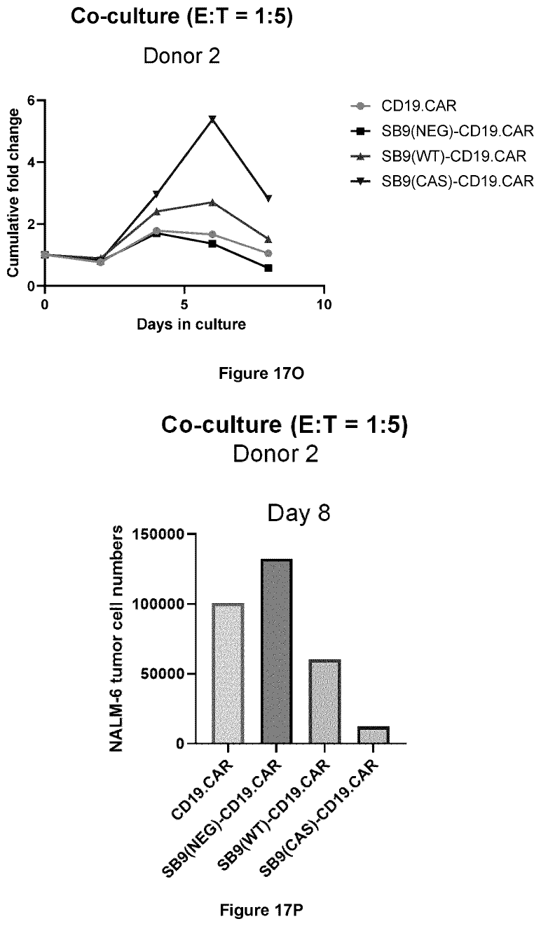

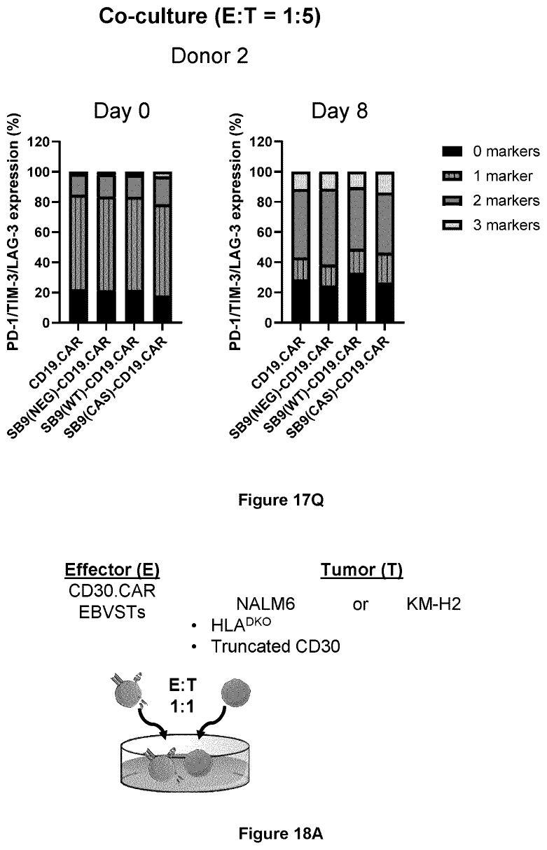

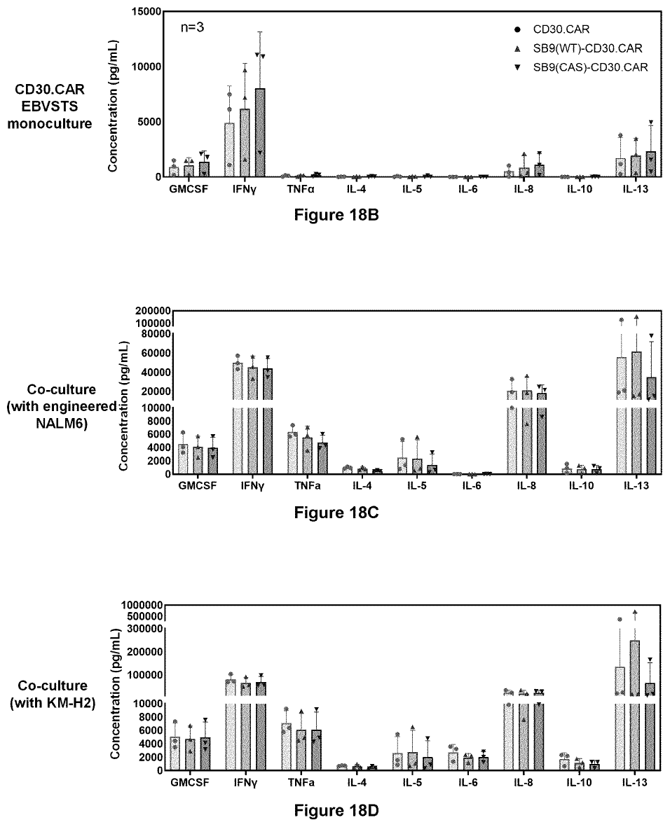

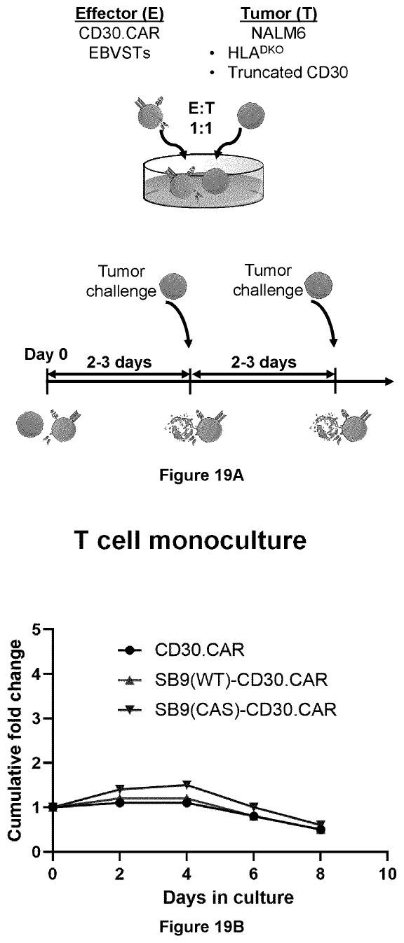

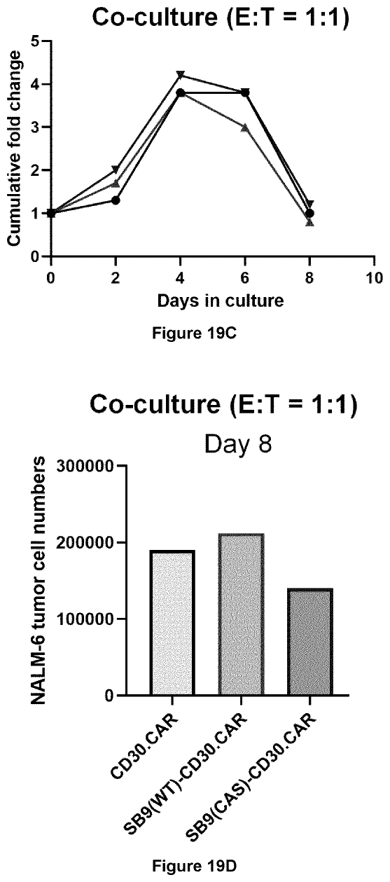

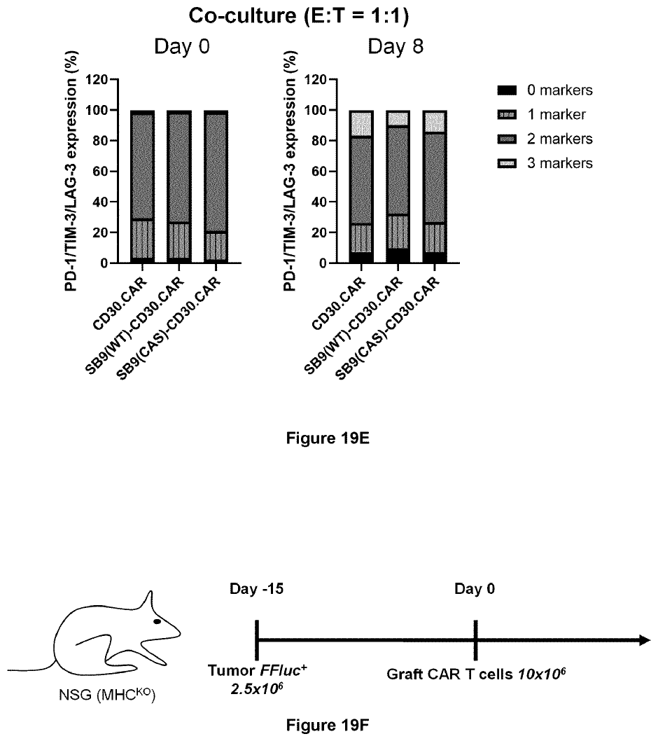

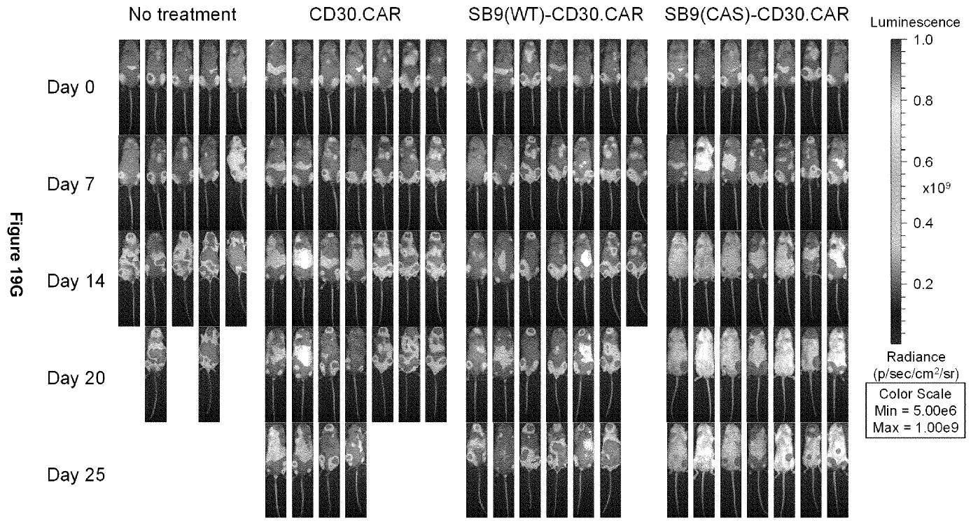

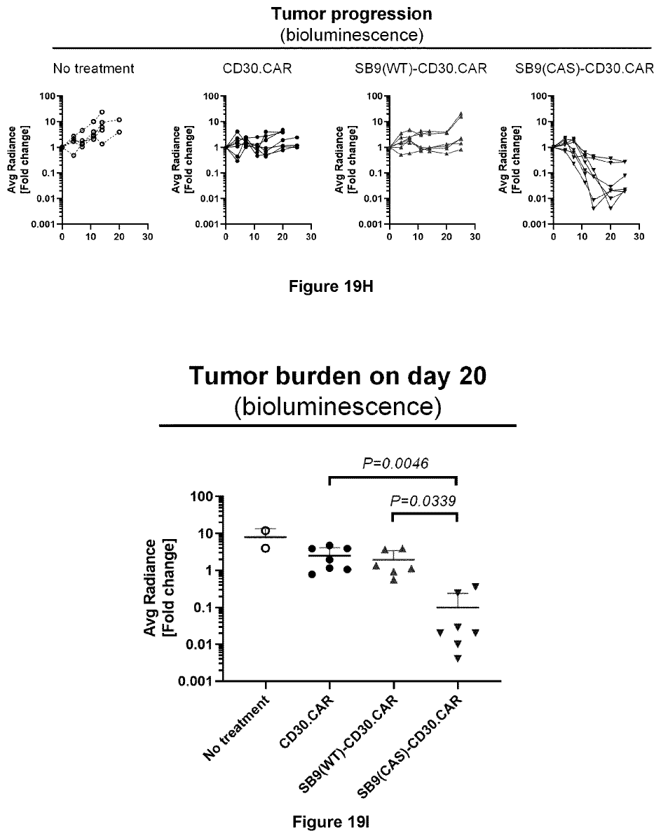

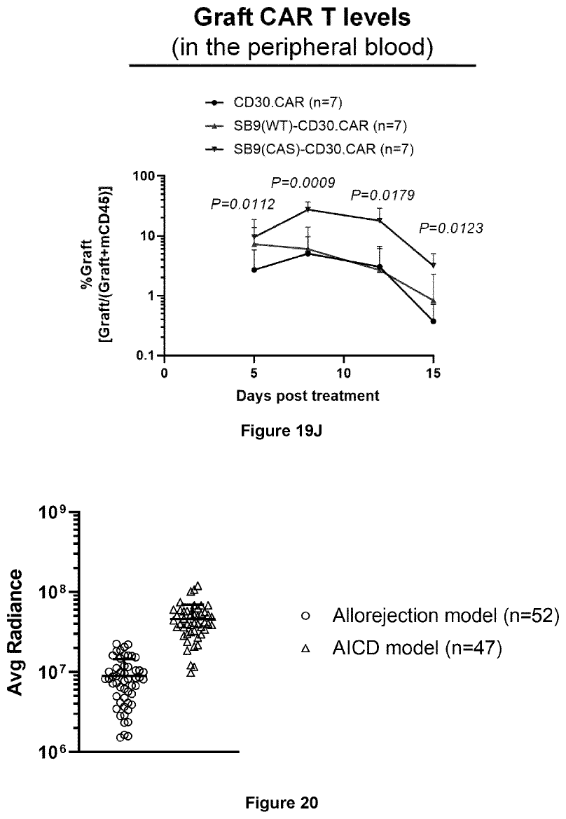

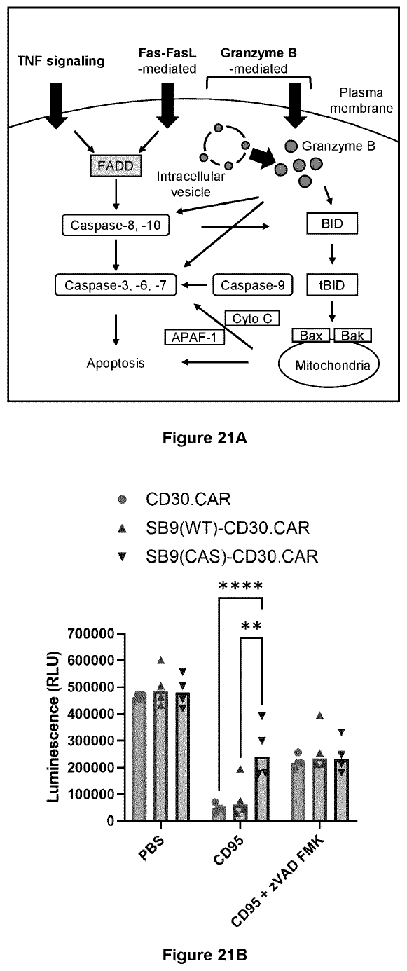

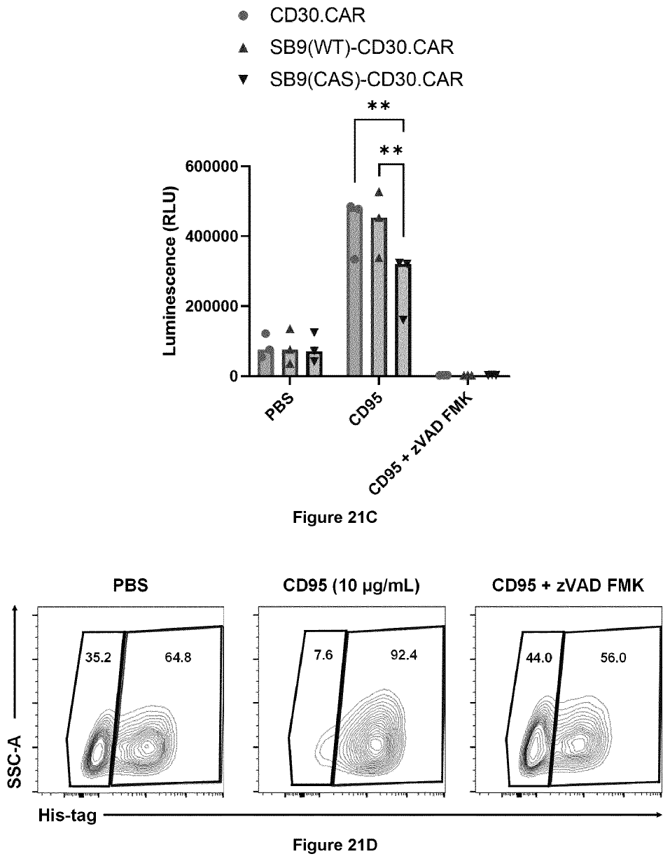

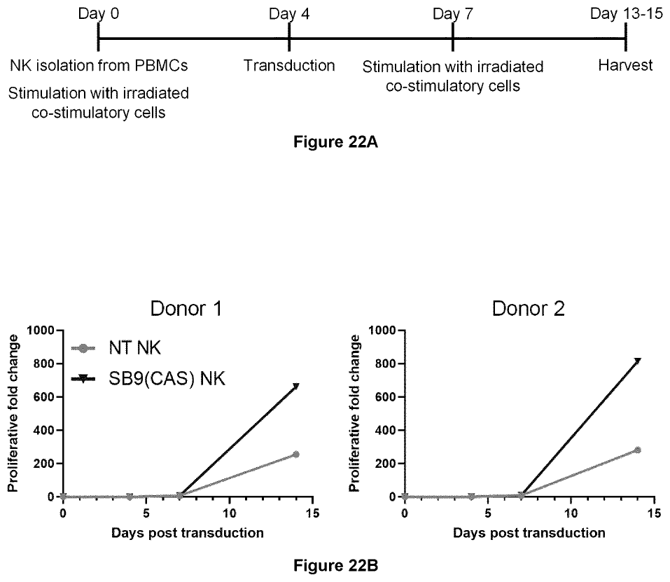

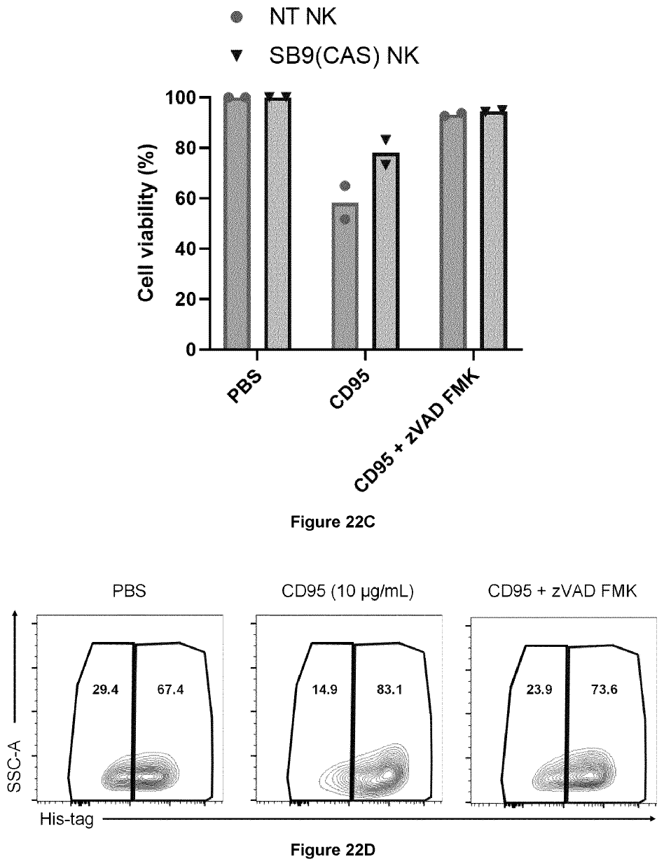

Amino acid substitutions of the glutamic acid at the P1 location (340E) in the reactive centre loop (RCL) of SB9 as well as 327T at the hinge region have been shown to affect SB9 interaction with GzmB and other caspases (13, 14). We were interested to study whether SB9 WT (SEQ ID NO: 1) and mutants: E340D (SB9(CAS)) (SEQ ID NO: 5), C341S; C342S (SB9(ROS)) (SEQ ID NO:6) can protect graft T cells from allogeneic rejection. E340A; T327R (SB9(NEG)) (SEQ ID NO: 47) had been previously described to have low interaction with GzmB and was used as a negative control. We initially focused our study on CD30.CAR ATCs over-expressing the different forms of SB9 (SB9-CD30.CAR ATCs). To first check whether CAR functionality was affected by SB9 over-expression, we performed killing assays of the KM-H2 Hodgkin lymphoma cell line using xCELLigence Real-Time Cell Analysis (RTCA). The over-expression of SB9 and its derivatives were 1.3-2.1 times compared to CD30.CAR ATC control (FIG. 10B) and the CD30.CAR expression was unaffected by SB9 over-expression (FIG. 10C and FIG. 10D). The SB9-CD30.CAR ATCs had similar CAR cytotoxicity and killing kinetics compared to the CD30.CAR ATC control (FIG. 9A).

To determine whether SB9 over-expression protects CAR T cells from allogeneic rejection, we set up mixed lymphocyte reaction (MLR) models in vitro using cells from 6 independent graft-host pairs. To ensure minimal graft-versus-host reactivity, we disrupted surface expression of TCRαβ using Crispr genome editing (TCRαβ KO T cells) that resulted in ≥98% efficiency (FIG. 10E). TCRαβ KO CD30.CAR ATC grafts were co-cultured in a 1:1 ratio with HLA-mismatched host CD30 KO T cells that were primed and boosted to recognize and kill graft cells (FIG. 11A, FIG. 11B and FIG. 9B). Graft and host cell numbers were determined by flow cytometry analysis and monitored daily over 3 days. While similar expansion rates were observed among different graft types growing in monoculture (FIG. 9C), a median of 82% and 90% of CD30.CAR ATC and SB9(NEG)-CD30.CAR ATCs were killed after 3 days in co-culture, respectively (FIG. 9D and FIG. 9F). Hence, SB9(NEG) did not have any protective function from allo-T cell killing, as we had expected. Similarly, SB9(ROS)-CD30.CAR ATC were also poorly protected from allogeneic rejection and a median of 76% of graft cells were killed in co-culture (FIG. 9D and FIG. 9F). On the other hand, SB9(WT)- and SB9(CAS)-CD30.CAR ATC resisted allogeneic rejection in all graft-host pairs studied (FIG. 9D and FIG. 9F). Notably, SB9(CAS)-CD30.CAR ATCs (median cell death=47%) had significantly lower cell death compared to CD30.CAR (median cell death=82%) and SB9(NEG)-CD30.CAR ATC (median cell death=90%) controls. Moreover, host T cells continued to survive and proliferate over the co-culture period and were not killed by SB9-CD30.CAR ATCs (FIG. 9E). At this point, we did not continue to pursue the study of SB9(ROS) as it did not provide allogeneic protection.

CD30.CAR EBVSTs have been tested in clinical trials (ClinicalTrials.gov identifier: NCT04288726) for the allogeneic treatment of Hodgkin's lymphoma and have demonstrated high efficacy and low graft-versus-host disease (GVHD). However, just like other allogeneic cell therapies, there was a lack of persistence of the cells in patients and allogeneic rejection might be a contributing factor. To study whether SB9 can be over-expressed in CD30.CAR EBVSTs and whether over-expression affects cell phenotype, we transduced CD30.CAR EBVSTs with SB9(WT), SB9(CAS) and SB9(CAS-ROS) (SEQ ID NO: 7), which is a triple mutant (E340D; C341S; C342S) that may incorporate the allogeneic protective effect of SB9(CAS) with protein stability of SB9(ROS). CD30.CAR EBVSTs transduced with different forms of SB9 (SB9-CD30.CAR EBVSTs) had 2 to 10-fold over-expression of SB9 (donor dependent) (FIG. 12B and FIG. 12C) and similar CD30.CAR expression (FIG. 12D and FIG. 12E). SB9-CD30.CAR EBVSTs also had similar proportions of EBV peptide responding cells, CD4/CD8 T cells, memory cells and activation/exhaustion status compared to CD30.CAR EBVSTs (FIG. 12F, FIG. 12G, FIG. 12H, FIG. 12I, FIG. 12J and FIG. 12K). In addition, we did not observe differences in cytokine production among SB9-enhanced CD30.CAR EBVSTs in monoculture and after stimulation with either engineered NALM6 or KM-H2 tumours (FIG. 18B, FIG. 18C and FIG. 18D).

To test whether SB9 over-expression protects CD30.CAR EBVSTs from allogeneic rejection, CD30.CAR EBVST grafts were co-cultured with CD30 KO allo-T cells, from 4 unique graft-host pairs, in a 1:1-4 ratio (FIG. 9G). Graft and host cell numbers were determined by flow cytometry analysis and monitored daily over 4 days. While CD30.CAR EBVSTs proliferated similarly in monoculture (FIG. 9H), a median of 86% and 84% of CD30.CAR EBVSTs and SB9(NEG)-CD30.CAR EBVSTs were killed in co-culture, respectively (FIG. 9I and FIG. 9K). In contrast, SB9(CAS)-, SB9(CAS-ROS)- and SB9(WT)-CD30.CAR EBVSTs resisted host cell killing (FIG. 9I and FIG. 9K). In particular, SB9(CAS)- and SB9(CAS-ROS)-CD30.CAR EBVSTs had a median cell death of 51% and 56%, respectively, and significantly resisted allogeneic rejection compared to CD30.CAR EBVSTs and SB9(NEG)-CD30.CAR EBVSTs (FIG. 9K). Furthermore, host T cells continued to survive and proliferate over the co-culture period and were not killed by SB9-CD30.CAR EBVSTs (FIG. 9J).

To determine whether SB9 protection was applicable beyond CD30.CAR T cells, we performed similar in vitro MLR assays for non-CAR expressing ATCs and EBVSTs. TCRαβ KO ATCs were co-cultured with HLA-mismatched host PBMCs in a 1:10-20 ratio for 12 days (FIG. 13A). While TCRαβ KO ATCs proliferated similarly in monoculture (FIG. 13B), ATCs, SB9(NEG)- and SB9(ROS)-ATCs did not survive beyond day 7 of co-culture (FIG. 13C). In contrast, SB9(WT)- and SB9(CAS)-ATCs continued to survive and reached peak proliferation between day 7-9 before they were eventually killed by the expanding host cells (FIG. 13C and FIG. 13D). On day 9 of co-culture, SB9(CAS)-ATCs had significantly resisted allogeneic rejection compared to CD30.CAR EBVSTs and SB9(NEG)-ATCs when tested in 5 independent HLA-mismatched graft-host pairs (FIG. 13E). A similar trend was observed when EBVSTs were co-cultured with allo-T cells, from 2 unique graft-host pairs, in a 1:1-4 ratio (FIG. 13F). SB9(CAS)-EBVSTs (mean cell death=43%) were able to resist allogeneic rejection-2-fold more than NT EBVSTs (mean cell death=90%) and SB9(NEG)-EBVSTs (mean cell death=89%) (FIG. 13H). Therefore, over-expression of SB9(CAS), may be a strategy to confer protection to graft T cells from allogeneic killing.

### Example 4: SerpinB9(CAS) Overexpression in CAR T Cells Enhances their Persistence and Antitumor Activity in In Vitro and In Vivo Allorejection Models

T cells have been shown to upregulate SB9 upon activation (14, 15). To evaluate whether activated T cells upregulate SB9 sufficiently to protect them from allogeneic rejection or whether SB9 over-expression is needed, we set up a tri-culture consisting of CD30.CAR EBVST graft cells and CD30 KO allo-T cells in the presence of engineered NALM6 tumor cells, which were added on day 0 (round 1) and day 2 (round 2) (FIG. 14A). To ensure that NALM6 cells were not killed by allo-T cells, we performed HLA class I and II KO by Crispr gene editing. In addition, to serve as targets for CD30.CAR EBVSTs, the NALM6 cells were engineered to over-express truncated CD30 (FIG. 15A). Then the tCD30 high, HLA I and II KO cells were further purified by magnetic bead isolation followed by either fluorescence-activated cell sorting (FACS sorting) or clonal selection to achieve >99% purity (FIG. 15A). A co-culture setup of allo-T cells and engineered NALM6 showed minimal killing of tumor by host T cells (FIG. 15B).

Activated CD30.CAR and SB9(NEG)-CD30.CAR EBVSTs were killed by allo-T cells at a median of 70% on day 3 of tri-culture (FIG. 14B). On the other hand, the over-expression of SB9(CAS) significantly protected graft cells from allogeneic rejection (median SB9(CAS)-CD30.CAR EBVSTs killed ˜45%) (FIG. 14B). Consequently, SB9(CAS)-CD30.CAR EBVSTs were able to eradicate engineered NALM6 cells from round 2 tumor challenge unlike CD30.CAR and SB9(NEG)-CD30.CAR EBVSTs which killed only a median of 60% tumor cells in tri-culture (FIG. 14C). Although SB9(WT) and SB9(CAS-ROS) also provided protection against allogeneic killing that led to increased engineered NALM6 control, statistical significance was not attained when compared to CD30.CAR and SB9(NEG)-CD30.CAR EBVSTs (FIG. 14B and FIG. 14C). Engineered NALM6 tumor added at round 1 were fully eliminated by CD30.CAR EBVSTs regardless of SB9 over-expression (data not shown). Moreover, similar findings were obtained when the study was repeated with CD19.CAR ATC and CD30.CAR ATC grafts (FIG. 15C, FIG. 15D, FIG. 15E, FIG. 15F). Therefore, the upregulation of endogenous SB9 by activated T cells were insufficient for graft cells to resist allogeneic rejection and the over-expression of SB9(CAS) was necessary to confer allogeneic protection.

To determine whether SB9 over-expression protects CD30.CAR EBVSTs from allogeneic rejection in vivo, we set up an allorejection mouse model, in which CAR T cells need to evade allogeneic rejection while protecting mice against cancer progression. The first model was set up by engrafting MHC KO NSG mice with engineered NALM6 cells (FIG. 14D). After 18 days, the CD30 KO allo-T cells (HLA-A2+) and CD30.CAR EBVST.eGFP-ffLuc graft cells (HLA-A2−) were co-infused intravenously (FIG. 14D). Tumor bearing mice treated with CD30.CAR EBVSTs with and without allo-T cells served as controls for allogeneic graft rejection (FIG. 16A). When allo-T cells were added, significantly lower bioluminescence signal was measured indicating that CD30.CAR EBVSTs succumbed to allogeneic rejection (FIG. 16B and FIG. 16C). Similarly, SB9(WT)-EBVSTs were rejected by allo-T cells (FIG. 14E and FIG. 14F). On the other hand, SB9(CAS)-CD30.CAR EBVSTs consistently persisted at significantly higher levels compared to CD30.CAR EBVSTs (FIG. 14E and FIG. 14F).

To evaluate tumor control, we set up the same allorejection mouse model but this round, using engineered NALM6.eGFP-ffluc cells that were engrafted for 15 days before graft CAR T and host cell treatment (FIG. 14G). Tumor bearing mice treated with CD30.CAR EBVSTs without allo-T cells served as a positive control for tumor clearance. Tumor burden was assessed by IVIS imaging and graft, and host cell numbers were established from flow cytometry analysis of blood samples that had been collected from facial vein. We observed a rebound of CD30 negative tumor after 15 days post treatment, hence experiments were terminated beyond that time point.

In the absence of allo-T cells, CD30.CAR EBVSTs persisted in all mice and controlled pre-established tumors (FIG. 16D, FIG. 16E, FIG. 16F and FIG. 16G). However, in the presence of allo-T cells, CD30.CAR EBVSTs were rejected and demonstrated poor tumor control (FIG. 16D, FIG. 16E, FIG. 16F, FIG. 16G, FIG. 14H, FIG. 14J and FIG. 14K). SB9(WT)-CD30.CAR EBVSTs were also unable to resist allogeneic rejection (FIG. 14H) but managed to demonstrate tumor regression by day 11 post treatment (FIG. 14J and FIG. 14K). In contrast, SB9(CAS)-CD30.CAR EBVSTs resisted allogeneic rejection significantly more than CD30.CAR EBVSTs and rapidly reduced tumor burden by day 7 post treatment (FIG. 14H, FIG. 14J and FIG. 14K). Notably, allo-T cell levels in the peripheral blood were unaltered after the infusion of SB9-CD30.CAR EBVSTs (FIG. 14I).

In all, the over-expression of SB9(CAS) enhanced the resistance of graft T cells against allogeneic rejection and improved anti-tumor efficacy in both in vitro and in vivo models. In addition, host T cell levels remained unaffected by the presence of SB9(CAS)-expressing graft T cells which alludes to the safety benefit of over-expressing SB9(CAS) as an allogeneic protection method.

### Example 5: SerpinB9(CAS)-Overexpressing CAR T Cells Show Survival Benefit During Serial In Vitro Tumor Cell Challenges and an In Vivo AICD Model

The survival and persistence of CAR T cells are also affected by chronic antigen exposure, which may result in T cell exhaustion and activation induced cell death of T cells (16, 17). We next set out to determine whether SB9 over-expression improves CAR T cell survival by reducing cell death under chronic antigen exposure conditions by performing serial co-culture assays of effector CAR ATCs (CD30.CAR and CD19.CAR ATCs) and engineered NALM6 tumor cells in vitro (FIG. 17A). Cells were counted every 2-3 days by flow cytometry and re-plated at fixed effector and tumor cell ratios in the absence of added cytokines. The experiment was terminated when any one effector cell type was unable to control tumor growth. CAR ATCs growing in monoculture served as controls.

In the absence of tumor cells, CD30.CAR ATCs with or without SB9 over-expression reduced in cell number over 8 days in culture and there was no observation of autonomous T cell growth (FIG. 17B and FIG. 17F). In the presence of tumor cells, effector cells proliferated and reached peak expansion on day 8-11 before reducing in cell numbers (FIG. 17C and FIG. 17G). Notably, the greatest expansion was observed for SB9(CAS)-CD30.CAR ATCs which expanded up to 20-fold more than CD30.CAR ATCs and SB9(NEG)-CD30.CAR ATCs on day 8-11 (FIG. 17C and FIG. 17G). Consequently, SB9(CAS)-CD30.CAR ATCs had improved engineered NALM6 killing compared to CD30.CAR ATCs and SB9(NEG)-CD30.CAR ATCs, which lost tumor control after 7 tumor challenges (FIG. 17D and FIG. 17H). The expression of PD-1, Lag-3 and Tim-3 were similar across effector cells throughout the course of the experiment, indicating similar exhaustion/activation status among effector cell types (FIG. 17E and FIG. 17I). Therefore, the enhanced tumor cytotoxicity of SB9(CAS)-CD30.CAR ATCs was likely due to the ability of effector cells to survive and expand by resisting cell death under chronic antigen exposure rather than exhaustion status of the cells.

To determine whether SB9(CAS) over-expression could also provide survival benefit in other CAR T cell systems, we repeated the serial co-culture experiments using CD19.CAR ATC effector cells.

Again, there was no observation of autonomous growth for CD19.CAR ATCs, regardless of SB9 over-expression, in monoculture (FIG. 17J and FIG. 17N). In the presence of tumor cells, the greatest expansion was also observed for SB9(CAS)-CD19.CAR ATCs which expanded up to 12-fold more than CD19.CAR ATCs and SB9(NEG)-CD30.CAR ATCs on day 6 (FIG. 17K and FIG. 17O). SB9(CAS)-CD19.CAR ATCs had improved tumor cell killing compared to CD19.CAR ATCs and SB9(NEG)-CD30.CAR ATCs which lost tumor control after 5 tumor challenges (FIG. 17L and FIG. 17P). As observed previously, the expression of PD-1, Lag-3 and Tim-3 were similar across effector cells throughout the course of the experiment (FIG. 17M and FIG. 17Q). In summary, SB9(CAS) over-expression enhances survival of different CAR T cells under chronic antigen exposure conditions and improves tumor cell killing. This result also highlights the potential of the use of SB9(CAS)-overexpressing T cells in the autologous cell therapies to achieve better expansion and persistence of graft cells in patients.

On the other hand, when CD30.CAR EBVSTs were used as effector cells in vitro, cell expansion and anti-tumor efficacy remained similar across effector cell types (FIG. 19C). A possible reason may be that CD30.CAR EBVSTs are less susceptible to activation-induced cell death (AICD) compared to CAR ATCs under the same chronic antigen exposure stress conditions.

To induce greater AICD in CD30.CAR EBVSTs, we intensified chronic antigen exposure stress by increasing CAR stimulation in an AICD B-cell acute lymphoblastic leukaemia (B-ALL) mouse model. The mice were engrafted with clonally selected engineered NALM6 cells to provide consistent CAR activation and to prevent early antigen escape. The current model also had a larger tumour burden, at time of CD30.CAR EBVST treatment, compared to the allorejection mouse model (FIG. 20). We found that SB9(CAS)-CD30.CAR EBVSTs significantly outperformed SB9(WT)- and CD30.CAR EBVSTs when we measured their ability to expand and kill tumour (FIG. 19G, FIG. 19H, FIG. 19I and FIG. 19J). Both SB9(WT)- and CD30.CAR EBVSTs failed to control tumours and some mice in those treatment groups had to be euthanized (FIG. 19G and FIG. 19H). In contrast, SB9(CAS)-CD30.CAR EBVSTs remarkably reduced tumours in all mice leading to their survival (FIG. 19G and FIG. 19H). Therefore, SB9(CAS) over-expression augments both survival and expansion of CD30.CAR EBVSTs to enhance their performance during chronic antigen exposure by tumour cells.

### Example 6: SerpinB9(CAS) but not SerpinB9(WT) Overexpression Protects CAR T Cells from FAS-Mediated Apoptosis

Allorejection and activation-induced cell death (AICD) are the results of apoptotic pathways involving GzmB and/or death receptors (FIG. 21A) (19). While SB9(WT) overexpression provided some alloprotective and expansion benefits, however, SB9(CAS) overexpression consistently resulted in superior alloprotection and expansion.

We next wanted to understand the superior protection afforded by SB9(CAS) overexpression by studying the resistance against Fas-mediated apoptosis in CD30.CAR EBVSTs. This was done by measuring cell viability and caspase 3/7 activity after treating the cells with an anti-CD95 (Fas) antibody. Indeed, SB9(CAS)-CD30.CAR EBVSTs demonstrated significantly improved cell viability compared to both SB9(WT)- and CD30.CAR EBVSTs and had similar number of viable cells as pan-caspase inhibitor, zVAD FMK, treated controls (FIG. 21B). We also demonstrated that the level of caspase 3/7 activity in CD95 treated SB9(CAS)-CD30.CAR EBVSTs was half that of SB9(WT)- and CD30.CAR EBVSTs at the 45 min timepoint (FIG. 21C), although the activity eventually increased over time to the same level as other conditions (data not shown).

To further probe the protective role of SB9(CAS) in Fas-mediated apoptosis, we included a His-tag at the C-terminus of SB9(CAS) to allow tracking of the transduced CD30.CAR EBVSTs by flow cytometry. Our data showed that upon treatment with anti-CD95, the SB9(CAS)-His-tag population was enriched whilst the SB9(CAS)-His-tag negative population diminished as a result of increased resistance against Fas mediated cytotoxicity (FIG. 21D). As a control, cells treated with zVAD FMK showed no change in the relative proportion of both populations of cells as compared to PBS treated cells (FIG. 21D). These results confirmed that SB9(CAS) but not SB9(WT) overexpression could protect CD30.CAR EBVSTs from Fas-mediated apoptosis and could therefore lead to more robust protection against allorejection and AICD.

In addition, SB9(CAS)-mediated protection against Fas-mediated apoptosis was also observed in SB9(CAS)-overexpressing NK cells (FIG. 22C and FIG. 22D). This may have contributed to larger expansion of SB9(CAS)-NK cells during the manufacturing process, which consisted of two activation steps (FIG. 22A and FIG. 22B). As CD95 is a member of a large family of death receptors that share a caspase mediated apoptotic pathway, it is likely that SB9(CAS) can similarly protect T and NK cells against apoptosis triggered by these death receptors.

In summary, the over-expression of SB9, or its molecular derivatives, in T cells is a promising solution to address the long-standing problem of allogeneic rejection of potentially curative T cell therapies, as well as to improve the survival of T cells under chronic antigen exposure. This is the first study describing allogeneic protection using SB9 over-expression in T cells. The over-expression of SB9 in mesenchymal stems cells (MSCs), for allogeneic protection, has been described previously (18), however, no studies of its application have been further reported. Most of the current strategies to convey allogeneic protection of T cells involve the elimination of activated host immune cells (1, 2, 4), which may result in elimination of pathogen-specific immune cells and lead to opportunistic infections. The strategy disclosed confers allogeneic protection of the therapy without harming host immunity. In addition, it has potential for wide application across different T cell therapies that also include the expression of engineered TCR or CAR.

### REFERENCES

A number of publications are cited herein in order to more fully describe and disclose the subject-matter of the present disclosure, and the state of the art to which the present disclosure pertains. Full citations for these references are provided below. The entirety of each of these references is incorporated herein.
1. Mo F, Watanabe N, McKenna M K, Hicks M J, Srinivasan M, Gomes-Silva D, et al. Engineered off-the-shelf therapeutic T cells resist host immune rejection. Nature Biotechnology. 2021; 39(1):56-63.
2. Quach D H, Becerra-Dominguez L, Rouce R H, Rooney C M. A strategy to protect off-the-shelf cell therapy products using virus-specific T-cells engineered to eliminate alloreactive T-cells. Journal of Translational Medicine. 2019; 17(1):240.
3. Lee J, Sheen J H, Lim O, Lee Y, Ryu J, Shin D, et al. Abrogation of HLA surface expression using CRISPR/Cas9 genome editing: a step toward universal T cell therapy. Scientific Reports. 2020; 10(1):17753.
4. Depil S, Duchateau P, Grupp S A, Mufti G, Poirot L. ‘Off-the-shelf allogeneic CAR T cells: development and challenges. Nat Rev Drug Discov. 2020; 19(3):185-99.
5. Locke F L, Malik S, Tees M T, Neelapu S S, Popplewell L, Abramson J S, et al. First-in-human data of ALLO-501A, an allogeneic chimeric antigen receptor (CAR) T-cell therapy and ALLO-647 in relapsed/refractory large B-cell lymphoma (R/R LBCL): ALPHA2 study. Journal of Clinical Oncology. 2021; 39(15_suppl):2529-.
6. Orive G, Echave M C, Pedraz J L, Golafshan N, Dolatshahi-Pirouz A, Paolone G, et al. Advances in cell-laden hydrogels for delivering therapeutics. Expert Opinion on Biological Therapy. 2019; 19(12):1219-22.
7. Heusel J W, Wesselschmidt R L, Shresta S, Russell J H, Ley T J. Cytotoxic lymphocytes require granzyme B for the rapid induction of DNA fragmentation and apoptosis in allogeneic target cells. Cell. 1994; 76(6):977-87.
8. Du W, Cao X. Cytotoxic Pathways in Allogeneic Hematopoietic Cell Transplantation. Front Immunol. 2018; 9:2979.
9. Krams S M, Villanueva J C, Quinn M B, Martinez O M. Expression of the cytotoxic T cell mediator granzyme B during liver allograft rejection. Transpl Immunol. 1995; 3(2):162-6.
10. Waterhouse N J, Sedelies K A, Browne K A, Wowk M E, Newbold A, Sutton V R, et al. A central role for Bid in granzyme B-induced apoptosis. J Biol Chem. 2005; 280(6):4476-82.
11. Thomas D A, Scorrano L, Putcha G V, Korsmeyer S J, Ley T J. Granzyme B can cause mitochondrial depolarization and cell death in the absence of BID, BAX, and BAK. Proc Natl Acad Sci USA. 2001; 98(26):14985-90.
12. Kaiserman D, Bird P I. Control of granzymes by serpins. Cell Death Differ. 2010; 17(4):586-95.
13. Bird C H, Sutton V R, Sun J, Hirst C E, Novak A, Kumar S, et al. Selective regulation of apoptosis: the cytotoxic lymphocyte serpin proteinase inhibitor 9 protects against granzyme B-mediated apoptosis without perturbing the Fas cell death pathway. Mol Cell Biol. 1998; 18(11):6387-98.
14. Mangan M S, Bird C H, Kaiserman D, Matthews A Y, Hitchen C, Steer D L, et al. A Novel Serpin Regulatory Mechanism: SerpinB9 IS REVERSIBLY INHIBITED B Y VICINAL DISULFIDE BOND FORMATION IN THE REACTIVE CENTER LOOP. J Biol Chem. 2016; 291(7):3626-38.
15. Hirst C E, Buzza M S, Bird C H, Warren H S, Cameron P U, Zhang M, et al. The intracellular granzyme B inhibitor, proteinase inhibitor 9, is up-regulated during accessory cell maturation and effector cell degranulation, and its overexpression enhances CTL potency. J Immunol. 2003; 170(2):805-15.
16. Zhan Y, Carrington E M, Zhang Y, Heinzel S, Lew A M. Life and Death of Activated T Cells: How Are They Different from Naïve T Cells? Frontiers in Immunology. 2017; 8.
17. Good C R, Aznar M A, Kuramitsu S, Samareh P, Agarwal S, Donahue G, et al. An NK-like CAR T cell transition in CAR T cell dysfunction. Cell. 2021:184(25):6081-100.e26.
18. El Haddad N, Moore R, Heathcote D, Mounayar M, Azzi J, Mfarrej B, et al. The novel role of SERPINB9 in cytotoxic protection of human mesenchymal stem cells. J Immunol. 2011; 187(5):2252-60.
19. Parihar R, Rivas C, Huynh M, Omer B, Lapteva N, Metelitsa L S, et al. N K Cells Expressing a Chimeric Activating Receptor Eliminate MDSCs and Rescue Impaired CAR-T Cell Activity against Solid Tumors. Cancer Immunol Res. 2019; 7(3):363-75.
20. Lapteva N, Parihar R, Rollins L A, Gee A P, Rooney C M. Large-Scale Culture and Genetic Modification of Human Natural Killer Cells for Cellular Therapy. Methods Mol Biol. 2016; 1441:195-202.

For standard molecular biology techniques, see Sambrook, J., Russel, D. W. Molecular Cloning, A Laboratory Manual. 3 ed. 2001, Cold Spring Harbor, New York: Cold Spring Harbor Laboratory Press.

1. An immune cell comprising modification to increase the expression or activity of SERPINB9.

2. The immune cell according to claim 1, wherein the immune cell comprises exogenous nucleic acid encoding a SERPINB9 polypeptide.

3. The immune cell according to claim 2, wherein the exogenous nucleic acid encoding a SERPINB9 polypeptide is, or is comprised in, an expression vector; optionally wherein the expression vector is a retroviral expression vector.

4. The immune cell according to claim 2 or claim 3, wherein the SERPINB9 polypeptide comprises, or consists of, the amino acid sequence of SEQ ID NO: 1, 4, 5, 6 or 7, or a variant thereof having at least 85% amino acid sequence identity to SEQ ID NO: 1, 4, 5, 6 or 7.

5. The immune cell according to any one of claims 1 to 4, wherein the immune cell is an effector immune cell; optionally wherein the effector immune cell is a T cell or a natural killer (NK) cell.

6. The immune cell according to any one of claims 1 to 5, wherein the immune cell comprises nucleic acid encoding a chimeric antigen receptor (CAR).

7. The immune cell according to claim 6, wherein the CAR comprises an antigen-binding domain that binds to a cancer-associated antigen selected from: CD30, CD19, CD20, CD22, B7H3, c-Met, ROR1R, CD4, CD7, CD38, BCMA, Mesothelin, EGFR, GPC3, MUC1, HER2, GD2, CEA, EpCAM, LeY and PSCA; optionally wherein the CAR comprises an antigen-binding domain that binds to CD30.

8. The immune cell according to any one of claims 1 to 7, wherein the immune cell is a virus-specific T cell or an activated T cell (ATC).

9. The immune cell according to claim 8, wherein the virus-specific T cell is specific for a virus selected from Epstein-Barr virus (EBV), adenovirus, cytomegalovius (CMV), human papilloma virus (HPV), influenza virus, measles virus, hepatitis B virus (HBV), hepatitis C virus (HCV), human immunodeficiency virus (HIV), lymphocytic choriomeningitis virus (LCMV), herpes simplex virus (HSV), BK virus (BKV) or varicella zoster virus (VZV); optionally wherein the virus is EBV.

10. A pharmaceutical composition, comprising an immune cell according to any one of claims 1 to 9, and a pharmaceutically-acceptable carrier, diluent, excipient or adjuvant.

11. An immune cell according to any one of claims 1 to 9, or a pharmaceutical composition according to claim 10, for use in a method of medical treatment or prophylaxis.

12. Use of an immune cell according to any one of claims 1 to 9, or of a pharmaceutical composition according to claim 10, in the manufacture of a medicament for use in a method of medical treatment or prophylaxis.

13. A method of treating or preventing a disease or condition in a subject, comprising administering to a subject a therapeutically- or prophylactically-effective quantity of an immune cell according to any one of claims 1 to 9, or of a pharmaceutical composition according to claim 10.

14. A method for reducing the activity of a serine protease or a caspase in a cell, comprising modifying the cell to increase the expression or activity of SERPINB9.

15. A method for increasing the resistance of a cell to the activity of a serine protease or a caspase, comprising modifying the cell to increase the expression or activity of SERPINB9.

16. A method for increasing the resistance of a cell to cell killing by granzyme B, comprising modifying the cell to increase the expression or activity of SERPINB9.

17. A method for increasing the resistance of a cell to apoptosis mediated by a death receptor, comprising modifying the cell to increase the expression or activity of SERPINB9.

18. The method according to any one of claims 14 to 17, wherein modifying the cell to increase the expression or activity of SERPINB9 comprises introducing nucleic acid encoding a SERPINB9 polypeptide into the cell.

19. The method according to claim 18, wherein the nucleic acid encoding a SERPINB9 polypeptide is, or is comprised in, an expression vector; optionally wherein the expression vector is a retroviral expression vector.

20. The method according to claim 18 or claim 19, wherein the SERPINB9 polypeptide comprises, or consists of, the amino acid sequence of SEQ ID NO: 1, 4, 5, 6 or 7, or a variant thereof having at least 85% amino acid sequence identity to SEQ ID NO: 1, 4, 5, 6 or 7.

21. The method according to any one of claims 14 to 20, wherein the cell is an effector immune cell; optionally wherein the effector immune cell is a T cell or a natural killer (NK) cell.

22. The method according to any one of claims 14 to 21, the cell comprises nucleic acid encoding a chimeric antigen receptor (CAR).

23. The method according to claim 22, wherein the CAR comprises an antigen-binding domain that binds to a cancer-associated antigen selected from: CD30, CD19, CD20, CD22, B7H3, c-Met, ROR1R, CD4, CD7, CD38, BCMA, Mesothelin, EGFR, GPC3, MUC1, HER2, GD2, CEA, EpCAM, LeY and PSCA; optionally wherein the CAR comprises an antigen-binding domain that binds to CD30.

24. The method according to any one of claims 14 to 23, wherein the cell is a virus-specific T cell.

25. The method according to claim 24, wherein the virus-specific T cell is specific for a virus selected from Epstein-Barr virus (EBV), adenovirus, cytomegalovius (CMV), human papilloma virus (HPV), influenza virus, measles virus, hepatitis B virus (HBV), hepatitis C virus (HCV), human immunodeficiency virus (HIV), lymphocytic choriomeningitis virus (LCMV), herpes simplex virus (HSV), BK virus (BKV) or varicella zoster virus (VZV); optionally wherein the virus is EBV.

26. An immune cell for use in treating or preventing a cancer in a subject, wherein:
the immune cell comprises nucleic acid encoding a CAR comprising: (i) an antigen-binding domain that binds to CD30 or CD19, (ii) a transmembrane domain, and (iii) a signalling domain comprising an immunoreceptor tyrosine-based activation motif (ITAM); and
the immune cell comprises modification to increase the expression or activity of SERPINB9.

27. Use of an immune cell in the manufacture of a medicament for use in treating or preventing a cancer in a subject, wherein:
the immune cell comprises nucleic acid encoding a CAR comprising: (i) an antigen-binding domain that binds to CD30 or CD19, (ii) a transmembrane domain, and (iii) a signalling domain comprising an immunoreceptor tyrosine-based activation motif (ITAM); and
the immune cell comprises modification to increase the expression or activity of SERPINB9.

28. A method of treating or preventing a cancer in a subject, comprising administering to a subject a therapeutically- or prophylactically-effective quantity of an immune cell, wherein:
the immune cell comprises nucleic acid encoding a CAR comprising: (i) an antigen-binding domain that binds to CD30 or CD19, (ii) a transmembrane domain, and (iii) a signalling domain comprising an immunoreceptor tyrosine-based activation motif (ITAM); and
the immune cell comprises modification to increase the expression or activity of SERPINB9.

29. The immune cell for use according to claim 26, the use according to claim 27, or the method according to claim 28, wherein the immune cell is a virus-specific T cell; optionally wherein the immune cell is an Epstein-Barr virus (EBV)-specific T cell.

30. An immune cell for use in treating or preventing a cancer in a subject, wherein:
the immune cell is a virus-specific T cell; optionally wherein the immune cell is an Epstein-Barr virus (EBV)-specific T cell; and
the immune cell comprises modification to increase the expression or activity of SERPINB9.

31. Use of an immune cell in the manufacture of a medicament for use in treating or preventing a cancer in a subject, wherein:
the immune cell is a virus-specific T cell; optionally wherein the immune cell is an Epstein-Barr virus (EBV)-specific T cell; and
the immune cell comprises modification to increase the expression or activity of SERPINB9.

32. A method of treating or preventing a cancer in a subject, comprising administering to a subject a therapeutically- or prophylactically-effective quantity of an immune cell, wherein:
the immune cell is a virus-specific T cell; optionally wherein the immune cell is an Epstein-Barr virus (EBV)-specific T cell; and
the immune cell comprises modification to increase the expression or activity of SERPINB9.

33. The immune cell for use according to claim 30, the use according to claim 31, or the method according to claim 32, wherein the immune cell comprises nucleic acid encoding a CAR comprising: (i) an antigen-binding domain that binds to CD30 or CD19, (ii) a transmembrane domain, and (iii) a signalling domain comprising an immunoreceptor tyrosine-based activation motif (ITAM).

34. The immune cell for use, the use, or the method according to any one of claims 26 to 33, wherein the subject is allogeneic with respect to the immune cell.

35. The immune cell for use, the use or the method according to any one of claims 26 to 34, wherein the immune cell comprises exogenous nucleic acid encoding a SERPINB9 polypeptide.

36. The immune cell for use, the use, or the method according to claim 35, wherein the exogenous nucleic acid encoding a SERPINB9 polypeptide is, or is comprised in, an expression vector; optionally wherein the expression vector is a retroviral expression vector.

37. The immune cell for use, the use, or the method according to claim 35 or claim 36, wherein the SERPINB9 polypeptide comprises, or consists of, the amino acid sequence of SEQ ID NO: 1, 4, 5, 6 or 7, or a variant thereof having at least 85% amino acid sequence identity to SEQ ID NO: 1, 4, 5, 6 or 7.


SEQUENCE LISTING (82 sequences)

SEQ ID NO: 1 - protein, 376 aa, Homo sapiens
METLSNASGTFAIRLLKILCQDNPSHNVFCSPVSISSALAMVLLGAKGNTATQMAQALSLNTEEDIHRAFQSLLTEVNKAGTQYLLRTANRLFGEKTCQFLSTFKESCLQFYHAELKELSFIRAAEESRKHINTWVSKKTEGKIEELLPGSSIDAETRLVLVNAIYFKGKWNEPFDETYTREMPFKINQEEQRPVQMMYQEATFKLAHVGEVRAQLLELPYARKELSLLVLLPDDGVELSTVEKSLTFEKLTAWTKPDCMKSTEVEVLLPKFKLQEDYDMESVLRHLGIVDAFQQGKADLSAMSAERDLCLSKFVHKSFVEVNEEGTEAAAASSCFVVAECCMESGPRFCADHPFLFFIRHNRANSILFCGRFSSP

SEQ ID NO: 2 - protein, 15 aa, Homo sapiens
SCFVVAECCMESGPR

SEQ ID NO: 3 - protein, 15 aa, Homo sapiens
  VARIANT 7: note = E or D
  VARIANT 8..9: note = C or S
SCFVVAXXXMESGPR

SEQ ID NO: 4 - protein, 376 aa, synthetic construct
  VARIANT 340: note = E or D
  VARIANT 341..342: note = C or S
METLSNASGTFAIRLLKILCQDNPSHNVFCSPVSISSALAMVLLGAKGNTATQMAQALSLNTEEDIHRAFQSLLTEVNKAGTQYLLRTANRLFGEKTCQFLSTFKESCLQFYHAELKELSFIRAAEESRKHINTWVSKKTEGKIEELLPGSSIDAETRLVLVNAIYFKGKWNEPFDETYTREMPFKINQEEQRPVQMMYQEATFKLAHVGEVRAQLLELPYARKELSLLVLLPDDGVELSTVEKSLTFEKLTAWTKPDCMKSTEVEVLLPKFKLQEDYDMESVLRHLGIVDAFQQGKADLSAMSAERDLCLSKFVHKSFVEVNEEGTEAAAASSCFVVAXXXMESGPRFCADHPFLFFIRHNRANSILFCGRFSSP

SEQ ID NO: 5 - protein, 376 aa, synthetic construct
METLSNASGTFAIRLLKILCQDNPSHNVFCSPVSISSALAMVLLGAKGNTATQMAQALSLNTEEDIHRAFQSLLTEVNKAGTQYLLRTANRLFGEKTCQFLSTFKESCLQFYHAELKELSFIRAAEESRKHINTWVSKKTEGKIEELLPGSSIDAETRLVLVNAIYFKGKWNEPFDETYTREMPFKINQEEQRPVQMMYQEATFKLAHVGEVRAQLLELPYARKELSLLVLLPDDGVELSTVEKSLTFEKLTAWTKPDCMKSTEVEVLLPKFKLQEDYDMESVLRHLGIVDAFQQGKADLSAMSAERDLCLSKFVHKSFVEVNEEGTEAAAASSCFVVADCCMESGPRFCADHPFLFFIRHNRANSILFCGRFSSP

SEQ ID NO: 6 - protein, 376 aa, synthetic construct
METLSNASGTFAIRLLKILCQDNPSHNVFCSPVSISSALAMVLLGAKGNTATQMAQALSLNTEEDIHRAFQSLLTEVNKAGTQYLLRTANRLFGEKTCQFLSTFKESCLQFYHAELKELSFIRAAEESRKHINTWVSKKTEGKIEELLPGSSIDAETRLVLVNAIYFKGKWNEPFDETYTREMPFKINQEEQRPVQMMYQEATFKLAHVGEVRAQLLELPYARKELSLLVLLPDDGVELSTVEKSLTFEKLTAWTKPDCMKSTEVEVLLPKFKLQEDYDMESVLRHLGIVDAFQQGKADLSAMSAERDLCLSKFVHKSFVEVNEEGTEAAAASSCFVVAESSMESGPRFCADHPFLFFIRHNRANSILFCGRFSSP

SEQ ID NO: 7 - protein, 376 aa, synthetic construct
METLSNASGTFAIRLLKILCQDNPSHNVFCSPVSISSALAMVLLGAKGNTATQMAQALSLNTEEDIHRAFQSLLTEVNKAGTQYLLRTANRLFGEKTCQFLSTFKESCLQFYHAELKELSFIRAAEESRKHINTWVSKKTEGKIEELLPGSSIDAETRLVLVNAIYFKGKWNEPFDETYTREMPFKINQEEQRPVQMMYQEATFKLAHVGEVRAQLLELPYARKELSLLVLLPDDGVELSTVEKSLTFEKLTAWTKPDCMKSTEVEVLLPKFKLQEDYDMESVLRHLGIVDAFQQGKADLSAMSAERDLCLSKFVHKSFVEVNEEGTEAAAASSCFVVADSSMESGPRFCADHPFLFFIRHNRANSILFCGRFSSP

SEQ ID NO: 8 - protein, 595 aa, Homo sapiens
MRVLLAALGLLFLGALRAFPQDRPFEDTCHGNPSHYYDKAVRRCCYRCPMGLFPTQQCPQRPTDCRKQCEPDYYLDEADRCTACVTCSRDDLVEKTPCAWNSSRVCECRPGMFCSTSAVNSCARCFFHSVCPAGMIVKFPGTAQKNTVCEPASPGVSPACASPENCKEPSSGTIPQAKPTPVSPATSSASTMPVRGGTRLAQEAASKLTRAPDSPSSVGRPSSDPGLSPTQPCPEGSGDCRKQCEPDYYLDEAGRCTACVSCSRDDLVEKTPCAWNSSRTCECRPGMICATSATNSCARCVPYPICAAETVTKPQDMAEKDTTFEAPPLGTQPDCNPTPENGEAPASTSPTQSLLVDSQASKTLPIPTSAPVALSSTGKPVLDAGPVLFWVILVLVVVVGSSAFLLCHRRACRKRIRQKLHLCYPVQTSQPKLELVDSRPRRSSTQLRSGASVTEPVAEERGLMSQPLMETCHSVGAAYLESLPLQDASPAGGPSSPRDLPEPRVSTEHTNNKIEKIYIMKADTVIVGTVKAELPEGRGLAGPAEPELEEELEADHTPHYPEQETEPPLGSCSDVMLSVEEEGKEDPLPTAASGK

SEQ ID NO: 9 - protein, 132 aa, Homo sapiens
MSQPLMETCHSVGAAYLESLPLQDASPAGGPSSPRDLPEPRVSTEHTNNKIEKIYIMKADTVIVGTVKAELPEGRGLAGPAEPELEEELEADHTPHYPEQETEPPLGSCSDVMLSVEEEGKEDPLPTAASGK

SEQ ID NO: 10 - protein, 483 aa, Homo sapiens
MFCSTSAVNSCARCFFHSVCPAGMIVKFPGTAQKNTVCEPASPGVSPACASPENCKEPSSGTIPQAKPTPVSPATSSASTMPVRGGTRLAQEAASKLTRAPDSPSSVGRPSSDPGLSPTQPCPEGSGDCRKQCEPDYYLDEAGRCTACVSCSRDDLVEKTPCAWNSSRTCECRPGMICATSATNSCARCVPYPICAAETVTKPQDMAEKDTTFEAPPLGTQPDCNPTPENGEAPASTSPTQSLLVDSQASKTLPIPTSAPVALSSTGKPVLDAGPVLFWVILVLVVVVGSSAFLLCHRRACRKRIRQKLHLCYPVQTSQPKLELVDSRPRRSSTLRSGASVTEPVAEERGLMSQPLMETCHSVGAAYLESLPLQDASPAGGPSSPRDLPEPRVSTEHTNNKIEKIYIMKADTVIVGTVKAELPEGRGLAGPAEPELEEELEADHTPHYPEQETEPPLGSCSDVMLSVEEEGKEDPLPTAASGK

SEQ ID NO: 11 - protein, 18 aa, Homo sapiens
MRVLLAALGLLFLGALRA

SEQ ID NO: 12 - protein, 367 aa, Homo sapiens
FPQDRPFEDTCHGNPSHYYDKAVRRCCYRCPMGLFPTQQCPQRPTDCRKQCEPDYYLDEADRCTACVTCSRDDLVEKTPCAWNSSRVCECRPGMFCSTSAVNSCARCFFHSVCPAGMIVKFPGTAQKNTVCEPASPGVSPACASPENCKEPSSGTIPQAKPTPVSPATSSASTMPVRGGTRLAQEAASKLTRAPDSPSSVGRPSSDPGLSPTQPCPEGSGDCRKQCEPDYYLDEAGRCTACVSCSRDDLVEKTPCAWNSSRTCECRPGMICATSATNSCARCVPYPICAAETVTKPQDMAEKDTTFEAPPLGTQPDCNPTPENGEAPASTSPTQSLLVDSQASKTLPIPTSAPVALSSTGKPVLDAG

SEQ ID NO: 13 - protein, 21 aa, Homo sapiens
PVLFWVILVLVVVVGSSAFLL

SEQ ID NO: 14 - protein, 189 aa, Homo sapiens
CHRRACRKRIRQKLHLCYPVQTSQPKLELVDSRPRRSSTQLRSGASVTEPVAEERGLMSQPLMETCHSVGAAYLESLPLQDASPAGGPSSPRDLPEPRVSTEHTNNKIEKIYIMKADTVIVGTVKAELPEGRGLAGPAEPELEEELEADHTPHYPEQETEPPLGSCSDVMLSVEEEGKEDPLPTAASGK

SEQ ID NO: 15 - protein, 8 aa, synthetic construct
GYTFTTYT

SEQ ID NO: 16 - protein, 8 aa, synthetic construct
INPSSGCS

SEQ ID NO: 17 - protein, 16 aa, synthetic construct
ARRADYGNYEYTWFAY

SEQ ID NO: 18 - protein, 6 aa, synthetic construct
QNVGTN

SEQ ID NO: 19 - skipped

SEQ ID NO: 20 - protein, 9 aa, synthetic construct
QQYHTYPLT

SEQ ID NO: 21 - protein, 123 aa, synthetic construct
QVQLQQSGAELARPGASVKMSCKASGYTFTTYTIHWVRRRPGHDLEWIGYINPSSGCSDYNQNFKGKTTLTADKSSNTAYMQLNSLTSEDSAVYYCARRADYGNYEYTWFAYWGQGTTVTVSS

SEQ ID NO: 22 - protein, 107 aa, synthetic construct
VIELTQSPKFMSTSVGDRVNVTYKASQNVGTNVAWFQQKPGQSPKVLIYSASYRYSGVPDRFTGSGSGTDFTLTISNVQSEDLAEYFCQQYHTYPLTFGGGTKLEIK

SEQ ID NO: 23 - protein, 15 aa, synthetic construct
SGGGSGGGGSGGGGS

SEQ ID NO: 24 - protein, 245 aa, synthetic construct
QVQLQQSGAELARPGASVKMSCKASGYTFTTYTIHWVRRRPGHDLEWIGYINPSSGCSDYNQNFKGKTTLTADKSSNTAYMQLNSLTSEDSAVYYCARRADYGNYEYTWFAYWGQGTTVTVSSSGGGSGGGGSGGGGSVIELTQSPKFMSTSVGDRVNVTYKASQNVGTNVAWFQQKPGQSPKVLIYSASYRYSGVPDRFTGSGSGTDFTLTISNVQSEDLAEYFCQQYHTYPLTFGGGTKLEIK

SEQ ID NO: 25 - protein, 151 aa, synthetic construct
ATSSASTMPVRGGTRLAQEAASKLTRAPDSPSSVGRPSSDPGLSPTQPCPEGSGDCRKQCEPDYYLDEAGRCTACVSCSRDDLVEKTPCAWNSSRTCECRPGMICATSATNSCARCVPYPICAAETVTKPQDMAEKDTTFEAPPLGTQPDC

SEQ ID NO: 26 - protein, 27 aa, Homo sapiens
FWVLVVVGGVLACYSLLVTVAFIIFWV

SEQ ID NO: 27 - protein, 21 aa, Homo sapiens
LCYLLDGILFIYGVILTALFL

SEQ ID NO: 28 - protein, 28 aa, Homo sapiens
IYIWAPLAGTCGVLLLSLVITLYCNHRN

SEQ ID NO: 29-30 - skipped (2 entries)

SEQ ID NO: 31 - protein, 112 aa, Homo sapiens
RVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 32 - protein, 41 aa, Homo sapiens
RSKRSRLLHSDYMNMTPRRPGPTRKHYQPYAPPRDFAAYRS

SEQ ID NO: 33 - protein, 41 aa, Homo sapiens
RSKRSRLLHSDYMNMTPRRPGPTRKHYQAYAAARDFAAYRS

SEQ ID NO: 34 - protein, 153 aa, synthetic construct
RSKRSRLLHSDYMNMTPRRPGPTRKHYQPYAPPRDFAAYRSRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 35 - protein, 12 aa, Homo sapiens
EPKSCDKTHTCP

SEQ ID NO: 36 - protein, 12 aa, Homo sapiens
EPKSPDKTHTCP

SEQ ID NO: 37 - protein, 220 aa, Homo sapiens
PCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 38 - protein, 223 aa, Homo sapiens
PCPAPPVAGPSVFLFPPKPKDTLMIARTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKKDPK

SEQ ID NO: 39 - protein, 232 aa, synthetic construct
EPKSPDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 40 - protein, 23 aa, synthetic construct
MDFQVQIFSFLLISASVIMSRMA

SEQ ID NO: 41 - protein, 666 aa, synthetic construct
QVQLQQSGAELARPGASVKMSCKASGYTFTTYTIHWVRRRPGHDLEWIGYINPSSGCSDYNQNFKGKTTLTADKSSNTAYMQLNSLTSEDSAVYYCARRADYGNYEYTWFAYWGQGTTVTVSSSGGGSGGGGSGGGGSVIELTQSPKFMSTSVGDRVNVTYKASQNVGTNVAWFQQKPGQSPKVLIYSASYRYSGVPDRFTGSGSGTDFTLTISNVQSEDLAEYFCQQYHTYPLTFGGGTKLEIKRSDPAEPKSPDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKKDPKFWVLVVVGGVLACYSLLVTVAFIIFWVRSKRSRLLHSDYMNMTPRRPGPTRKHYQPYAPPRDFAAYRSRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 42 - protein, 689 aa, synthetic construct
MDFQVQIFSFLLISASVIMSRMAQVQLQQSGAELARPGASVKMSCKASGYTFTTYTIHWVRRRPGHDLEWIGYINPSSGCSDYNQNFKGKTTLTADKSSNTAYMQLNSLTSEDSAVYYCARRADYGNYEYTWFAYWGQGTTVTVSSSGGGSGGGGSGGGGSVIELTQSPKFMSTSVGDRVNVTYKASQNVGTNVAWFQQKPGQSPKVLIYSASYRYSGVPDRFTGSGSGTDFTLTISNVQSEDLAEYFCQQYHTYPLTFGGGTKLEIKRSDPAEPKSPDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKKDPKFWVLVVVGGVLACYSLLVTVAFIIFWVRSKRSRLLHSDYMNMTPRRPGPTRKHYQPYAPPRDFAAYRSRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 43 - protein, 1103 aa, synthetic construct
METLSNASGTFAIRLLKILCQDNPSHNVFCSPVSISSALAMVLLGAKGNTATQMAQALSLNTEEDIHRAFQSLLTEVNKAGTQYLLRTANRLFGEKTCQFLSTFKESCLQFYHAELKELSFIRAAEESRKHINTWVSKKTEGKIEELLPGSSIDAETRLVLVNAIYFKGKWNEPFDETYTREMPFKINQEEQRPVQMMYQEATFKLAHVGEVRAQLLELPYARKELSLLVLLPDDGVELSTVEKSLTFEKLTAWTKPDCMKSTEVEVLLPKFKLQEDYDMESVLRHLGIVDAFQQGKADLSAMSAERDLCLSKFVHKSFVEVNEEGTEAAAASSCFVVAECCMESGPRFCADHPFLFFIRHNRANSILFCGRFSSPRKRRGSGTPDPWGSGATNFSLLKQAGDVEENPGPLELEMDFQVQIFSFLLISASVIMSRMAQVQLQQSGAELARPGASVKMSCKASGYTFTTYTIHWVRRRPGHDLEWIGYINPSSGCSDYNQNFKGKTTLTADKSSNTAYMQLNSLTSEDSAVYYCARRADYGNYEYTWFAYWGQGTTVTVSSSGGGSGGGGSGGGGSVIELTQSPKFMSTSVGDRVNVTYKASQNVGTNVAWFQQKPGQSPKVLIYSASYRYSGVPDRFTGSGSGTDFTLTISNVQSEDLAEYFCQQYHTYPLTFGGGTKLEIKRSDPAEPKSPDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKKDPKFWVLVVVGGVLACYSLLVTVAFIIFWVRSKRSRLLHSDYMNMTPRRPGPTRKHYQPYAPPRDFAAYRSRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 44 - protein, 1103 aa, synthetic construct
METLSNASGTFAIRLLKILCQDNPSHNVFCSPVSISSALAMVLLGAKGNTATQMAQALSLNTEEDIHRAFQSLLTEVNKAGTQYLLRTANRLFGEKTCQFLSTFKESCLQFYHAELKELSFIRAAEESRKHINTWVSKKTEGKIEELLPGSSIDAETRLVLVNAIYFKGKWNEPFDETYTREMPFKINQEEQRPVQMMYQEATFKLAHVGEVRAQLLELPYARKELSLLVLLPDDGVELSTVEKSLTFEKLTAWTKPDCMKSTEVEVLLPKFKLQEDYDMESVLRHLGIVDAFQQGKADLSAMSAERDLCLSKFVHKSFVEVNEEGTEAAAASSCFVVADCCMESGPRFCADHPFLFFIRHNRANSILFCGRFSSPRKRRGSGTPDPWGSGATNFSLLKQAGDVEENPGPLELEMDFQVQIFSFLLISASVIMSRMAQVQLQQSGAELARPGASVKMSCKASGYTFTTYTIHWVRRRPGHDLEWIGYINPSSGCSDYNQNFKGKTTLTADKSSNTAYMQLNSLTSEDSAVYYCARRADYGNYEYTWFAYWGQGTTVTVSSSGGGSGGGGSGGGGSVIELTQSPKFMSTSVGDRVNVTYKASQNVGTNVAWFQQKPGQSPKVLIYSASYRYSGVPDRFTGSGSGTDFTLTISNVQSEDLAEYFCQQYHTYPLTFGGGTKLEIKRSDPAEPKSPDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKKDPKFWVLVVVGGVLACYSLLVTVAFIIFWVRSKRSRLLHSDYMNMTPRRPGPTRKHYQPYAPPRDFAAYRSRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 45 - protein, 1103 aa, synthetic construct
METLSNASGTFAIRLLKILCQDNPSHNVFCSPVSISSALAMVLLGAKGNTATQMAQALSLNTEEDIHRAFQSLLTEVNKAGTQYLLRTANRLFGEKTCQFLSTFKESCLQFYHAELKELSFIRAAEESRKHINTWVSKKTEGKIEELLPGSSIDAETRLVLVNAIYFKGKWNEPFDETYTREMPFKINQEEQRPVQMMYQEATFKLAHVGEVRAQLLELPYARKELSLLVLLPDDGVELSTVEKSLTFEKLTAWTKPDCMKSTEVEVLLPKFKLQEDYDMESVLRHLGIVDAFQQGKADLSAMSAERDLCLSKFVHKSFVEVNEEGTEAAAASSCFVVAESSMESGPRFCADHPFLFFIRHNRANSILFCGRFSSPRKRRGSGTPDPWGSGATNFSLLKQAGDVEENPGPLELEMDFQVQIFSFLLISASVIMSRMAQVQLQQSGAELARPGASVKMSCKASGYTFTTYTIHWVRRRPGHDLEWIGYINPSSGCSDYNQNFKGKTTLTADKSSNTAYMQLNSLTSEDSAVYYCARRADYGNYEYTWFAYWGQGTTVTVSSSGGGSGGGGSGGGGSVIELTQSPKFMSTSVGDRVNVTYKASQNVGTNVAWFQQKPGQSPKVLIYSASYRYSGVPDRFTGSGSGTDFTLTISNVQSEDLAEYFCQQYHTYPLTFGGGTKLEIKRSDPAEPKSPDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKKDPKFWVLVVVGGVLACYSLLVTVAFIIFWVRSKRSRLLHSDYMNMTPRRPGPTRKHYQPYAPPRDFAAYRSRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 46 - protein, 1103 aa, synthetic construct
METLSNASGTFAIRLLKILCQDNPSHNVFCSPVSISSALAMVLLGAKGNTATQMAQALSLNTEEDIHRAFQSLLTEVNKAGTQYLLRTANRLFGEKTCQFLSTFKESCLQFYHAELKELSFIRAAEESRKHINTWVSKKTEGKIEELLPGSSIDAETRLVLVNAIYFKGKWNEPFDETYTREMPFKINQEEQRPVQMMYQEATFKLAHVGEVRAQLLELPYARKELSLLVLLPDDGVELSTVEKSLTFEKLTAWTKPDCMKSTEVEVLLPKFKLQEDYDMESVLRHLGIVDAFQQGKADLSAMSAERDLCLSKFVHKSFVEVNEEGTEAAAASSCFVVADSSMESGPRFCADHPFLFFIRHNRANSILFCGRFSSPRKRRGSGTPDPWGSGATNFSLLKQAGDVEENPGPLELEMDFQVQIFSFLLISASVIMSRMAQVQLQQSGAELARPGASVKMSCKASGYTFTTYTIHWVRRRPGHDLEWIGYINPSSGCSDYNQNFKGKTTLTADKSSNTAYMQLNSLTSEDSAVYYCARRADYGNYEYTWFAYWGQGTTVTVSSSGGGSGGGGSGGGGSVIELTQSPKFMSTSVGDRVNVTYKASQNVGTNVAWFQQKPGQSPKVLIYSASYRYSGVPDRFTGSGSGTDFTLTISNVQSEDLAEYFCQQYHTYPLTFGGGTKLEIKRSDPAEPKSPDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKKDPKFWVLVVVGGVLACYSLLVTVAFIIFWVRSKRSRLLHSDYMNMTPRRPGPTRKHYQPYAPPRDFAAYRSRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 47 - protein, 376 aa, synthetic construct
METLSNASGTFAIRLLKILCQDNPSHNVFCSPVSISSALAMVLLGAKGNTATQMAQALSLNTEEDIHRAFQSLLTEVNKAGTQYLLRTANRLFGEKTCQFLSTFKESCLQFYHAELKELSFIRAAEESRKHINTWVSKKTEGKIEELLPGSSIDAETRLVLVNAIYFKGKWNEPFDETYTREMPFKINQEEQRPVQMMYQEATFKLAHVGEVRAQLLELPYARKELSLLVLLPDDGVELSTVEKSLTFEKLTAWTKPDCMKSTEVEVLLPKFKLQEDYDMESVLRHLGIVDAFQQGKADLSAMSAERDLCLSKFVHKSFVEVNEEGREAAAASSCFVVAACCMESGPRFCADHPFLFFIRHNRANSILFCGRFSSP

SEQ ID NO: 48 - protein, 1090 aa, synthetic construct
METLSNASGTFAIRLLKILCQDNPSHNVFCSPVSISSALAMVLLGAKGNTATQMAQALSLNTEEDIHRAFQSLLTEVNKAGTQYLLRTANRLFGEKTCQFLSTFKESCLQFYHAELKELSFIRAAEESRKHINTWVSKKTEGKIEELLPGSSIDAETRLVLVNAIYFKGKWNEPFDETYTREMPFKINQEEQRPVQMMYQEATFKLAHVGEVRAQLLELPYARKELSLLVLLPDDGVELSTVEKSLTFEKLTAWTKPDCMKSTEVEVLLPKFKLQEDYDMESVLRHLGIVDAFQQGKADLSAMSAERDLCLSKFVHKSFVEVNEEGTEAAAASSCFVVADCCMESGPRFCADHPFLFFIRHNRANSILFCGRFSSPTRGSGEGRGSLLTCGDVEENPGPLEMDFQVQIFSFLLISASVIMSRMAQVQLQQSGAELARPGASVKMSCKASGYTFTTYTIHWVRRRPGHDLEWIGYINPSSGCSDYNQNFKGKTTLTADKSSNTAYMQLNSLTSEDSAVYYCARRADYGNYEYTWFAYWGQGTTVTVSSSGGGSGGGGSGGGGSVIELTQSPKFMSTSVGDRVNVTYKASQNVGTNVAWFQQKPGQSPKVLIYSASYRYSGVPDRFTGSGSGTDFTLTISNVQSEDLAEYFCQQYHTYPLTFGGGTKLEIKRSDPAEPKSPDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKKDPKFWVLVVVGGVLACYSLLVTVAFIIFWVRSKRSRLLHSDYMNMTPRRPGPTRKHYQPYAPPRDFAAYRSRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 49 - protein, 25 aa, synthetic construct
SPADLSPGASSVTPPAPAREPGHSP

SEQ ID NO: 50 - protein, 482 aa, synthetic construct
MDFQVQIFSFLLISASVIMSRMAQVQLQQSGAELARPGASVKMSCKASGYTFTTYTIHWVRRRPGHDLEWIGYINPSSGCSDYNQNFKGKTTLTADKSSNTAYMQLNSLTSEDSAVYYCARRADYGNYEYTWFAYWGQGTTVTVSSSGGGSGGGGSGGGGSVIELTQSPKFMSTSVGDRVNVTYKASQNVGTNVAWFQQKPGQSPKVLIYSASYRYSGVPDRFTGSGSGTDFTLTISNVQSEDLAEYFCQQYHTYPLTFGGGTKLEIKRSDPASPADLSPGASSVTPPAPAREPGHSPKDPKFWVLVVVGGVLACYSLLVTVAFIIFWVRSKRSRLLHSDYMNMTPRRPGPTRKHYQPYAPPRDFAAYRSRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 51 - protein, 459 aa, synthetic construct
QVQLQQSGAELARPGASVKMSCKASGYTFTTYTIHWVRRRPGHDLEWIGYINPSSGCSDYNQNFKGKTTLTADKSSNTAYMQLNSLTSEDSAVYYCARRADYGNYEYTWFAYWGQGTTVTVSSSGGGSGGGGSGGGGSVIELTQSPKFMSTSVGDRVNVTYKASQNVGTNVAWFQQKPGQSPKVLIYSASYRYSGVPDRFTGSGSGTDFTLTISNVQSEDLAEYFCQQYHTYPLTFGGGTKLEIKRSDPASPADLSPGASSVTPPAPAREPGHSPKDPKFWVLVVVGGVLACYSLLVTVAFIIFWVRSKRSRLLHSDYMNMTPRRPGPTRKHYQPYAPPRDFAAYRSRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 52 - protein, 556 aa, Homo sapiens [sequence omitted]

SEQ ID NO: 53 - protein, 557 aa, Homo sapiens
MPPPRLLFFLLFLTPMEVRPEEPLVVKVEEGDNAVLQCLKGTSDGPTQQLTWSRESPLKPFLKLSLGLPGLGIHMRPLAIWLFIFNVSQQMGGFYLCQPGPPSEKAWQPGWTVNVEGSGELFRWNVSDLGGLGCGLKNRSSEGPSSPSGKLMSPKLYVWAKDRPEIWEGEPPCLPPRDSLNQSLSQDLTMAPGSTLWLSCGVPPDSVSRGPLSWTHVHPKGPKSLLSLELKDDRPARDMWVMETGLLLPRATAQDAGKYYCHRGNLTMSFHLEITARPVLWHWLLRTGGWKVSAVTLAYLIFCLCSLVGILHLQRALVLRRKRKRMTDPTRRFFKVTPPPGSGPQNQYGNVLSLPTPTSGLGRAQRWAAGLGGTAPSYGNPSSDVQADGALGSRSPPGVGPEEEEGEGYEEPDSEEDSEFYENDSNLGQDQLSQDGSGYENPEDEPLGPEDEDSFSNAESYENEDEELTQPVARTMDFLSPHGSAWDPSREATSLAGSQSYEDMRGILYAAPQLRSIRGQPGPNHEEDADSYENMDNPDGPDPAWGGGGRMGTWSTR

SEQ ID NO: 54 - protein, 19 aa, Homo sapiens
MPPPRLLFFLLFLTPMEVR

SEQ ID NO: 55 - protein, 272 aa, Homo sapiens
PEEPLVVKVEEGDNAVLQCLKGTSDGPTQQLTWSRESPLKPFLKLSLGLPGLGIHMRPLAIWLFIFNVSQQMGGFYLCQPGPPSEKAWQPGWTVNVEGSGELFRWNVSDLGGLGCGLKNRSSEGPSSPSGKLMSPKLYVWAKDRPEIWEGEPPCLPPRDSLNQSLSQDLTMAPGSTLWLSCGVPPDSVSRGPLSWTHVHPKGPKSLLSLELKDDRPARDMWVMETGLLLPRATAQDAGKYYCHRGNLTMSFHLEITARPVLWHWLLRTGGWK

SEQ ID NO: 56 - protein, 22 aa, Homo sapiens
VSAVTLAYLIFCLCSLVGILHL

SEQ ID NO: 57 - protein, 243 aa, Homo sapiens
QRALVLRRKRKRMTDPTRRFFKVTPPPGSGPQNQYGNVLSLPTPTSGLGRAQRWAAGLGGTAPSYGNPSSDVQADGALGSRSPPGVGPEEEEGEGYEEPDSEEDSEFYENDSNLGQDQLSQDGSGYENPEDEPLGPEDEDSFSNAESYENEDEELTQPVARTMDFLSPHGSAWDPSREATSLGSQSYEDMRGILYAAPQLRSIRGQPGPNHEEDADSYENMDNPDGPDPAWGGGGRMGTWSTR

SEQ ID NO: 58 - protein, 8 aa, synthetic construct
GVSLPDYG

SEQ ID NO: 59 - protein, 7 aa, synthetic construct
IWGSETT

SEQ ID NO: 60 - protein, 14 aa, synthetic construct
AKHYYYGGSYAMDY

SEQ ID NO: 61 - protein, 6 aa, synthetic construct
QDISKY

SEQ ID NO: 62 - skipped

SEQ ID NO: 63 - protein, 9 aa, synthetic construct
QQGNTLPYT

SEQ ID NO: 64 - protein, 120 aa, synthetic construct
EVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSS

SEQ ID NO: 65 - protein, 107 aa, synthetic construct
DIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEIT

SEQ ID NO: 66 - protein, 18 aa, synthetic construct
GSTSGSGKPGSGEGSTKG

SEQ ID NO: 67 - protein, 245 aa, synthetic construct
DIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEITGSTSGSGKPGSGEGSTKGEVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSS

SEQ ID NO: 68 - protein, 39 aa, synthetic construct
IEVMYPPPYLDNEKSNGTIIHVKGKHLCPSPLFPGPSKP

SEQ ID NO: 69 - protein, 489 aa, synthetic construct
MLLLVTSLLLCELPHPAFLLIPDIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEITGSTSGSGKPGSGEGSTKGEVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSSAAAIEVMYPPPYLDNEKSNGTIIHVKGKHLCPSPLFPGPSKPFWVLVVVGGVLACYSLLVTVAFIIFWVRSKRSRLLHSDYMNMTPRRPGPTRKHYQPYAPPRDFAAYRSRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 70 - protein, 467 aa, synthetic construct
DIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEITGSTSGSGKPGSGEGSTKGEVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSSAAAIEVMYPPPYLDNEKSNGTIIHVKGKHLCPSPLFPGPSKPFWVLVVVGGVLACYSLLVTVAFIIFWVRSKRSRLLHSDYMNMTPRRPGPTRKHYQPYAPPRDFAAYRSRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 71 - protein, 4 aa, synthetic construct
NPGP

SEQ ID NO: 72 - protein, 4 aa, synthetic construct
GGGS

SEQ ID NO: 73 - DNA, 20 bp, synthetic construct
  source 1..20: mol_type = other DNA
cgtgagtaaacctgaatctt

SEQ ID NO: 74 - DNA, 20 bp, synthetic construct
  source 1..20: mol_type = other DNA
aagtcaacttcaatgtcgga

SEQ ID NO: 75 - DNA, 20 bp, synthetic construct
  source 1..20: mol_type = other DNA
agtcgctcactggtcccact

SEQ ID NO: 76 - DNA, 20 bp, synthetic construct
  source 1..20: mol_type = other DNA
ccgtggacagtgaatccact

SEQ ID NO: 77 - DNA, 20 bp, synthetic construct
  source 1..20: mol_type = other DNA
ctctcaccgatcacttcatc

SEQ ID NO: 78 - DNA, 20 bp, synthetic construct
  source 1..20: mol_type = other DNA
agagtctctcagctggtaca

SEQ ID NO: 79 - DNA, 20 bp, synthetic construct
  source 1..20: mol_type = other DNA
gcagtatctggagtcattga

SEQ ID NO: 80 - DNA, 20 bp, synthetic construct
  source 1..20: mol_type = other DNA
aggtctggaccgggtagcac

SEQ ID NO: 81 - DNA, 20 bp, synthetic construct
  source 1..20: mol_type = other DNA
gctgtgtcgggaacagccct

SEQ ID NO: 82 - DNA, 20 bp, synthetic construct
  source 1..20: mol_type = other DNA
tcgacattcgcagacacggg